(19) United States
(12) Patent Application Publication
(10) Pub. No.: US 2024/0344063 A1
(43) Pub. Date: Oct. 17, 2024

(54) COMPOSITIONS AND METHODS FOR INHIBITING LPA EXPRESSION

(71) Applicant: Dicerna Pharmaceuticals, Inc., Lexington, MA (US)

(72) Inventors: Bob Dale BROWN, Littleton, MA (US); Henryk T. DUDEK, Belmont, MA (US); Marc ABRAMS, Natick, MA (US); Wen HAN, Boston, MA (US); Anton TURANOV, Revere, MA (US)

(21) Appl. No.: 18/040,302

(22) PCT Filed: Aug. 5, 2021
(86) PCT No.: PCT/US2021/071109

§ 371 Date: Oct. 2, 2023

Related U.S. Application Data
(60) Provisional application No. 63/074,779, filed on Sep. 4, 2020.
(60) Provisional application No. 63/061,676, filed on Aug. 5, 2020.

(51) Int. Cl. C12N 15/113

(52) U.S. Cl. CPC C12N 15/113; C12N 2310/14; C12N 2310/315; C12N 2310/321; C12N 2310/322; C12N 2310/351; C12N 2310/531

(57) ABSTRACT

Oligonucleotides are provided herein that inhibit apolipoprotein(a) (LPA) expression. Also provided are compositions including the same and uses thereof, particularly uses relating to treating diseases, disorders and/or conditions associated with LPA expression.

### TECHNICAL FIELD

The disclosure relates to oligonucleotides that inhibit apolipoprotein(a) (“LPA”) expression and uses thereof, particularly uses relating to treating diseases, disorders and/or conditions associated with LPA expression.

### REFERENCE TO SEQUENCE LISTING

A Sequence Listing is submitted concurrently with the specification as an ASCII formatted text file, with a file name of DRNA_C002WO_ST25.txt, a creation date of Aug. 5, 2021, and a size of 238 kilobytes. The information in the electronic format of the Sequence Listing is part of the specification and is hereby incorporated herein by reference in its entirety.

### BACKGROUND

Lipoprotein(a) (Lp(a)) is a heterogeneous low density lipoprotein (LDL)-like particle containing a lipid core and apolipoprotein B (apoB-100) with a unique constituent, apolipoprotein (a) (apo(a)), that is attached to apoB-100 through a disulfide bond. The apo(a) gene (LPA) is expressed predominantly in the liver and expression is restricted to human and non-human primates. Lp(a) levels in humans are genetically defined and do not change significantly with diet, exercise, or other lifestyle changes. LPA varies in length depending upon the number of Kringle KIV2 domains present and its expression is inversely correlated with the number of domains present. Normal Lp(a) levels range from 0.1-25 mg/dl, with about 25% of the population in the United States of America having Lp(a) levels of 30 mg/dl or higher. Analysis of Lp(a) levels in multiple studies have implicated high Lp(a) levels as an independent risk factor for cardiovascular disease, stroke, and other related disorders including atherosclerotic stenosis. In addition, genome-wide association analyses have also implicated LPA as a genetic risk factor for diseases such as atherosclerotic stenosis. When therapeutic lipoprotein apheresis is used to lower both Lp(a) and LDL levels in hyperlipidemic patients, significant reductions of cardiovascular events have been observed.

Therefore, there exists a need for therapeutics and treatments related to these and other LPA-related diseases.

### BRIEF SUMMARY OF THE INVENTION

Embodiments of the disclosure relate to compositions and methods for treating a disease, disorder and/or condition related to LPA expression. The disclosure is based, in part, on the discovery and development of oligonucleotides that selectively inhibit and/or reduce LPA expression in the liver. Accordingly, target sequences within LPA mRNA were identified and RNAi oligonucleotides that bind to these target sequences and inhibit LPA mRNA expression were generated. As demonstrated herein, the RNAi oligonucleotides inhibited monkey and human LPA expression in the liver. Without being bound by theory, the RNAi oligonucleotides described herein are useful for treating a disease, disorder or condition associated with LPA expression (e.g., cardiometabolic diseases, atherosclerosis, dyslipidemia, NAFLD and NASH).

Accordingly, in some embodiments, the present disclosure provides an RNAi oligonucleotide for reducing LPA expression, the oligonucleotide comprising a sense strand and an antisense strand, wherein the sense strand and the antisense strand form a duplex region, wherein the antisense strand comprises a region of complementarity to a LPA mRNA target sequence of any one of SEQ ID NOs: 4-387, and wherein the region of complementarity is at least 15 contiguous nucleotides in length.

In any of the foregoing or related embodiments, the sense strand is 15 to 50 nucleotides in length. In some embodiments, the sense strand is 18 to 36 nucleotides in length.

In any of the foregoing or related aspects, the antisense strand is 15 to 30 nucleotides in length.

In any of the foregoing or related aspects, the antisense strand is 22 nucleotides in length and wherein antisense strand and the sense strand form a duplex region of at least 19 nucleotides in length, optionally at least 20 nucleotides in length.

In any of the foregoing or related aspects, the region of complementarity is at least 19 contiguous nucleotides in length, optionally at least 20 nucleotides in length.

In any of the foregoing or related aspects, the 3′ end of the sense strand comprises a stem-loop set forth as S1-L-S2, wherein S1 is complementary to S2, and wherein L forms a loop between S1 and S2 of 3-5 nucleotides in length.

In some aspects, the disclosure provides an RNAi oligonucleotide for reducing LPA expression, the oligonucleotide comprising a sense strand of 15 to 50 nucleotides in length and an antisense strand, wherein the sense strand and the antisense strand form a duplex region, wherein the wherein the antisense strand comprises a region of complementarity to a LPA mRNA target sequence of any one of SEQ ID NOs: 4-387, and wherein the region of complementarity is at least 15 contiguous nucleotides in length.

In other aspects, the disclosure provides an RNAi oligonucleotide for reducing LPA expression, the oligonucleotide comprising a sense strand of 15 to 50 nucleotides in length and an antisense strand of 15 to 30 nucleotides in length, wherein the sense strand and the antisense strand form a duplex region, wherein the antisense strand comprises a region of complementarity to a LPA mRNA target sequence of any one of SEQ ID NOs: 4-387, and wherein the region of complementarity is at least 15 contiguous nucleotides in length.

In yet other aspects, the disclosure provides an RNAi oligonucleotide for reducing LPA expression, the oligonucleotide comprising a sense strand of 15 to 50 nucleotides in length and an antisense strand, wherein the sense strand and the antisense strand form a duplex region, wherein the antisense strand comprises a region of complementarity to a LPA mRNA target sequence of any one of SEQ ID NOs: 4-387, and wherein the region of complementarity is 19 contiguous nucleotides in length, optionally 20 nucleotides in length.

In further aspects, the disclosure provides an RNAi oligonucleotide for reducing LPA expression, the oligonucleotide comprising a sense strand of 18 to 36 nucleotides in length and an antisense strand, wherein the sense strand and the antisense strand form a duplex region, wherein the antisense strand comprises a region of complementarity to a LPA mRNA target sequence of any one of SEQ ID NOs: 4-387, and wherein the region of complementarity is 19 contiguous nucleotides in length, optionally 20 nucleotides in length.

In other aspects, the disclosure provides an RNAi oligonucleotide for reducing LPA expression, the oligonucleotide comprising a sense strand of 18 to 36 nucleotides in length and an antisense strand of 22 nucleotides in length, wherein the sense strand and the antisense strand form a duplex region, wherein the antisense strand comprises a region of complementarity to a LPA mRNA target sequence of any one of SEQ ID NOs: 4-387, and wherein the region of complementarity is 19 contiguous nucleotides in length, optionally 20 nucleotides in length.

In some aspects, the disclosure provides an RNAi oligonucleotide for reducing LPA expression, the oligonucleotide comprising a sense strand of 18 to 36 nucleotides in length and an antisense strand of 22 nucleotides in length, wherein the sense strand and the antisense strand form a duplex region, wherein the 3′ end of the sense strand comprises a stem-loop set forth as S1-L-S2, wherein S1 is complementary to S2, and wherein L forms a loop between S1 and S2 of 3-5 nucleotides in length, wherein the antisense strand comprises a region of complementarity to a LPA mRNA target sequence of any one of SEQ ID NOs: 4-387, and wherein the region of complementarity is 19 contiguous nucleotides in length, optionally 20 nucleotides in length.

In other aspects, the disclosure provides an RNAi oligonucleotide for reducing LPA expression, the oligonucleotide comprising a sense strand of 36 nucleotides in length and an antisense strand of 22 nucleotides in length, wherein the sense strand and the antisense strand form a duplex region, wherein the 3′ end of the sense strand comprises a stem-loop set forth as S1-L-S2, wherein S1 is complementary to S2, and wherein L forms a loop between S1 and S2 of 3-5 nucleotides in length, wherein the antisense strand comprises a region of complementarity to a LPA mRNA target sequence of any one of SEQ ID NOs: 4-387, and wherein the region of complementarity is 19 contiguous nucleotides in length, optionally 20 nucleotides in length.

In yet other aspects, the disclosure provides an RNAi oligonucleotide for reducing LPA expression, the oligonucleotide comprising a sense strand of 36 nucleotides in length and an antisense strand of 22 nucleotides in length, wherein the sense strand and the antisense strand form a duplex region of at least 19 nucleotides in length, optionally 20 nucleotides in length, wherein the 3′ end of the sense strand comprises a stem-loop set forth as S1-L-S2, wherein S1 is complementary to S2, and wherein L forms a loop between S1 and S2 of 3-5 nucleotides in length, wherein the antisense strand comprises a region of complementarity to a LPA mRNA target sequence of any one of SEQ ID NOs: 4-387, and wherein the region of complementarity is 19 contiguous nucleotides in length, optionally 20 nucleotides in length.

In any of the foregoing or related aspects, L is a triloop or a tetraloop. In some embodiments, L is a tetraloop. In some embodiments, the tetraloop comprises the sequence 5′-GAAA-3′.

In any of the foregoing or related embodiments, the S1 and S2 are 1-10 nucleotides in length and have the same length. In some embodiments, S1 and S2 are 1 nucleotide, 2 nucleotides, 3 nucleotides, 4 nucleotides, 5 nucleotides, 6 nucleotides, 7 nucleotides, 8 nucleotides, 9 nucleotides, or 10 nucleotides in length. In some embodiments, S1 and S2 are 6 nucleotides in length. In some embodiments, the stem-loop comprises the sequence 5′-GCAGCCGAAAGGCUGC-3′ (SEQ ID NO: 1197).

In any of the foregoing or related embodiments, the antisense strand comprises a 3′ overhang sequence of one or more nucleotides in length. In some embodiments, the 3′ overhang sequence is 2 nucleotides in length, optionally wherein the 3′ overhang sequence is GG.

In any of the foregoing or related embodiments, the oligonucleotide comprises at least one modified nucleotide. In some embodiments, the modified nucleotide comprises a 2′-modification. In some embodiments, the 2′-modification is a modification selected from 2′-aminoethyl, 2′-fluoro, 2′-O-methyl, 2′-O-methoxyethyl, and 2′-deoxy-2′-fluoro-β-d-arabinonucleic acid. In some embodiments, all nucleotides comprising the oligonucleotide are modified, optionally wherein the modification is a 2′-modification selected from 2′-fluoro and 2′-O-methyl.

In any of the foregoing or related embodiments, the oligonucleotide comprises at least one modified internucleotide linkage. In some embodiments, the at least one modified internucleotide linkage is a phosphorothioate linkage.

In any of the foregoing or related embodiments, the 4′-carbon of the sugar of the 5′-nucleotide of the antisense strand comprises a phosphate analog. In some embodiments, the phosphate analog is oxymethylphosphonate, vinylphosphonate or malonyl phosphonate, optionally wherein the phosphate analog is a 4′-phosphate analog comprising 5′-methoxyphosphonate-4′-oxy.

In any of the foregoing or related embodiments, at least one nucleotide of the oligonucleotide is conjugated to one or more targeting ligands. In some embodiments, each targeting ligand comprises a carbohydrate, amino sugar, cholesterol, polypeptide, or lipid. In some embodiments, each targeting ligand comprises a N-acetylgalactosamine (GalNAc) moiety. In some embodiments, the GalNAc moiety is a monovalent GalNAc moiety, a bivalent GalNAc moiety, a trivalent GalNAc moiety or a tetravalent GalNAc moiety. In some embodiments, up to 4 nucleotides of L of the stem-loop are each conjugated to a monovalent GalNAc moiety.

In any of the foregoing or related embodiments, the sense strand comprises a nucleotide sequence of any one of SEQ ID NOs: 388, 389, 390, 391, 392, 393, 394, 395, 396, 397, 398, 399, 400, 401, 402, and 403.

In any of the foregoing or related embodiments, the antisense strand comprises a nucleotide sequence of any one of SEQ ID NOs: 788, 789, 790, 791, 792, 793, 794, 795, 796, 797, 798, 799, 800, 801, 802, and 803.

In any of the foregoing or related embodiments, the sense strand and antisense strands comprise nucleotide sequences selected from the group consisting of:
(a) SEQ ID NOs: 393 and 793, respectively;
(b) SEQ ID NOs: 388 and 788, respectively;
(c) SEQ ID NOs: 389 and 789, respectively;
(d) SEQ ID NOs: 390 and 790, respectively;
(e) SEQ ID NOs: 391 and 791, respectively;
(f) SEQ ID NOs: 392 and 792, respectively;
(g) SEQ ID NOs: 394 and 794, respectively;
(h) SEQ ID NOs: 395 and 795, respectively;
(i) SEQ ID NOs: 396 and 796, respectively;
(j) SEQ ID NOs: 397 and 797, respectively;
(k) SEQ ID NOs: 398 and 798, respectively;
(l) SEQ ID NOs: 399 and 799, respectively;
(m) SEQ ID NOs: 400 and 800, respectively;
(n) SEQ ID NOs: 401 and 801, respectively;
(o) SEQ ID NOs: 402 and 802, respectively; and
(p) SEQ ID NOs: 403 and 803, respectively.

In some embodiments, the sense strand comprises a nucleotide sequence as set forth in SEQ ID NO: 393, wherein the antisense strand comprises a nucleotide sequence as set forth in SEQ ID NO: 793. In some embodiments, the sense strand comprises a nucleotide sequence as set forth in SEQ ID NO: 388, wherein the antisense strand comprises a nucleotide sequence as set forth in SEQ ID NO: 788. In some embodiments, the sense strand comprises a nucleotide sequence as set forth in SEQ ID NO: 389, wherein the antisense strand comprises a nucleotide sequence as set forth in SEQ ID NO: 789. In some embodiments, the sense strand comprises a nucleotide sequence as set forth in SEQ ID NO: 390, wherein the antisense strand comprises a nucleotide sequence as set forth in SEQ ID NO: 790. In some embodiments, the sense strand comprises a nucleotide sequence as set forth in SEQ ID NO: 391, wherein the antisense strand comprises a nucleotide sequence as set forth in SEQ ID NO: 791. In some embodiments, the sense strand comprises a nucleotide sequence as set forth in SEQ ID NO: 392, wherein the antisense strand comprises a nucleotide sequence as set forth in SEQ ID NO: 792. In some embodiments, the sense strand comprises a nucleotide sequence as set forth in SEQ ID NO: 394, wherein the antisense strand comprises a nucleotide sequence as set forth in SEQ ID NO: 794. In some embodiments, the sense strand comprises a nucleotide sequence as set forth in SEQ ID NO: 395, wherein the antisense strand comprises a nucleotide sequence as set forth in SEQ ID NO: 795. In some embodiments, the sense strand comprises a nucleotide sequence as set forth in SEQ ID NO: 396, wherein the antisense strand comprises a nucleotide sequence as set forth in SEQ ID NO: 796. In some embodiments, the sense strand comprises a nucleotide sequence as set forth in SEQ ID NO: 397, wherein the antisense strand comprises a nucleotide sequence as set forth in SEQ ID NO: 797. In some embodiments, the sense strand comprises a nucleotide sequence as set forth in SEQ ID NO: 398, wherein the antisense strand comprises a nucleotide sequence as set forth in SEQ ID NO: 798. In some embodiments, the sense strand comprises a nucleotide sequence as set forth in SEQ ID NO: 399, wherein the antisense strand comprises a nucleotide sequence as set forth in SEQ ID NO: 799. In some embodiments, the sense strand comprises a nucleotide sequence as set forth in SEQ ID NO: 400, wherein the antisense strand comprises a nucleotide sequence as set forth in SEQ ID NO: 800. In some embodiments, the sense strand comprises a nucleotide sequence as set forth in SEQ ID NO: 401, wherein the antisense strand comprises a nucleotide sequence as set forth in SEQ ID NO: 801. In some embodiments, the sense strand comprises a nucleotide sequence as set forth in SEQ ID NO: 402, wherein the antisense strand comprises a nucleotide sequence as set forth in SEQ ID NO: 802. In some embodiments, the sense strand comprises a nucleotide sequence as set forth in SEQ ID NO: 403, wherein the antisense strand comprises a nucleotide sequence as set forth in SEQ ID NO: 803.

In some embodiments, the disclosure provides an RNAi oligonucleotide for reducing LPA expression, the oligonucleotide comprising a sense strand and an antisense strand, wherein the sense strand and the antisense strand form a duplex region, wherein all nucleotides comprising the sense strand and antisense strand are modified, wherein the antisense strand comprises a region of complementarity to a LPA mRNA target sequence of any one of SEQ ID NOs: 4-387, and wherein the region of complementarity is at least 15 contiguous nucleotides in length.

In further embodiments, the disclosure provides an RNAi oligonucleotide for reducing LPA expression, the oligonucleotide comprising a sense strand and an antisense strand, wherein the sense strand and the antisense strand form a duplex region, wherein all nucleotides comprising the sense strand and antisense strand are modified, wherein the 4′-carbon of the sugar of the 5′-nucleotide of the antisense strand comprises a phosphate analog, wherein the antisense strand comprises a region of complementarity to a LPA mRNA target sequence of any one of SEQ ID NOs: 4-387, and wherein the region of complementarity is at least 15 contiguous nucleotides in length.

In other embodiments, the disclosure provides an RNAi oligonucleotide for reducing LPA expression, the oligonucleotide comprising a sense strand and an antisense strand, wherein the sense strand and the antisense strand form a duplex region, wherein all nucleotides comprising the sense strand and antisense strand are modified, wherein the 4′-carbon of the sugar of the 5′-nucleotide of the antisense strand comprises a phosphate analog, wherein the antisense strand comprises a region of complementarity to a LPA mRNA target sequence of any one of SEQ ID NOs: 4-387, and wherein the region of complementarity is at least 15 contiguous nucleotides in length.

In some embodiments, the disclosure provides an RNAi oligonucleotide for reducing LPA expression, the oligonucleotide comprising a sense strand and an antisense strand, wherein the sense strand and the antisense strand form a duplex region, wherein all nucleotides comprising the sense strand and the antisense strand are modified, wherein the antisense strand and the sense strand comprise one or more 2′-fluoro and 2′-O-methyl modified nucleotides and at least one phosphorothioate linkage, wherein the 4′-carbon of the sugar of the 5′-nucleotide of the antisense strand comprises a phosphate analog, wherein the antisense strand comprises a region of complementarity to a LPA mRNA target sequence of any one of SEQ ID NOs: 4-387, and wherein the region of complementarity is at least 15 contiguous nucleotides in length.

In some embodiments, the disclosure provides a method for treating a subject having a disease, disorder or condition associated with LPA expression, the method comprising administering to the subject a therapeutically effective amount of the RNAi oligonucleotide of any one of the preceding claims, or pharmaceutical composition thereof, thereby treating the subject.

In other embodiments, the disclosure provides a pharmaceutical composition comprising a RNAi oligonucleotide described herein, and a pharmaceutically acceptable carrier, delivery agent or excipient.

In other embodiments, the disclosure provides a method of delivering an oligonucleotide to a subject, the method comprising administering a pharmaceutical composition described herein to the subject.

In another embodiments, the disclosure provides a method for reducing LPA expression in a cell, a population of cells or a subject, the method comprising the step of:
i. contacting the cell or the population of cells with a RNAi oligonucleotide or pharmaceutical composition described herein; or
ii. administering to the subject a RNAi oligonucleotide or pharmaceutical composition described herein. In some embodiments, reducing LPA expression comprises reducing an amount or level of LPA mRNA, an amount or level of LPA protein, or both. In some embodiments, the subject has a disease, disorder or condition associated with LPA expression. In some embodiments, the disease, disorder, or condition associated with LPA expression is a cardiometabolic disease, optionally atherosclerosis, dyslipidemia, NAFLD and NASH. In some embodiments, the RNAi oligonucleotide, or pharmaceutical composition, is administered in combination with a second composition or therapeutic agent.

In another aspect, the disclosure provides a method for treating a subject having a disease, disorder or condition associated with LPA expression, the method comprising administering to the subject a therapeutically effective amount of an RNAi oligonucleotide comprising a sense strand and an antisense strand, wherein the sense strand and the antisense strand form a duplex region, wherein the antisense strand comprises a region of complementarity to a LPA mRNA target sequence of any one of SEQ ID NOs: 4-387, and wherein the region of complementarity is at least 15 contiguous nucleotides in length.

In another aspect, the disclosure provides a method for treating a subject having a disease, disorder or condition associated with LPA expression, the method comprising administering to the subject a therapeutically effective amount of an RNAi oligonucleotide comprising a sense strand and an antisense strand selected from a row set forth in Table 5, or pharmaceutical composition thereof, thereby treating the subject.

In other embodiments, the disclosure provides a method for treating a subject having a disease, disorder or condition associated with LPA expression, the method comprising administering to the subject a therapeutically effective amount of an RNAi oligonucleotide comprising a sense strand and an antisense strand, wherein the sense strand and antisense strands comprise nucleotide sequences selected from the group consisting of:
(a) SEQ ID NOs: 393 and 793, respectively;
(b) SEQ ID NOs: 388 and 788, respectively;
(c) SEQ ID NOs: 389 and 789, respectively;
(d) SEQ ID NOs: 390 and 790, respectively;
(e) SEQ ID NOs: 391 and 791, respectively;
(f) SEQ ID NOs: 392 and 792, respectively;
(g) SEQ ID NOs: 394 and 794, respectively;
(h) SEQ ID NOs: 395 and 795, respectively;
(i) SEQ ID NOs: 396 and 796, respectively;
(j) SEQ ID NOs: 397 and 797, respectively;
(k) SEQ ID NOs: 398 and 798, respectively;
(l) SEQ ID NOs: 399 and 799, respectively;
(m) SEQ ID NOs: 400 and 800, respectively;
(n) SEQ ID NOs: 401 and 801, respectively;
(o) SEQ ID NOs: 402 and 802, respectively; and
(p) SEQ ID NOs: 403 and 803, respectively.

In some embodiments, the sense strand comprises a nucleotide sequence as set forth in SEQ ID NO: 393, wherein the antisense strand comprises a nucleotide sequence as set forth in SEQ ID NO: 793. In some embodiments, the sense strand comprises a nucleotide sequence as set forth in SEQ ID NO: 388, wherein the antisense strand comprises a nucleotide sequence as set forth in SEQ ID NO: 788. In some embodiments, the sense strand comprises a nucleotide sequence as set forth in SEQ ID NO: 389, wherein the antisense strand comprises a nucleotide sequence as set forth in SEQ ID NO: 789. In some embodiments, the sense strand comprises a nucleotide sequence as set forth in SEQ ID NO: 390, wherein the antisense strand comprises a nucleotide sequence as set forth in SEQ ID NO: 790. In some embodiments, the sense strand comprises a nucleotide sequence as set forth in SEQ ID NO: 391, wherein the antisense strand comprises a nucleotide sequence as set forth in SEQ ID NO: 791. In some embodiments, the sense strand comprises a nucleotide sequence as set forth in SEQ ID NO: 392, wherein the antisense strand comprises a nucleotide sequence as set forth in SEQ ID NO: 792. In some embodiments, the sense strand comprises a nucleotide sequence as set forth in SEQ ID NO: 394, wherein the antisense strand comprises a nucleotide sequence as set forth in SEQ ID NO: 794. In some embodiments, the sense strand comprises a nucleotide sequence as set forth in SEQ ID NO: 395, wherein the antisense strand comprises a nucleotide sequence as set forth in SEQ ID NO: 795. In some embodiments, the sense strand comprises a nucleotide sequence as set forth in SEQ ID NO: 396, wherein the antisense strand comprises a nucleotide sequence as set forth in SEQ ID NO: 796. In some embodiments, the sense strand comprises a nucleotide sequence as set forth in SEQ ID NO: 397, wherein the antisense strand comprises a nucleotide sequence as set forth in SEQ ID NO: 797. In some embodiments, the sense strand comprises a nucleotide sequence as set forth in SEQ ID NO: 398, wherein the antisense strand comprises a nucleotide sequence as set forth in SEQ ID NO: 798. In some embodiments, the sense strand comprises a nucleotide sequence as set forth in SEQ ID NO: 399, wherein the antisense strand comprises a nucleotide sequence as set forth in SEQ ID NO: 799. In some embodiments, the sense strand comprises a nucleotide sequence as set forth in SEQ ID NO: 400, wherein the antisense strand comprises a nucleotide sequence as set forth in SEQ ID NO: 800. In some embodiments, the sense strand comprises a nucleotide sequence as set forth in SEQ ID NO: 401, wherein the antisense strand comprises a nucleotide sequence as set forth in SEQ ID NO: 801. In some embodiments, the sense strand comprises a nucleotide sequence as set forth in SEQ ID NO: 402, wherein the antisense strand comprises a nucleotide sequence as set forth in SEQ ID NO: 802. In some embodiments, the sense strand comprises a nucleotide sequence as set forth in SEQ ID NO: 403, wherein the antisense strand comprises a nucleotide sequence as set forth in SEQ ID NO: 803.

In some embodiments, the disease, disorder, or condition associated with LPA expression is a cardiometabolic disease, optionally atherosclerosis, dyslipidemia, NAFLD and NASH.

In some embodiments, the disclosure provides use of an RNAi oligonucleotide or pharmaceutical composition described herein, in the manufacture of a medicament for the treatment of a disease, disorder or condition associated with LPA expression, optionally for the treatment of a cardiometabolic disease, optionally atherosclerosis, dyslipidemia, NAFLD and NASH.

In some embodiments, the disclosure provides use of an RNAi oligonucleotide or pharmaceutical composition described herein, for use, or adaptable for use, in the treatment of a disease, disorder or condition associated with LPA expression, optionally for the treatment of a cardiometabolic disease, optionally atherosclerosis, dyslipidemia, NAFLD and NASH.

In other embodiments, the disclosure provides a kit comprising an RNAi oligonucleotide described herein, an optional pharmaceutically acceptable carrier, and a package insert comprising instructions for administration to a subject having a disease, disorder or condition associated with LPA expression.

In any of the foregoing or related embodiments, the disease, disorder, or condition associated with LPA expression is a cardiometabolic disease, optionally atherosclerosis, dyslipidemia, NAFLD and NASH.

### BRIEF DESCRIPTION OF THE DRAWINGS

FIGS. 1-4 provide graphs depicting the percent (%) of LPA mRNA in HEK293-LPA cells transfected with the indicated DsiRNAs relative to the % of LPA mRNA control mock-treated cells.

FIG. 5 provides a graph depicting the percent (%) of LPA mRNA in HepG2-LPA cells transfected with the indicated DsiRNAs relative to the % of LPA mRNA control mock-treated cells.

FIGS. 6-7 provide graphs depicting the percent (%) of LPA mRNA in HEK293-LPA cells transfected with the indicated DsiRNAs relative to the % of LPA mRNA control mock-treated cells.

FIGS. 8-9 provide graphs depicting the percent (%) of LPA mRNA in liver samples from mice treated with the indicated GalNAc-conjugated LPA oligonucleotides relative to mice treated with phosphate buffered saline (PBS).

FIG. 10 provides a schematic depicting the structure and chemical modification patterns of generic N-Acetylgalactosamine (GalNAc)-conjugated LPA oligonucleotides.

FIGS. 11A-11C provide graphs depicting the percent (%) of LPA mRNA in liver samples from non-human primates (NHPs) treated with the indicated GalNAc-conjugated LPA oligonucleotides relative to NHPs treated with PBS on day 28 (FIG. 11A), day 56 (FIG. 11B) and day 84 (FIG. 11C) following treatment.

FIG. 11D provides a graph depicting the percent (%) of PLG mRNA in liver samples from NHPs treated with the indicated GalNAc-conjugated LPA oligonucleotides relative to NHPs treated with PBS on day 28.

FIG. 12 provides a graph depicting the mean percent (%) of apo(a) protein in serum from NHPs treated with the indicated GalNAc-conjugated LPA oligonucleotides relative to NHPs treated with PBS over time.

### DETAILED DESCRIPTION

### I. Definitions

As used herein, “about,” as applied to one or more values of interest, refers to a value that is similar to a stated reference value. In certain embodiments, “about” refers to a range of values that fall within 25%, 20%, 19%, 18%1, 17%, 16%, 15%, 14%, 13%, 12%, 11%, 10%, 9%, 8%, 7%, 6%, 5%, 4%, 3%, 2%, 1% or less in either direction (greater than or less than) of the stated reference value unless otherwise stated or otherwise evident from the context (except where such number would exceed 100% of a possible value).

As used herein, “administer,” “administering,” “administration” and the like refers to providing a substance (e.g., an oligonucleotide) to a subject in a manner that is pharmacologically useful (e.g., to treat a condition in the subject).

As used herein, the term “apolipoprotein(a)” and abbreviated as “apo(a)”, refers to the apolipoprotein(a) polypeptide, which is a member of the apolipoprotein class of polypeptides that bind lipids to form lipoproteins. Apo(a) is a polymorphic glycoprotein encoded by the LPA gene in humans. LPA mRNA and apo(a) polypeptide are expressed predominantly in the liver. Lipoprotein(a) (abbreviated as Lp(a)) is a class of lipoprotein formed in the liver and comprises a single copy of apolipoprotein (apo) B-100 (Apo-B100) covalently tethered to apo(a). In humans, apo(a) includes at least 10 subtypes of KIV repeats, composed of 1 copy each of KIV₁, multiple copies of KIV₂, and 1 copy each of KIV₃-KIV₁₀, KV, and an inactive protease-like domain. The presence of apo(a) distinguishes Lp(a) from all other lipoprotein classes (Marcovina et al., (1995) Clin Chem. 41(2):246-55). For the purposes of the disclosure, “apolipoprotein(a)” or “apo(a)” refers to the apo(a) polypeptide from any vertebrate or mammal including, but not limited to, human, mouse, primate, monkey, bovine, chicken, rodent, rat, porcine, ovine and guinea pig. “Apo(a)” also refers to fragments and variants of native apo(a) that maintain at least one in vivo or in vitro activity of a native apo(a). Apo(a) encompasses full-length, unprocessed precursor forms of Apo(a), as well as mature forms resulting from post-translational processing. An exemplary sequence of a human LPA mRNA transcript is publicly available (GenBank Accession No. NM_005577.3) and disclosed herein (SEQ ID NO: 1). An exemplary sequence of cynomolgus monkey LPA mRNA is publicly available (GenBank Accession No. XM_015448517.1) and disclosed herein (SEQ ID NO: 2).

As used herein, “asialoglycoprotein receptor” or “ASGPR” refers to a bipartite C-type lectin formed by a major 48 kDa subunit (ASGPR-1) and minor 40 kDa subunit (ASGPR-2). ASGPR is primarily expressed on the sinusoidal surface of hepatocyte cells and has a major role in binding, internalizing and subsequent clearing of circulating glycoproteins that contain terminal galactose or GalNAc residues (asialoglycoproteins).

As used herein, “attenuate,” “attenuating,” “attenuation” and the like refers to reducing or effectively halting. As a non-limiting example, one or more of the treatments herein may reduce or effectively halt the onset or progression of cardiometabolic diseases including atherosclerosis, dyslipidemia, NAFLD and NASH in a subject. This attenuation may be exemplified by, for example, a decrease in one or more aspects (e.g., symptoms, tissue characteristics, and cellular, inflammatory or immunological activity, etc.) of cardiometabolic diseases including atherosclerosis, dyslipidemia, NAFLD and NASH, no detectable progression (worsening) of one or more aspects of cardiometabolic diseases including atherosclerosis, dyslipidemia, NAFLD and NASH, or no detectable aspects of cardiometabolic diseases including atherosclerosis, dyslipidemia, NAFLD and NASH in a subject when they might otherwise be expected.

As used herein, “complementary” refers to a structural relationship between two nucleotides (e.g., on two opposing nucleic acids or on opposing regions of a single nucleic acid strand) that permits the two nucleotides to form base pairs with one another. For example, a purine nucleotide of one nucleic acid that is complementary to a pyrimidine nucleotide of an opposing nucleic acid may base pair together by forming hydrogen bonds with one another. In some embodiments, complementary nucleotides can base pair in the Watson-Crick manner or in any other manner that allows for the formation of stable duplexes. In some embodiments, two nucleic acids may have regions of multiple nucleotides that are complementary with each other to form regions of complementarity, as described herein.

As used herein, “deoxyribonucleotide” refers to a nucleotide having a hydrogen in place of a hydroxyl at the 2′ position of its pentose sugar when compared with a ribonucleotide. A modified deoxyribonucleotide is a deoxyribonucleotide having one or more modifications or substitutions of atoms other than at the 2′ position, including modifications or substitutions in or of the sugar, phosphate group or base.

As used herein, “double-stranded oligonucleotide” or “ds oligonucleotide” refers to an oligonucleotide that is substantially in a duplex form. In some embodiments, the complementary base-pairing of duplex region(s) of a ds oligonucleotide is formed between antiparallel sequences of nucleotides of covalently separate nucleic acid strands. In some embodiments, complementary base-pairing of duplex region(s) of a ds oligonucleotide is formed between antiparallel sequences of nucleotides of nucleic acid strands that are covalently linked. In some embodiments, complementary base-pairing of duplex region(s) of a ds oligonucleotide is formed from single nucleic acid strand that is folded (e.g., via a hairpin) to provide complementary antiparallel sequences of nucleotides that base pair together. In some embodiments, a ds oligonucleotide comprises two covalently separate nucleic acid strands that are fully duplexed with one another. However, in some embodiments, a ds oligonucleotide comprises two covalently separate nucleic acid strands that are partially duplexed (e.g., having overhangs at one or both ends). In some embodiments, a ds oligonucleotide comprises antiparallel sequence of nucleotides that are partially complementary, and thus, may have one or more mismatches, which may include internal mismatches or end mismatches.

As used herein, “duplex,” in reference to nucleic acids (e.g., oligonucleotides), refers to a structure formed through complementary base pairing of two antiparallel sequences of nucleotides.

As used herein, “excipient” refers to a non-therapeutic agent that may be included in a composition, for example, to provide or contribute to a desired consistency or stabilizing effect.

As used herein, “hepatocyte” or “hepatocytes” refers to cells of the parenchymal tissues of the liver. These cells make up about 70%-85% of the liver's mass and manufacture serum albumin, FBN and the prothrombin group of clotting factors (except for Factors 3 and 4). Markers for hepatocyte lineage cells include, but are not limited to, transthyretin (Ttr), glutamine synthetase (Glul), hepatocyte nuclear factor 1a (Hnf1a) and hepatocyte nuclear factor 4a (Hnf4a). Markers for mature hepatocytes may include, but are not limited to, cytochrome P450 (Cyp3a11), fumarylacetoacetate hydrolase (Fah), glucose 6-phosphate (G6p), albumin (Alb) and OC2-2F8. See, e.g., Huch et al. (2013) Nature 494:247-250.

As used herein, a “hepatotoxic agent” refers to a chemical compound, virus or other substance that is itself toxic to the liver or can be processed to form a metabolite that is toxic to the liver. Hepatotoxic agents may include, but are not limited to, carbon tetrachloride (CCl₄), acetaminophen (paracetamol), vinyl chloride, arsenic, chloroform, nonsteroidal anti-inflammatory drugs (such as aspirin and phenylbutazone).

As used herein, “labile linker” refers to a linker that can be cleaved (e.g., by acidic pH). A “fairly stable linker” refers to a linker that cannot be cleaved.

As used herein, “liver inflammation” or “hepatitis” refers to a physical condition in which the liver becomes swollen, dysfunctional and/or painful, especially as a result of injury or infection, as may be caused by exposure to a hepatotoxic agent. Symptoms may include jaundice (yellowing of the skin or eyes), fatigue, weakness, nausea, vomiting, appetite reduction and weight loss. Liver inflammation, if left untreated, may progress to fibrosis, cirrhosis, liver failure or liver cancer.

As used herein, “liver fibrosis” or “fibrosis of the liver” refers to an excessive accumulation in the liver of extracellular matrix proteins, which could include collagens (I, III, and IV), FBN, undulin, elastin, laminin, hyaluronan and proteoglycans resulting from inflammation and liver cell death. Liver fibrosis, if left untreated, may progress to cirrhosis, liver failure or liver cancer.

As used herein, “loop” refers to a unpaired region of a nucleic acid (e.g., oligonucleotide) that is flanked by two antiparallel regions of the nucleic acid that are sufficiently complementary to one another, such that under appropriate hybridization conditions (e.g., in a phosphate buffer, in a cells), the two antiparallel regions, which flank the unpaired region, hybridize to form a duplex (referred to as a “stem”).

As used herein, “modified internucleotide linkage” refers to an internucleotide linkage having one or more chemical modifications when compared with a reference internucleotide linkage comprising a phosphodiester bond. In some embodiments, a modified nucleotide is a non-naturally occurring linkage. Typically, a modified internucleotide linkage confers one or more desirable properties to a nucleic acid in which the modified internucleotide linkage is present. For example, a modified nucleotide may improve thermal stability, resistance to degradation, nuclease resistance, solubility, bioavailability, bioactivity, reduced immunogenicity, etc.

As used herein, “modified nucleotide” refers to a nucleotide having one or more chemical modifications when compared with a corresponding reference nucleotide selected from: adenine ribonucleotide, guanine ribonucleotide, cytosine ribonucleotide, uracil ribonucleotide, adenine deoxyribonucleotide, guanine deoxyribonucleotide, cytosine deoxyribonucleotide and thymidine deoxyribonucleotide. In some embodiments, a modified nucleotide is a non-naturally occurring nucleotide. In some embodiments, a modified nucleotide has one or more chemical modification in its sugar, nucleobase and/or phosphate group. In some embodiments, a modified nucleotide has one or more chemical moieties conjugated to a corresponding reference nucleotide. Typically, a modified nucleotide confers one or more desirable properties to a nucleic acid in which the modified nucleotide is present. For example, a modified nucleotide may improve thermal stability, resistance to degradation, nuclease resistance, solubility, bioavailability, bioactivity, reduced immunogenicity, etc.

As used herein, “nicked tetraloop structure” refers to a structure of a RNAi oligonucleotide that is characterized by separate sense (passenger) and antisense (guide) strands, in which the sense strand has a region of complementarity with the antisense strand, and in which at least one of the strands, generally the sense strand, has a tetraloop configured to stabilize an adjacent stem region formed within the at least one strand.

As used herein, “oligonucleotide” refers to a short nucleic acid (e.g., less than about 100 nucleotides in length). An oligonucleotide may be single-stranded (ss) or ds. An oligonucleotide may or may not have duplex regions. As a set of non-limiting examples, an oligonucleotide may be, but is not limited to, a small interfering RNA (siRNA), microRNA (miRNA), short hairpin RNA (shRNA), dicer substrate interfering RNA (dsiRNA), antisense oligonucleotide, short siRNA or ss siRNA. In some embodiments, a ds oligonucleotide is an RNAi oligonucleotide.

As used herein, “overhang” refers to terminal non-base pairing nucleotide(s) resulting from one strand or region extending beyond the terminus of a complementary strand with which the one strand or region forms a duplex. In some embodiments, an overhang comprises one or more unpaired nucleotides extending from a duplex region at the 5′ terminus or 3′ terminus of a ds oligonucleotide. In certain embodiments, the overhang is a 3′ or 5′ overhang on the antisense strand or sense strand of a ds oligonucleotides.

As used herein, “phosphate analog” refers to a chemical moiety that mimics the electrostatic and/or steric properties of a phosphate group. In some embodiments, a phosphate analog is positioned at the 5′ terminal nucleotide of an oligonucleotide in place of a 5′-phosphate, which is often susceptible to enzymatic removal. In some embodiments, a 5′ phosphate analog contains a phosphatase-resistant linkage. Examples of phosphate analogs include, but are not limited to, 5′ phosphonates, such as 5′ methylenephosphonate (5′-MP) and 5′-(E)-vinylphosphonate (5′-VP). In some embodiments, an oligonucleotide has a phosphate analog at a 4′-carbon position of the sugar (referred to as a “4′-phosphate analog”) at a 5′-terminal nucleotide. An example of a 4′-phosphate analog is oxymethylphosphonate, in which the oxygen atom of the oxymethyl group is bound to the sugar moiety (e.g., at its 4′-carbon) or analog thereof. See, e.g., US Provisional Patent Application Nos. 62/383,207 (filed on 2 Sep. 2016) and 62/393,401 (filed on 12 Sep. 2016). Other modifications have been developed for the 5′ end of oligonucleotides (see, e.g., Intl. Patent Application No. WO 2011/133871; U.S. Pat. No. 8,927,513; and Prakash et al. (2015) Nucleic Acids Res. 43:2993-3011).

As used herein, “reduced expression” of a gene (e.g., LPA) refers to a decrease in the amount or level of RNA transcript (e.g., LPA mRNA) or protein encoded by the gene and/or a decrease in the amount or level of activity of the gene in a cell, a population of cells, a sample or a subject, when compared to an appropriate reference (e.g., a reference cell, population of cells, sample or subject). For example, the act of contacting a cell with an oligonucleotide herein (e.g., an oligonucleotide comprising an antisense strand having a nucleotide sequence that is complementary to a nucleotide sequence comprising LPA mRNA) may result in a decrease in the amount or level of LPA mRNA, apo(a) protein and/or apo(a) activity (e.g., via inactivation and/or degradation of LPA mRNA by the RNAi pathway) when compared to a cell that is not treated with the ds oligonucleotide. Similarly, and as used herein, “reducing expression” refers to an act that results in reduced expression of a gene (e.g., LPA). As used herein, “reduction of LPA expression” refers to a decrease in the amount or level of LPA mRNA, apo(a) protein and/or apo(a) activity in a cell, a population of cells, a sample or a subject when compared to an appropriate reference (e.g., a reference cell, population of cells, sample, or subject).

As used herein, “region of complementarity” refers to a sequence of nucleotides of a nucleic acid (e.g., a ds oligonucleotide) that is sufficiently complementary to an antiparallel sequence of nucleotides to permit hybridization between the two sequences of nucleotides under appropriate hybridization conditions (e.g., in a phosphate buffer, in a cell, etc.). In some embodiments, an oligonucleotide herein comprises a targeting sequence having a region of complementary to a mRNA target sequence.

As used herein, “ribonucleotide” refers to a nucleotide having a ribose as its pentose sugar, which contains a hydroxyl group at its 2′ position. A modified ribonucleotide is a ribonucleotide having one or more modifications or substitutions of atoms other than at the 2′ position, including modifications or substitutions in or of the ribose, phosphate group or base.

As used herein, “RNAi oligonucleotide” refers to either (a) a ds oligonucleotide having a sense strand (passenger) and antisense strand (guide), in which the antisense strand or part of the antisense strand is used by the Argonaute 2 (Ago2) endonuclease in the cleavage of a target mRNA (e.g., LPA mRNA) or (b) a ss oligonucleotide having a single antisense strand, where that antisense strand (or part of that antisense strand) is used by the Ago2 endonuclease in the cleavage of a target mRNA (e.g., LPA mRNA).

As used herein, “strand” refers to a single, contiguous sequence of nucleotides linked together through internucleotide linkages (e.g., phosphodiester linkages or phosphorothioate linkages). In some embodiments, a strand has two free ends (e.g., a 5′ end and a 3′ end).

As used herein, “subject” means any mammal, including mice, rabbits and humans. In one embodiment, the subject is a human or NHP. Moreover, “individual” or “patient” may be used interchangeably with “subject.”

As used herein, “synthetic” refers to a nucleic acid or other molecule that is artificially synthesized (e.g., using a machine (e.g., a solid-state nucleic acid synthesizer)) or that is otherwise not derived from a natural source (e.g., a cell or organism) that normally produces the molecule.

As used herein, “targeting ligand” refers to a molecule (e.g., a carbohydrate, amino sugar, cholesterol, polypeptide or lipid) that selectively binds to a cognate molecule (e.g., a receptor) of a tissue or cell of interest and that is conjugatable to another substance for purposes of targeting the other substance to the tissue or cell of interest. For example, in some embodiments, a targeting ligand may be conjugated to an oligonucleotide for purposes of targeting the oligonucleotide to a specific tissue or cell of interest. In some embodiments, a targeting ligand selectively binds to a cell surface receptor. Accordingly, in some embodiments, a targeting ligand when conjugated to an oligonucleotide facilitates delivery of the oligonucleotide into a particular cell through selective binding to a receptor expressed on the surface of the cell and endosomal internalization by the cell of the complex comprising the oligonucleotide, targeting ligand and receptor. In some embodiments, a targeting ligand is conjugated to an oligonucleotide via a linker that is cleaved following or during cellular internalization such that the oligonucleotide is released from the targeting ligand in the cell.

As used herein, “tetraloop” refers to a loop that increases stability of an adjacent duplex formed by hybridization of flanking sequences of nucleotides. The increase in stability is detectable as an increase in melting temperature (Tₘ) of an adjacent stem duplex that is higher than the Tₘ of the adjacent stem duplex expected, on average, from a set of loops of comparable length consisting of randomly selected sequences of nucleotides. For example, a tetraloop can confer a Tₘ of at least about 50° C., at least about 55° C., at least about 56° C., at least about 58° C., at least about 60° C., at least about 65° C. or at least about 75° C. in 10 mM NaHPO₄ to a hairpin comprising a duplex of at least 2 base pairs (bp) in length. In some embodiments, a tetraloop may stabilize a bp in an adjacent stem duplex by stacking interactions. In addition, interactions among the nucleotides in a tetraloop include, but are not limited to, non-Watson-Crick base pairing, stacking interactions, hydrogen bonding and contact interactions (Cheong et al. (1990) NATURE 346:680-82; Heus & Pardi (1991) SCIENCE 253:191-94). In some embodiments, a tetraloop comprises or consists of 3 to 6 nucleotides and is typically 4 to 5 nucleotides. In certain embodiments, a tetraloop comprises or consists of 3, 4, 5 or 6 nucleotides, which may or may not be modified (e.g., which may or may not be conjugated to a targeting moiety). In one embodiment, a tetraloop consists of 4 nucleotides.

Any nucleotide may be used in the tetraloop and standard IUPAC-IUB symbols for such nucleotides may be used as described in Cornish-Bowden (1985) Nucleic Acids Res. 13:3021-3030. For example, the letter “N” may be used to mean that any base may be in that position, the letter “R” may be used to show that A (adenine) or G (guanine) may be in that position, and “B” may be used to show that C (cytosine), G (guanine), T (thymine) or U (uracil) may be in that position. Examples of tetraloops include the UNCG family of tetraloops (e.g., UUCG), the GNRA family of tetraloops (e.g., GAAA), and the CUUG tetraloop (Woese et al. (1990) Proc. Natl. Acad. Sci. USA 87:8467-8471; Antao et al. (1991) Nucleic Acids Res. 19:5901-5905). Examples of DNA tetraloops include the d(GNNA) family of tetraloops (e.g., d(GTTA), the d(GNRA)) family of tetraloops, the d(GNAB) family of tetraloops, the d(CNNG) family of tetraloops, and the d(TNCG) family of tetraloops (e.g., d(TTCG)). See, e.g., Nakano et al. (2002) Biochem. 41:4281-14292; Shinji et al. (2000) Nippon Kagakkai Koen Yokoshu 78:731. In some embodiments, the tetraloop is contained within a nicked tetraloop structure.

As used herein, “treat” or “treating” refers to the act of providing care to a subject in need thereof, for example, by administering a therapeutic agent (e.g., an oligonucleotide herein) to the subject, for purposes of improving the health and/or well-being of the subject with respect to an existing condition (e.g., a disease, disorder) or to prevent or decrease the likelihood of the occurrence of a condition. In some embodiments, treatment involves reducing the frequency or severity of at least one sign, symptom or contributing factor of a condition (e.g., disease, disorder) experienced by a subject.

### II. Oligonucleotide Inhibitors of LPA Expression

The disclosure provides, inter alia, oligonucleotides that inhibit LPA expression. In some embodiments, an oligonucleotide that inhibits LPA expression herein is targeted to an LPA mRNA.

i. LPA Target Sequences

In some embodiments, the oligonucleotide is targeted to a target sequence comprising an LPA mRNA. In some embodiments, the oligonucleotide, or a portion, fragment or strand thereof (e.g., an antisense strand or a guide strand of a ds oligonucleotide) binds or anneals to a target sequence comprising an LPA mRNA, thereby inhibiting LPA expression. In some embodiments, the oligonucleotide is targeted to an LPA target sequence for the purpose of inhibiting LPA expression in vivo. In some embodiments, the amount or extent of inhibition of LPA expression by an oligonucleotide targeted to an LPA target sequence correlates with the potency of the oligonucleotide. In some embodiments, the amount or extent of inhibition of LPA expression by an oligonucleotide targeted to an LPA target sequence correlates with the amount or extent of therapeutic benefit in a subject or patient having a disease, disorder or condition associated with the expression of LPA treated with the oligonucleotide.

Through examination and analysis of the nucleotide sequence of LPA mRNAs encoding apo(a), including mRNAs of multiple different species (e.g., human, cynomolgus monkey, and rhesus monkey; see, e.g., Example 1) and as a result of in vitro and in vivo testing (see, e.g., Example 2 and Example 3), it has been discovered that certain nucleotide sequences of LPA mRNA are more amenable than others to oligonucleotide-based inhibition of LPA expression and are thus useful as target sequences for the oligonucleotides herein. In some embodiments, a sense strand of an oligonucleotide (e.g., a ds oligonucleotide) described herein (e.g., in Table 5) comprises an LPA target sequence. In some embodiments, a portion or region of the sense strand of a ds oligonucleotide described herein (e.g., in Table 5) comprises an LPA target sequence. In some embodiments, an LPA target sequence comprises, or consists of, a sequence of any one of SEQ ID Nos: 4-387.

ii. LPA Targeting Sequences

In some embodiments, the oligonucleotides herein have regions of complementarity to LPA mRNA (e.g., within a target sequence of LPA mRNA) for purposes of targeting the LPA mRNA in cells and inhibiting LPA expression. In some embodiments, the oligonucleotides herein comprise an LPA targeting sequence (e.g., an antisense strand or a guide strand of a ds oligonucleotide) having a region of complementarity that binds or anneals to an LPA target sequence by complementary (Watson-Crick) base pairing. The targeting sequence or region of complementarity is generally of a suitable length and base content to enable binding or annealing of the oligonucleotide (or a strand thereof) to an LPA mRNA for purposes of inhibiting its expression. In some embodiments, the targeting sequence or region of complementarity is at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29 or at least about 30 nucleotides in length. In some embodiments, the targeting sequence or region of complementarity is about 12 to about 30 (e.g., 12 to 30, 12 to 22, 15 to 25, 17 to 21, 18 to 27, 19 to 27, or 15 to 30) nucleotides in length. In some embodiments, the targeting sequence or region of complementarity is about 12, 13, 14, 15, 16, 17, 18, 19, 20, 21, 22, 23, 24, 25, 26, 27, 28, 29 or 30 nucleotides in length. In some embodiments, the targeting sequence or region of complementarity is 18 nucleotides in length. In some embodiments, the targeting sequence or region of complementarity is 19 nucleotides in length. In some embodiments, the targeting sequence or region of complementarity is 20 nucleotides in length. In some embodiments, the targeting sequence or region of complementarity is 21 nucleotides in length. In some embodiments, the targeting sequence or region of complementarity is 22 nucleotides in length. In some embodiments, the targeting sequence or region of complementarity is 23 nucleotides in length. In some embodiments, the targeting sequence or region of complementarity is 24 nucleotides in length.

In some embodiments, an oligonucleotide herein comprises a targeting sequence or a region of complementarity (e.g., an antisense strand or a guide strand of a double-stranded oligonucleotide) that is fully complementary to an LPA target sequence. In some embodiments, the targeting sequence or region of complementarity is partially complementary to an LPA target sequence. In some embodiments, the oligonucleotide comprises a targeting sequence or region of complementarity that is fully complementary to a sequence of any one of SEQ ID NOs: 4-387. In some embodiments, the oligonucleotide comprises a targeting sequence or region of complementarity that is partially complementary to a sequence of any one of SEQ ID NOs: 4-387.

In some embodiments, the oligonucleotide herein comprises a targeting sequence or region of complementarity that is complementary to a contiguous sequence of nucleotides comprising an LPA mRNA, wherein the contiguous sequence of nucleotides is about 12 to about 30 nucleotides in length (e.g., 12 to 30, 12 to 28, 12 to 26, 12 to 24, 12 to 20, 12 to 18, 12 to 16, 14 to 22, 16 to 20, 18 to 20 or 18 to 19 nucleotides in length). In some embodiments, the oligonucleotide comprises a targeting sequence or region of complementarity that is complementary to a contiguous sequence of nucleotides comprising an LPA mRNA, wherein the contiguous sequence of nucleotides is 10, 11, 12, 13, 14, 15, 16, 17, 18, 19 or 20 nucleotides in length. In some embodiments, the oligonucleotide comprises a targeting sequence or region of complementarity that is complementary to a contiguous sequence of nucleotides comprising an LPA mRNA, wherein the contiguous sequence of nucleotides is 19 nucleotides in length. In some embodiments, the oligonucleotide comprises a targeting sequence or region of complementarity that is complementary to a contiguous sequence of nucleotides comprising an LPA mRNA, wherein the contiguous sequence of nucleotides is 20 nucleotides in length. In some embodiments, the oligonucleotide comprises a targeting sequence or a region of complementarity that is complementary to a contiguous sequence of nucleotides of any one of SEQ ID NOs: 4-387, optionally wherein the contiguous sequence of nucleotides is 19 nucleotides in length.

In some embodiments, a targeting sequence or region of complementarity of an oligonucleotide that is complementary to contiguous nucleotides of a sequence as set forth in any one of SEQ ID NOs: 4-387 and spans the entire length of an antisense strand. In some embodiments, a region of complementarity of an oligonucleotide that is complementary to contiguous nucleotides of a sequence as set forth in any one of SEQ ID NOs: 4-387 and spans a portion of the entire length of an antisense strand. In some embodiments, an oligonucleotide herein comprises a region of complementarity (e.g., on an antisense strand of a ds oligonucleotide) that is at least partially (e.g., fully) complementary to a contiguous stretch of nucleotides spanning nucleotides 1-20 of a sequence as set forth in any one of SEQ ID NOs: 4-387.

In some embodiments, an oligonucleotide herein comprises a targeting sequence or region of complementarity having one or more base pair (bp) mismatches with the corresponding LPA target sequence. In some embodiments, the targeting sequence or region of complementarity may have up to about 1, up to about 2, up to about 3, up to about 4, up to about 5, etc. mismatches with the corresponding LPA target sequence provided that the ability of the targeting sequence or region of complementarity to bind or anneal to the LPA mRNA under appropriate hybridization conditions and/or the ability of the oligonucleotide to reduce or inhibit LPA expression is maintained. Alternatively, in some embodiments, the targeting sequence or region of complementarity comprises no more than 1, no more than 2, no more than 3, no more than 4, or no more than 5 mismatches with the corresponding LPA target sequence provided that the ability of the targeting sequence or region of complementarity to bind or anneal to the LPA mRNA under appropriate hybridization conditions and/or the ability of the oligonucleotide to reduce or inhibit LPA expression is maintained. In some embodiments, the oligonucleotide comprises a targeting sequence or region of complementarity having 1 mismatch with the corresponding target sequence. In some embodiments, the oligonucleotide comprises a targeting sequence or region of complementarity having 2 mismatches with the corresponding target sequence. In some embodiments, the oligonucleotide comprises a targeting sequence or region of complementarity having 3 mismatches with the corresponding target sequence. In some embodiments, the oligonucleotide comprises a targeting sequence or region of complementarity having 4 mismatches with the corresponding target sequence. In some embodiments, the oligonucleotide comprises a targeting sequence or region of complementarity having 5 mismatches with the corresponding target sequence. In some embodiments, the oligonucleotide comprises a targeting sequence or region of complementarity more than one mismatch (e.g., 2, 3, 4, 5 or more mismatches) with the corresponding target sequence, wherein at least 2 (e.g., all) of the mismatches are positioned consecutively (e.g., 2, 3, 4, 5 or more mismatches in a row), or wherein the mismatches are interspersed in any position throughout the targeting sequence or region of complementarity. In some embodiments, the oligonucleotide comprises a targeting sequence or region of complementarity more than one mismatch (e.g., 2, 3, 4, 5 or more mismatches) with the corresponding target sequence, wherein at least 2 (e.g., all) of the mismatches are positioned consecutively (e.g., 2, 3, 4, 5 or more mismatches in a row), or wherein at least one or more non-mismatched base pair is located between the mismatches, or a combination thereof.

iii. Types of Oligonucleotides

A variety of oligonucleotide types and/or structures are useful for targeting LPA mRNA in the methods herein including, but not limited to, RNAi oligonucleotides, antisense oligonucleotides, miRNAs, etc. Any of the oligonucleotide types described herein or elsewhere are contemplated for use as a framework to incorporate an LPA mRNA targeting sequence herein for the purposes of inhibiting LPA expression.

In some embodiments, the oligonucleotides herein inhibit LPA expression by engaging with RNA interference (RNAi) pathways upstream or downstream of Dicer involvement. For example, RNAi oligonucleotides have been developed with each strand having sizes of about 19-25 nucleotides with at least one 3′ overhang of 1 to 5 nucleotides (see, e.g., U.S. Pat. No. 8,372,968). Longer oligonucleotides also have been developed that are processed by Dicer to generate active RNAi products (see, e.g., U.S. Pat. No. 8,883,996). Further work produced extended ds oligonucleotides where at least one end of at least one strand is extended beyond a duplex targeting region, including structures where one of the strands includes a thermodynamically-stabilizing tetraloop structure (see, e.g., U.S. Pat. Nos. 8,513,207 and 8,927,705, as well as Intl. Patent Application Publication No. WO 2010/033225). Such structures may include ss extensions (on one or both sides of the molecule) as well as ds extensions.

In some embodiments, the oligonucleotides herein engage with the RNAi pathway downstream of the involvement of Dicer (e.g., Dicer cleavage). In some embodiments, the oligonucleotide has an overhang (e.g., of 1, 2, or 3 nucleotides in length) in the 3′ end of the sense strand. In some embodiments, the oligonucleotide (e.g., siRNA) comprises a 21-nucleotide guide strand that is antisense to a target mRNA (e.g., LPA mRNA) and a complementary passenger strand, in which both strands anneal to form a 19-bp duplex and 2 nucleotide overhangs at either or both 3′ ends. Longer oligonucleotide designs also are contemplated including oligonucleotides having a guide strand of 23 nucleotides and a passenger strand of 21 nucleotides, where there is a blunt end on the right side of the molecule (3′ end of passenger strand/5′ end of guide strand) and a two nucleotide 3′-guide strand overhang on the left side of the molecule (5′ end of the passenger strand/3′ end of the guide strand). In such molecules, there is a21 bp duplex region. See, e.g., U.S. Pat. Nos. 9,012,138; 9,012,621 and 9,193,753.

In some embodiments, the oligonucleotides herein comprise sense and antisense strands that are both in the range of about 17 to 26 (e.g., 17 to 26, 20 to 25 or 21-23) nucleotides in length. In some embodiments, an oligonucleotide herein comprises a sense and antisense strand that are both in the range of about 19-22 nucleotides in length. In some embodiments, the sense and antisense strands are of equal length. In some embodiments, an oligonucleotide comprises sense and antisense strands, such that there is a 3′-overhang on either the sense strand or the antisense strand, or both the sense and antisense strand. In some embodiments, for oligonucleotides that have sense and antisense strands that are both in the range of about 21-23 nucleotides in length, a 3′ overhang on the sense, antisense, or both sense and antisense strands is 1 or 2 nucleotides in length. In some embodiments, the oligonucleotide has a guide strand of 22 nucleotides and a passenger strand of 20 nucleotides, where there is a blunt end on the right side of the molecule (3′ end of passenger strand/5′ end of guide strand) and a 2 nucleotide 3′-guide strand overhang on the left side of the molecule (5′ end of the passenger strand/3′ end of the guide strand). In such molecules, there is a 20 bp duplex region.

Other oligonucleotide designs for use with the compositions and methods herein include: 16-mer siRNAs (see, e.g., NUCLEIC ACIDS IN CHEMISTRY AND BIOLOGY, Blackburn (ed.), Royal Society of Chemistry, 2006), shRNAs (e.g., having 19 bp or shorter stems; see, e.g., Moore et al. (2010) METHODS MOL. BIOL. 629:141-58), blunt siRNAs (e.g., of 19 bps in length; see, e.g., Kraynack & Baker (2006) RNA 12:163-76), asymmetrical siRNAs (aiRNA; see, e.g., Sun et al. (2008) NAT. BIOTECHNOL. 26:1379-82), asymmetric shorter-duplex siRNA (see, e.g., Chang et al. (2009) MOL. THER. 17:725-32), fork siRNAs (see, e.g., Hohjoh (2004) FEBS LETT. 557:193-198), ss siRNAs (Elsner (2012) NAT. BIOTECHNOL. 30:1063), dumbbell-shaped circular siRNAs (see, e.g., Abe et al. (2007) J. AM. CHEM. SOC. 129:15108-09), and small internally segmented interfering RNA (siRNA; see, e.g., Bramsen et al. (2007) NUCLEIC ACIDS RES. 35:5886-97). Further non-limiting examples of an oligonucleotide structures that may be used in some embodiments to reduce or inhibit the expression of LPA are microRNA (miRNA), short hairpin RNA (shRNA) and short siRNA (see, e.g., Hamilton et al. (2002) EMBO J. 21:4671-79; see also, US Patent Application Publication No. 2009/0099115).

Still, in some embodiments, an oligonucleotide for reducing or inhibiting LPA expression herein is single-stranded (ss). Such structures may include but are not limited to ss RNAi molecules. Recent efforts have demonstrated the activity of ss RNAi molecules (see, e.g., Matsui et al. (2016) Mol. Ther. 24:946-955). However, in some embodiments, oligonucleotides herein are antisense oligonucleotides (ASOs). An antisense oligonucleotide is a ss oligonucleotide that has a nucleobase sequence which, when written or depicted in the 5′ to 3′ direction, comprises the reverse complement of a targeted segment of a particular nucleic acid and is suitably modified (e.g., as a gapmer) so as to induce RNaseH-mediated cleavage of its target RNA in cells or (e.g., as a mixmer) so as to inhibit translation of the target mRNA in cells. ASOs for use herein may be modified in any suitable manner known in the art including, for example, as shown in U.S. Pat. No. 9,567,587 (including, e.g., length, sugar moieties of the nucleobase (pyrimidine, purine), and alterations of the heterocyclic portion of the nucleobase). Further, ASOs have been used for decades to reduce expression of specific target genes (see, e.g., Bennett et al. (2017) Annu. Rev. Pharmacol. 57:81-105).

iv. Double-Stranded Oligonucleotides

The disclosure provides double-stranded (ds) oligonucleotides for targeting LPA mRNA and inhibiting LPA expression (e.g., via the RNAi pathway) comprising a sense strand (also referred to herein as a passenger strand) and an antisense strand (also referred to herein as a guide strand). In some embodiments, the sense strand and antisense strand are separate strands and are not covalently linked. In some embodiments, the sense strand and antisense strand are covalently linked.

In some embodiments, the sense strand has a first region (R1) and a second region (R2), wherein R2 comprises a first subregion (S1), a tetraloop (L) or triloop (triL), and a second subregion (S2), wherein L or triL is located between S1 and S2, and wherein S1 and S2 form a second duplex (D2). D2 may have various lengths. In some embodiments, D2 is about 1-6 bp in length. In some embodiments, D2 is 2-6, 3-6, 4-6, 5-6, 1-5, 2-5, 3-5 or 4-5 bp in length. In some embodiments, D2 is 1, 2, 3, 4, 5 or 6 bp in length. In some embodiments, D2 is 6 bp in length.

In some embodiments, R1 of the sense strand and the antisense strand form a first duplex (D1). In some embodiments, D1 is at least about 15 (e.g., at least 15, at least 16, at least 17, at least 18, at least 19, at least 20 or at least 21) nucleotides in length. In some embodiments, D1 is in the range of about 12 to 30 nucleotides in length (e.g., 12 to 30, 12 to 27, 15 to 22, 18 to 22, 18 to 25, 18 to 27, 18 to 30 or 21 to 30 nucleotides in length). In some embodiments, D1 is at least 12 nucleotides in length (e.g., at least 12, at least 15, at least 20, at least 25, or at least 30 nucleotides in length). In some embodiments, D1 is 12, 13, 14, 15, 16, 17, 18, 19, 20, 21, 22, 23, 24, 25, 26, 27, 28, 29 or 30 nucleotides in length. In some embodiments, D1 is 20 nucleotides in length. In some embodiments, D1 comprising sense strand and antisense strand does not span the entire length of the sense strand and/or antisense strand. In some embodiments, D1 comprising the sense strand and antisense strand spans the entire length of either the sense strand or antisense strand or both. In certain embodiments, D1 comprising the sense strand and antisense strand spans the entire length of both the sense strand and the antisense strand.

In some embodiments, a ds oligonucleotide herein comprises a sense strand having a sequence of any one of SEQ ID NOs: 388-403 and an antisense strand comprising a complementary sequence selected from SEQ ID NOs: 788-803, as is arranged Table 3. In some embodiments, the sense strand comprises the sequence of SEQ ID NO: 393 and the antisense strand comprises the sequence of SEQ ID NO: 793.

It should be appreciated that, in some embodiments, sequences presented in the Sequence Listing may be referred to in describing the structure of an oligonucleotide (e.g., a ds oligonucleotide) or other nucleic acid. In such embodiments, the actual oligonucleotide or other nucleic acid may have one or more alternative nucleotides (e.g., an RNA counterpart of a DNA nucleotide or a DNA counterpart of an RNA nucleotide) and/or one or more modified nucleotides and/or one or more modified internucleotide linkages and/or one or more other modification when compared with the specified sequence while retaining essentially same or similar complementary properties as the specified sequence.

In some embodiments, a ds oligonucleotide herein comprises a 25-nucleotide sense strand and a 27-nucleotide antisense strand that when acted upon by a Dicer enzyme results in an antisense strand that is incorporated into the mature RISC. In some embodiments, the sense strand of the ds oligonucleotide is longer than 27 nucleotides (e.g., 28, 29, 30, 31, 32, 33, 34, 35, 36, 37, 38, 39 or 40 nucleotides). In some embodiments, the sense strand of the ds oligonucleotide is longer than 25 nucleotides (e.g., 26, 27, 28, 29 or 30 nucleotides).

In some embodiments, the ds oligonucleotides herein have one 5′ end that is thermodynamically less stable when compared to the other 5′ end. In some embodiments, an asymmetric ds oligonucleotide is provided that comprises a blunt end at the 3′ end of a sense strand and a 3′-overhang at the 3′ end of an antisense strand. In some embodiments, the 3′-overhang on the antisense strand is about 1-8 nucleotides in length (e.g., 1, 2, 3, 4, 5, 6, 7 or 8 nucleotides in length). Typically, a ds oligonucleotide for RNAi has a two-nucleotide overhang on the 3′ end of the antisense (guide) strand. However, other overhangs are possible. In some embodiments, an overhang is a 3′-overhang comprising a length of between 1 and 6 nucleotides, optionally 1 to 5, 1 to 4, 1 to 3, 1 to 2, 2 to 6, 2 to 5, 2 to 4, 2 to 3, 3 to 6, 3 to 5, 3 to 4, 4 to 6, 4 to 5, 5 to 6 nucleotides, or 1, 2, 3, 4, 5 or 6 nucleotides. However, in some embodiments, the overhang is a 5′-overhang comprising a length of between 1 and 6 nucleotides, optionally 1 to 5, 1 to 4, 1 to 3, 1 to 2, 2 to 6, 2 to 5, 2 to 4, 2 to 3, 3 to 6, 3 to 5, 3 to 4, 4 to 6, 4 to 5, 5 to 6 nucleotides, or 1, 2, 3, 4, 5 or 6 nucleotides.

In some embodiments, two terminal nucleotides on the 3′ end of an antisense strand are modified. In some embodiments, the two terminal nucleotides on the 3′ end of the antisense strand are complementary with the target mRNA (e.g., LPA mRNA). In some embodiments, the two terminal nucleotides on the 3′ end of the antisense strand are not complementary with the target mRNA. In some embodiments, two terminal nucleotides on each 3′ end of an oligonucleotide in the nicked tetraloop structure are GG. Typically, one or both of the two terminal GG nucleotides on each 3′ end of a ds oligonucleotide is not complementary with the target mRNA.

In some embodiments, there is one or more (e.g., 1, 2, 3, 4 or 5) mismatch(s) between a sense and antisense strand. If there is more than one mismatch between a sense and antisense strand, they may be positioned consecutively (e.g., 2, 3 or more in a row), or interspersed throughout the region of complementarity. In some embodiments, the 3′ end of the sense strand contains one or more mismatches. In one embodiment, two mismatches are incorporated at the 3′ end of the sense strand. In some embodiments, base mismatches, or destabilization of segments at the 3′ end of the sense strand of the oligonucleotide improves or increases the potency of the ds oligonucleotide.

a. Antisense Strands

In some embodiments, an oligonucleotide (e.g., ads oligonucleotide) disclosed herein for targeting LPA mRNA and inhibiting LPA expression comprises an antisense strand comprising or consisting of a sequence as set forth in any one of SEQ ID NOs: 404-803. In some embodiments, an oligonucleotide herein comprises an antisense strand comprising or consisting of at least about 12 (e.g., at least 12, at least 13, at least 14, at least 15, at least 16, at least 17, at least 18, at least 19, at least 20, at least 21, at least 22 or at least 23) contiguous nucleotides of a sequence as set forth in any one of SEQ ID NOs: 404-803.

In some embodiments, an oligonucleotide (e.g., a ds oligonucleotide) herein comprises an antisense strand of up to about 40 nucleotides in length (e.g., up to 40, up to 35, up to 30, up to 27, up to 25, up to 21, up to 19, up to 17 or up to 12 nucleotides in length). In some embodiments, an oligonucleotide may have an antisense strand of at least about 12 nucleotides in length (e.g., at least 12, at least 15, at least 19, at least 21, at least 22, at least 25, at least 27, at least 30, at least 35 or at least 38 nucleotides in length). In some embodiments, an oligonucleotide may have an antisense strand in a range of about 12 to about 40 (e.g., 12 to 40, 12 to 36, 12 to 32, 12 to 28, 15 to 40, 15 to 36, 15 to 32, 15 to 28, 17 to 22, 17 to 25, 19 to 27, 19 to 30, 20 to 40, 22 to 40, 25 to 40 or 32 to 40) nucleotides in length. In some embodiments, an oligonucleotide may have an antisense strand of 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides in length.

In some embodiments, an antisense strand of an oligonucleotide is referred to as a “guide strand.” For example, an antisense strand that engages with RNA-induced silencing complex (RISC) and binds to an Argonaute protein such as Ago2, or engages with or binds to one or more similar factors, and directs silencing of a target gene, the antisense strand is referred to as a guide strand. In some embodiments, a sense strand complementary to a guide strand is referred to as a “passenger strand.”

b. Sense Strands

In some embodiments, an oligonucleotide (e.g., a ds oligonucleotide) herein for targeting LPA mRNA and inhibiting LPA expression comprises or consists of a sense strand sequence as set forth in in any one of SEQ ID NOs: 4-403. In some embodiments, an oligonucleotide has a sense strand that comprises or consists of at least about 12 (e.g., at least 13, at least 14, at least 15, at least 16, at least 17, at least 18, at least 19, at least 20, at least 21, at least 22 or at least 23) contiguous nucleotides of a sequence as set forth in in any one of SEQ ID NOs: 4-403.

In some embodiments, an oligonucleotide (e.g., a ds oligonucleotide) herein comprises a sense strand (or passenger strand) of up to about 40 nucleotides in length (e.g., up to 40, up to 36, up to 30, up to 27, up to 25, up to 21, up to 19, up to 17 or up to 12 nucleotides in length). In some embodiments, an oligonucleotide may have a sense strand of at least about 12 nucleotides in length (e.g., at least 12, at least 15, at least 19, at least 21, at least 25, at least 27, at least 30, at least 36 or at least 38 nucleotides in length). In some embodiments, an oligonucleotide may have a sense strand in a range of about 12 to about 40 (e.g., 12 to 40, 12 to 36, 12 to 32, 12 to 28, 15 to 40, 15 to 36, 15 to 32, 15 to 28, 17 to 21, 17 to 25, 19 to 27, 19 to 30, 20 to 40, 22 to 40, 25 to 40 or 32 to 40) nucleotides in length. In some embodiments, an oligonucleotide may have a sense strand of 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides in length.

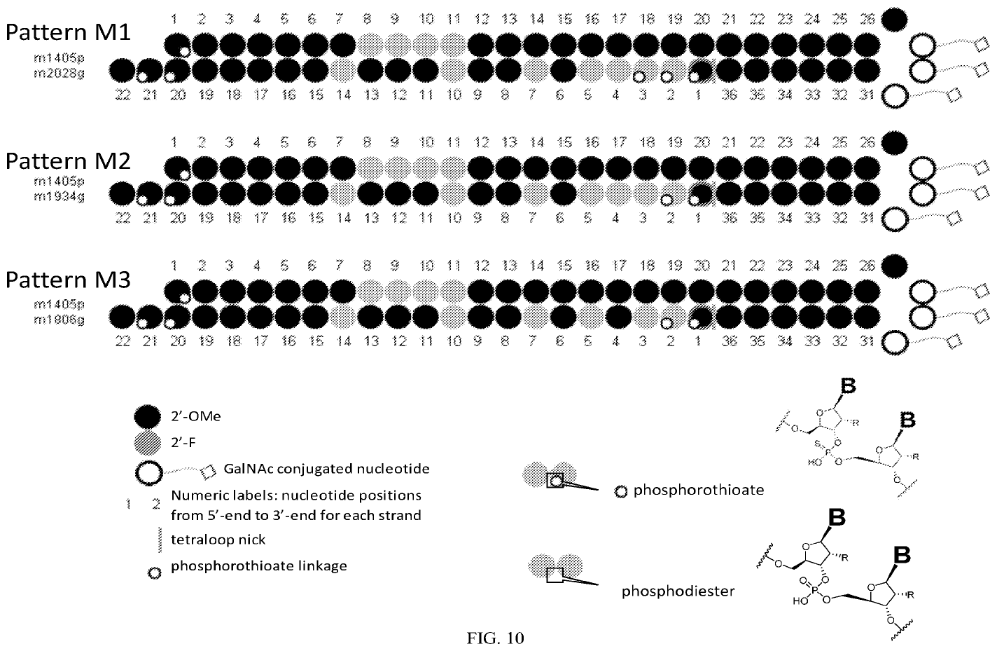

In some embodiments, a sense strand comprises a stem-loop structure at its 3′ end. In some embodiments, a sense strand comprises a stem-loop structure at its 5′ end. In some embodiments, a stem is a duplex of 2, 3, 4, 5, 6, 7, 8, 9, 10, 11, 12, 13 or 14 bp in length. In some embodiments, a stem-loop provides the oligonucleotide protection against degradation (e.g., enzymatic degradation) and facilitates or improves targeting and/or delivery to a target cell, tissue, or organ (e.g, the liver), or both. For example, in some embodiments, the loop of a stem-loop provides nucleotides comprising one or more modifications that facilitate, improve, or increase targeting to a target mRNA (e.g., an LPA mRNA), inhibition of target gene expression (e.g., LPA expression), and/or delivery to a target cell, tissue, or organ (e.g., the liver), or both. In some embodiments, the stem-loop itself or modification(s) to the stem-loop do not substantially affect the inherent gene expression inhibition activity of the oligonucleotide, but facilitates, improves, or increases stability (e.g., provides protection against degradation) and/or delivery of the oligonucleotide to a target cell, tissue, or organ (e.g., the liver). In certain embodiments, an oligonucleotide comprises a sense strand comprising (e.g., at its 3′ end) a stem-loop set forth as: S1-L-S2, in which S1 is complementary to S2, and in which L forms a single-stranded loop between S1 and S2 of up to about 10 nucleotides in length (e.g., 3, 4, 5, 6, 7, 8, 9 or 10 nucleotides in length). In some embodiments, the loop (L) is 4 nucleotides in length. FIG. 10 depicts a non-limiting example of such an oligonucleotide. In some embodiments, a loop (L) of a stem-loop having the structure S1-L-S2 as described above is a tetraloop (e.g., within a nicked tetraloop structure). In some embodiments, the tetraloop comprises ribonucleotides, deoxyribonucleotides, modified nucleotides, delivery ligands, and combinations thereof.

v. Oligonucleotide Modifications
a. Sugar Modifications

In some embodiments, a modified sugar (also referred herein to a sugar analog) includes a modified deoxyribose or ribose moiety in which, for example, one or more modifications occur at the 2′, 3′, 4′ and/or 5′ carbon position of the sugar. In some embodiments, a modified sugar may also include non-natural alternative carbon structures such as those present in locked nucleic acids (“LNA”; see, e.g., Koshkin et al. (1998) TETRAHEDON 54:3607-30), unlocked nucleic acids (“UNA”; see, e.g., Snead et al. (2013) MOL. THER-NUCL. ACIDS 2:e103) and bridged nucleic acids (“BNA”; see, e.g., Imanishi & Obika (2002) CHEM COMMUN. (CAMB) 21:1653-59).

In some embodiments, a nucleotide modification in a sugar comprises a 2′-modification. In some embodiments, a 2′-modification may be 2′-O-propargyl, 2′-O-propylamin, 2′-amino, 2′-ethyl, 2′-fluoro (2′-F), 2′-aminoethyl (EA), 2′-O-methyl (2′-OMe), 2′-O-methoxyethyl (2′-MOE), 2′-O-[2-(methylamino)-2-oxoethyl] (2′-O-NMA) or 2′-deoxy-2′-fluoro-β-d-arabinonucleic acid (2′-FANA). In some embodiments, the modification is 2′-F, 2′-OMe or 2′-MOE. In some embodiments, a modification in a sugar comprises a modification of the sugar ring, which may comprise modification of one or more carbons of the sugar ring. For example, a modification of a sugar of a nucleotide may comprise a 2′-oxygen of a sugar is linked to a 1′-carbon or 4′-carbon of the sugar, or a 2′-oxygen is linked to the 1′-carbon or 4′-carbon via an ethylene or methylene bridge. In some embodiments, a modified nucleotide has an acyclic sugar that lacks a 2′-carbon to 3′-carbon bond. In some embodiments, a modified nucleotide has a thiol group, e.g., in the 4′ position of the sugar.

In some embodiments, the oligonucleotide described herein comprises at least about 1 modified nucleotide (e.g., at least 1, at least 5, at least 10, at least 15, at least 20, at least 25, at least 30, at least 35, at least 40, at least 45, at least 50, at least 55, at least 60, or more). In some embodiments, the sense strand of the oligonucleotide comprises at least about 1 modified nucleotide (e.g., at least 1, at least 5, at least 10, at least 15, at least 20, at least 25, at least 30, at least 35, or more). In some embodiments, the antisense strand of the oligonucleotide comprises at least about 1 modified nucleotide (e.g., at least 1, at least 5, at least 10, at least 15, at least 20, or more).

In some embodiments, all the nucleotides of the sense strand of the oligonucleotide are modified. In some embodiments, all the nucleotides of the antisense strand of the oligonucleotide are modified. In some embodiments, all the nucleotides of the oligonucleotide (i.e., both the sense strand and the antisense strand) are modified. In some embodiments, the modified nucleotide comprises a 2′-modification (e.g., a 2′-F or 2′-OMe, 2′-MOE, and 2′-deoxy-2′-fluoro-β-d-arabinonucleic acid). In some embodiments, the modified nucleotide comprises a 2′-modification (e.g., a 2′-F or 2′-OMe)

The disclosure provides oligonucleotides having different modification patterns. In some embodiments, the modified oligonucleotides comprise a sense strand sequence having a modification pattern as set forth in any one of Tables 3 and 4 (as well as FIG. 10) and an antisense strand having a modification pattern as set forth in any one of Tables 3 and 4 (as well as FIG. 10). In some embodiments, for these oligonucleotides, one or more of positions 8, 9, 10 or 11 of the sense strand is modified with a 2′—F group. In other embodiments, for these oligonucleotides, the sugar moiety at each of nucleotides at positions 1-7 and 12-20 in the sense strand is modified with a 2′-OMe.

In some embodiments, the antisense strand has 3 nucleotides that are modified at the 2′-position of the sugar moiety with a 2′-F. In some embodiments, the sugar moiety at positions 2, 5 and 14 and optionally up to 3 of the nucleotides at positions 1, 3, 7 and 10 of the antisense strand are modified with a 2′-F. In other embodiments, the sugar moiety at each of the positions at positions 2, 5 and 14 of the antisense strand is modified with the 2′-F. In other embodiments, the sugar moiety at each of the positions at positions 1, 2, 5 and 14 of the antisense strand is modified with the 2′-F. In still other embodiments, the sugar moiety at each of the positions at positions 1, 2, 3, 5, 7 and 14 of the antisense strand is modified with the 2′-F. In yet another embodiment, the sugar moiety at each of the positions at positions 1, 2, 3, 5, 10 and 14 of the antisense strand is modified with the 2′-F. In another embodiment, the sugar moiety at each of the positions at positions 2, 3, 5, 7, 10 and 14 of the antisense strand is modified with the 2′-F.

### b. 5′ Terminal Phosphates

In some embodiments, 5′-terminal phosphate groups of an RNAi oligonucleotide enhance the interaction with Ago2. However, oligonucleotides comprising a 5′-phosphate group may be susceptible to degradation via phosphatases or other enzymes, which can limit their bioavailability in vivo. In some embodiments, an oligonucleotide (e.g., a ds oligonucleotide) herein includes analogs of 5′ phosphates that are resistant to such degradation. In some embodiments, the phosphate analog is oxymethylphosphonate, vinylphosphonate or malonyl phosphonate, or a combination thereof. In certain embodiments, the 3′ end of an oligonucleotide strand is attached to chemical moiety that mimics the electrostatic and steric properties of a natural 5′-phosphate group (“phosphate mimic”).

In some embodiments, an oligonucleotide has a phosphate analog at a 4′-carbon position of the sugar (referred to as a “4′-phosphate analog”). See, e.g., Intl. Patent Application Publication No. WO 2018/045317. In some embodiments, an oligonucleotide herein comprises a 4′-phosphate analog at a 5′-terminal nucleotide. In some embodiments, a phosphate analog is an oxymethylphosphonate, in which the oxygen atom of the oxymethyl group is bound to the sugar moiety (e.g., at its 4′-carbon) or analog thereof. In other embodiments, a4′-phosphate analog is a thiomethylphosphonate or an aminomethylphosphonate, in which the sulfur atom of the thiomethyl group or the nitrogen atom of the amino methyl group is bound to the 4′-carbon of the sugar moiety or analog thereof. In certain embodiments, a 4′-phosphate analog is an oxymethylphosphonate. In some embodiments, an oxymethylphosphonate is represented by the formula —O—CH₂—PO(OH)₂ or —O—CH₂—PO(OR)₂, in which R is independently selected from H, CH₃, an alkyl group, CH₂CH₂CN, CH₂OCOC(CH₃)₃, CH₂OCH₂CH₂Si(CH₃)₃ or a protecting group. In certain embodiments, the alkyl group is CH₂CH₃. More typically, R is independently selected from H, CH₃ or CH₂CH₃.

c. Modified Intranucleoside Linkages

In some embodiments, an oligonucleotide comprises a modified internucleoside linkage. In some embodiments, phosphate modifications or substitutions result in an oligonucleotide that comprises at least about 1 (e.g., at least 1, at least 2, at least 3 or at least 5) modified internucleotide linkage. In some embodiments, any one of the oligonucleotides disclosed herein comprises about 1 to about 10 (e.g., 1 to 10, 2 to 8, 4 to 6, 3 to 10, 5 to 10, 1 to 5, 1 to 3 or 1 to 2) modified internucleotide linkages. In some embodiments, any one of the oligonucleotides disclosed herein comprises 1, 2, 3, 4, 5, 6, 7, 8, 9 or 10 modified internucleotide linkages.

A modified internucleotide linkage may be a phosphorodithioate linkage, a phosphorothioate linkage, a phosphotriester linkage, a thionoalkylphosphonate linkage, a thionalkylphosphotriester linkage, a phosphoramidite linkage, a phosphonate linkage or a boranophosphate linkage. In some embodiments, at least one modified internucleotide linkage of any one of the oligonucleotides as disclosed herein is a phosphorothioate linkage.

In some embodiments, the oligonucleotide described herein has a phosphorothioate linkage between one or more of positions 1 and 2 of the sense strand, positions 1 and 2 of the antisense strand, positions 2 and 3 of the antisense strand, positions 3 and 4 of the antisense strand, positions 20 and 21 of the antisense strand, and positions 21 and 22 of the antisense strand. In some embodiments, the oligonucleotide described herein has a phosphorothioate linkage between each of positions 1 and 2 of the sense strand, positions 1 and 2 of the antisense strand, positions 2 and 3 of the antisense strand, positions 20 and 21 of the antisense strand, and positions 21 and 22 of the antisense strand.

d. Base Modifications

In some embodiments, oligonucleotides herein have one or more modified nucleobases. In some embodiments, modified nucleobases (also referred to herein as base analogs) are linked at the 1′ position of a nucleotide sugar moiety. In certain embodiments, a modified nucleobase is a nitrogenous base. In certain embodiments, a modified nucleobase does not contain nitrogen atom. See, e.g., US Patent Application Publication No. 2008/0274462. In some embodiments, a modified nucleotide comprises a universal base. However, in certain embodiments, a modified nucleotide does not contain a nucleobase (abasic).

In some embodiments, a universal base is a heterocyclic moiety located at the 1′ position of a nucleotide sugar moiety in a modified nucleotide, or the equivalent position in a nucleotide sugar moiety substitution, that, when present in a duplex, can be positioned opposite more than one type of base without substantially altering structure of the duplex. In some embodiments, compared to a reference single-stranded nucleic acid (e.g., oligonucleotide) that is fully complementary to a target nucleic acid, a single-stranded nucleic acid containing a universal base forms a duplex with the target nucleic acid that has a lower Tₘ than a duplex formed with the complementary nucleic acid. However, in some embodiments, when compared to a reference single-stranded nucleic acid in which the universal base has been replaced with a base to generate a single mismatch, the single-stranded nucleic acid containing the universal base forms a duplex with the target nucleic acid that has a higher Tₘ than a duplex formed with the nucleic acid comprising the mismatched base.

Non-limiting examples of universal-binding nucleotides include, but are not limited to, inosine, 1-β-D-ribofuranosyl-5-nitroindole and/or 1-β-D-ribofuranosyl-3-nitropyrrole (see, US Patent Application Publication No. 2007/0254362; Van Aerschot et al. (1995) NUCLEIC ACIDS RES. 23:4363-70; Loakes et al. (1995) NUCLEIC ACIDS RES. 23:2361-66; and Loakes & Brown (1994) NUCLEIC ACIDS RES. 22:4039-43).

e. Reversible Modifications

While certain modifications to protect an oligonucleotide from the in vivo environment before reaching target cells can be made, they can reduce the potency or activity of the oligonucleotide once it reaches the cytosol of the target cell. Reversible modifications can be made such that the molecule retains desirable properties outside of the cell, which are then removed upon entering the cytosolic environment of the cell. Reversible modification can be removed, for example, by the action of an intracellular enzyme or by the chemical conditions inside of a cell (e.g., through reduction by intracellular glutathione).

In some embodiments, a reversibly modified nucleotide comprises a glutathione-sensitive moiety. Typically, nucleic acid molecules have been chemically modified with cyclic disulfide moieties to mask the negative charge created by the internucleotide diphosphate linkages and improve cellular uptake and nuclease resistance. See US Patent Application Publication No. 2011/0294869, Intl. Patent Application Publication Nos. WO 2014/088920 and WO 2015/188197, and Meade et al. (2014) NAT. BIOTECHNOL. 32:1256-63. This reversible modification of the internucleotide diphosphate linkages is designed to be cleaved intracellularly by the reducing environment of the cytosol (e.g. glutathione). Earlier examples include neutralizing phosphotriester modifications that were reported to be cleavable inside cells (see, Dellinger et al. (2003) J. AM. CHEM. SOC. 125:940-50).

In some embodiments, such a reversible modification allows protection during in vivo administration (e.g., transit through the blood and/or lysosomal/endosomal compartments of a cell) where the oligonucleotide will be exposed to nucleases and other harsh environmental conditions (e.g., pH). When released into the cytosol of a cell where the levels of glutathione are higher compared to extracellular space, the modification is reversed, and the result is a cleaved oligonucleotide. Using reversible, glutathione-sensitive moieties, it is possible to introduce sterically larger chemical groups into the oligonucleotide of interest when compared to the options available using irreversible chemical modifications. This is because these larger chemical groups will be removed in the cytosol and, therefore, should not interfere with the biological activity of the oligonucleotides inside the cytosol of a cell. As a result, these larger chemical groups can be engineered to confer various advantages to the nucleotide or oligonucleotide, such as nuclease resistance, lipophilicity, charge, thermal stability, specificity and reduced immunogenicity. In some embodiments, the structure of the glutathione-sensitive moiety can be engineered to modify the kinetics of its release.

In some embodiments, a glutathione-sensitive moiety is attached to the sugar of the nucleotide. In some embodiments, a glutathione-sensitive moiety is attached to the 2′-carbon of the sugar of a modified nucleotide. In some embodiments, the glutathione-sensitive moiety is located at the 5′-carbon of a sugar, particularly when the modified nucleotide is the 5′-terminal nucleotide of the oligonucleotide. In some embodiments, the glutathione-sensitive moiety is located at the 3′-carbon of sugar, particularly when the modified nucleotide is the 3′-terminal nucleotide of the oligonucleotide. In some embodiments, the glutathione-sensitive moiety comprises a sulfonyl group. See, e.g., U.S. Provisional Patent Application No. 62/378,635, entitled Compositions Comprising Reversibly Modified Oligonucleotides and Uses Thereof, which was filed on Aug. 23, 2016.

vi. Targeting Ligands

In some embodiments, it is desirable to target the oligonucleotides of the disclosure to one or more cells or one or more organs. Such a strategy can help to avoid undesirable effects in other organs or avoid undue loss of the oligonucleotide to cells, tissue or organs that would not benefit from the oligonucleotide. Accordingly, in some embodiments, oligonucleotides disclosed herein are modified to facilitate targeting and/or delivery to a tissue, cell or organ (e.g., to facilitate delivery of the oligonucleotide to the liver). In certain embodiments, oligonucleotides disclosed herein are modified to facilitate delivery of the oligonucleotide to the hepatocytes of the liver. In some embodiments, an oligonucleotide comprises at least one nucleotide (e.g., 1, 2, 3, 4, 5, 6 or more nucleotides) conjugated to one or more targeting ligand(s).

In some embodiments, the targeting ligand comprises a carbohydrate, amino sugar, cholesterol, peptide, polypeptide, protein or part of a protein (e.g., an antibody or antibody fragment), or lipid. In some embodiments, the targeting ligand is an aptamer. For example, a targeting ligand may be an RGD peptide that is used to target tumor vasculature or glioma cells, CREKA peptide to target tumor vasculature or stoma, transferring, lactoferrin, or an aptamer to target transferrin receptors expressed on CNS vasculature, or an anti-EGFR antibody to target EGFR on glioma cells. In certain embodiments, the targeting ligand is one or more GalNAc moieties.

In some embodiments, 1 or more (e.g., 1, 2, 3, 4, 5 or 6) nucleotides of an oligonucleotide are each conjugated to a separate targeting ligand. In some embodiments, 2 to 4 nucleotides of an oligonucleotide are each conjugated to a separate targeting ligand. In some embodiments, targeting ligands are conjugated to 2 to 4 nucleotides at either ends of the sense or antisense strand (e.g., targeting ligands are conjugated to a 2 to 4 nucleotide overhang or extension on the 5′ or 3′ end of the sense or antisense strand) such that the targeting ligands resemble bristles of a toothbrush and the oligonucleotide resembles a toothbrush. For example, an oligonucleotide may comprise a stem-loop at either the 5′ or 3′ end of the sense strand and 1, 2, 3 or 4 nucleotides of the loop of the stem may be individually conjugated to a targeting ligand. In some embodiments, an oligonucleotide (e.g., a ds oligonucleotide) provided by the disclosure comprises a stem-loop at the 3′ end of the sense strand, wherein the loop of the stem-loop comprises a triloop or a tetraloop, and wherein the 3 or 4 nucleotides comprising the triloop or tetraloop, respectfully, are individually conjugated to a targeting ligand.

GalNAc is a high affinity ligand for the ASGPR, which is primarily expressed on the sinusoidal surface of hepatocyte cells and has a major role in binding, internalizing and subsequent clearing circulating glycoproteins that contain terminal galactose or GalNAc residues (asialoglycoproteins). Conjugation (either indirect or direct) of GalNAc moieties to oligonucleotides of the instant disclosure can be used to target these oligonucleotides to the ASGPR expressed on cells. In some embodiments, an oligonucleotide of the instant disclosure is conjugated to at least one or more GalNAc moieties, wherein the GalNAc moieties target the oligonucleotide to an ASGPR expressed on human liver cells (e.g. human hepatocytes). In some embodiments, the GalNAc moiety target the oligonucleotide to the liver.

In some embodiments, an oligonucleotide of the instant disclosure is conjugated directly or indirectly to a monovalent GalNAc. In some embodiments, the oligonucleotide is conjugated directly or indirectly to more than one monovalent GalNAc (i.e., is conjugated to 2, 3 or 4 monovalent GalNAc moieties, and is typically conjugated to 3 or 4 monovalent GalNAc moieties). In some embodiments, an oligonucleotide is conjugated to one or more bivalent GalNAc, trivalent GalNAc or tetravalent GalNAc moieties.

In some embodiments, 1 or more (e.g., 1, 2, 3, 4, 5 or 6) nucleotides of an oligonucleotide are each conjugated to a GalNAc moiety. In some embodiments, 2 to 4 nucleotides of a tetraloop are each conjugated to a separate GalNAc. In some embodiments, 1 to 3 nucleotides of a triloop are each conjugated to a separate GalNAc. In some embodiments, targeting ligands are conjugated to 2 to 4 nucleotides at either ends of the sense or antisense strand (e.g., ligands are conjugated to a 2 to 4 nucleotide overhang or extension on the 5′ or 3′ end of the sense or antisense strand) such that the GalNAc moieties resemble bristles of a toothbrush and the oligonucleotide resembles a toothbrush. In some embodiments, GalNAc moieties are conjugated to a nucleotide of the sense strand. For example, 4 GalNAc moieties can be conjugated to nucleotides in the tetraloop of the sense strand where each GalNAc moiety is conjugated to 1 nucleotide.

In some embodiments, an oligonucleotide herein comprises a monovalent GalNAc attached to a guanine nucleotide referred to as [ademG-GalNAc] or 2′-aminodiethoxymethanol-Guanine-GalNAc, as depicted below:

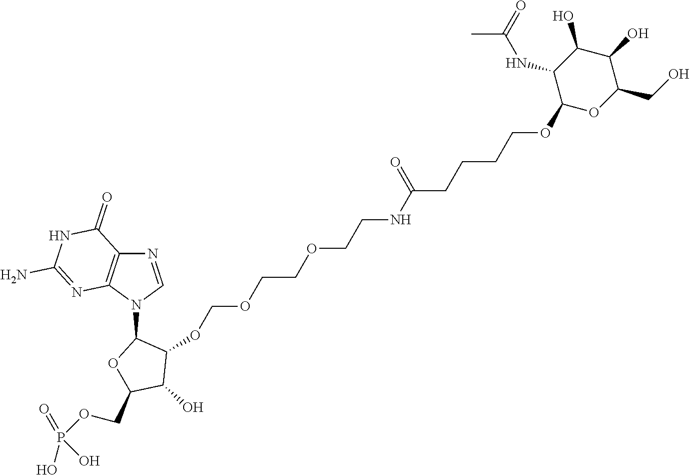 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(=O)[nH]c(N)nc32)O[C@H](CO)[C@H](O)[C@@H]1O]

In some embodiments, an oligonucleotide herein comprises a monovalent GalNAc attached to an adenine nucleotide, referred to as [ademA-GalNAc] or 2′-aminodiethoxymethanol-Adenine-GalNAc, as depicted below:

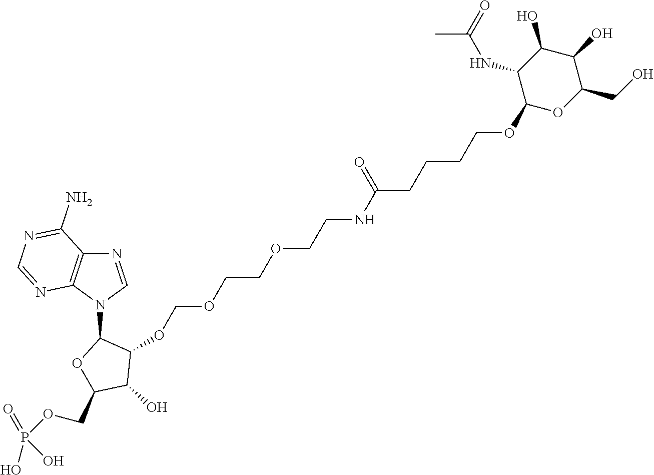

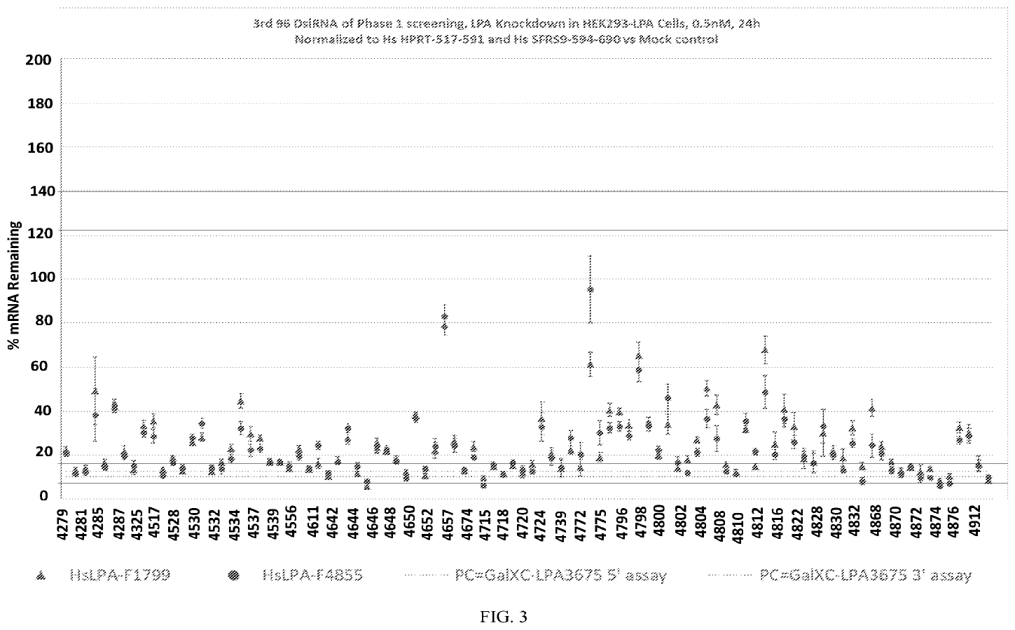

An example of such conjugation is shown below for a loop comprising from 5′ to 3′ the nucleotide sequence GAAA (L=linker, X=heteroatom) stem attachment points are shown. Such a loop may be present, for example, at positions 27-30 of the sense strand listed in Table 5 and as shown in FIG. 3. In the chemical formula,

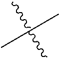

is used to describe an attachment point to the oligonucleotide strand.

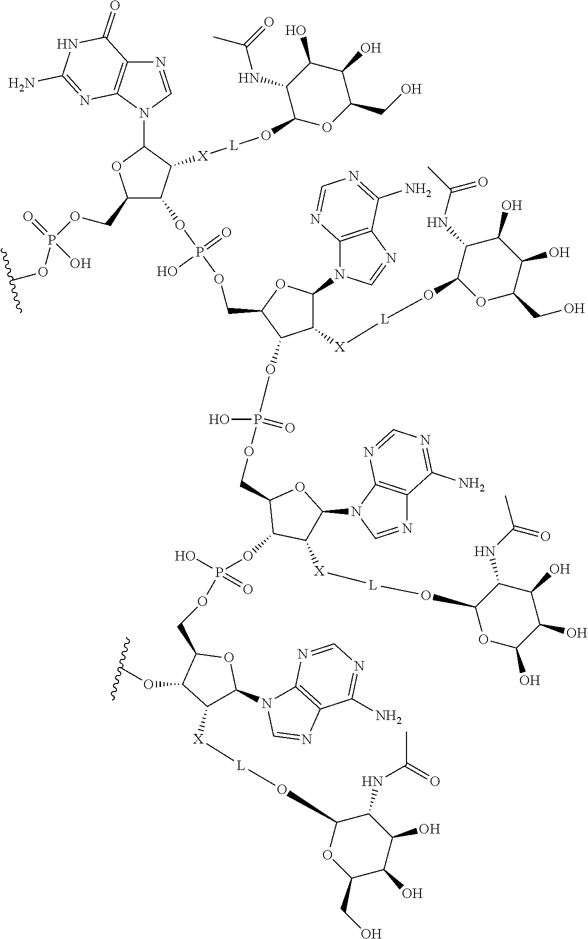

Appropriate methods or chemistry (e.g., click chemistry) can be used to link a targeting ligand to a nucleotide. In some embodiments, a targeting ligand is conjugated to a nucleotide using a click linker. In some embodiments, an acetal-based linker is used to conjugate a targeting ligand to a nucleotide of any one of the oligonucleotides described herein. Acetal-based linkers are disclosed, for example, in Intl. Patent Application Publication No. WO 2016/100401. In some embodiments, the linker is a labile linker. However, in other embodiments, the linker is stable. An example is shown below for a loop comprising from 5′ to 3′ the nucleotides GAAA, in which GalNAc moieties are attached to nucleotides of the loop using an acetal linker. Such a loop may be present, for example, at positions 27-30 of the any one of the sense strand listed in Tables 3 or 4 and as shown in FIG. 10. In the chemical formula,

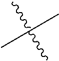

is an attachment point to the oligonucleotide strand.

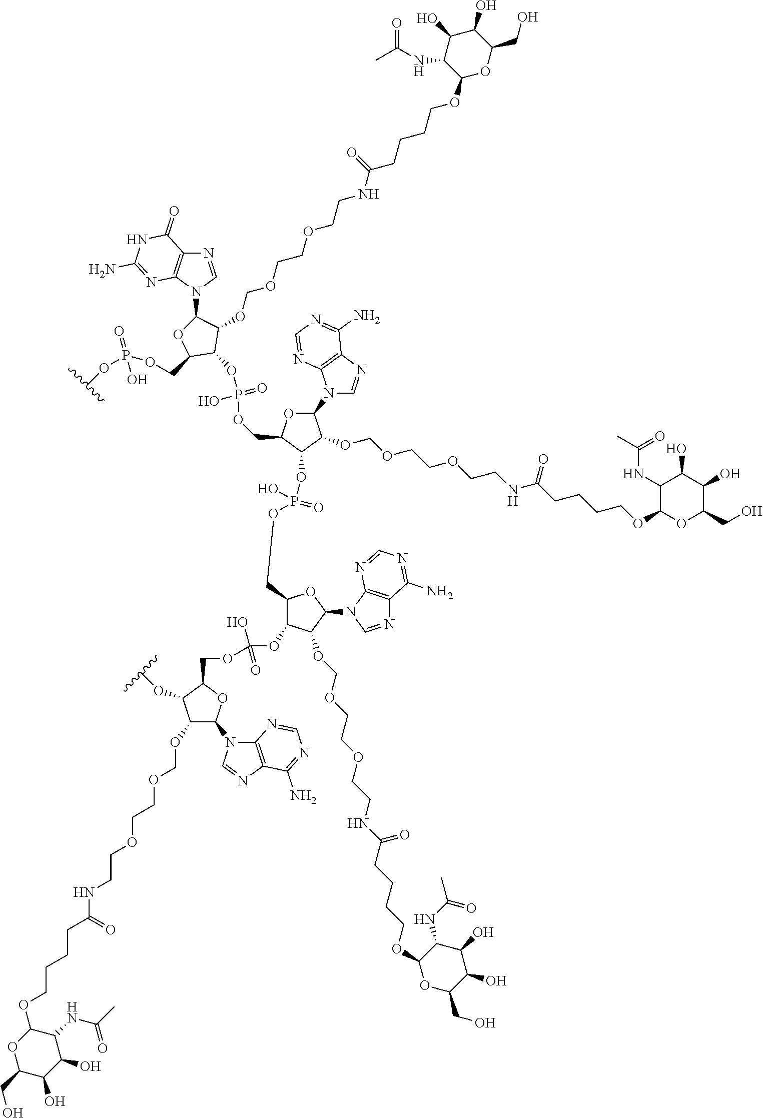

As mentioned, various appropriate methods or chemistry synthetic techniques (e.g., click chemistry) can be used to link a targeting ligand to a nucleotide. In some embodiments, a targeting ligand is conjugated to a nucleotide using a click linker. In some embodiments, an acetal-based linker is used to conjugate a targeting ligand to a nucleotide of any one of the oligonucleotides described herein. Acetal-based linkers are disclosed, for example, in Intl. Patent Application Publication No. WO 2016/100401. In some embodiments, the linker is a labile linker. However, in other embodiments, the linker is a stable linker.

In some embodiments, a duplex extension (e.g., of up to 3, 4, 5 or 6 bp in length) is provided between a targeting ligand (e.g., a GalNAc moiety) and a ds oligonucleotide. In some embodiments, the oligonucleotides herein do not have a GalNAc conjugated thereto.

### III. Formulations

Various formulations have been developed to facilitate oligonucleotide use. For example, oligonucleotides can be delivered to a subject or a cellular environment using a formulation that minimizes degradation, facilitates delivery and/or uptake, or provides another beneficial property to the oligonucleotides in the formulation. In some embodiments, an oligonucleotide is formulated in buffer solutions such as phosphate buffered saline solutions, liposomes, micellar structures and capsids.

Formulations of oligonucleotides with cationic lipids can be used to facilitate transfection of the oligonucleotides into cells. For example, cationic lipids, such as lipofectin, cationic glycerol derivatives, and polycationic molecules (e.g., polylysine, can be used. Suitable lipids include Oligofectamine, Lipofectamine (Life Technologies), NC388 (Ribozyme Pharmaceuticals, Inc., Boulder, Colo.), or FuGene 6 (Roche) all of which can be used according to the manufacturer's instructions.

Accordingly, in some embodiments, a formulation comprises a lipid nanoparticle. In some embodiments, an excipient comprises a liposome, a lipid, a lipid complex, a microsphere, a microparticle, a nanosphere or a nanoparticle, or may be otherwise formulated for administration to the cells, tissues, organs, or body of a subject in need thereof (see, e.g., Remington: THE SCIENCE AND PRACTICE OF PHARMACY, 22nd edition, Pharmaceutical Press, 2013).

In some embodiments, the formulations herein comprise an excipient. In some embodiments, an excipient confers to a composition improved stability, improved absorption, improved solubility and/or therapeutic enhancement of the active ingredient. In some embodiments, an excipient is a buffering agent (e.g., sodium citrate, sodium phosphate, a tris base, or sodium hydroxide) or a vehicle (e.g., a buffered solution, petrolatum, dimethyl sulfoxide or mineral oil). In some embodiments, an oligonucleotide is lyophilized for extending its shelf-life and then made into a solution before use (e.g., administration to a subject). Accordingly, an excipient in a composition comprising any one of the oligonucleotides described herein may be a lyoprotectant (e.g., mannitol, lactose, polyethylene glycol or polyvinylpyrrolidone) or a collapse temperature modifier (e.g., dextran, Ficoll™ or gelatin).

In some embodiments, a pharmaceutical composition is formulated to be compatible with its intended route of administration. Examples of routes of administration include parenteral (e.g., intravenous, intramuscular, intraperitoneal, intradermal, subcutaneous), oral (e.g., inhalation), transdermal (e.g., topical), transmucosal and rectal administration.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). The carrier can be a solvent or dispersion medium containing, for example, water, ethanol, polyol (e.g., glycerol, propylene glycol, and liquid polyethylene glycol, and the like), and suitable mixtures thereof. In many cases, it will be preferable to include isotonic agents, for example, sugars, polyalcohols such as mannitol, sorbitol, sodium chloride in the composition. Sterile injectable solutions can be prepared by incorporating the oligonucleotides in a required amount in a selected solvent with one or a combination of ingredients enumerated above, as required, followed by filtered sterilization.

In some embodiments, a composition may contain at least about 0.1% of the therapeutic agent or more, although the percentage of the active ingredient(s) may be between about 1% to about 80% or more of the weight or volume of the total composition. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

Even though several embodiments are directed to liver-targeted delivery of any of the oligonucleotides herein, targeting of other tissues is also contemplated.

### IV. Methods of Use

i. Reducing LPA Expression in Cells

The disclosure provides methods for contacting or delivering to a cell or population of cells an effective amount any of the oligonucleotides (e.g., a ds oligonucleotide) herein for purposes of reducing LPA expression. In some embodiments, a reduction of LPA expression is determined by measuring a reduction in the amount or level of LPA mRNA, apo(a) protein, or apo(a) activity in a cell. The methods can include the steps described herein, and these maybe be, but not necessarily, carried out in the sequence as described. Other sequences, however, also are conceivable. Moreover, individual or multiple steps bay be carried out either in parallel and/or overlapping in time and/or individually or in multiply repeated steps. Furthermore, the methods may include additional, unspecified steps.

Methods herein are useful in any appropriate cell type. In some embodiments, a cell is any cell that expresses mRNA (e.g., hepatocytes, macrophages, monocyte-derived cells, prostate cancer cells, cells of the brain, endocrine tissue, bone marrow, lymph nodes, lung, gall bladder, liver, duodenum, small intestine, pancreas, kidney, gastrointestinal tract, bladder, adipose and soft tissue and skin). In some embodiments, the cell is a primary cell obtained from a subject. In some embodiments, the primary cell has undergone a limited number of passages such that the cell substantially maintains is natural phenotypic properties. In some embodiments, a cell to which the oligonucleotide is delivered is ex vivo or in vitro (i.e., can be delivered to a cell in culture or to an organism in which the cell resides).

In some embodiments, the oligonucleotides herein are delivered to a cell or population of cells using a nucleic acid delivery method known in the art including, but not limited to, injection of a solution containing the oligonucleotide, bombardment by particles covered by the oligonucleotide, exposing the cell or population of cells to a solution containing the oligonucleotide, or electroporation of cell membranes in the presence of the oligonucleotide. Other methods known in the art for delivering oligonucleotides to cells may be used, such as lipid-mediated carrier transport, chemical-mediated transport, and cationic liposome transfection such as calcium phosphate, and others.

In some embodiments, reduction of LPA expression is determined by an assay or technique that evaluates one or more molecules, properties or characteristics of a cell or population of cells associated with LPA expression (e.g., using an LPA expression biomarker) or by an assay or technique that evaluates molecules that are directly indicative of LPA expression in a cell or population of cells (e.g., LPA mRNA or apo(a) protein). In some embodiments, the extent to which an oligonucleotide herein reduces LPA expression is evaluated by comparing LPA expression in a cell or population of cells contacted with the oligonucleotide to a control cell or population of cells (e.g., a cell or population of cells not contacted with the oligonucleotide or contacted with a control oligonucleotide). In some embodiments, a control amount or level of LPA expression in a control cell or population of cells is predetermined, such that the control amount or level need not be measured in every instance the assay or technique is performed. The predetermined level or value can take a variety of forms. In some embodiments, a predetermined level or value can be single cut-off value, such as a median or mean.

In some embodiments, contacting or delivering an oligonucleotide (e.g., a ds oligonucleotide) herein to a cell or a population of cells results in a reduction in LPA expression. In some embodiments, the reduction in LPA expression is relative to a control amount or level of LPA expression in cell or population of cells not contacted with the oligonucleotide or contacted with a control oligonucleotide. In some embodiments, the reduction in LPA expression is about 1% or lower, about 5% or lower, about 10% or lower, about 15% or lower, about 20% or lower, about 25% or lower, about 30% or lower, about 35% or lower, about 40% or lower, about 45% or lower, about 50% or lower, about 55% or lower, about 60% or lower, about 70% or lower, about 80% or lower, or about 90% or lower relative to a control amount or level of LPA expression. In some embodiments, the control amount or level of LPA expression is an amount or level of LPA mRNA and/or apo(a) protein in a cell or population of cells that has not been contacted with an oligonucleotide herein. In some embodiments, the effect of delivery of an oligonucleotide to a cell or population of cells according to a method herein is assessed after any finite period or amount of time (e.g., minutes, hours, days, weeks, months). For example, in some embodiments, LPA expression is determined in a cell or population of cells at least about 4 hours, about 8 hours, about 12 hours, about 18 hours, about 24 hours; or at least about 1 day, about 2 days, about 3 days, about 4 days, about 5 days, about 6 days, about 7 days, about 8 days, about 9 days, about 10 days, about 11 days, about 12 days, about 13 days, about 14 days, about 21 days, about 28 days, about 35 days, about 42 days, about 49 days, about 56 days, about 63 days, about 70 days, about 77 days, or about 84 days or more after contacting or delivering the oligonucleotide to the cell or population of cells. In some embodiments, LPA expression is determined in a cell or population of cells at least about 1 month, about 2 months, about 3 months, about 4 months, about 5 months, or about 6 months or more after contacting or delivering the oligonucleotide to the cell or population of cells.

In some embodiments, an oligonucleotide is delivered in the form of a transgene that is engineered to express in a cell the oligonucleotide or strands comprising the oligonucleotide (e.g., its sense and antisense strands). In some embodiments, an oligonucleotide is delivered using a transgene engineered to express any oligonucleotide disclosed herein. Transgenes may be delivered using viral vectors (e.g., adenovirus, retrovirus, vaccinia virus, poxvirus, adeno-associated virus or herpes simplex virus) or non-viral vectors (e.g., plasmids or synthetic mRNAs). In some embodiments, transgenes can be injected directly to a subject.

ii. Medical Use

The disclosure also provides oligonucleotides for use, or adaptable for use, to treat a subject (e.g., a human having a disease, disorder or condition associated with LPA expression) that would benefit from reducing LPA expression. In some embodiments, the disclosure provides oligonucleotides for use, or adapted for use, to treat a subject having a disease, disorder or condition associated with expression of LPA. The disclosure also provides oligonucleotides for use, or adaptable for use, in the manufacture of a medicament or pharmaceutical composition for treating a disease, disorder or condition associated with LPA expression. In some embodiments, the oligonucleotides for use, or adaptable for use, target LPA mRNA and reduce LPA expression (e.g., via the RNAi pathway). In some embodiments, the oligonucleotides for use, or adaptable for use, target LPA mRNA and reduce the amount or level of LPA mRNA, apo(a) protein and/or apo (a) activity.

In addition, in some embodiments of the methods herein, a subject having a disease, disorder or condition associated with LPA expression or is predisposed to the same is selected for treatment with an oligonucleotide (e.g., a ds oligonucleotide) herein. In some embodiments, the method comprises selecting an individual having a marker (e.g., a biomarker) for a disease, disorder or condition associated with LPA expression, or predisposed to the same, such as, but not limited to, LPA mRNA, apo(a) protein, lipoprotein(a), or a combination thereof. Likewise, and as detailed below, some embodiments of the methods provided by the disclosure include steps such as measuring or obtaining a baseline value for a marker of LPA expression (e.g., lipoprotein(a)), and then comparing such obtained value to one or more other baseline values or values obtained after the subject is administered the oligonucleotide to assess the effectiveness of treatment.

iii. Methods of Treatment

The disclosure also provides methods of treating a subject having, suspected of having, or at risk of developing a disease, disorder or condition associated with LPA expression with an oligonucleotide herein. In some embodiments, the disclosure provides methods of treating or attenuating the onset or progression of a disease, disorder or condition associated with LPA expression using the oligonucleotides herein. In other embodiments, the disclosure provides methods to achieve one or more therapeutic benefits in a subject having a disease, disorder or condition associated with LPA expression using the oligonucleotides herein. In some embodiments of the methods herein, the subject is treated by administering a therapeutically effective amount of any one or more of the oligonucleotides herein. In some embodiments, treatment comprises reducing LPA expression. In some embodiments, the subject is treated therapeutically. In some embodiments, the subject is treated prophylactically.

In some embodiments of the methods herein, an oligonucleotide herein, or a pharmaceutical composition comprising the oligonucleotide, is administered to a subject having a disease, disorder or condition associated with LPA expression such that LPA expression is reduced in the subject, thereby treating the subject. In some embodiments, an amount or level of LPA mRNA is reduced in the subject. In some embodiments, an amount or level of apo(a) protein is reduced in the subject. In some embodiments, an amount or level of lipoprotein(a) is reduced in the subject. In some embodiments, an amount or level of apo(a) activity is reduced in the subject. In some embodiments, an amount or level of triglyceride (TG) (e.g., one or more TG(s) or total TGs) is reduced in the subject. In some embodiments, an amount or level of cholesterol (e.g., total cholesterol, LDL cholesterol, and/or HDL cholesterol) is reduced in the subject. In some embodiments, an amount or level of low-density lipoprotein (LDL) cholesterol is reduced in the subject. In some embodiments, an amount or activity of OxPL is reduced or altered in the subject. In some embodiments, an amount or activity of LDL-C is reduced or altered in the subject. In some embodiments, an amount or activity of apoB-100 is reduced or altered in the subject. In some embodiments, any combination of the following is reduced or altered in the subject: LPA expression, an amount or level of LPA mRNA, an amount or level of apo(a) protein, an amount or level of apo(a) activity, an amount or level of TG, an amount or level of cholesterol, an amount or activity of OxPL, an amount or activity of LDL-C, and/or an amount or activity of apoB-100.

In some embodiments of the methods herein, an oligonucleotide herein, or a pharmaceutical composition comprising the oligonucleotide, is administered to a subject having a disease, disorder or condition associated with LPA expression such that LPA expression is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to LPA expression prior to administration of the oligonucleotide or pharmaceutical composition. In some embodiments, LPA expression is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to LPA expression in a subject (e.g., a reference or control subject) not receiving the oligonucleotide or pharmaceutical composition or receiving a control oligonucleotide, pharmaceutical composition or treatment.

In some embodiments of the methods herein, an oligonucleotide herein, or a pharmaceutical composition comprising the oligonucleotide, is administered to a subject having a disease, disorder or condition associated with LPA expression such that an amount or level of LPA mRNA is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of LPA mRNA prior to administration of the oligonucleotide or pharmaceutical composition. In some embodiments, an amount or level of LPA mRNA is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of LPA mRNA in a subject (e.g., a reference or control subject) not receiving the oligonucleotide or pharmaceutical composition or receiving a control oligonucleotide, pharmaceutical composition or treatment.

In some embodiments of the methods herein, an oligonucleotide herein, or a pharmaceutical composition comprising the oligonucleotide, is administered to a subject having a disease, disorder or condition associated with LPA expression such that an amount or level of apo(a) protein is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of apo(a) protein prior to administration of the oligonucleotide or pharmaceutical composition. In some embodiments, an amount or level of apo(a) protein is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of apo(a) protein in a subject (e.g., a reference or control subject) not receiving the oligonucleotide or pharmaceutical composition or receiving a control oligonucleotide, pharmaceutical composition or treatment.

In some embodiments of the methods herein, an oligonucleotide herein, or a pharmaceutical composition comprising the oligonucleotide, is administered to a subject having a disease, disorder or condition associated with LPA expression such that an amount or level of apo(a) activity is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of apo(a) activity prior to administration of the oligonucleotide or pharmaceutical composition. In some embodiments, an amount or level of apo(a) activity is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of apo(a) activity in a subject (e.g., a reference or control subject) not receiving the oligonucleotide or pharmaceutical composition or receiving a control oligonucleotide, pharmaceutical composition or treatment.

In some embodiments of the methods herein, an oligonucleotide herein, or a pharmaceutical composition comprising the oligonucleotide, is administered to a subject having a disease, disorder or condition associated with LPA expression such that an amount or level of lipoprotein(a) is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of lipoprotein(a) prior to administration of the oligonucleotide or pharmaceutical composition. In some embodiments, an amount or level of lipoprotein(a) is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of lipoprotein(a) in a subject (e.g., a reference or control subject) not receiving the oligonucleotide or pharmaceutical composition or receiving a control oligonucleotide, pharmaceutical composition or treatment.

Lipoprotein(a) levels range widely in human adults with plasma levels ranging from <0.1 mg/dL to >200 mg/dL, thus exhibiting up to three orders of magnitude difference among individuals (Schmidt et al., (2016) J Lipid Res. 57(8):1339-1359). Lipoprotein(a) levels <30 mg/dl are considered optimal in the United States and Canada (Anderson et al., (2016) CAN J CARDIOL 32:1263-82). The European Atherosclerosis Society (EAS) has proposed <50 mg/dL as optimal, and lipoprotein(a) levels >60 mg/dl are used as a cutoff for the reimbursement of apheresis in Germany and the United Kingdom (Tsimikas (2017) J AM COLL CARDIOL. 69(6):692-711). In some embodiments, a subject selected for treatment or treated with an oligonucleotide herein is identified or determined to have an amount or level of lipoprotein(a) of about 30 mg/dL or greater. In some embodiments, a subject selected for treatment or treated with an oligonucleotide herein is identified or determined to have an amount or level of lipoprotein(a) of >30 mg/dL. In some embodiments, a subject selected for treatment or treated with an oligonucleotide herein is identified or determined to have an amount or level of lipoprotein(a) of about 50 mg/dL or greater. In some embodiments, a subject selected for treatment or treated with an oligonucleotide herein is identified or determined to have an amount or level of lipoprotein(a) of about 60 mg/dL or greater. In some embodiments, a subject selected for treatment or treated with an oligonucleotide herein is identified or determined to have an amount or level of lipoprotein(a) in the range of 30 mg/dL to 300 mg/dL.

Generally, a normal or desirable TG range for a human patient is <150 mg/dL of blood, with <100 being considered ideal. In some embodiments, the patient selected for treatment or treated is identified or determined to have an amount or level of TG of ≥150 mg/dL. In some embodiments, the patient selected for treatment or treated is identified or determined to have an amount or level of TG in the range of 150 to 199 mg/dL, which is considered borderline high TG levels. In some embodiments, the patient selected for treatment or treated is identified or determined to have an amount or level of TG in the range of 200 to 499 mg/dL, which is considered high TG levels. In some embodiments, the patient selected for treatment or treated is identified or determined to have an amount or level of TG in the range of 500 mg/dL or higher (i.e., ≥500 mg/dL), which is considered very high TG levels. In some embodiments, the patient selected for treatment or treated is identified or determined to have an amount or level of TG which is ≥150 mg/dL, ≥200 mg/dL or ≥500 mg/dL. In some embodiments, the patient selected for treatment or treated is identified or determined to have an amount of level of TG of 200 to 499 mg/dL, or 500 mg/dL or higher. In some embodiments, the patient selected for treatment or treated is identified or determined to have an amount or level of TG which is ≥200 mg/dL.

In some embodiments of the methods herein, an oligonucleotide herein, or a pharmaceutical composition comprising the oligonucleotide, is administered to a subject having a disease, disorder or condition associated with LPA expression such that an amount or level of cholesterol (e.g., total cholesterol, LDL cholesterol, and/or HDL cholesterol) is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of cholesterol prior to administration of the oligonucleotide or pharmaceutical composition. In some embodiments, an amount or level of cholesterol is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of cholesterol in a subject (e.g., a reference or control subject) not receiving the oligonucleotide or pharmaceutical composition or receiving a control oligonucleotide, pharmaceutical composition or treatment.

Generally, a normal or desirable cholesterol range (total cholesterol) for an adult human patient is <200 mg/dL of blood. In some embodiments, the patient selected for treatment or treated is identified or determined to have an amount or level of cholesterol of ≥200 mg/dL. In some embodiments, the patient selected for treatment or treated is identified or determined to have an amount or level of cholesterol in the range of 200 to 239 mg/dL, which is considered borderline high cholesterol levels. In some embodiments, the patient selected for treatment or treated is identified or determined to have an amount or level of cholesterol in the range of 240 mg/dL and higher (i.e., ≥240 mg/dL), which is considered high cholesterol levels. In some embodiments, the patient selected from treatment or treated is identified or determined to have an amount or level of cholesterol of 200 to 239 mg/dL, or 240 mg/dL or higher. In some embodiments, the patient selected for treatment or treated is identified or determined to have an amount or level of cholesterol which is ≥200 mg/dL or ≥240 mg/dL or higher.

In some embodiments of the methods herein, an oligonucleotide herein, or a pharmaceutical composition comprising the oligonucleotide, is administered to a subject having a disease, disorder, or condition associated with LPA expression such that an amount or level of LDL cholesterol is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of LDL cholesterol prior to administration of the oligonucleotide or pharmaceutical composition. In some embodiments, an amount or level of LDL cholesterol is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of LDL cholesterol in a subject (e.g., a reference or control subject) not receiving the oligonucleotide or pharmaceutical composition or receiving a control oligonucleotide, pharmaceutical composition or treatment.

Generally, a normal or desirable LDL cholesterol range for an adult human patient is <100 mg/dL of blood. In some embodiments, the patient selected for treatment or treated is identified or determined to have an amount or level of cholesterol of ≥100 mg/dL. In some embodiments, the patient selected for treatment or treated is identified or determined to have an amount or level of LDL cholesterol in the range of 100 to 129 mg/dL, which is considered above optimal. In some embodiments, the patient selected for treatment or treated is identified or determined to have an amount or level of LDL cholesterol in the range of 130 to 159 mg/dL, which is considered borderline high levels. In some embodiments, the patient selected for treatment or treated is identified or determined to have an amount or level of LDL cholesterol in the range of 160 to 189 mg/dL, which is considered high LDL cholesterol levels. In some embodiments, the patient selected for treatment or treated is identified or determined to have an amount or level of LDL cholesterol in the range of 190 mg/dL and higher (i.e., ≥190 mg/dL), which is considered very high LDL cholesterol levels. In some embodiments, the patient selected for treatment or treated is identified or determined to have an amount or level of LDL cholesterol which is ≥100 mg/dL, ≥130 mg/dL, ≥160 mg/dL, or ≥190 mg/dL or higher, preferably ≥160 mg/dL, or ≥190 mg/dL or higher. In some embodiments, the patient selected for treatment or treated is identified or determined to have an amount or level of LDL cholesterol of 100 to 129 mg/dL, 130 to 159 mg/dL, 160 to 189 mg/dL, or 190 mg/dL and higher.

Suitable methods for determining LPA expression, an amount or level of LPA mRNA, an amount or level of apo(a) protein, an amount or level of apo(a) activity, an amount or level of lipoprotein(a), and/or an amount or level of OxPL, LDL-C, apoB-100, TG and/or LDL cholesterol in the subject, or in a sample from the subject, are known in the art. Further, the Examples set forth herein illustrate exemplary methods for determining LPA expression.

In some embodiments, LPA expression, the amount or level of LPA mRNA, apo(a) protein, apo(a) activity, OxPL, LDL-C, apoB-100, TG, LDL cholesterol, or any combination thereof, is reduced in a cell (e.g., a hepatocyte), a population or a group of cells (e.g., an organoid), an organ (e.g., liver), blood or a fraction thereof (e.g., plasma), a tissue (e.g., liver tissue), a sample (e.g., a liver biopsy sample), or any other biological material obtained or isolated from the subject. In some embodiments, LPA expression, the amount or level of LPA mRNA, apo(a) protein, apo(a) activity, OxPL, LDL-C, apoB-100, TG, LDL cholesterol, or any combination thereof, is reduced in more than one type of cell (e.g., a hepatocyte and one or more other type(s) of cell), more than one groups of cells, more than one organ (e.g., liver and one or more other organ(s)), more than one fraction of blood (e.g., plasma and one or more other blood fraction(s)), more than one type of tissue (e.g., liver tissue and one or more other type(s) of tissue), more than one type of sample (e.g., a liver biopsy sample and one or more other type(s) of biopsy sample) obtained or isolated from the subject.

Examples of a disease, disorder or condition associated with LPA expression include, but are not limited to, Berger's disease, peripheral artery disease, coronary artery disease, metabolic syndrome, acute coronary syndrome, aortic valve stenosis, aortic valve regurgitation, aortic dissection, retinal artery occlusion, cerebrovascular disease, mesenteric ischemia, superior mesenteric artery occlusion, renal artery stenosis, stable/unstable angina, acute coronary syndrome, heterozygous or homozygous familial hypercholesterolemia, hyperapobetalipoproteinemia, cerebrovascular atherosclerosis, cerebrovascular disease, and venous thrombosis, or a combination thereof.

Because of their high specificity, the oligonucleotides herein specifically target mRNAs of target genes of cells, tissues, or organs (e.g., liver). In preventing disease, the target gene may be one which is required for initiation or maintenance of the disease or which has been identified as being associated with a higher risk of contracting the disease. In treating disease, the oligonucleotide can be brought into contact with the cells or tissue exhibiting or responsible for mediating the disease. For example, an oligonucleotide substantially identical to all or part of a wild-type (i.e., native) or mutated gene associated with a disorder or condition associated with LPA expression may be brought into contact with or introduced into a cell or tissue type of interest such as a hepatocyte or other liver cell.

In some embodiments, the target gene may be a target gene from any mammal, such as a human. Any gene may be silenced according to the method described herein.

Methods described herein are typically involve administering to a subject a therapeutically effective amount of an oligonucleotide herein, that is, an amount capable of producing a desirable therapeutic result. A therapeutically acceptable amount may be an amount that can therapeutically treat a disease or disorder. The appropriate dosage for any one subject will depend on certain factors, including the subject's size, body surface area, age, the particular composition to be administered, the active ingredient(s) in the composition, time and route of administration, general health, and other drugs being administered concurrently.

In some embodiments, a subject is administered any one of the compositions herein either enterally (e.g., orally, by gastric feeding tube, by duodenal feeding tube, via gastrostomy or rectally), parenterally (e.g., subcutaneous injection, intravenous injection or infusion, intra-arterial injection or infusion, intraosseous infusion, intramuscular injection, intracerebral injection, intracerebroventricular injection, intrathecal), topically (e.g., epicutaneous, inhalational, via eye drops, or through a mucous membrane), or by direct injection into a target organ (e.g., the liver of a subject). Typically, oligonucleotides herein are administered intravenously or subcutaneously.

As a non-limiting set of examples, the oligonucleotides herein would typically be administered quarterly (once every three months), bi-monthly (once every two months), monthly or weekly. For example, the oligonucleotides may be administered every week or at intervals of two, or three weeks. Alternatively, the oligonucleotides may be administered daily. In some embodiments, a subject is administered one or more loading doses of the oligonucleotide followed by one or more maintenance doses of the oligonucleotide.

In some embodiments, the subject to be treated is a human or non-human primate or other mammalian subject. Other exemplary subjects include domesticated animals such as dogs and cats; livestock such as horses, cattle, pigs, sheep, goats, and chickens; and animals such as mice, rats, guinea pigs, and hamsters.

### V. Kits

In some embodiments, the disclosure provides a kit comprising an oligonucleotide herein, and instructions for use. In some embodiments, the kit comprises an oligonucleotide herein, and a package insert containing instructions for use of the kit and/or any component thereof. In some embodiments, the kit comprises, in a suitable container, an oligonucleotide herein, one or more controls, and various buffers, reagents, enzymes and other standard ingredients well known in the art. In some embodiments, the container comprises at least one vial, well, test tube, flask, bottle, syringe or other container means, into which the oligonucleotide is placed, and in some instances, suitably aliquoted. In some embodiments where an additional component is provided, the kit contains additional containers into which this component is placed. The kits can also include a means for containing the oligonucleotide and any other reagent in close confinement for commercial sale. Such containers may include injection or blow-molded plastic containers into which the desired vials are retained. Containers and/or kits can include labeling with instructions for use and/or warnings.

In some embodiments, a kit comprises an oligonucleotide herein, and a pharmaceutically acceptable carrier, or a pharmaceutical composition comprising the oligonucleotide and instructions for treating or delaying progression of a disease, disorder or condition associated with LPA expression in a subject in need thereof.

### EXAMPLES

While the disclosure has been described with reference to the specific embodiments set forth in the following Examples, it should be understood by those skilled in the art that various changes may be made, and equivalents may be substituted without departing from the true spirit and scope of the disclosure. Further, the following Examples are offered by way of illustration and are not intended to limit the scope of the disclosure in any manner. In addition, modifications may be made to adapt to a situation, material, composition of matter, process, process step or steps, to the objective, spirit and scope of the disclosure. All such modifications are intended to be within the scope of the disclosure. Standard techniques well known in the art or the techniques specifically described below are utilized.

### Example 1: Preparation of Double-Stranded RNAi Oligonucleotides

### Oligonucleotide Synthesis and Purification

The ds RNAi oligonucleotides described in the foregoing Examples are chemically synthesized using methods described herein. Generally, ds RNAi oligonucleotides are synthesized using solid phase oligonucleotide synthesis methods as described for 19-23mer siRNAs (see, e.g., Scaringe et al. (1990) NUCLEIC ACIDS RES. 18:5433-41 and Usman et al. (1987) J. AM. CHEM. SOC. 109:7845-45; see also, U.S. Pat. Nos. 5,804,683; 5,831,071; 5,998,203; 6,008,400; 6,111,086; 6,117,657; 6,353,098; 6,362,323; 6,437,117 and 6,469,158).

Individual RNA strands are synthesized and HPLC purified according to standard methods (Integrated DNA Technologies; Coralville, IA). For example, RNA oligonucleotides are synthesized using solid phase phosphoramidite chemistry, deprotected and desalted on NAP-5 columns (Amersham Pharmacia Biotech; Piscataway, NJ) using standard techniques (Damha & Olgivie (1993) METHODS MOL. BIOL. 20:81-114; Wincott et al. (1995) NUCLEIC ACIDS RES. 23:2677-84). The oligomers are purified using ion-exchange high performance liquid chromatography (IE-HPLC) on an Amersham Source 15Q column (1.0 cm×25 cm; Amersham Pharmacia Biotech) using a 15 min step-linear gradient. The gradient varies from 90:10 Buffers A:B to 52:48 Buffers A:B, where Buffer A is 100 mM Tris pH 8.5 and Buffer B is 100 mM Tris pH 8.5, 1 M NaCl. Samples are monitored at 260 nm and peaks corresponding to the full-length oligonucleotide species are collected, pooled, desalted on NAP-5 columns, and lyophilized.

The purity of each oligomer is determined by capillary electrophoresis (CE) on a Beckman PACE 5000 (Beckman Coulter, Inc.; Fullerton, CA). The CE capillaries have a 100 μm inner diameter and contain ssDNA 100R Gel (Beckman-Coulter). Typically, about 0.6 nmole of oligonucleotide is injected into a capillary, is run in an electric field of 444 V/cm and is detected by UV absorbance at 260 nm. Denaturing Tris-Borate-7 M-urea running buffer is purchased from Beckman-Coulter. Oligoribonucleotides are obtained that are at least 90% pure as assessed by CE for use in experiments described below. Compound identity is verified by matrix-assisted laser desorption ionization time-of-flight (MALDI-TOF) mass spectroscopy on a Voyager DE™ Biospectometry Work Station (Applied Biosystems; Foster City, CA) following the manufacturer's recommended protocol. Relative molecular masses of all oligomers are obtained, often within 0.2% of expected molecular mass.

### Preparation of Duplexes

ssRNA oligomers are resuspended (e.g., at 100 μM concentration) in duplex buffer consisting of 100 mM potassium acetate, 30 mM HEPES, pH 7.5. Complementary sense and antisense strands are mixed in equal molar amounts to yield a final solution of, for example, 50 μM duplex. Samples are heated to 100° C. for 5′ in RNA buffer (IDT) and are allowed to cool to room temperature before use. The ds RNA oligonucleotides are stored at −20° C. ss RNA oligomers are stored lyophilized or in nuclease-free water at −80° C.

### Example 2: RNAi Oligonucleotide Inhibition of LPA Expression In Vitro

LPA mRNA Target Sequence Identification

To identify RNAi oligonucleotide inhibitors of LPA expression, a computer-based algorithm was used to computationally identify LPA mRNA target sequences suitable for assaying inhibition of LPA expression by the RNAi pathway. The algorithm provides RNAi oligonucleotide guide (antisense) strand sequences each having a region of complementarity to a suitable LPA target sequence of human LPA mRNA (e.g., SEQ ID NO: 1; Table 1). Some of the guide strand sequences identified by the algorithm are also complementary to the corresponding LPA target sequence of monkey LPA mRNA (SEQ ID NO: 2; Table 1). RNAi oligonucleotides (formatted as DsiRNA oligonucleotides) were generated (Table 2), each with a unique guide strand having a region of complementarity to an LPA target sequence identified by the algorithm. The passenger (sense) strands of the DsiRNAs provided in Table 2 comprise a unique human LPA mRNA target sequence identified by the algorithm.

TABLE 1
Sequences of Human and NHP (Monkey) mRNA

| Species | GenBank Ref Seq # | SEQ ID NO: |
|---|---|---|
| Human (Hs) | NM_005577.3 | 1 |
| Cynomolgus monkey (Mf) | XM_015448517.1 | 2 |
| Rhesus monkey | XM_028847001.1 | 3 |

TABLE 2
DsiRNAs Targeting Human LPA mRNA and
Controls Evaluated in Cells

| | | SEQ | | SEQ | | SEQ |
|---|---|---|---|---|---|---|
| | | ID | Guide | ID | Target | ID |
| | Passenger (Sense) | NO: | (Antisense) | NO: | Sequence | NO: |
| Dsi | | | | | | |
| RNA | | | | | | |
| LPA- | CUGAGCAAAGCCAUGUGGU | 4 | UCCUGUACCACAUG | 404 | CUGAGCAAAG | 804 |
| 125 | ACAGGA | | GCUUUGCUCAGGU | | CCAUGUGGU | |
| LPA- | AGCAAAGCCAUGUGGUCCA | 5 | CAAUCUUGGACCAC | 405 | AGCAAAGCCA | 805 |
| 128 | AGAUTG | | AUGGCUUUGCUCA | | UGUGGUCCA | |
| LPA- | AAGCCAUGUGGUCCAGGAU | 6 | GUAGCUAUCCUGGA | 406 | AAGCCAUGUG | 806 |
| 132 | AGCUAC | | CCACAUGGCUUUG | | GUCCAGGAU | |
| LPA- | AGCCAUGUGGUCCAGGAUU | 7 | GGUAGUAAUCCUGG | 407 | AGCCAUGUGG | 807 |
| 133 | ACUACC | | ACCACAUGGCUUU | | UCCAGGAUU | |
| LPA- | GCCAUGUGGUCCAGGAUUG | 8 | UGGUAUCAAUCCUG | 408 | GCCAUGUGGU | 808 |
| 134 | AUACCA | | GACCACAUGGCUU | | CCAGGAUUG | |
| LPA- | CCAUGUGGUCCAGGAUUGC | 9 | AUGGUUGCAAUCCU | 409 | CCAUGUGGUC | 809 |
| 135 | AACCAT | | GGACCACAUGGCU | | CAGGAUUGC | |
| LPA- | CAUGUGGUCCAGGAUUGCU | 10 | CAUGGUAGCAAUCC | 410 | CAUGUGGUCC | 810 |
| 136 | ACCATG | | UGGACCACAUGGC | | AGGAUUGCU | |
| LPA- | AUGUGGUCCAGGAUUGCUA | 11 | CCAUGUUAGCAAUC | 411 | AUGUGGUCCA | 811 |
| 137 | ACAUGG | | CUGGACCACAUGG | | GGAUUGCUA | |
| LPA- | UGUGGUCCAGGAUUGCUAC | 12 | ACCAUUGUAGCAAU | 412 | UGUGGUCCAG | 812 |
| 138 | AAUGGT | | CCUGGACCACAUG | | GAUUGCUAC | |
| LPA- | GGUGAUGGACAGAGUUAUC | 13 | UGCCUUGAUAACUC | 413 | GGUGAUGGAC | 813 |
| 160 | AAGGCA | | UGUCCAUCACCAU | | AGAGUUAUC | |
| LPA- | UCCACCACUGUCACAGGAA | 14 | AGGUCUUUCCUGUG | 414 | UCCACCACUG | 814 |
| 190 | AGACCT | | ACAGUGGUGGAGU | | UCACAGGAA | |
| LPA- | CCACCACUGUCACAGGAAG | 15 | CAGGUUCUUCCUGU | 415 | CCACCACUGU | 815 |
| 191 | AACCTG | | GACAGUGGUGGAG | | CACAGGAAG | |
| LPA- | CUGUCACAGGAAGGACCUG | 16 | GCUUGUCAGGUCCU | 416 | CUGUCACAGG | 816 |
| 197 | ACAAGC | | UCCUGUGACAGUG | | AAGGACCUG | |
| LPA- | GGAAGGACCUGCCAAGCUU | 17 | AUGACUAAGCUUGG | 417 | GGAAGGACCU | 817 |
| 205 | AGUCAT | | CAGGUCCUUCCUG | | GCCAAGCUU | |
| LPA- | GAAGGACCUGCCAAGCUUG | 18 | GAUGAUCAAGCUUG | 418 | GAAGGACCUG | 818 |
| 206 | AUCATC | | GCAGGUCCUUCCU | | CCAAGCUUG | |
| LPA- | AGGACCUGCCAAGCUUGGU | 19 | UAGAUUACCAAGCU | 419 | AGGACCUGCC | 819 |
| 208 | AAUCTA | | UGGCAGGUCCUUC | | AAGCUUGGU | |
| LPA- | GGACCUGCCAAGCUUGGUC | 20 | AUAGAUGACCAAGC | 420 | GGACCUGCCA | 820 |
| 209 | AUCUAT | | UUGGCAGGUCCUU | | AGCUUGGUC | |
| LPA- | GACCUGCCAAGCUUGGUCA | 21 | CAUAGUUGACCAAG | 421 | GACCUGCCAA | 821 |
| 210 | ACUATG | | CUUGGCAGGUCCU | | GCUUGGUCA | |
| LPA- | ACCUGCCAAGCUUGGUCAU | 22 | UCAUAUAUGACCAA | 422 | ACCUGCCAAG | 822 |
| 211 | AUAUGA | | GCUUGGCAGGUCC | | CUUGGUCAU | |
| LPA- | CCUGCCAAGCUUGGUCAUC | 23 | GUCAUUGAUGACCA | 423 | CCUGCCAAGC | 823 |
| 212 | AAUGAC | | AGCUUGGCAGGUC | | UUGGUCAUC | |
| LPA- | AGCUUGGUCAUCUAUGACA | 24 | AUGUGUUGUCAUAG | 424 | AGCUUGGUCA | 824 |
| 219 | ACACAT | | AUGACCAAGCUUG | | UCUAUGACA | |
| LPA- | GUCAUCUAUGACACCACAU | 25 | AUGUUUAUGUGGUG | 425 | GUCAUCUAUG | 825 |
| 225 | AAACAT | | UCAUAGAUGACCA | | ACACCACAU | |
| LPA- | CACAGAAAACUACCCAAAU | 26 | GCCAGUAUUUGGGU | 426 | CACAGAAAAC | 826 |
| 258 | ACUGGC | | AGUUUUCUGUGGU | | UACCCAAAU | |
| LPA- | AGAAAACUACCCAAAUGCU | 27 | CAAGCUAGCAUUUG | 427 | AGAAAACUAC | 827 |
| 261 | AGCUTG | | GGUAGUUUUCUGU | | CCAAAUGCU | |
| LPA- | AAAACUACCCAAAUGCUGG | 28 | AUCAAUCCAGCAUU | 428 | AAAACUACCC | 828 |
| 263 | AUUGAT | | UGGGUAGUUUUCU | | AAAUGCUGG | |
| LPA- | ACCCAAAUGCUGGCUUGAU | 29 | UUCAUUAUCAAGCC | 429 | ACCCAAAUGC | 829 |
| 269 | AAUGAA | | AGCAUUUGGGUAG | | UGGCUUGAU | |
| LPA- | CCCAAAUGCUGGCUUGAUC | 30 | GUUCAUGAUCAAGC | 430 | CCCAAAUGCU | 830 |
| 270 | AUGAAC | | CAGCAUUUGGGUA | | GGCUUGAUC | |
| LPA- | GAACUACUGCAGGAAUCCA | 31 | AGCAUUUGGAUUCC | 431 | GAACUACUGC | 831 |
| 291 | AAUGCT | | UGCAGUAGUUCAU | | AGGAAUCCA | |
| LPA- | UACUGCAGGAAUCCAGAUG | 32 | CCACAUCAUCUGGA | 432 | UACUGCAGGA | 832 |
| 295 | AUGUGG | | UUCCUGCAGUAGU | | AUCCAGAUG | |
| LPA- | ACUGCAGGAAUCCAGAUGC | 33 | GCCACUGCAUCUGG | 433 | ACUGCAGGAA | 833 |
| 296 | AGUGGC | | AUUCCUGCAGUAG | | UCCAGAUGC | |
| LPA- | UGCAGGAAUCCAGAUGCUG | 34 | CUGCCUCAGCAUCU | 434 | UGCAGGAAUC | 834 |
| 298 | AGGCAG | | GGAUUCCUGCAGU | | CAGAUGCUG | |
| LPA- | AGGUGGGAGUACUGCAACC | 35 | GCGUCUGGUUGCAG | 435 | AGGUGGGAGU | 835 |
| 355 | AGACGC | | UACUCCCACCUGA | | ACUGCAACC | |
| LPA- | AAUGCUCAGACGCAGAAGG | 36 | GCAGUUCCUUCUGC | 436 | AAUGCUCAGA | 836 |
| 380 | AACUGC | | GUCUGAGCAUUGC | | CGCAGAAGG | |
| LPA- | GACUGUUACCCCGGUUCCA | 37 | UAGGCUUGGAACCG | 437 | GACUGUUACC | 837 |
| 417 | AGCCTA | | GGGUAACAGUCGG | | CCGGUUCCA | |
| LPA- | ACUGUUACCCCGGUUCCAA | 38 | CUAGGUUUGGAACC | 438 | ACUGUUACCC | 838 |
| 418 | ACCUAG | | GGGGUAACAGUCG | | CGGUUCCAA | |
| LPA- | CUGUUACCCCGGUUCCAAG | 39 | UCUAGUCUUGGAAC | 439 | CUGUUACCCC | 839 |
| 419 | ACUAGA | | CGGGGUAACAGUC | | GGUUCCAAG | |
| LPA- | UGUUACCCCGGUUCCAAGC | 40 | CUCUAUGCUUGGAA | 440 | UGUUACCCCG | 840 |
| 420 | AUAGAG | | CCGGGGUAACAGU | | GUUCCAAGC | |
| LPA- | GUUACCCCGGUUCCAAGCC | 41 | CCUCUUGGCUUGGA | 441 | GUUACCCCGG | 841 |
| 421 | AAGAGG | | ACCGGGGUAACAG | | UUCCAAGCC | |
| LPA- | UUACCCCGGUUCCAAGCCU | 42 | GCCUCUAGGCUUGG | 442 | UUACCCCGGU | 842 |
| 422 | AGAGGC | | AACCGGGGUAACA | | UCCAAGCCU | |
| LPA- | UACCCCGGUUCCAAGCCUA | 43 | AGCCUUUAGGCUUG | 443 | UACCCCGGUU | 843 |
| 423 | AAGGCT | | GAACCGGGGUAAC | | CCAAGCCUA | |
| LPA- | GUGCUACCAUGGUAAUGGA | 44 | ACUCUUUCCAUUAC | 444 | GUGCUACCAU | 844 |
| 492 | AAGAGT | | CAUGGUAGCACUC | | GGUAAUGGA | |
| LPA- | UGCUACCAUGGUAAUGGAC | 45 | AACUCUGUCCAUUA | 445 | UGCUACCAUG | 845 |
| 493 | AGAGTT | | CCAUGGUAGCACU | | GUAAUGGAC | |
| LPA- | GCUACCAUGGUAAUGGACA | 46 | UAACUUUGUCCAUU | 446 | GCUACCAUGG | 846 |
| 494 | AAGUTA | | ACCAUGGUAGCAC | | UAAUGGACA | |
| LPA- | CUACCAUGGUAAUGGACAG | 47 | AUAACUCUGUCCAU | 447 | CUACCAUGGU | 847 |
| 495 | AGUUAT | | UACCAUGGUAGCA | | AAUGGACAG | |
| LPA- | UACCAUGGUAAUGGACAGA | 48 | GAUAAUUCUGUCCA | 448 | UACCAUGGUA | 848 |
| 496 | AUUATC | | UUACCAUGGUAGC | | AUGGACAGA | |
| LPA- | ACCAUGGUAAUGGACAGAG | 49 | CGAUAUCUCUGUCC | 449 | ACCAUGGUAA | 849 |
| 497 | AUAUCG | | AUUACCAUGGUAG | | UGGACAGAG | |
| LPA- | CCAUGGUAAUGGACAGAGU | 50 | UCGAUUACUCUGUC | 450 | CCAUGGUAAU | 850 |
| 498 | AAUCGA | | CAUUACCAUGGUA | | GGACAGAGU | |
| LPA- | CAUGGUAAUGGACAGAGUU | 51 | CUCGAUAACUCUGU | 451 | CAUGGUAAUG | 851 |
| 499 | AUCGAG | | CCAUUACCAUGGU | | GACAGAGUU | |
| LPA- | AUGGUAAUGGACAGAGUUA | 52 | CCUCGUUAACUCUG | 452 | AUGGUAAUGG | 852 |
| 500 | ACGAGG | | UCCAUUACCAUGG | | ACAGAGUUA | |
| LPA- | UGGUAAUGGACAGAGUUAU | 53 | GCCUCUAUAACUCU | 453 | UGGUAAUGGA | 853 |
| 501 | AGAGGC | | GUCCAUUACCAUG | | CAGAGUUAU | |
| LPA- | GGUAAUGGACAGAGUUAUC | 54 | UGCCUUGAUAACUC | 454 | GGUAAUGGAC | 854 |
| 502 | AAGGCA | | UGUCCAUUACCAU | | AGAGUUAUC | |
| LPA- | GUAAUGGACAGAGUUAUCG | 55 | GUGCCUCGAUAACU | 455 | GUAAUGGACA | 855 |
| 503 | AGGCAC | | CUGUCCAUUACCA | | GAGUUAUCG | |
| LPA- | GGCACAUACUCCACCACUG | 56 | CUGUGUCAGUGGUG | 456 | GGCACAUACU | 856 |
| 523 | ACACAG | | GAGUAUGUGCCUC | | CCACCACUG | |
| LPA- | CUUGGUCAUCUAUGACACC | 57 | GAGUGUGGUGUCAU | 457 | CUUGGUCAUC | 857 |
| 563 | ACACTC | | AGAUGACCAAGCU | | UAUGACACC | |
| LPA- | GUCAUCUAUGACACCACAC | 58 | AUGCGUGUGUGGUG | 458 | GUCAUCUAUG | 858 |
| 567 | ACGCAT | | UCAUAGAUGACCA | | ACACCACAC | |
| LPA- | UCAUCUAUGACACCACACU | 59 | UAUGCUAGUGUGGU | 459 | UCAUCUAUGA | 859 |
| 568 | AGCATA | | GUCAUAGAUGACC | | CACCACACU | |
| LPA- | CAUCUAUGACACCACACUC | 60 | CUAUGUGAGUGUGG | 460 | CAUCUAUGAC | 860 |
| 569 | ACAUAG | | UGUCAUAGAUGAC | | ACCACACUC | |
| LPA- | GCACAUACUCCACCACUGU | 61 | CCAGUUACAGUGGU | 461 | GCACAUACUC | 861 |
| 1208 | AACUGG | | GGAGUAUGUGCCU | | CACCACUGU | |
| LPA- | AGCCCCUUAUUGUUAUACG | 62 | AUCCCUCGUAUAAC | 462 | AGCCCCUUAU | 862 |
| 2715 | AGGGAT | | AAUAAGGGGCUGC | | UGUUAUACG | |
| LPA- | GCCCCUUAUUGUUAUACGA | 63 | GAUCCUUCGUAUAA | 463 | GCCCCUUAUU | 863 |
| 2716 | AGGATC | | CAAUAAGGGGCUG | | GUUAUACGA | |
| LPA- | CCAAGCCUAGAGGCUCCUU | 64 | GUUCAUAAGGAGCC | 464 | CCAAGCCUAG | 864 |
| 2827 | AUGAAC | | UCUAGGCUUGGAA | | AGGCUCCUU | |
| LPA- | AGGCUCCUUCUGAACAAGC | 65 | GUUGGUGCUUGUUC | 465 | AGGCUCCUUC | 865 |
| 2837 | ACCAAC | | AGAAGGAGCCUCU | | UGAACAAGC | |
| LPA- | AUGGACAGAGUUAUCAAGG | 66 | UAUGUUCCUUGAUA | 466 | AUGGACAGAG | 866 |
| 2900 | AACATA | | ACUCUGUCCAUUU | | UUAUCAAGG | |
| LPA- | UGGACAGAGUUAUCAAGGC | 67 | GUAUGUGCCUUGAU | 467 | UGGACAGAGU | 867 |
| 2901 | ACAUAC | | AACUCUGUCCAUU | | UAUCAAGGC | |
| LPA- | GGACAGAGUUAUCAAGGCA | 68 | AGUAUUUGCCUUGA | 468 | GGACAGAGUU | 868 |
| 2902 | AAUACT | | UAACUCUGUCCAU | | AUCAAGGCA | |
| LPA- | GACAGAGUUAUCAAGGCAC | 69 | AAGUAUGUGCCUUG | 469 | GACAGAGUUA | 869 |
| 2903 | AUACTT | | AUAACUCUGUCCA | | UCAAGGCAC | |
| LPA- | ACAGAGUUAUCAAGGCACA | 70 | GAAGUUUGUGCCUU | 470 | ACAGAGUUAU | 870 |
| 2904 | AACUTC | | GAUAACUCUGUCC | | CAAGGCACA | |
| LPA- | CAGAGUUAUCAAGGCACAU | 71 | UGAAGUAUGUGCCU | 471 | CAGAGUUAUC | 871 |
| 2905 | ACUUCA | | UGAUAACUCUGUC | | AAGGCACAU | |
| LPA- | UACCCAAAUGCUGGCUUGA | 72 | UCUUGUUCAAGCCA | 472 | UACCCAAAUG | 872 |
| 3004 | ACAAGA | | GCAUUUGGGUAGU | | CUGGCUUGA | |
| LPA- | CCAAAUGCUGGCUUGAUCA | 73 | AGUUCUUGAUCAAG | 473 | CCAAAUGCUG | 873 |
| 3007 | AGAACT | | CCAGCAUUUGGGU | | GCUUGAUCA | |
| LPA- | UCAAGAACUACUGCCGAAA | 74 | UCUGGUUUUCGGCA | 474 | UCAAGAACUA | 874 |
| 3023 | ACCAGA | | GUAGUUCUUGAUC | | CUGCCGAAA | |
| LPA- | CAAGAACUACUGCCGAAAU | 75 | AUCUGUAUUUCGGC | 475 | CAAGAACUAC | 875 |
| 3024 | ACAGAT | | AGUAGUUCUUGAU | | UGCCGAAAU | |
| LPA- | AAGAACUACUGCCGAAAUC | 76 | GAUCUUGAUUUCGG | 476 | AAGAACUACU | 876 |
| 3025 | AAGATC | | CAGUAGUUCUUGA | | GCCGAAAUC | |
| LPA- | GAACUACUGCCGAAAUCCA | 77 | AGGAUUUGGAUUUC | 477 | GAACUACUGC | 877 |
| 3027 | AAUCCT | | GGCAGUAGUUCUU | | CGAAAUCCA | |
| LPA- | CUACUGCCGAAAUCCAGAU | 78 | CACAGUAUCUGGAU | 478 | CUACUGCCGA | 878 |
| 3030 | ACUGTG | | UUCGGCAGUAGUU | | AAUCCAGAU | |
| LPA- | UGUGGCAGCCCCUUGGUGU | 79 | UGUAUUACACCAAG | 479 | UGUGGCAGCC | 879 |
| 3051 | AAUACA | | GGGCUGCCACAGG | | CCUUGGUGU | |
| LPA- | GUGGCAGCCCCUUGGUGUU | 80 | UUGUAUAACACCAA | 480 | GUGGCAGCCC | 880 |
| 3052 | AUACAA | | GGGGCUGCCACAG | | CUUGGUGUU | |
| LPA- | UGGCAGCCCCUUGGUGUUA | 81 | GUUGUUUAACACCA | 481 | UGGCAGCCCC | 881 |
| 3053 | AACAAC | | AGGGGCUGCCACA | | UUGGUGUUA | |
| LPA- | GGCAGCCCCUUGGUGUUAU | 82 | UGUUGUAUAACACC | 482 | GGCAGCCCCU | 882 |
| 3054 | ACAACA | | AAGGGGCUGCCAC | | UGGUGUUAU | |
| LPA- | GCAGCCCCUUGGUGUUAUA | 83 | CUGUUUUAUAACAC | 483 | GCAGCCCCUU | 883 |
| 3055 | AAACAG | | CAAGGGGCUGCCA | | GGUGUUAUA | |
| LPA- | CAGCCCCUUGGUGUUAUAC | 84 | UCUGUUGUAUAACA | 484 | CAGCCCCUUG | 884 |
| 3056 | AACAGA | | CCAAGGGGCUGCC | | GUGUUAUAC | |
| LPA- | AGCCCCUUGGUGUUAUACA | 85 | AUCUGUUGUAUAAC | 485 | AGCCCCUUGG | 885 |
| 3057 | ACAGAT | | ACCAAGGGGCUGC | | UGUUAUACA | |
| LPA- | GCCCCUUGGUGUUAUACAA | 86 | GAUCUUUUGUAUAA | 486 | GCCCCUUGGU | 886 |
| 3058 | AAGATC | | CACCAAGGGGCUG | | GUUAUACAA | |
| LPA- | CCCCUUGGUGUUAUACAAC | 87 | GGAUCUGUUGUAUA | 487 | CCCCUUGGUG | 887 |
| 3059 | AGAUCC | | ACACCAAGGGGCU | | UUAUACAAC | |
| LPA- | GGUGGGAGUACUGCAACCU | 88 | CGUGUUAGGUUGCA | 488 | GGUGGGAGUA | 888 |
| 3092 | AACACG | | GUACUCCCACCUG | | CUGCAACCU | |
| LPA- | GUGGGAGUACUGCAACCUG | 89 | UCGUGUCAGGUUGC | 489 | GUGGGAGUAC | 889 |
| 3093 | ACACGA | | AGUACUCCCACCU | | UGCAACCUG | |
| LPA- | GGAGUACUGCAACCUGACA | 90 | GCAUCUUGUCAGGU | 490 | GGAGUACUGC | 890 |
| 3096 | AGAUGC | | UGCAGUACUCCCA | | AACCUGACA | |
| LPA- | GAGUACUGCAACCUGACAC | 91 | AGCAUUGUGUCAGG | 491 | GAGUACUGCA | 891 |
| 3097 | AAUGCT | | UUGCAGUACUCCC | | ACCUGACAC | |
| LPA- | GUACUGCAACCUGACACGA | 92 | UGAGCUUCGUGUCA | 492 | GUACUGCAAC | 892 |
| 3099 | AGCUCA | | GGUUGCAGUACUC | | CUGACACGA | |
| LPA- | UACUGCAACCUGACACGAU | 93 | CUGAGUAUCGUGUC | 493 | UACUGCAACC | 893 |
| 3100 | ACUCAG | | AGGUUGCAGUACU | | UGACACGAU | |
| LPA- | ACUGCAACCUGACACGAUG | 94 | UCUGAUCAUCGUGU | 494 | ACUGCAACCU | 894 |
| 3101 | AUCAGA | | CAGGUUGCAGUAC | | GACACGAUG | |
| LPA- | CUGCAACCUGACACGAUGC | 95 | AUCUGUGCAUCGUG | 495 | CUGCAACCUG | 895 |
| 3102 | ACAGAT | | UCAGGUUGCAGUA | | ACACGAUGC | |
| LPA- | UGCAACCUGACACGAUGCU | 96 | CAUCUUAGCAUCGU | 496 | UGCAACCUGA | 896 |
| 3103 | AAGATG | | GUCAGGUUGCAGU | | CACGAUGCU | |
| LPA- | CAACCUGACACGAUGCUCA | 97 | UGCAUUUGAGCAUC | 497 | CAACCUGACA | 897 |
| 3105 | AAUGCA | | GUGUCAGGUUGCA | | CGAUGCUCA | |
| LPA- | ACCUGACACGAUGCUCAGA | 98 | UCUGCUUCUGAGCA | 498 | ACCUGACACG | 898 |
| 3107 | AGCAGA | | UCGUGUCAGGUUG | | AUGCUCAGA | |
| LPA- | CCUGACACGAUGCUCAGAU | 99 | UUCUGUAUCUGAGC | 499 | CCUGACACGA | 899 |
| 3108 | ACAGAA | | AUCGUGUCAGGUU | | UGCUCAGAU | |
| LPA- | CUGACACGAUGCUCAGAUG | 100 | AUUCUUCAUCUGAG | 500 | CUGACACGAU | 900 |
| 3109 | AAGAAT | | CAUCGUGUCAGGU | | GCUCAGAUG | |
| LPA- | UGACACGAUGCUCAGAUGC | 101 | CAUUCUGCAUCUGA | 501 | UGACACGAUG | 901 |
| 3110 | AGAATG | | GCAUCGUGUCAGG | | CUCAGAUGC | |
| LPA- | GACACGAUGCUCAGAUGCA | 102 | CCAUUUUGCAUCUG | 502 | GACACGAUGC | 902 |
| 3111 | AAAUGG | | AGCAUCGUGUCAG | | UCAGAUGCA | |
| LPA- | ACACGAUGCUCAGAUGCAG | 103 | UCCAUUCUGCAUCU | 503 | ACACGAUGCU | 903 |
| 3112 | AAUGGA | | GAGCAUCGUGUCA | | CAGAUGCAG | |
| LPA- | CACGAUGCUCAGAUGCAGA | 104 | GUCCAUUCUGCAUC | 504 | CACGAUGCUC | 904 |
| 3113 | AUGGAC | | UGAGCAUCGUGUC | | AGAUGCAGA | |
| LPA- | UGCUACUACCAUUAUGGAC | 105 | AACUCUGUCCAUAA | 505 | UGCUACUACC | 905 |
| 3229 | AGAGTT | | UGGUAGUAGCAGU | | AUUAUGGAC | |
| LPA- | GCUACUACCAUUAUGGACA | 106 | UAACUUUGUCCAUA | 506 | GCUACUACCA | 906 |
| 3230 | AAGUTA | | AUGGUAGUAGCAG | | UUAUGGACA | |
| LPA- | CUACUACCAUUAUGGACAG | 107 | GUAACUCUGUCCAU | 507 | CUACUACCAU | 907 |
| 3231 | AGUUAC | | AAUGGUAGUAGCA | | UAUGGACAG | |
| LPA- | UACUACCAUUAUGGACAGA | 108 | GGUAAUUCUGUCCA | 508 | UACUACCAUU | 908 |
| 3232 | AUUACC | | UAAUGGUAGUAGC | | AUGGACAGA | |
| LPA- | ACUACCAUUAUGGACAGAG | 109 | CGGUAUCUCUGUCC | 509 | ACUACCAUUA | 909 |
| 3233 | AUACCG | | AUAAUGGUAGUAG | | UGGACAGAG | |
| LPA- | CUACCAUUAUGGACAGAGU | 110 | UCGGUUACUCUGUC | 510 | CUACCAUUAU | 910 |
| 3234 | AACCGA | | CAUAAUGGUAGUA | | GGACAGAGU | |
| LPA- | UACCAUUAUGGACAGAGUU | 111 | CUCGGUAACUCUGU | 511 | UACCAUUAUG | 911 |
| 3235 | ACCGAG | | CCAUAAUGGUAGU | | GACAGAGUU | |
| LPA- | ACCAUUAUGGACAGAGUUA | 112 | CCUCGUUAACUCUG | 512 | ACCAUUAUGG | 912 |
| 3236 | ACGAGG | | UCCAUAAUGGUAG | | ACAGAGUUA | |
| LPA- | GAGGCACAUACUCCACCAC | 113 | GUGACUGUGGUGGA | 513 | GAGGCACAUA | 913 |
| 3257 | AGUCAC | | GUAUGUGCCUCGG | | CUCCACCAC | |
| LPA- | CUCCACCACUGUCACAGGA | 114 | AGUUCUUCCUGUGA | 514 | CUCCACCACU | 914 |
| 3267 | AGAACT | | CAGUGGUGGAGUA | | GUCACAGGA | |
| LPA- | ACAGGAAGAACUUGCCAAG | 115 | ACCAAUCUUGGCAA | 515 | ACAGGAAGAA | 915 |
| 3280 | AUUGGT | | GUUCUUCCUGUGA | | CUUGCCAAG | |
| LPA- | CAGGAAGAACUUGCCAAGC | 116 | GACCAUGCUUGGCA | 516 | CAGGAAGAAC | 916 |
| 3281 | AUGGTC | | AGUUCUUCCUGUG | | UUGCCAAGC | |
| LPA- | AGGAAGAACUUGCCAAGCU | 117 | UGACCUAGCUUGGC | 517 | AGGAAGAACU | 917 |
| 3282 | AGGUCA | | AAGUUCUUCCUGU | | UGCCAAGCU | |
| LPA- | GGAAGAACUUGCCAAGCUU | 118 | AUGACUAAGCUUGG | 518 | GGAAGAACUU | 918 |
| 3283 | AGUCAT | | CAAGUUCUUCCUG | | GCCAAGCUU | |
| LPA- | GAAGAACUUGCCAAGCUUG | 119 | GAUGAUCAAGCUUG | 519 | GAAGAACUUG | 919 |
| 3284 | AUCATC | | GCAAGUUCUUCCU | | CCAAGCUUG | |
| LPA- | AAGAACUUGCCAAGCUUGG | 120 | AGAUGUCCAAGCUU | 520 | AAGAACUUGC | 920 |
| 3285 | ACAUCT | | GGCAAGUUCUUCC | | CAAGCUUGG | |
| LPA- | AGAACUUGCCAAGCUUGGU | 121 | UAGAUUACCAAGCU | 521 | AGAACUUGCC | 921 |
| 3286 | AAUCTA | | UGGCAAGUUCUUC | | AAGCUUGGU | |
| LPA- | GAACUUGCCAAGCUUGGUC | 122 | AUAGAUGACCAAGC | 522 | GAACUUGCCA | 922 |
| 3287 | AUCUAT | | UUGGCAAGUUCUU | | AGCUUGGUC | |
| LPA- | AACUUGCCAAGCUUGGUCA | 123 | CAUAGUUGACCAAG | 523 | AACUUGCCAA | 923 |
| 3288 | ACUATG | | CUUGGCAAGUUCU | | GCUUGGUCA | |
| LPA- | ACUUGCCAAGCUUGGUCAU | 124 | UCAUAUAUGACCAA | 524 | ACUUGCCAAG | 924 |
| 3289 | AUAUGA | | GCUUGGCAAGUUC | | CUUGGUCAU | |
| LPA- | CUUGCCAAGCUUGGUCAUC | 125 | GUCAUUGAUGACCA | 525 | CUUGCCAAGC | 925 |
| 3290 | AAUGAC | | AGCUUGGCAAGUU | | UUGGUCAUC | |
| LPA- | UUGCCAAGCUUGGUCAUCU | 126 | UGUCAUAGAUGACC | 526 | UUGCCAAGCU | 926 |
| 3291 | AUGACA | | AAGCUUGGCAAGU | | UGGUCAUCU | |
| LPA- | UGCCAAGCUUGGUCAUCUA | 127 | GUGUCUUAGAUGAC | 527 | UGCCAAGCUU | 927 |
| 3292 | AGACAC | | CAAGCUUGGCAAG | | GGUCAUCUA | |
| LPA- | GCUUGGUCAUCUAUGACAC | 128 | GGUGUUGUGUCAUA | 528 | GCUUGGUCAU | 928 |
| 3298 | AACACC | | GAUGACCAAGCUU | | CUAUGACAC | |
| LPA- | UUGGUCAUCUAUGACACCA | 129 | CUGGUUUGGUGUCA | 529 | UUGGUCAUCU | 929 |
| 3300 | AACCAG | | UAGAUGACCAAGC | | AUGACACCA | |
| LPA- | UGGUCAUCUAUGACACCAC | 130 | GCUGGUGUGGUGUC | 530 | UGGUCAUCUA | 930 |
| 3301 | ACCAGC | | AUAGAUGACCAAG | | UGACACCAC | |
| LPA- | GUCAUCUAUGACACCACAC | 131 | AUGCUUGUGUGGUG | 531 | GUCAUCUAUG | 931 |
| 3303 | AAGCAT | | UCAUAGAUGACCA | | ACACCACAC | |
| LPA- | CAUCUAUGACACCACACCA | 132 | CUAUGUUGGUGUGG | 532 | CAUCUAUGAC | 932 |
| 3305 | ACAUAG | | UGUCAUAGAUGAC | | ACCACACCA | |
| LPA- | AUCUAUGACACCACACCAG | 133 | ACUAUUCUGGUGUG | 533 | AUCUAUGACA | 933 |
| 3306 | AAUAGT | | GUGUCAUAGAUGA | | CCACACCAG | |
| LPA- | CUAUGACACCACACCAGCA | 134 | CGACUUUGCUGGUG | 534 | CUAUGACACC | 934 |
| 3308 | AAGUCG | | UGGUGUCAUAGAU | | ACACCAGCA | |
| LPA- | GUCGGACCCCAGAAAACUA | 135 | UUUGGUUAGUUUUC | 535 | GUCGGACCCC | 935 |
| 3329 | ACCAAA | | UGGGGUCCGACUA | | AGAAAACUA | |
| LPA- | UCGGACCCCAGAAAACUAC | 136 | AUUUGUGUAGUUUU | 536 | UCGGACCCCA | 936 |
| 3330 | ACAAAT | | CUGGGGUCCGACU | | GAAAACUAC | |
| LPA- | GAAAACUACCCAAAUGCUG | 137 | UCAGGUCAGCAUUU | 537 | GAAAACUACC | 937 |
| 3340 | ACCUGA | | GGGUAGUUUUCUG | | CAAAUGCUG | |
| LPA- | GCUGAGAUUCGCCCUUGGU | 138 | UGUAAUACCAAGGG | 538 | GCUGAGAUUC | 938 |
| 3391 | AUUACA | | CGAAUCUCAGCAU | | GCCCUUGGU | |
| LPA- | CUGAGAUUCGCCCUUGGUG | 139 | GUGUAUCACCAAGG | 539 | CUGAGAUUCG | 939 |
| 3392 | AUACAC | | GCGAAUCUCAGCA | | CCCUUGGUG | |
| LPA- | GAGAUUCGCCCUUGGUGUU | 140 | UGGUGUAACACCAA | 540 | GAGAUUCGCC | 940 |
| 3394 | ACACCA | | GGGCGAAUCUCAG | | CUUGGUGUU | |
| LPA- | AGAUUCGCCCUUGGUGUUA | 141 | AUGGUUUAACACCA | 541 | AGAUUCGCCC | 941 |
| 3395 | AACCAT | | AGGGCGAAUCUCA | | UUGGUGUUA | |
| LPA- | UUCGCCCUUGGUGUUACAC | 142 | UCCAUUGUGUAACA | 542 | UUCGCCCUUG | 942 |
| 3398 | AAUGGA | | CCAAGGGCGAAUC | | GUGUUACAC | |
| LPA- | CUUGGUGUUACACCAUGGA | 143 | CUGGGUUCCAUGGU | 543 | CUUGGUGUUA | 943 |
| 3404 | ACCCAG | | GUAACACCAAGGG | | CACCAUGGA | |
| LPA- | UUGGUGUUACACCAUGGAU | 144 | ACUGGUAUCCAUGG | 544 | UUGGUGUUAC | 944 |
| 3405 | ACCAGT | | UGUAACACCAAGG | | ACCAUGGAU | |
| LPA- | UGGUGUUACACCAUGGAUC | 145 | CACUGUGAUCCAUG | 545 | UGGUGUUACA | 945 |
| 3406 | ACAGTG | | GUGUAACACCAAG | | CCAUGGAUC | |
| LPA- | GGUGUUACACCAUGGAUCC | 146 | ACACUUGGAUCCAU | 546 | GGUGUUACAC | 946 |
| 3407 | AAGUGT | | GGUGUAACACCAA | | CAUGGAUCC | |
| LPA- | UGUUACACCAUGGAUCCCA | 147 | UGACAUUGGGAUCC | 547 | UGUUACACCA | 947 |
| 3409 | AUGUCA | | AUGGUGUAACACC | | UGGAUCCCA | |
| LPA- | GAAUCAAGUGUCCUUGCAA | 148 | UGAGAUUUGCAAGG | 548 | GAAUCAAGUG | 948 |
| 3472 | AUCUCA | | ACACUUGAUUCUG | | UCCUUGCAA | |
| LPA- | AAUCAAGUGUCCUUGCAAC | 149 | GUGAGUGUUGCAAG | 549 | AAUCAAGUGU | 949 |
| 3473 | ACUCAC | | GACACUUGAUUCU | | CCUUGCAAC | |
| LPA- | AUCAAGUGUCCUUGCAACU | 150 | CGUGAUAGUUGCAA | 550 | AUCAAGUGUC | 950 |
| 3474 | AUCACG | | GGACACUUGAUUC | | CUUGCAACU | |
| LPA- | AUGGACAGAGUUAUCGAGG | 151 | AAUGAUCCUCGAUA | 551 | AUGGACAGAG | 951 |
| 3584 | AUCATT | | ACUCUGUCCAUCA | | UUAUCGAGG | |
| LPA- | UGGACAGAGUUAUCGAGGC | 152 | GAAUGUGCCUCGAU | 552 | UGGACAGAGU | 952 |
| 3585 | ACAUTC | | AACUCUGUCCAUC | | UAUCGAGGC | |
| LPA- | ACACCACACUGGCAUCAGA | 153 | UUGUCUUCUGAUGC | 553 | ACACCACACU | 953 |
| 3655 | AGACAA | | CAGUGUGGUGUCA | | GGCAUCAGA | |
| LPA- | UUGGUGUUAUACCAUGGAU | 154 | AUUGGUAUCCAUGG | 554 | UUGGUGUUAU | 954 |
| 3747 | ACCAAT | | UAUAACACCAAGG | | ACCAUGGAU | |
| LPA- | UGGUGUUAUACCAUGGAUC | 155 | CAUUGUGAUCCAUG | 555 | UGGUGUUAUA | 955 |
| 3748 | ACAATG | | GUAUAACACCAAG | | CCAUGGAUC | |
| LPA- | GGUGUUAUACCAUGGAUCC | 156 | ACAUUUGGAUCCAU | 556 | GGUGUUAUAC | 956 |
| 3749 | AAAUGT | | GGUAUAACACCAA | | CAUGGAUCC | |
| LPA- | GUGUUAUACCAUGGAUCCC | 157 | GACAUUGGGAUCCA | 557 | GUGUUAUACC | 957 |
| 3750 | AAUGTC | | UGGUAUAACACCA | | AUGGAUCCC | |
| LPA- | UCAGAUGGGAGUACUGCAA | 158 | GUCAGUUUGCAGUA | 558 | UCAGAUGGGA | 958 |
| 3773 | ACUGAC | | CUCCCAUCUGACA | | GUACUGCAA | |
| LPA- | GAUGGGAGUACUGCAACCU | 159 | UGUGUUAGGUUGCA | 559 | GAUGGGAGUA | 959 |
| 3776 | AACACA | | GUACUCCCAUCUG | | CUGCAACCU | |
| LPA- | AUGGGAGUACUGCAACCUG | 160 | UUGUGUCAGGUUGC | 560 | AUGGGAGUAC | 960 |
| 3777 | ACACAA | | AGUACUCCCAUCU | | UGCAACCUG | |
| LPA- | UGGGAGUACUGCAACCUGA | 161 | AUUGUUUCAGGUUG | 561 | UGGGAGUACU | 961 |
| 3778 | AACAAT | | CAGUACUCCCAUC | | GCAACCUGA | |
| LPA- | GGGAGUACUGCAACCUGAC | 162 | CAUUGUGUCAGGUU | 562 | GGGAGUACUG | 962 |
| 3779 | ACAATG | | GCAGUACUCCCAU | | CAACCUGAC | |
| LPA- | GGCUGUUUCUGAACAAGCA | 163 | CGUUGUUGCUUGUU | 563 | GGCUGUUUCU | 963 |
| 3840 | ACAACG | | CAGAAACAGCCGU | | GAACAAGCA | |
| LPA- | GUUUCUGAACAAGCACCAA | 164 | GCUCCUUUGGUGCU | 564 | GUUUCUGAAC | 964 |
| 3844 | AGGAGC | | UGUUCAGAAACAG | | AAGCACCAA | |
| LPA- | CUCCACCACUGUUACAGGA | 165 | UGUCCUUCCUGUAA | 565 | CUCCACCACU | 965 |
| 3927 | AGGACA | | CAGUGGUGGAGAA | | GUUACAGGA | |
| LPA- | UCCACCACUGUUACAGGAA | 166 | AUGUCUUUCCUGUA | 566 | UCCACCACUG | 966 |
| 3928 | AGACAT | | ACAGUGGUGGAGA | | UUACAGGAA | |
| LPA- | CCACCACUGUUACAGGAAG | 167 | CAUGUUCUUCCUGU | 567 | CCACCACUGU | 967 |
| 3929 | AACATG | | AACAGUGGUGGAG | | UACAGGAAG | |
| LPA- | GACACCACACUGGCAUCAG | 168 | GGUUCUCUGAUGCC | 568 | GACACCACAC | 968 |
| 3972 | AGAACC | | AGUGUGGUGUCAU | | UGGCAUCAG | |
| LPA- | ACACCACACUGGCAUCAGA | 169 | UGGUUUUCUGAUGC | 569 | ACACCACACU | 969 |
| 3973 | AAACCA | | CAGUGUGGUGUCA | | GGCAUCAGA | |
| LPA- | AGAAUACUACCCAAAUGGU | 170 | CAGGCUACCAUUUG | 570 | AGAAUACUAC | 970 |
| 3999 | AGCCTG | | GGUAGUAUUCUGU | | CCAAAUGGU | |
| LPA- | GAAUACUACCCAAAUGGUG | 171 | UCAGGUCACCAUUU | 571 | GAAUACUACC | 971 |
| 4000 | ACCUGA | | GGGUAGUAUUCUG | | CAAAUGGUG | |
| LPA- | AAUACUACCCAAAUGGUGG | 172 | GUCAGUCCACCAUU | 572 | AAUACUACCC | 972 |
| 4001 | ACUGAC | | UGGGUAGUAUUCU | | AAAUGGUGG | |
| LPA- | UCCUUCUGAAGAAGCACCA | 173 | UUCAGUUGGUGCUU | 573 | UCCUUCUGAA | 973 |
| 4185 | ACUGAA | | CUUCAGAAGGAAG | | GAAGCACCA | |
| LPA- | CCUUCUGAAGAAGCACCAA | 174 | UUUCAUUUGGUGCU | 574 | CCUUCUGAAG | 974 |
| 4186 | AUGAAA | | UCUUCAGAAGGAA | | AAGCACCAA | |
| LPA- | CUUCUGAAGAAGCACCAAC | 175 | UUUUCUGUUGGUGC | 575 | CUUCUGAAGA | 975 |
| 4187 | AGAAAA | | UUCUUCAGAAGGA | | AGCACCAAC | |
| LPA- | UUCUGAAGAAGCACCAACU | 176 | GUUUUUAGUUGGUG | 576 | UUCUGAAGAA | 976 |
| 4188 | AAAAAC | | CUUCUUCAGAAGG | | GCACCAACU | |
| LPA- | UCUGAAGAAGCACCAACUG | 177 | UGUUUUCAGUUGGU | 577 | UCUGAAGAAG | 977 |
| 4189 | AAAACA | | GCUUCUUCAGAAG | | CACCAACUG | |
| LPA- | CUGAAGAAGCACCAACUGA | 178 | CUGUUUUCAGUUGG | 578 | CUGAAGAAGC | 978 |
| 4190 | AAACAG | | UGCUUCUUCAGAA | | ACCAACUGA | |
| LPA- | UGAAGAAGCACCAACUGAA | 179 | GCUGUUUUCAGUUG | 579 | UGAAGAAGCA | 979 |
| 4191 | AACAGC | | GUGCUUCUUCAGA | | CCAACUGAA | |
| LPA- | GAAGAAGCACCAACUGAAA | 180 | UGCUGUUUUCAGUU | 580 | GAAGAAGCAC | 980 |
| 4192 | ACAGCA | | GGUGCUUCUUCAG | | CAACUGAAA | |
| LPA- | AAGAAGCACCAACUGAAAA | 181 | GUGCUUUUUUCAGU | 581 | AAGAAGCACC | 981 |
| 4193 | AAGCAC | | UGGUGCUUCUUCA | | AACUGAAAA | |
| LPA- | AGAAGCACCAACUGAAAAC | 182 | AGUGCUGUUUUCAG | 582 | AGAAGCACCA | 982 |
| 4194 | AGCACT | | UUGGUGCUUCUUC | | ACUGAAAAC | |
| LPA- | GAAGCACCAACUGAAAACA | 183 | CAGUGUUGUUUUCA | 583 | GAAGCACCAA | 983 |
| 4195 | ACACTG | | GUUGGUGCUUCUU | | CUGAAAACA | |
| LPA- | AAGCACCAACUGAAAACAG | 184 | CCAGUUCUGUUUUC | 584 | AAGCACCAAC | 984 |
| 4196 | AACUGG | | AGUUGGUGCUUCU | | UGAAAACAG | |
| LPA- | AGGUGAUGGACAGAGUUAU | 185 | GCCUCUAUAACUCU | 585 | AGGUGAUGGA | 985 |
| 4239 | AGAGGC | | GUCCAUCACCUCG | | CAGAGUUAU | |
| LPA- | CUCCACCACUAUCACAGGA | 186 | UGUUCUUCCUGUGA | 586 | CUCCACCACU | 986 |
| 4269 | AGAACA | | UAGUGGUGGAGAG | | AUCACAGGA | |
| LPA- | UCCACCACUAUCACAGGAA | 187 | AUGUUUUUCCUGUG | 587 | UCCACCACUA | 987 |
| 4270 | AAACAT | | AUAGUGGUGGAGA | | UCACAGGAA | |
| LPA- | CCACCACUAUCACAGGAAG | 188 | CAUGUUCUUCCUGU | 588 | CCACCACUAU | 988 |
| 4271 | AACATG | | GAUAGUGGUGGAG | | CACAGGAAG | |
| LPA- | CACCACUAUCACAGGAAGA | 189 | ACAUGUUCUUCCUG | 589 | CACCACUAUC | 989 |
| 4272 | ACAUGT | | UGAUAGUGGUGGA | | ACAGGAAGA | |
| LPA- | ACCACUAUCACAGGAAGAA | 190 | GACAUUUUCUUCCU | 590 | ACCACUAUCA | 990 |
| 4273 | AAUGTC | | GUGAUAGUGGUGG | | CAGGAAGAA | |
| LPA- | CCACUAUCACAGGAAGAAC | 191 | UGACAUGUUCUUCC | 591 | CCACUAUCAC | 991 |
| 4274 | AUGUCA | | UGUGAUAGUGGUG | | AGGAAGAAC | |
| LPA- | CACUAUCACAGGAAGAACA | 192 | CUGACUUGUUCUUC | 592 | CACUAUCACA | 992 |
| 4275 | AGUCAG | | CUGUGAUAGUGGU | | GGAAGAACA | |
| LPA- | ACUAUCACAGGAAGAACAU | 193 | ACUGAUAUGUUCUU | 593 | ACUAUCACAG | 993 |
| 4276 | AUCAGT | | CCUGUGAUAGUGG | | GAAGAACAU | |
| LPA- | CUAUCACAGGAAGAACAUG | 194 | GACUGUCAUGUUCU | 594 | CUAUCACAGG | 994 |
| 4277 | ACAGTC | | UCCUGUGAUAGUG | | AAGAACAUG | |
| LPA- | UAUCACAGGAAGAACAUGU | 195 | AGACUUACAUGUUC | 595 | UAUCACAGGA | 995 |
| 4278 | AAGUCT | | UUCCUGUGAUAGU | | AGAACAUGU | |
| LPA- | AUCACAGGAAGAACAUGUC | 196 | AAGACUGACAUGUU | 596 | AUCACAGGAA | 996 |
| 4279 | AGUCTT | | CUUCCUGUGAUAG | | GAACAUGUC | |
| LPA- | UCACAGGAAGAACAUGUCA | 197 | CAAGAUUGACAUGU | 597 | UCACAGGAAG | 997 |
| 4280 | AUCUTG | | UCUUCCUGUGAUA | | AACAUGUCA | |
| LPA- | CACAGGAAGAACAUGUCAG | 198 | CCAAGUCUGACAUG | 598 | CACAGGAAGA | 998 |
| 4281 | ACUUGG | | UUCUUCCUGUGAU | | ACAUGUCAG | |
| LPA- | ACAGGAAGAACAUGUCAGU | 199 | ACCAAUACUGACAU | 599 | ACAGGAAGAA | 999 |
| 4282 | AUUGGT | | GUUCUUCCUGUGA | | CAUGUCAGU | |
| LPA- | GGAAGAACAUGUCAGUCUU | 200 | ACGACUAAGACUGA | 600 | GGAAGAACAU | 1000 |
| 4285 | AGUCGT | | CAUGUUCUUCCUG | | GUCAGUCUU | |
| LPA- | GAAGAACAUGUCAGUCUUG | 201 | GACGAUCAAGACUG | 601 | GAAGAACAUG | 1001 |
| 4286 | AUCGTC | | ACAUGUUCUUCCU | | UCAGUCUUG | |
| LPA- | AAGAACAUGUCAGUCUUGG | 202 | AGACGUCCAAGACU | 602 | AAGAACAUGU | 1002 |
| 4287 | ACGUCT | | GACAUGUUCUUCC | | CAGUCUUGG | |
| LPA- | AGAACAUGUCAGUCUUGGU | 203 | UAGACUACCAAGAC | 603 | AGAACAUGUC | 1003 |
| 4288 | AGUCTA | | UGACAUGUUCUUC | | AGUCUUGGU | |
| LPA- | GGCAUCGGAGGAUCCCAUU | 204 | UAGUAUAAUGGGAU | 604 | GGCAUCGGAG | 1004 |
| 4325 | AUACTA | | CCUCCGAUGCCAA | | GAUCCCAUU | |
| LPA- | ACUAUCCAAAUGCUGGCCU | 205 | CUGGUUAGGCCAGC | 605 | ACUAUCCAAA | 1005 |
| 4346 | AACCAG | | AUUUGGAUAGUAU | | UGCUGGCCU | |
| LPA- | GCACAGAGGCUCCUUCUGA | 206 | GCUUGUUCAGAAGG | 606 | GCACAGAGGC | 1006 |
| 4517 | ACAAGC | | AGCCUCUGUGCUU | | UCCUUCUGA | |
| LPA- | UCCUUCUGAACAAGCACCA | 207 | CUCAGUUGGUGCUU | 607 | UCCUUCUGAA | 1007 |
| 4527 | ACUGAG | | GUUCAGAAGGAGC | | CAAGCACCA | |
| LPA- | CCUUCUGAACAAGCACCAC | 208 | UCUCAUGUGGUGCU | 608 | CCUUCUGAAC | 1008 |
| 4528 | AUGAGA | | UGUUCAGAAGGAG | | AAGCACCAC | |
| LPA- | CUUCUGAACAAGCACCACC | 209 | UUCUCUGGUGGUGC | 609 | CUUCUGAACA | 1009 |
| 4529 | AGAGAA | | UUGUUCAGAAGGA | | AGCACCACC | |
| LPA- | UUCUGAACAAGCACCACCU | 210 | UUUCUUAGGUGGUG | 610 | UUCUGAACAA | 1010 |
| 4530 | AAGAAA | | CUUGUUCAGAAGG | | GCACCACCU | |
| LPA- | UCUGAACAAGCACCACCUG | 211 | UUUUCUCAGGUGGU | 611 | UCUGAACAAG | 1011 |
| 4531 | AGAAAA | | GCUUGUUCAGAAG | | CACCACCUG | |
| LPA- | CUGAACAAGCACCACCUGA | 212 | CUUUUUUCAGGUGG | 612 | CUGAACAAGC | 1012 |
| 4532 | AAAAAG | | UGCUUGUUCAGAA | | ACCACCUGA | |
| LPA- | UGAACAAGCACCACCUGAG | 213 | GCUUUUCUCAGGUG | 613 | UGAACAAGCA | 1013 |
| 4533 | AAAAGC | | GUGCUUGUUCAGA | | CCACCUGAG | |
| LPA- | GAACAAGCACCACCUGAGA | 214 | GGCUUUUCUCAGGU | 614 | GAACAAGCAC | 1014 |
| 4531 | AAAGCC | | GGUGCUUGUUCAG | | CACCUGAGA | |
| LPA- | AACAAGCACCACCUGAGAA | 215 | GGGCUUUUCUCAGG | 615 | AACAAGCACC | 1015 |
| 4535 | AAGCCC | | UGGUGCUUGUUCA | | ACCUGAGAA | |
| LPA- | CAAGCACCACCUGAGAAAA | 216 | CAGGGUUUUUCUCA | 616 | CAAGCACCAC | 1016 |
| 4537 | ACCCTG | | GGUGGUGCUUGUU | | CUGAGAAAA | |
| LPA- | AAGCACCACCUGAGAAAAG | 217 | ACAGGUCUUUUCUC | 617 | AAGCACCACC | 1017 |
| 4538 | ACCUGT | | AGGUGGUGCUUGU | | UGAGAAAAG | |
| LPA- | AGCACCACCUGAGAAAAGC | 218 | CACAGUGCUUUUCU | 618 | AGCACCACCU | 1018 |
| 4539 | ACUGTG | | CAGGUGGUGCUUG | | GAGAAAAGC | |
| LPA- | CUGAGAAAAGCCCUGUGGU | 219 | UCCUGUACCACAGG | 619 | CUGAGAAAAG | 1019 |
| 4547 | ACAGGA | | GCUUUUCUCAGGU | | CCCUGUGGU | |
| LPA- | GCCCUGUGGUCCAGGAUUG | 220 | UGGUAUCAAUCCUG | 620 | GCCCUGUGGU | 1020 |
| 4556 | AUACCA | | GACCACAGGGCUU | | CCAGGAUUG | |
| LPA- | CUGUGGUCCAGGAUUGCUA | 221 | CCAUGUUAGCAAUC | 621 | CUGUGGUCCA | 1021 |
| 4559 | ACAUGG | | CUGGACCACAGGG | | GGAUUGCUA | |
| LPA- | CUCCACCACUGUCACAGGA | 222 | GGUCCUUCCUGUGA | 622 | CUCCACCACU | 1022 |
| 4611 | AGGACC | | CAGUGGUGGAGGA | | GUCACAGGA | |
| LPA- | UCCACCACUGUCACAGGAA | 223 | AGGUCUUUCCUGUG | 623 | UCCACCACUG | 1023 |
| 4612 | AGACCT | | ACAGUGGUGGAGG | | UCACAGGAA | |
| LPA- | UCUUGGUCAUCUAUGAUAC | 224 | AGUGUUGUAUCAUA | 624 | UCUUGGUCAU | 1024 |
| 4642 | AACACT | | GAUGACCAAGAUU | | CUAUGAUAC | |
| LPA- | CUUGGUCAUCUAUGAUACC | 225 | CAGUGUGGUAUCAU | 625 | CUUGGUCAUC | 1025 |
| 4643 | ACACTG | | AGAUGACCAAGAU | | UAUGAUACC | |
| LPA- | UUGGUCAUCUAUGAUACCA | 226 | CCAGUUUGGUAUCA | 626 | UUGGUCAUCU | 1026 |
| 4644 | AACUGG | | UAGAUGACCAAGA | | AUGAUACCA | |
| LPA- | UGGUCAUCUAUGAUACCAC | 227 | GCCAGUGUGGUAUC | 627 | UGGUCAUCUA | 1027 |
| 4645 | ACUGGC | | AUAGAUGACCAAG | | UGAUACCAC | |
| LPA- | GGUCAUCUAUGAUACCACA | 228 | UGCCAUUGUGGUAU | 628 | GGUCAUCUAU | 1028 |
| 4646 | AUGGCA | | CAUAGAUGACCAA | | GAUACCACA | |
| LPA- | GUCAUCUAUGAUACCACAC | 229 | AUGCCUGUGUGGUA | 629 | GUCAUCUAUG | 1029 |
| 4647 | AGGCAT | | UCAUAGAUGACCA | | AUACCACAC | |
| LPA- | UCAUCUAUGAUACCACACU | 230 | GAUGCUAGUGUGGU | 630 | UCAUCUAUGA | 1030 |
| 4648 | AGCATC | | AUCAUAGAUGACC | | UACCACACU | |
| LPA- | CAUCUAUGAUACCACACUG | 231 | UGAUGUCAGUGUGG | 631 | CAUCUAUGAU | 1031 |
| 4649 | ACAUCA | | UAUCAUAGAUGAC | | ACCACACUG | |
| LPA- | AUCUAUGAUACCACACUGG | 232 | CUGAUUCCAGUGUG | 632 | AUCUAUGAUA | 1032 |
| 4650 | AAUCAG | | GUAUCAUAGAUGA | | CCACACUGG | |
| LPA- | UCUAUGAUACCACACUGGC | 233 | UCUGAUGCCAGUGU | 633 | UCUAUGAUAC | 1033 |
| 4651 | AUCAGA | | GGUAUCAUAGAUG | | CACACUGGC | |
| LPA- | CUAUGAUACCACACUGGCA | 234 | CUCUGUUGCCAGUG | 634 | CUAUGAUACC | 1034 |
| 4652 | ACAGAG | | UGGUAUCAUAGAU | | ACACUGGCA | |
| LPA- | UGAUACCACACUGGCAUCA | 235 | GUCCUUUGAUGCCA | 635 | UGAUACCACA | 1035 |
| 4655 | AAGGAC | | GUGUGGUAUCAUA | | CUGGCAUCA | |
| LPA- | AUACCACACUGGCAUCAGA | 236 | GGGUCUUCUGAUGC | 636 | AUACCACACU | 1036 |
| 4657 | AGACCC | | CAGUGUGGUAUCA | | GGCAUCAGA | |
| LPA- | AGAGGACCCCAGAAAACUA | 237 | UUUGGUUAGUUUUC | 637 | AGAGGACCCC | 1037 |
| 4673 | ACCAAA | | UGGGGUCCUCUGA | | AGAAAACUA | |
| LPA- | GAGGACCCCAGAAAACUAC | 238 | AUUUGUGUAGUUUU | 638 | GAGGACCCCA | 1038 |
| 4674 | ACAAAT | | CUGGGGUCCUCUG | | GAAAACUAC | |
| LPA- | AGAACUACUGCAGGAAUCC | 239 | GAAUCUGGAUUCCU | 639 | AGAACUACUG | 1039 |
| 4712 | AGAUTC | | GCAGUAGUUCUCG | | CAGGAAUCC | |
| LPA- | ACUACUGCAGGAAUCCAGA | 240 | CCAGAUUCUGGAUU | 640 | ACUACUGCAG | 1040 |
| 4715 | AUCUGG | | CCUGCAGUAGUUC | | GAAUCCAGA | |
| LPA- | UACUGCAGGAAUCCAGAUU | 241 | UCCCAUAAUCUGGA | 641 | UACUGCAGGA | 1041 |
| 4717 | AUGGGA | | UUCCUGCAGUAGU | | AUCCAGAUU | |
| LPA- | ACUGCAGGAAUCCAGAUUC | 242 | UUCCCUGAAUCUGG | 642 | ACUGCAGGAA | 1042 |
| 4718 | AGGGAA | | AUUCCUGCAGUAG | | UCCAGAUUC | |
| LPA- | CUGCAGGAAUCCAGAUUCU | 243 | UUUCCUAGAAUCUG | 643 | CUGCAGGAAU | 1043 |
| 4719 | AGGAAA | | GAUUCCUGCAGUA | | CCAGAUUCU | |
| LPA- | UGCAGGAAUCCAGAUUCUG | 244 | GUUUCUCAGAAUCU | 644 | UGCAGGAAUC | 1044 |
| 4720 | AGAAAC | | GGAUUCCUGCAGU | | CAGAUUCUG | |
| LPA- | GCAGGAAUCCAGAUUCUGG | 245 | UGUUUUCCAGAAUC | 645 | GCAGGAAUCC | 1045 |
| 4721 | AAAACA | | UGGAUUCCUGCAG | | AGAUUCUGG | |
| LPA- | GGAAUCCAGAUUCUGGGAA | 246 | GGUUGUUUCCCAGA | 646 | GGAAUCCAGA | 1046 |
| 4724 | ACAACC | | AUCUGGAUUCCUG | | UUCUGGGAA | |
| LPA- | GGGAAACAACCCUGGUGUU | 247 | UUGUGUAACACCAG | 647 | GGGAAACAAC | 1047 |
| 4738 | ACACAA | | GGUUGUUUCCCAG | | CCUGGUGUU | |
| LPA- | GGAAACAACCCUGGUGUUA | 248 | GUUGUUUAACACCA | 648 | GGAAACAACC | 1048 |
| 4739 | AACAAC | | GGGUUGUUUCCCA | | CUGGUGUUA | |
| LPA- | UGUGUGAGGUGGGAGUACU | 249 | GAUUGUAGUACUCC | 649 | UGUGUGAGGU | 1049 |
| 4771 | ACAATC | | CACCUCACACACG | | GGGAGUACU | |
| LPA- | GUGUGAGGUGGGAGUACUG | 250 | AGAUUUCAGUACUC | 650 | GUGUGAGGUG | 1050 |
| 4772 | AAAUCT | | CCACCUCACACAC | | GGAGUACUG | |
| LPA- | GUGAGGUGGGAGUACUGCA | 251 | UCAGAUUGCAGUAC | 651 | GUGAGGUGGG | 1051 |
| 4774 | AUCUGA | | UCCCACCUCACAC | | AGUACUGCA | |
| LPA- | UGAGGUGGGAGUACUGCAA | 252 | GUCAGUUUGCAGUA | 652 | UGAGGUGGGA | 1052 |
| 4775 | ACUGAC | | CUCCCACCUCACA | | GUACUGCAA | |
| LPA- | CUGACACAAUGCUCAGAAA | 253 | AUUCUUUUUCUGAG | 653 | CUGACACAAU | 1053 |
| 4795 | AAGAAT | | CAUUGUGUCAGAU | | GCUCAGAAA | |
| LPA- | UGACACAAUGCUCAGAAAC | 254 | GAUUCUGUUUCUGA | 654 | UGACACAAUG | 1054 |
| 4796 | AGAATC | | GCAUUGUGUCAGA | | CUCAGAAAC | |
| LPA- | GACACAAUGCUCAGAAACA | 255 | UGAUUUUGUUUCUG | 655 | GACACAAUGC | 1055 |
| 4797 | AAAUCA | | AGCAUUGUGUCAG | | UCAGAAACA | |
| LPA- | ACACAAUGCUCAGAAACAG | 256 | CUGAUUCUGUUUCU | 656 | ACACAAUGCU | 1056 |
| 4798 | AAUCAG | | GAGCAUUGUGUCA | | CAGAAACAG | |
| LPA- | CACAAUGCUCAGAAACAGA | 257 | CCUGAUUCUGUUUC | 657 | CACAAUGCUC | 1057 |
| 4799 | AUCAGG | | UGAGCAUUGUGUC | | AGAAACAGA | |
| LPA- | ACAAUGCUCAGAAACAGAA | 258 | ACCUGUUUCUGUUU | 658 | ACAAUGCUCA | 1058 |
| 4800 | ACAGGT | | CUGAGCAUUGUGU | | GAAACAGAA | |
| LPA- | CAAUGCUCAGAAACAGAAU | 259 | CACCUUAUUCUGUU | 659 | CAAUGCUCAG | 1059 |
| 4801 | AAGGTG | | UCUGAGCAUUGUG | | AAACAGAAU | |
| LPA- | AAUGCUCAGAAACAGAAUC | 260 | ACACCUGAUUCUGU | 660 | AAUGCUCAGA | 1060 |
| 4802 | AGGUGT | | UUCUGAGCAUUGU | | AACAGAAUC | |
| LPA- | AUGCUCAGAAACAGAAUCA | 261 | GACACUUGAUUCUG | 661 | AUGCUCAGAA | 1061 |
| 4803 | AGUGTC | | UUUCUGAGCAUUG | | ACAGAAUCA | |
| LPA- | UGCUCAGAAACAGAAUCAG | 262 | GGACAUCUGAUUCU | 662 | UGCUCAGAAA | 1062 |
| 4804 | AUGUCC | | GUUUCUGAGCAUU | | CAGAAUCAG | |
| LPA- | CUCAGAAACAGAAUCAGGU | 263 | UAGGAUACCUGAUU | 663 | CUCAGAAACA | 1063 |
| 4806 | AUCCTA | | CUGUUUCUGAGCA | | GAAUCAGGU | |
| LPA- | CAGAAACAGAAUCAGGUGU | 264 | UCUAGUACACCUGA | 664 | CAGAAACAGA | 1064 |
| 4808 | ACUAGA | | UUCUGUUUCUGAG | | AUCAGGUGU | |
| LPA- | AGAAACAGAAUCAGGUGUC | 265 | CUCUAUGACACCUG | 665 | AGAAACAGAA | 1065 |
| 4809 | AUAGAG | | AUUCUGUUUCUGA | | UCAGGUGUC | |
| LPA- | GAAACAGAAUCAGGUGUCC | 266 | UCUCUUGGACACCU | 666 | GAAACAGAAU | 1066 |
| 4810 | AAGAGA | | GAUUCUGUUUCUG | | CAGGUGUCC | |
| LPA- | AAACAGAAUCAGGUGUCCU | 267 | GUCUCUAGGACACC | 667 | AAACAGAAUC | 1067 |
| 4811 | AGAGAC | | UGAUUCUGUUUCU | | AGGUGUCCU | |
| LPA- | AACAGAAUCAGGUGUCCUA | 268 | AGUCUUUAGGACAC | 668 | AACAGAAUCA | 1068 |
| 4812 | AAGACT | | CUGAUUCUGUUUC | | GGUGUCCUA | |
| LPA- | CAGAAUCAGGUGUCCUAGA | 269 | GGAGUUUCUAGGAC | 669 | CAGAAUCAGG | 1069 |
| 4814 | AACUCC | | ACCUGAUUCUGUU | | UGUCCUAGA | |
| LPA- | GAAUCAGGUGUCCUAGAGA | 270 | UGGGAUUCUCUAGG | 670 | GAAUCAGGUG | 1070 |
| 4816 | AUCCCA | | ACACCUGAUUCUG | | UCCUAGAGA | |
| LPA- | AUCAGGUGUCCUAGAGACU | 271 | AGUGGUAGUCUCUA | 671 | AUCAGGUGUC | 1071 |
| 4818 | ACCACT | | GGACACCUGAUUC | | CUAGAGACU | |
| LPA- | GGUGUCCUAGAGACUCCCA | 272 | CAACAUUGGGAGUC | 672 | GGUGUCCUAG | 1072 |
| 4822 | AUGUTG | | UCUAGGACACCUG | | AGACUCCCA | |
| LPA- | CCUAGAGACUCCCACUGUU | 273 | UGGAAUAACAGUGG | 673 | CCUAGAGACU | 1073 |
| 4827 | AUUCCA | | GAGUCUCUAGGAC | | CCCACUGUU | |
| LPA- | CUAGAGACUCCCACUGUUG | 274 | CUGGAUCAACAGUG | 674 | CUAGAGACUC | 1074 |
| 4828 | AUCCAG | | GGAGUCUCUAGGA | | CCACUGUUG | |
| LPA- | UAGAGACUCCCACUGUUGU | 275 | ACUGGUACAACAGU | 675 | UAGAGACUCC | 1075 |
| 4829 | ACCAGT | | GGGAGUCUCUAGG | | CACUGUUGU | |
| LPA- | AGAGACUCCCACUGUUGUU | 276 | AACUGUAACAACAG | 676 | AGAGACUCCC | 1076 |
| 4830 | ACAGTT | | UGGGAGUCUCUAG | | ACUGUUGUU | |
| LPA- | GAGACUCCCACUGUUGUUC | 277 | GAACUUGAACAACA | 677 | GAGACUCCCA | 1077 |
| 4831 | AAGUTC | | GUGGGAGUCUCUA | | CUGUUGUUC | |
| LPA- | AGACUCCCACUGUUGUUCC | 278 | GGAACUGGAACAAC | 678 | AGACUCCCAC | 1078 |
| 4832 | AGUUCC | | AGUGGGAGUCUCU | | UGUUGUUCC | |
| LPA- | GCUCAUUCUGAAGCAGCAC | 279 | CAGUUUGUGCUGCU | 679 | GCUCAUUCUG | 1079 |
| 4867 | AAACTG | | UCAGAAUGAGCCU | | AAGCAGCAC | |
| LPA- | CUCAUUCUGAAGCAGCACC | 280 | UCAGUUGGUGCUGC | 680 | CUCAUUCUGA | 1080 |
| 4868 | AACUGA | | UUCAGAAUGAGCC | | AGCAGCACC | |
| LPA- | UCAUUCUGAAGCAGCACCA | 281 | CUCAGUUGGUGCUG | 681 | UCAUUCUGAA | 1081 |
| 4869 | ACUGAG | | CUUCAGAAUGAGC | | GCAGCACCA | |
| LPA- | CAUUCUGAAGCAGCACCAA | 282 | GCUCAUUUGGUGCU | 682 | CAUUCUGAAG | 1082 |
| 4870 | AUGAGC | | GCUUCAGAAUGAG | | CAGCACCAA | |
| LPA- | AUUCUGAAGCAGCACCAAC | 283 | UGCUCUGUUGGUGC | 683 | AUUCUGAAGC | 1083 |
| 4871 | AGAGCA | | UGCUUCAGAAUGA | | AGCACCAAC | |
| LPA- | UUCUGAAGCAGCACCAACU | 284 | UUGCUUAGUUGGUG | 684 | UUCUGAAGCA | 1084 |
| 4872 | AAGCAA | | CUGCUUCAGAAUG | | GCACCAACU | |
| LPA- | UCUGAAGCAGCACCAACUG | 285 | UUUGCUCAGUUGGU | 685 | UCUGAAGCAG | 1085 |
| 4873 | AGCAAA | | GCUGCUUCAGAAU | | CACCAACUG | |
| LPA- | CUGAAGCAGCACCAACUGA | 286 | GUUUGUUCAGUUGG | 686 | CUGAAGCAGC | 1086 |
| 4874 | ACAAAC | | UGCUGCUUCAGAA | | ACCAACUGA | |
| LPA- | UGAAGCAGCACCAACUGAG | 287 | GGUUUUCUCAGUUG | 687 | UGAAGCAGCA | 1087 |
| 4875 | AAAACC | | GUGCUGCUUCAGA | | CCAACUGAG | |
| LPA- | GAAGCAGCACCAACUGAGC | 288 | GGGUUUGCUCAGUU | 688 | GAAGCAGCAC | 1088 |
| 4876 | AAACCC | | GGUGCUGCUUCAG | | CAACUGAGC | |
| LPA- | AAGCAGCACCAACUGAGCA | 289 | GGGGUUUGCUCAGU | 689 | AAGCAGCACC | 1089 |
| 4877 | AACCCC | | UGGUGCUGCUUCA | | AACUGAGCA | |
| LPA- | CAGUGCUACCAUGGUAAUG | 290 | UCUGGUCAUUACCA | 690 | CAGUGCUACC | 1090 |
| 4912 | ACCAGA | | UGGUAGCACUGCC | | AUGGUAAUG | |
| LPA- | AGUGCUACCAUGGUAAUGG | 291 | CUCUGUCCAUUACC | 691 | AGUGCUACCA | 1091 |
| 4913 | ACAGAG | | AUGGUAGCACUGC | | UGGUAAUGG | |
| LPA- | ACAUUCUCCACCACUGUCA | 292 | UUCCUUUGACAGUG | 692 | ACAUUCUCCA | 1092 |
| 4948 | AAGGAA | | GUGGAGAAUGUGC | | CCACUGUCA | |
| LPA- | CACUGUCACAGGAAGGACA | 293 | UUGACUUGUCCUUC | 693 | CACUGUCACA | 1093 |
| 4959 | AGUCAA | | CUGUGACAGUGGU | | GGAAGGACA | |
| LPA- | ACUGUCACAGGAAGGACAU | 294 | AUUGAUAUGUCCUU | 694 | ACUGUCACAG | 1094 |
| 4960 | AUCAAT | | CCUGUGACAGUGG | | GAAGGACAU | |
| LPA- | CUGUCACAGGAAGGACAUG | 295 | GAUUGUCAUGUCCU | 695 | CUGUCACAGG | 1095 |
| 4961 | ACAATC | | UCCUGUGACAGUG | | AAGGACAUG | |
| LPA- | UGUCACAGGAAGGACAUGU | 296 | AGAUUUACAUGUCC | 696 | UGUCACAGGA | 1096 |
| 4962 | AAAUCT | | UUCCUGUGACAGU | | AGGACAUGU | |
| LPA- | GUCACAGGAAGGACAUGUC | 297 | AAGAUUGACAUGUC | 697 | GUCACAGGAA | 1097 |
| 4963 | AAUCTT | | CUUCCUGUGACAG | | GGACAUGUC | |
| LPA- | UCACAGGAAGGACAUGUCA | 298 | CAAGAUUGACAUGU | 698 | UCACAGGAAG | 1098 |
| 4964 | AUCUTG | | CCUUCCUGUGACA | | GACAUGUCA | |
| LPA- | ACAGGAAGGACAUGUCAAU | 299 | ACCAAUAUUGACAU | 699 | ACAGGAAGGA | 1099 |
| 4966 | AUUGGT | | GUCCUUCCUGUGA | | CAUGUCAAU | |
| LPA- | CAGGAAGGACAUGUCAAUC | 300 | GACCAUGAUUGACA | 700 | CAGGAAGGAC | 1100 |
| 4967 | AUGGTC | | UGUCCUUCCUGUG | | AUGUCAAUC | |
| LPA- | AGGAAGGACAUGUCAAUCU | 301 | UGACCUAGAUUGAC | 701 | AGGAAGGACA | 1101 |
| 4968 | AGGUCA | | AUGUCCUUCCUGU | | UGUCAAUCU | |
| LPA- | GGAAGGACAUGUCAAUCUU | 302 | AUGACUAAGAUUGA | 702 | GGAAGGACAU | 1102 |
| 4969 | AGUCAT | | CAUGUCCUUCCUG | | GUCAAUCUU | |
| LPA- | GAAGGACAUGUCAAUCUUG | 303 | GAUGAUCAAGAUUG | 703 | GAAGGACAUG | 1103 |
| 4970 | AUCATC | | ACAUGUCCUUCCU | | UCAAUCUUG | |
| LPA- | AAGGACAUGUCAAUCUUGG | 304 | GGAUGUCCAAGAUU | 704 | AAGGACAUGU | 1104 |
| 4971 | ACAUCC | | GACAUGUCCUUCC | | CAAUCUUGG | |
| LPA- | AGGACAUGUCAAUCUUGGU | 305 | UGGAUUACCAAGAU | 705 | AGGACAUGUC | 1105 |
| 4972 | AAUCCA | | UGACAUGUCCUUC | | AAUCUUGGU | |
| LPA- | GGACAUGUCAAUCUUGGUC | 306 | AUGGAUGACCAAGA | 706 | GGACAUGUCA | 1106 |
| 4973 | AUCCAT | | UUGACAUGUCCUU | | AUCUUGGUC | |
| LPA- | GACAUGUCAAUCUUGGUCA | 307 | CAUGGUUGACCAAG | 707 | GACAUGUCAA | 1107 |
| 4974 | ACCATG | | AUUGACAUGUCCU | | UCUUGGUCA | |
| LPA- | ACAUGUCAAUCUUGGUCAU | 308 | UCAUGUAUGACCAA | 708 | ACAUGUCAAU | 1108 |
| 4975 | ACAUGA | | GAUUGACAUGUCC | | CUUGGUCAU | |
| LPA- | CAUGUCAAUCUUGGUCAUC | 309 | GUCAUUGAUGACCA | 709 | CAUGUCAAUC | 1109 |
| 4976 | AAUGAC | | AGAUUGACAUGUC | | UUGGUCAUC | |
| LPA- | AUGUCAAUCUUGGUCAUCC | 310 | UGUCAUGGAUGACC | 710 | AUGUCAAUCU | 1110 |
| 4977 | AUGACA | | AAGAUUGACAUGU | | UGGUCAUCC | |
| LPA- | UGUCAAUCUUGGUCAUCCA | 311 | GUGUCUUGGAUGAC | 711 | UGUCAAUCUU | 1111 |
| 4978 | AGACAC | | CAAGAUUGACAUG | | GGUCAUCCA | |
| LPA- | GUCAAUCUUGGUCAUCCAU | 312 | GGUGUUAUGGAUGA | 712 | GUCAAUCUUG | 1112 |
| 4979 | AACACC | | CCAAGAUUGACAU | | GUCAUCCAU | |
| LPA- | UCAAUCUUGGUCAUCCAUG | 313 | UGGUGUCAUGGAUG | 713 | UCAAUCUUGG | 1113 |
| 4980 | ACACCA | | ACCAAGAUUGACA | | UCAUCCAUG | |
| LPA- | CAAUCUUGGUCAUCCAUGA | 314 | GUGGUUUCAUGGAU | 714 | CAAUCUUGGU | 1114 |
| 4981 | AACCAC | | GACCAAGAUUGAC | | CAUCCAUGA | |
| LPA- | AAUCUUGGUCAUCCAUGAC | 315 | UGUGGUGUCAUGGA | 715 | AAUCUUGGUC | 1115 |
| 4982 | ACCACA | | UGACCAAGAUUGA | | AUCCAUGAC | |
| LPA- | AUCUUGGUCAUCCAUGACA | 316 | GUGUGUUGUCAUGG | 716 | AUCUUGGUCA | 1116 |
| 4983 | ACACAC | | AUGACCAAGAUUG | | UCCAUGACA | |
| LPA- | UGACAAUGAACUACUGCAG | 317 | GGAUUUCUGCAGUA | 717 | UGACAAUGAA | 1117 |
| 5048 | AAAUCC | | GUUCAUUGUCAGG | | CUACUGCAG | |
| LPA- | GACAAUGAACUACUGCAGG | 318 | UGGAUUCCUGCAGU | 718 | GACAAUGAAC | 1118 |
| 5049 | AAUCCA | | AGUUCAUUGUCAG | | UACUGCAGG | |
| LPA- | ACAAUGAACUACUGCAGGA | 319 | CUGGAUUCCUGCAG | 719 | ACAAUGAACU | 1119 |
| 5050 | AUCCAG | | UAGUUCAUUGUCA | | ACUGCAGGA | |
| LPA- | CAAUGAACUACUGCAGGAA | 320 | UCUGGUUUCCUGCA | 720 | CAAUGAACUA | 1120 |
| 5051 | ACCAGA | | GUAGUUCAUUGUC | | CUGCAGGAA | |
| LPA- | AAUGAACUACUGCAGGAAU | 321 | AUCUGUAUUCCUGC | 721 | AAUGAACUAC | 1121 |
| 5052 | ACAGAT | | AGUAGUUCAUUGU | | UGCAGGAAU | |
| LPA- | AUGAACUACUGCAGGAAUC | 322 | CAUCUUGAUUCCUG | 722 | AUGAACUACU | 1122 |
| 5053 | AAGATG | | CAGUAGUUCAUUG | | GCAGGAAUC | |
| LPA- | UGAACUACUGCAGGAAUCC | 323 | GCAUCUGGAUUCCU | 723 | UGAACUACUG | 1123 |
| 5054 | AGAUGC | | GCAGUAGUUCAUU | | CAGGAAUCC | |
| LPA- | CUACUGCAGGAAUCCAGAU | 324 | AUCGGUAUCUGGAU | 724 | CUACUGCAGG | 1124 |
| 5058 | ACCGAT | | UCCUGCAGUAGUU | | AAUCCAGAU | |
| LPA- | CAGGCCCUUGGUGUUUUAC | 325 | UCCAUUGUAAAACA | 725 | CAGGCCCUUG | 1125 |
| 5084 | AAUGGA | | CCAAGGGCCUGUA | | GUGUUUUAC | |
| LPA- | CUUGGUGUUUUACCAUGGA | 326 | CUGGGUUCCAUGGU | 726 | CUUGGUGUUU | 1126 |
| 5090 | ACCCAG | | AAAACACCAAGGG | | UACCAUGGA | |
| LPA- | UUGGUGUUUUACCAUGGAC | 327 | GCUGGUGUCCAUGG | 727 | UUGGUGUUUU | 1127 |
| 5091 | ACCAGC | | UAAAACACCAAGG | | ACCAUGGAC | |
| LPA- | UGGUGUUUUACCAUGGACC | 328 | UGCUGUGGUCCAUG | 728 | UGGUGUUUUA | 1128 |
| 5092 | ACAGCA | | GUAAAACACCAAG | | CCAUGGACC | |
| LPA- | GGUGUUUUACCAUGGACCC | 329 | AUGCUUGGGUCCAU | 729 | GGUGUUUUAC | 1129 |
| 5093 | AAGCAT | | GGUAAAACACCAA | | CAUGGACCC | |
| LPA- | GUGUUUUACCAUGGACCCC | 330 | GAUGCUGGGGUCCA | 730 | GUGUUUUACC | 1130 |
| 5094 | AGCATC | | UGGUAAAACACCA | | AUGGACCCC | |
| LPA- | GUUUUACCAUGGACCCCAG | 331 | CUGAUUCUGGGGUC | 731 | GUUUUACCAU | 1131 |
| 5096 | AAUCAG | | CAUGGUAAAACAC | | GGACCCCAG | |
| LPA- | GGAGUACUGCAACCUGACG | 332 | GCAUCUCGUCAGGU | 732 | GGAGUACUGC | 1132 |
| 5124 | AGAUGC | | UGCAGUACUCCCA | | AACCUGACG | |
| LPA- | GAGUACUGCAACCUGACGC | 333 | AGCAUUGCGUCAGG | 733 | GAGUACUGCA | 1133 |
| 5125 | AAUGCT | | UUGCAGUACUCCC | | ACCUGACGC | |
| LPA- | GUACUGCAACCUGACGCGA | 334 | UGAGCUUCGCGUCA | 734 | GUACUGCAAC | 1134 |
| 5127 | AGCUCA | | GGUUGCAGUACUC | | CUGACGCGA | |
| LPA- | UACUGCAACCUGACGCGAU | 335 | CUGAGUAUCGCGUC | 735 | UACUGCAACC | 1135 |
| 5128 | ACUCAG | | AGGUUGCAGUACU | | UGACGCGAU | |
| LPA- | UGCAACCUGACGCGAUGCU | 336 | UGUCUUAGCAUCGC | 736 | UGCAACCUGA | 1136 |
| 5131 | AAGACA | | GUCAGGUUGCAGU | | CGCGAUGCU | |
| LPA- | CCUGACGCGAUGCUCAGAC | 337 | UUCUGUGUCUGAGC | 737 | CCUGACGCGA | 1137 |
| 5136 | ACAGAA | | AUCGCGUCAGGUU | | UGCUCAGAC | |
| LPA- | CUGACGCGAUGCUCAGACA | 338 | CUUCUUUGUCUGAG | 738 | CUGACGCGAU | 1138 |
| 5137 | AAGAAG | | CAUCGCGUCAGGU | | GCUCAGACA | |
| LPA- | GAUGCUCAGACACAGAAGG | 339 | ACAGUUCCUUCUGU | 739 | GAUGCUCAGA | 1139 |
| 5144 | AACUGT | | GUCUGAGCAUCGC | | CACAGAAGG | |
| LPA- | AUGCUCAGACACAGAAGGG | 340 | CACAGUCCCUUCUG | 740 | AUGCUCAGAC | 1140 |
| 5145 | ACUGTG | | UGUCUGAGCAUCG | | ACAGAAGGG | |
| LPA- | AGACACAGAAGGGACUGUG | 341 | AGCGAUCACAGUCC | 741 | AGACACAGAA | 1141 |
| 5151 | AUCGCT | | CUUCUGUGUCUGA | | GGGACUGUG | |
| LPA- | GCAUCCUCUUCAUUUGAUU | 342 | UCCCAUAAUCAAAU | 742 | GCAUCCUCUU | 1142 |
| 5467 | AUGGGA | | GAAGAGGAUGCAC | | CAUUUGAUU | |
| LPA- | CAUCCUCUUCAUUUGAUUG | 343 | UUCCCUCAAUCAAA | 743 | CAUCCUCUUC | 1143 |
| 5468 | AGGGAA | | UGAAGAGGAUGCA | | AUUUGAUUG | |
| LPA- | AUCCUCUUCAUUUGAUUGU | 344 | CUUCCUACAAUCAA | 744 | AUCCUCUUCA | 1144 |
| 5469 | AGGAAG | | AUGAAGAGGAUGC | | UUUGAUUGU | |
| LPA- | UCCUCUUCAUUUGAUUGUG | 345 | GCUUCUCACAAUCA | 745 | UCCUCUUCAU | 1145 |
| 5470 | AGAAGC | | AAUGAAGAGGAUG | | UUGAUUGUG | |
| LPA- | CCUCUUCAUUUGAUUGUGG | 346 | GGCUUUCCACAAUC | 746 | CCUCUUCAUU | 1146 |
| 5471 | AAAGCC | | AAAUGAAGAGGAU | | UGAUUGUGG | |
| LPA- | CUUCAUUUGAUUGUGGGAA | 347 | UGAGGUUUCCCACA | 747 | CUUCAUUUGA | 1147 |
| 5474 | ACCUCA | | AUCAAAUGAAGAG | | UUGUGGGAA | |
| LPA- | UUCAUUUGAUUGUGGGAAG | 348 | UUGAGUCUUCCCAC | 748 | UUCAUUUGAU | 1148 |
| 5475 | ACUCAA | | AAUCAAAUGAAGA | | UGUGGGAAG | |
| LPA- | UCAUUUGAUUGUGGGAAGC | 349 | CUUGAUGCUUCCCA | 749 | UCAUUUGAUU | 1149 |
| 5476 | AUCAAG | | CAAUCAAAUGAAG | | GUGGGAAGC | |
| LPA- | CAUUUGAUUGUGGGAAGCC | 350 | ACUUGUGGCUUCCC | 750 | CAUUUGAUUG | 1150 |
| 5477 | ACAAGT | | ACAAUCAAAUGAA | | UGGGAAGCC | |
| LPA- | AUUUGAUUGUGGGAAGCCU | 351 | CACUUUAGGCUUCC | 751 | AUUUGAUUGU | 1151 |
| 5478 | AAAGTG | | CACAAUCAAAUGA | | GGGAAGCCU | |
| LPA- | GUGGGAAGCCUCAAGUGGA | 352 | UUCGGUUCCACUUG | 752 | GUGGGAAGCC | 1152 |
| 5486 | ACCGAA | | AGGCUUCCCACAA | | UCAAGUGGA | |
| LPA- | AAGAAAUGUCCUGGAAGCA | 353 | CUACAUUGCUUCCA | 753 | AAGAAAUGUC | 1153 |
| 5509 | AUGUAG | | GGACAUUUCUUCG | | CUGGAAGCA | |
| LPA- | AGAAAUGUCCUGGAAGCAU | 354 | CCUACUAUGCUUCC | 754 | AGAAAUGUCC | 1154 |
| 5510 | AGUAGG | | AGGACAUUUCUUC | | UGGAAGCAU | |
| LPA- | GAAAUGUCCUGGAAGCAUU | 355 | CCCUAUAAUGCUUC | 755 | GAAAUGUCCU | 1155 |
| 5511 | AUAGGG | | CAGGACAUUUCUU | | GGAAGCAUU | |
| LPA- | AAUGUCCUGGAAGCAUUGU | 356 | CCCCCUACAAUGCU | 756 | AAUGUCCUGG | 1156 |
| 5513 | AGGGGG | | UCCAGGACAUUUC | | AAGCAUUGU | |
| LPA- | AUGUCCUGGAAGCAUUGUA | 357 | CCCCCUUACAAUGC | 757 | AUGUCCUGGA | 1157 |
| 5514 | AGGGGG | | UUCCAGGACAUUU | | AGCAUUGUA | |
| LPA- | AGAACAAGGUUUGGAAAGC | 358 | AGAAGUGCUUUCCA | 758 | AGAACAAGGU | 1158 |
| 5581 | ACUUCT | | AACCUUGUUCUGA | | UUGGAAAGC | |
| LPA- | GAACAAGGUUUGGAAAGCA | 359 | CAGAAUUGCUUUCC | 759 | GAACAAGGUU | 1159 |
| 5582 | AUUCTG | | AAACCUUGUUCUG | | UGGAAAGCA | |
| LPA- | AACAAGGUUUGGAAAGCAC | 360 | ACAGAUGUGCUUUC | 760 | AACAAGGUUU | 1160 |
| 5583 | AUCUGT | | CAAACCUUGUUCU | | GGAAAGCAC | |
| LPA- | ACAAGGUUUGGAAAGCACU | 361 | CACAGUAGUGCUUU | 761 | ACAAGGUUUG | 1161 |
| 5584 | ACUGTG | | CCAAACCUUGUUC | | GAAAGCACU | |
| LPA- | CAAGGUUUGGAAAGCACUU | 362 | CCACAUAAGUGCUU | 762 | CAAGGUUUGG | 1162 |
| 5585 | AUGUGG | | UCCAAACCUUGUU | | AAAGCACUU | |
| LPA- | AAGGUUUGGAAAGCACUUC | 363 | UCCACUGAAGUGCU | 763 | AAGGUUUGGA | 1163 |
| 5586 | AGUGGA | | UUCCAAACCUUGU | | AAGCACUUC | |
| LPA- | AGGUUUGGAAAGCACUUCU | 364 | CUCCAUAGAAGUGC | 764 | AGGUUUGGAA | 1164 |
| 5587 | AUGGAG | | UUUCCAAACCUUG | | AGCACUUCU | |
| LPA- | UGGAAAGCACUUCUGUGGA | 365 | GGUGCUUCCACAGA | 765 | UGGAAAGCAC | 1165 |
| 5592 | AGCACC | | AGUGCUUUCCAAA | | UUCUGUGGA | |
| LPA- | GUGGAGGCACCUUAAUAUC | 366 | UCUGGUGAUAUUAA | 766 | GUGGAGGCAC | 1166 |
| 5606 | ACCAGA | | GGUGCCUCCACAG | | CUUAAUAUC | |
| LPA- | CUUAAUAUCCCCAGAGUGG | 367 | CAGCAUCCACUCUG | 767 | CUUAAUAUCC | 1167 |
| 5616 | AUGCTG | | GGGAUAUUAAGGU | | CCAGAGUGG | |
| LPA- | UAAUAUCCCCAGAGUGGGU | 36 | GUCAGUACCCACUC | 768 | UAAUAUCCCC | 1168 |
| 5618 | ACUGAC | | UGGGGAUAUUAAG | | AGAGUGGGU | |
| LPA- | AGAGUGGGUGCUGACUGCU | 369 | GUGAGUAGCAGUCA | 769 | AGAGUGGGUG | 1169 |
| 5628 | ACUCAC | | GCACCCACUCUGG | | CUGACUGCU | |
| LPA- | CAAGGUCAUCCUGGGUGCA | 370 | UUGGUUUGCACCCA | 770 | CAAGGUCAUC | 1170 |
| 5685 | AACCAA | | GGAUGACCUUGUA | | CUGGGUGCA | |
| LPA- | CCUGGGUGCACACCAAGAA | 371 | GUUCAUUUCUUGGU | 771 | CCUGGGUGCA | 1171 |
| 5694 | AUGAAC | | GUGCACCCAGGAU | | CACCAAGAA | |
| LPA- | GUGCACACCAAGAAGUGAA | 372 | UCGAGUUUCACUUC | 772 | GUGCACACCA | 1172 |
| 5699 | ACUCGA | | UUGGUGUGCACCC | | AGAAGUGAA | |
| LPA- | AGCAGAUAUUGCCUUGCUA | 373 | UAGCUUUAGCAAGG | 773 | AGCAGAUAUU | 1173 |
| 5775 | AAGCTA | | CAAUAUCUGCUUG | | GCCUUGCUA | |
| LPA- | GCAGAUAUUGCCUUGCUAA | 374 | UUAGCUUUAGCAAG | 774 | GCAGAUAUUG | 1174 |
| 5776 | AGCUAA | | GCAAUAUCUGCUU | | CCUUGCUAA | |
| LPA- | CAGAUAUUGCCUUGCUAAA | 375 | CUUAGUUUUAGCAA | 775 | CAGAUAUUGC | 1175 |
| 5777 | ACUAAG | | GGCAAUAUCUGCU | | CUUGCUAAA | |
| LPA- | AGAUAUUGCCUUGCUAAAG | 376 | GCUUAUCUUUAGCA | 776 | AGAUAUUGCC | 1176 |
| 5778 | AUAAGC | | AGGCAAUAUCUGC | | UUGCUAAAG | |
| LPA- | GAUAUUGCCUUGCUAAAGC | 377 | UGCUUUGCUUUAGC | 777 | GAUAUUGCCU | 1177 |
| 5779 | AAAGCA | | AAGGCAAUAUCUG | | UGCUAAAGC | |
| LPA- | AUAUUGCCUUGCUAAAGCU | 378 | CUGCUUAGCUUUAG | 778 | AUAUUGCCUU | 1178 |
| 5780 | AAGCAG | | CAAGGCAAUAUCU | | GCUAAAGCU | |
| LPA- | UAUUGCCUUGCUAAAGCUA | 379 | CCUGCUUAGCUUUA | 779 | UAUUGCCUUG | 1179 |
| 5781 | AGCAGG | | GCAAGGCAAUAUC | | CUAAAGCUA | |
| LPA- | UCAUCACUGACAAAGUAAU | 380 | GCUGGUAUUACUUU | 780 | UCAUCACUGA | 1180 |
| 5813 | ACCAGC | | GUCAGUGAUGACG | | CAAAGUAAU | |
| LPA- | GGACUGAAUGUUACAUCAC | 381 | CAGCCUGUGAUGUA | 781 | GGACUGAAUG | 1181 |
| 5873 | AGGCTG | | ACAUUCAGUCCUG | | UUACAUCAC | |
| LPA- | GACUGAAUGUUACAUCACU | 382 | CCAGCUAGUGAUGU | 782 | GACUGAAUGU | 1182 |
| 5874 | AGCUGG | | AACAUUCAGUCCU | | UACAUCACU | |
| LPA- | ACUGAAUGUUACAUCACUG | 383 | CCCAGUCAGUGAUG | 783 | ACUGAAUGUU | 1183 |
| 5875 | ACUGGG | | UAACAUUCAGUCC | | ACAUCACUG | |
| LPA- | CUGAAUGUUACAUCACUGG | 384 | CCCCAUCCAGUGAU | 784 | CUGAAUGUUA | 1184 |
| 5876 | AUGGGG | | GUAACAUUCAGUC | | CAUCACUGG | |
| LPA- | UGAAUGUUACAUCACUGGC | 385 | UCCCCUGCCAGUGA | 785 | UGAAUGUUAC | 1185 |
| 5877 | AGGGGA | | UGUAACAUUCAGU | | AUCACUGGC | |
| LPA- | AAUGUUACAUCACUGGCUG | 386 | UCUCCUCAGCCAGU | 786 | AAUGUUACAU | 1186 |
| 5879 | AGGAGA | | GAUGUAACAUUCA | | CACUGGCUG | |
| LPA- | GAAACCCAAGGUACCUUUG | 387 | CAGUCUCAAAGGUA | 787 | GAAACCCAAG | 1187 |
| 5902 | AGACTG | | CCUUGGGUUUCUC | | GUACCUUUG | |
| Control | | | | | | |
| Gal | GACAACAGAAUAUUAUCCA | 1188 | UUGGAUAAUAUUCU | 1189 | GACAACAGAA | 1190 |
| XC-LPA- | AGCAGCCGAAAGGCUGC | | GUUGUCGG | | UAUUAUCCA | |
| 3675 | | | | | | |
| NC1 | CGUUAAUCGCGUAUAAUAC | 1191 | AUACGCGUAUUAUA | 1192 | N/A | |
| | GCGUAT | | CGCGAUUAACGAC | | | |
| NC5 | CAUAUUGCGCGUAUAGUCG | 1193 | CUAACGCGACUAUA | 1194 | N/A | |
| | CGUUAG | | CGCGCAAUAUGGU | | | |
| NC7 | GGCGCGUAUAGUCGCGCGU | 1195 | GACUAUACGCGCGA | 1196 | N/A | |
| | AUAGTC | | CUAUACGCGCCUC | | | |

### In Vitro Cell-Based Assays

The ability of each of the DsiRINAs listed in Table 2 to inhibit LPA expression was determined using in vitro cell-based assays. Briefly, human embryonic kidney 293 (HEK293) or HepG2 cells stably expressing a human LPA gene were transfected with each of the DsiRNAs listed in Table 2 at 0.5 nM in separate wells of a multi-well cell-culture plate. Cells were maintained for 24 hours following transfection, and then the amount of remaining LPA mRNA from the transfected cells was determined using TAQMAN®-based qPCR assays. Two qPCR assays, a 3′ assay and a 5′ assay, were used to determine LPA mRNA levels as measured using PCR probes conjugated to 6-carboxyfluorescein (6-FAM).

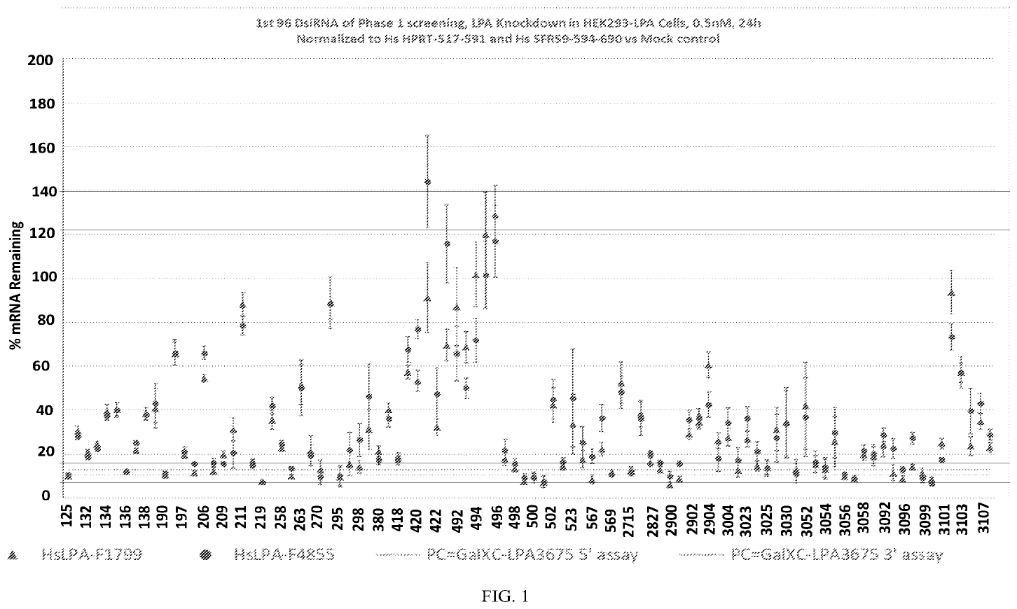

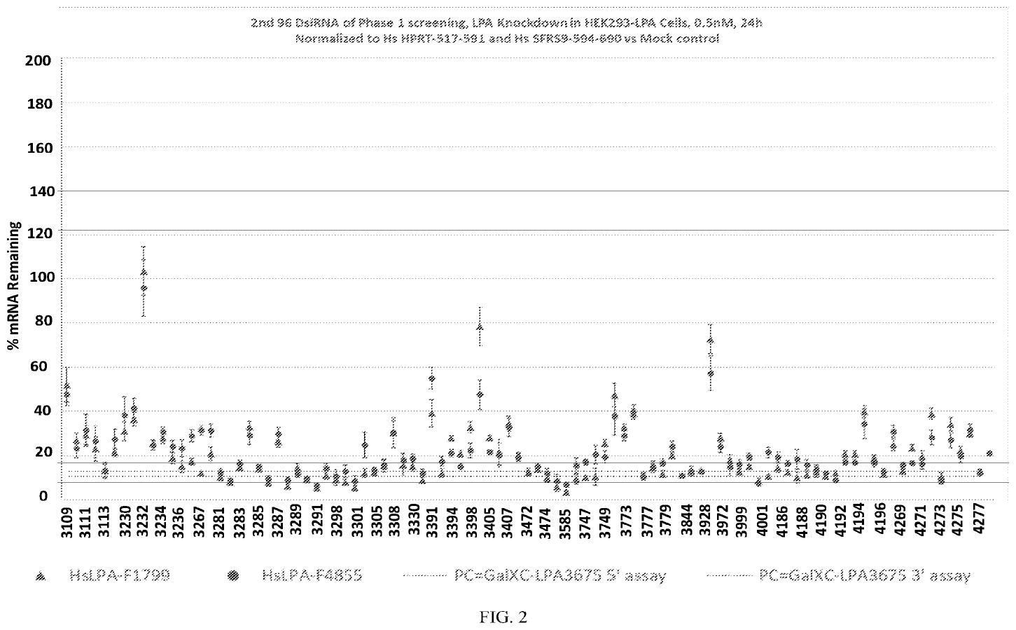

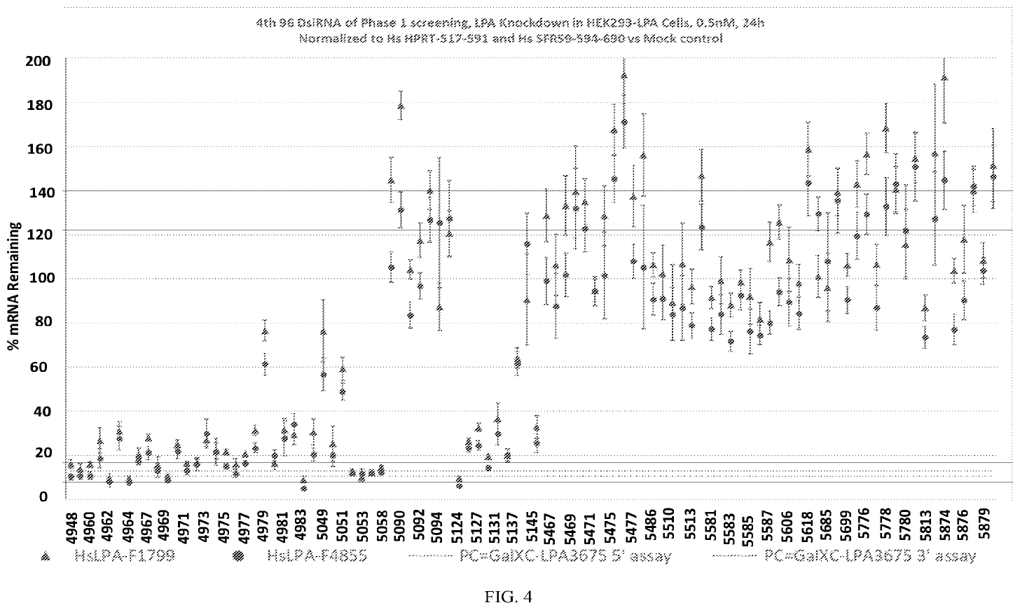

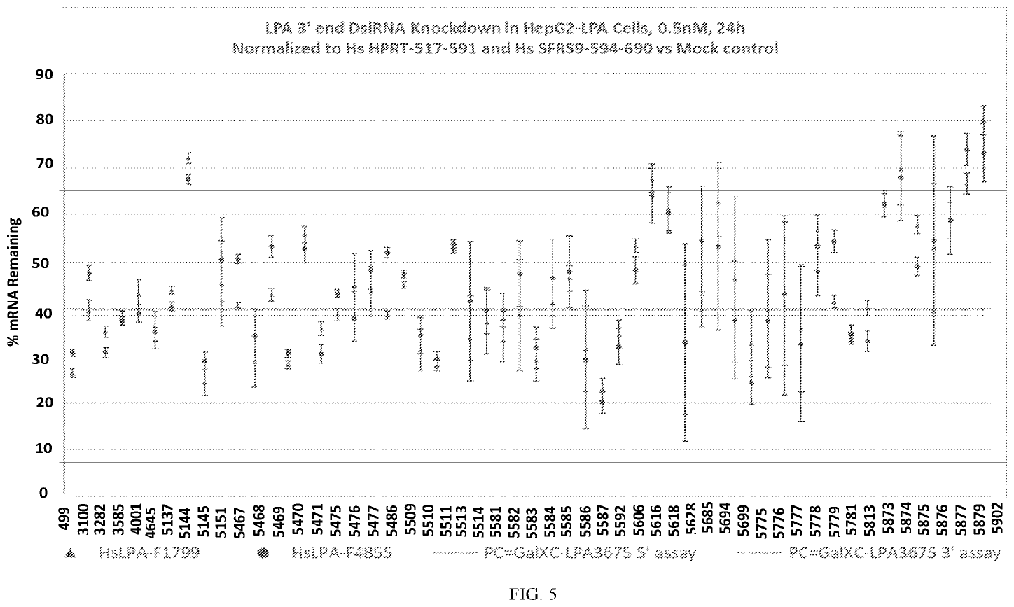

The results of the HEK293 and HepG2 cell-based assays to evaluate the ability of the DsiRNAs listed in Table 2 to inhibit LPA expression are shown in FIGS. 1-4 and FIG. 5, respectively. Cells transfected with a GalNAc-conjugated LPA oligonucleotide (GalXC-LPA-3675 SEQ ID NO: 1188 and 1189) were used as a positive control. DsiRNAs that resulted in less than or equal to about 15%-20% LPA mRNA remaining in DsiRNA-transfected cells when compared to mock-transfected cells were generally considered to comprise sequences that provide a suitable amount of knockdown or reduction of target mRNA expression for further evaluation. In FIGS. 1-5, the percent of LPA mRNA remaining in cells transfected with DsiRNAs, as indicated, relative to time-matched control cells is shown (3′ assay=circle shapes; 5′ assay=triangle shapes).

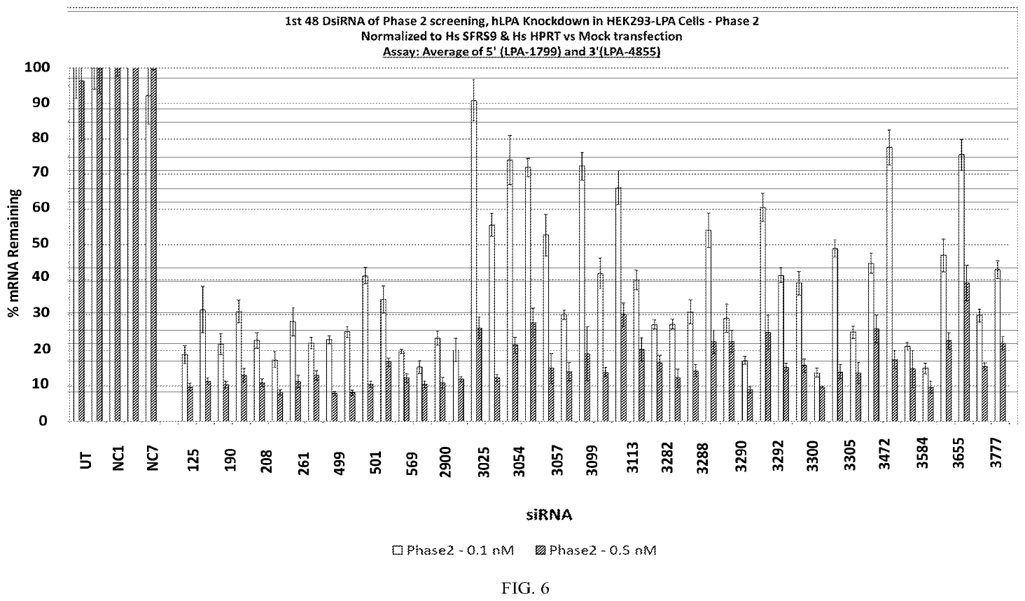

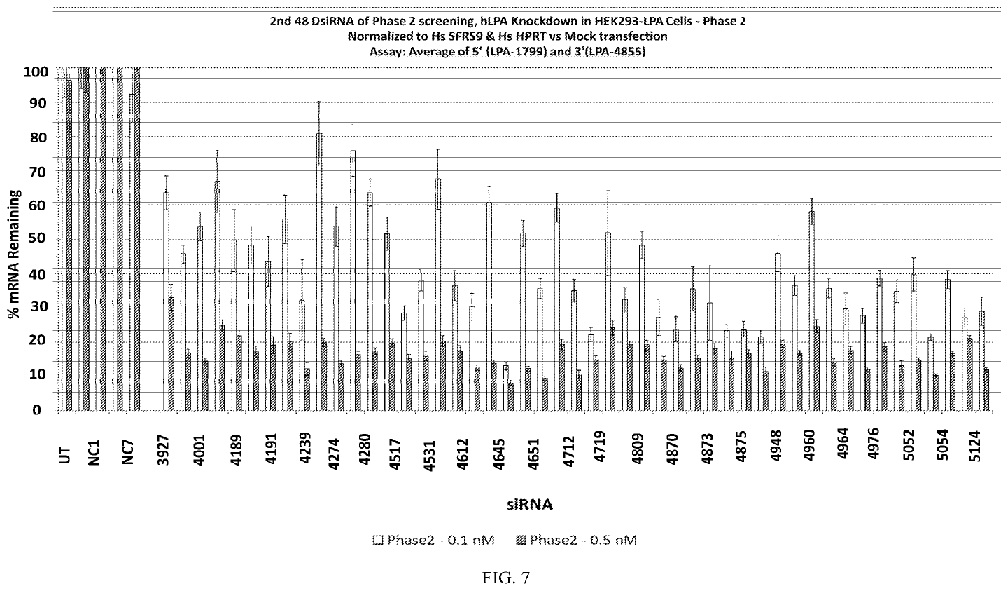

To further evaluate the DsiRNA hits, a subset of the DsiRNAs listed in Table 2 were tested to determine their ability to inhibit LPA expression using in vitro cell-based assays at two different DsiRNA concentrations (FIG. 6 and FIG. 7). Briefly, HEK293 cells stably expressing a human LPA gene were transfected with DsiRNAs at 0.1 nM and 0.5 nM in separate wells of a multi-well cell-culture plate. Cells were maintained for 24 hr. following transfection, and then the amount of remaining LPA mRNA from the transfected cells was determined using TAQMAN®-based qPCR assays. Two qPCR assays, a 3′ assay and a 5′ assay, were used to determine LPA mRNA levels as measured using PCR probes conjugated to hexachloro-fluorescein (HEX). Untransfected cells (UT), mock-transfected cells (Mock), and cells transfected with control oligonucleotides (NC1, SEQ ID NOs: 1191 and 1192; NC5, SEQ ID NO: 1193 and 1194; and NC7, SEQ ID NO: 1195 and 1196) were used as negative controls. As shown in FIGS. 6 and 7, the percent of LPA mRNA remaining in HEK293 cells transfected with the indicated DsiRNAs is an average of the LPA mRNA levels from the 3′ assay and 5′ assay and is normalized to time-matched, mock-transfected control HEK293 cells.

Taken together, these results show that DsiRNAs designed to target human LPA mRNA inhibit LPA expression in cells, as determined by a reduced amount of LPA mRNA in DsiRNA-transfected cells relative to control cells. These results demonstrate that the nucleotide sequences comprising the DsiRNA are useful for generating RNAi oligonucleotides to inhibit LPA expression. Further, these results demonstrate that multiple LPA mRNA target sequences are suitable for the RNAi-mediated inhibition of LPA expression.

### Example 3: RNAi Oligonucleotide Inhibition of LPA Expression In Vivo

Of the DsiRNAs screened in the cell-based assays described in Example 2, the nucleotide sequences of 14 DsiRNAs were selected for further evaluation in vivo. Briefly, the nucleotide sequences of the 14 selected DsiRNAs were used to generate 14 corresponding double-stranded RNAi oligonucleotides comprising a nicked tetraloop GalNAc-conjugated structure (referred to herein as “GalNAc-conjugated LPA oligonucleotides”) having a 36-mer passenger strand and a 22-mer guide strand (Table 3). Further, the nucleotide sequences comprising the passenger strand and guide strand of the GalNAc-conjugated LPA oligonucleotides have a distinct pattern of modified nucleotides and phosphorothioate linkages (e.g., see FIG. 10 for a schematic of the generic structure and chemical modification patterns (M1, M2, and M3) of the GalNAc-conjugated LPA oligonucleotides). The three adenosine nucleotides comprising the tetraloop are each conjugated to a GalNAc moiety (CAS #: 14131-60-3).

TABLE 3
GalNAc-Conjugated LPA Oligonucleotides Evaluated in Mice

| | | SEQ ID NO | SEQ ID NO |
|---|---|---|---|
| Oligonucleotide | DP# | (Sense) | (Antisense) |
| LPA-0190-M1 | DP15791P:DP15790G | 388 | 788 |
| LPA-0501-M1 | DP15634P:DP15633G | 389 | 789 |
| LPA-3100-M1 | DP15639P:DP15638G | 390 | 790 |
| LPA-3286-M1 | DP15643P:DP15642G | 391 | 791 |
| LPA-3288-M1 | DP15645P:DP15644G | 392 | 792 |
| LPA-3291-M1 | DP15647P:DP15646G | 393 | 793 |
| LPA-3584-M1 | DP15651P:DP15650G | 394 | 794 |
| LPA-3585-M1 | DP15653P:DP15652G | 395 | 795 |
| LPA-4645-M1 | DP15657P:DP15656G | 396 | 796 |
| LPA-4717-M1 | DP15801P:DP15800G | 397 | 797 |
| LPA-5510-M1 | DP15815P:DP15814G | 398 | 798 |
| LPA-3750-M1 | DP13346P:DP13385G | 399 | 799 |
| LPA-2900-M2 | DP13351P:DP14623G | 400 | 800 |
| LPA-3675-M2 | DP13346P:DP14624G | 401 | 801 |
| LPA-2900-M3 | DP13351P:DP13387G | 402 | 802 |
| LPA-3675-M3 | DP13346P:DP13385G | 403 | 803 |

### Mouse Studies

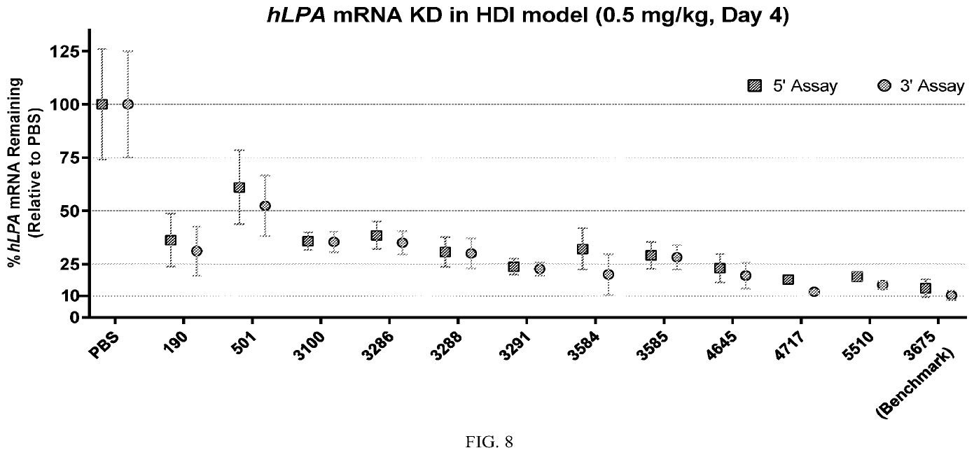

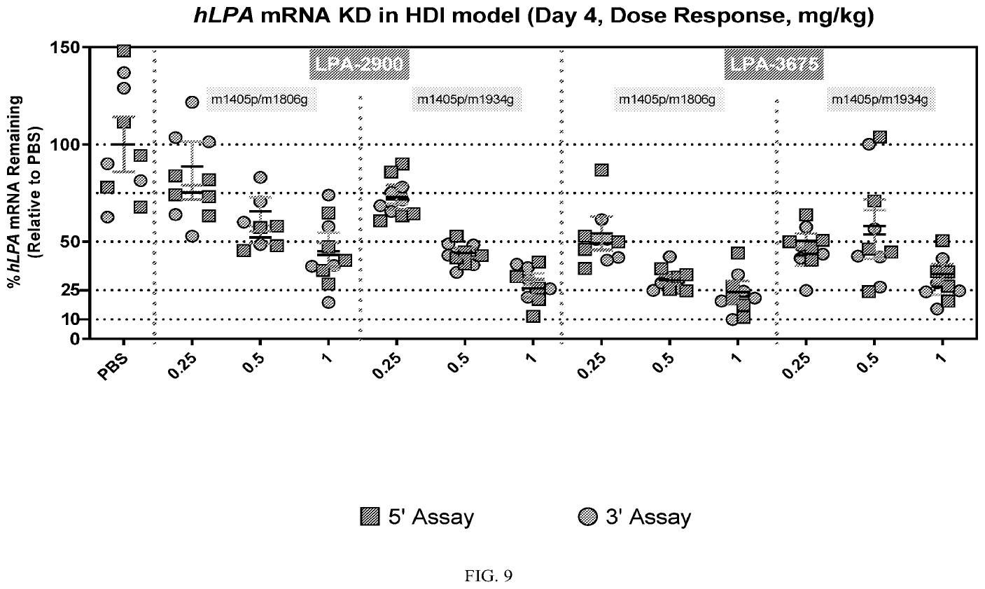

The GalNAc-conjugated LPA oligonucleotides listed in Table 3 were evaluated in an HDI mouse model, wherein HDI mice were engineered to transiently express human LPA mRNA in hepatocytes. The GalNAc-conjugated LPA oligonucleotide LPA-3675-M2 was used as a benchmark control. Briefly, 6-8-week-old female CD-1 mice (n=5) were treated subcutaneously with the indicated GalNAc-conjugated LPA oligonucleotides at a dose level of 0.5 mg/kg (FIG. 8) or at a dose level of 0.25 mg/kg, 0.5 mg/kg, and 1 mg/kg (FIG. 9). Three days later (72 h), the mice were hydrodynamically injected (HDI) with a DNA plasmid encoding the full human LPA gene under control of a ubiquitous cytomegalovirus (CMV) promoter sequence. One day after introduction of the DNA plasmid, liver samples from mice were collected. Total RNA derived from these mice were subjected to qRT-PCR analysis for LPA mRNA, relative to mice treated only with an identical volume of PBS. The values were normalized for transfection efficiency using the NeoR gene included on the plasmid.

As shown in FIG. 8, the indicated GalNAc-conjugated LPA oligonucleotides inhibited LPA expression, as determined by a reduction in the amount of LPA mRNA in liver samples from oligonucleotide-treated HDI mice relative to mice treated with PBS. To further evaluate the ability of GalNAc-conjugated LPA oligonucleotides to inhibit LPA expression, two of the GalNAc-conjugated LPA oligonucleotide sequences (LPA-2900 and LPA-3675) each having a different chemical modification pattern (M2 and M3) were tested for their ability to inhibit LPA expression in the HDI mice described above at three different concentrations (0.25 mg/kg, 0.5 mg/kg, and 1.0 mg/kg). As shown in FIG. 9, the indicated GalNAc-conjugated LPA oligonucleotides inhibited LPA expression in HDI mice in a dose-dependent manner.

Taken together, these results show that GalNAc-conjugated LPA oligonucleotides designed to target human LPA mRNA inhibit LPA expression in mice, as determined by a reduction in the amount of LPA mRNA in HDI mouse livers relative to control mice treated with PBS. Based on these results, 10 of the 14 GalNAc-conjugated LPA oligonucleotides evaluated in HDI mice were selected for evaluation of their ability to inhibit LPA expression in non-human primates (NHPs). The 10 GalNAc-conjugated LPA oligonucleotides listed in Table 4 comprise chemically modified nucleotides having pattern M1, M2, or M3 as described in FIG. 10.

TABLE 4
GalNAc-Conjugated LPA Oligonucleotides Evaluated in NHPs

| | | SEQ ID NO | SEQ ID NO |
|---|---|---|---|
| Oligonucleotide | DP# | (Sense) | (Antisense) |
| LPA-0190-M1 | DP15791P:DP15790G | 388 | 788 |
| LPA-3100-M1 | DP15639P:DP15638G | 390 | 790 |
| LPA-3288-M1 | DP15645P:DP15644G | 392 | 792 |
| LPA-3291-M1 | DP15647P:DP15646G | 393 | 793 |
| LPA-3585-M1 | DP15653P:DP15652G | 395 | 795 |
| LPA-4645-M1 | DP15657P:DP15656G | 396 | 796 |
| LPA-4717-M1 | DP15801P:DP15800G | 397 | 797 |
| LPA-5510-M1 | DP15815P:DP15814G | 398 | 798 |
| LPA-2900-M2 | DP13351P:DP14623G | 400 | 800 |
| LPA-3675-M3 | DP13346P:DP13385G | 403 | 803 |

### Non-Human Primate (NHP) Studies

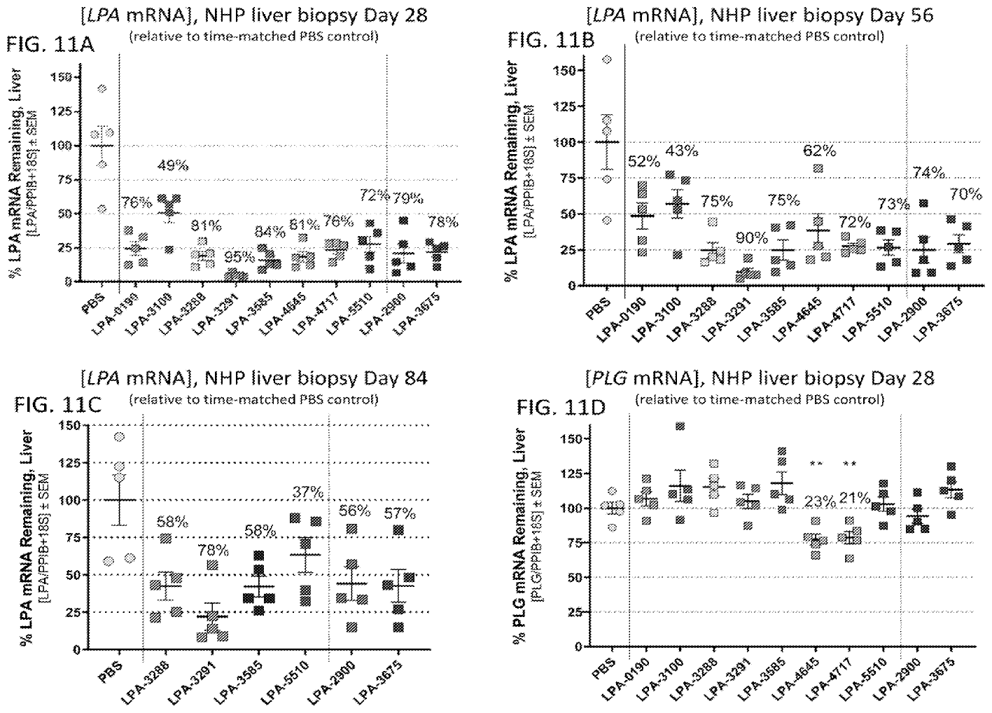

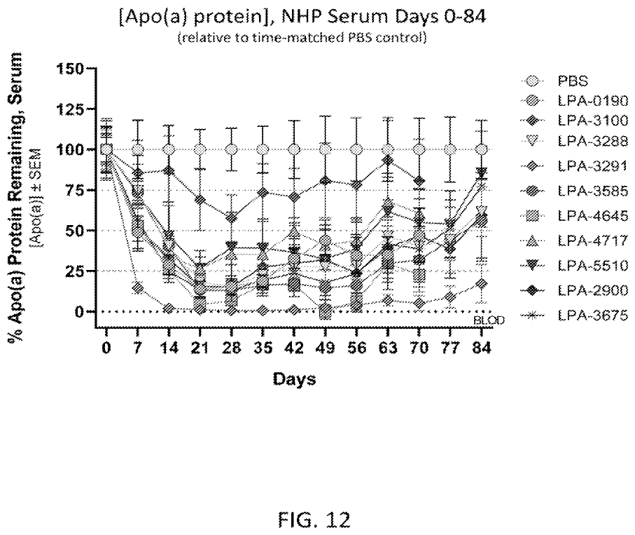

The GalNAc-conjugated LPA oligonucleotides listed in Table 4 were evaluated in cynomolgus monkeys (Macaca fascicularis). In this study, the monkeys are grouped so that their mean body weights (about 5.4 kg) are comparable between the control and experimental groups. Each cohort contains two male and three female subjects. The GalNAc-conjugated LPA oligonucleotides were administered subcutaneously on Study Day 0. Blood samples were collected on Study Days −8, −5 and 0, and weekly after dosing. Ultrasound-guided core needle liver biopsies were collected on Study Days 28, 56 and 84. At each time point, total RNA derived from the liver biopsy samples was subjected to qRT-PCR analysis to measure LPA mRNA in oligonucleotide-treated monkeys relative to monkeys treated with a comparable volume of PBS. To normalize the data, the measurements were made relative to the geometric mean of two reference genes, PPIB and 18S rRNA. As shown in FIG. 11A (Day 28), FIG. 11B (Day 56), and FIG. 11C (Day 84), treatment of NHPs with the GalNAc-conjugated LPA oligonucleotides listed in Table 4 inhibited LPA expression in the liver, as determined by a reduction in the amount of LPA mRNA in liver samples from oligonucleotide-treated NHPs relative to NHPs treated with PBS. The amount of plasminogen (PLG) mRNA in the liver samples of treated NHPs was also determined and is shown in FIG. 11D. From the same NHP study, inhibition of LPA expression was also determined by measuring apo(a) protein serum from treated NHPs by ELISA. As shown in FIG. 12, a significant reduction in serum apo(a) protein was observed in NHPs treated with GalNAc-conjugated LPA oligonucleotides compared to NHPs treated with PBS. Values from three pre-dose samples are averaged and set to 100%, and data are reported as relative values compared to the pre-dose average. Taken together, these results demonstrate that treatment of NHPs with GalNAc-conjugated LPA oligonucleotides reduced the amount of LPA mRNA in the liver and reduced the amount of apo(a) protein in the serum.

Taken together, these results show that GalNAc-conjugated LPA oligonucleotides designed to target human LPA mRNA inhibit LPA expression in vivo (as determined by the reduction of the amount of LPA mRNA and apo(a) protein in treated animals).

### SEQUENCE LISTING

The following nucleic and/or amino acid sequences are referred to in the disclosure above and are provided below for reference.

TABLE 5
LPA Oligonucleotide Sequences (Unmodified)

| | | SEQ | | SEQ |
|---|---|---|---|---|
| | Sequence | ID | Sequence | ID |
| Oligonucleotide | (Sense Strand) | NO: | (Antisense Strand) | NO: |
| LPA-125 | CUGAGCAAAGCCAUGUGGUAC | 4 | UCCUGUACCACAUGGCUUUGCUCA | 404 |
| | AGGA | | GGU | |
| LPA-128 | AGCAAAGCCAUGUGGUCCAAG | 5 | CAAUCUUGGACCACAUGGCUUUGC | 405 |
| | AUTG | | UCA | |
| LPA-132 | AAGCCAUGUGGUCCAGGAUAG | 6 | GUAGCUAUCCUGGACCACAUGGCU | 406 |
| | CUAC | | UUG | |
| LPA-133 | AGCCAUGUGGUCCAGGAUUAC | 7 | GGUAGUAAUCCUGGACCACAUGGC | 407 |
| | UACC | | UUU | |
| LPA-134 | GCCAUGUGGUCCAGGAUUGAU | 8 | UGGUAUCAAUCCUGGACCACAUGG | 408 |
| | ACCA | | CUU | |
| LPA-135 | CCAUGUGGUCCAGGAUUGCAA | 9 | AUGGUUGCAAUCCUGGACCACAUG | 409 |
| | CCAT | | GCU | |
| LPA-136 | CAUGUGGUCCAGGAUUGCUAC | 10 | CAUGGUAGCAAUCCUGGACCACAU | 410 |
| | CATG | | GGC | |
| LPA-137 | AUGUGGUCCAGGAUUGCUAAC | 11 | CCAUGUUAGCAAUCCUGGACCACA | 411 |
| | AUGG | | UGG | |
| LPA-138 | UGUGGUCCAGGAUUGCUACAA | 12 | ACCAUUGUAGCAAUCCUGGACCAC | 412 |
| | UGGT | | AUG | |
| LPA-160 | GGUGAUGGACAGAGUUAUCAA | 13 | UGCCUUGAUAACUCUGUCCAUCAC | 413 |
| | GGCA | | CAU | |
| LPA-190 | UCCACCACUGUCACAGGAAAG | 14 | AGGUCUUUCCUGUGACAGUGGUGG | 414 |
| | ACCT | | AGU | |
| LPA-191 | CCACCACUGUCACAGGAAGAA | 15 | CAGGUUCUUCCUGUGACAGUGGUG | 415 |
| | CCTG | | GAG | |
| LPA-197 | CUGUCACAGGAAGGACCUGAC | 16 | GCUUGUCAGGUCCUUCCUGUGACA | 416 |
| | AAGC | | GUG | |
| LPA-205 | GGAAGGACCUGCCAAGCUUAG | 17 | AUGACUAAGCUUGGCAGGUCCUUC | 417 |
| | UCAT | | CUG | |
| LPA-206 | GAAGGACCUGCCAAGCUUGAU | 18 | GAUGAUCAAGCUUGGCAGGUCCUU | 418 |
| | CATC | | CCU | |
| LPA-208 | AGGACCUGCCAAGCUUGGUAA | 19 | UAGAUUACCAAGCUUGGCAGGUCC | 419 |
| | UCTA | | UUC | |
| LPA-209 | GGACCUGCCAAGCUUGGUCAU | 20 | AUAGAUGACCAAGCUUGGCAGGUC | 420 |
| | CUAT | | CUU | |
| LPA-210 | GACCUGCCAAGCUUGGUCAAC | 21 | CAUAGUUGACCAAGCUUGGCAGGU | 421 |
| | UATG | | CCU | |
| LPA-211 | ACCUGCCAAGCUUGGUCAUAU | 22 | UCAUAUAUGACCAAGCUUGGCAGG | 422 |
| | AUGA | | UCC | |
| LPA-212 | CCUGCCAAGCUUGGUCAUCAA | 23 | GUCAUUGAUGACCAAGCUUGGCAG | 423 |
| | UGAC | | GUC | |
| LPA-219 | AGCUUGGUCAUCUAUGACAAC | 24 | AUGUGUUGUCAUAGAUGACCAAGC | 424 |
| | ACAT | | UUG | |
| LPA-225 | GUCAUCUAUGACACCACAUAA | 25 | AUGUUUAUGUGGUGUCAUAGAUGA | 425 |
| | ACAT | | CCA | |
| LPA-258 | CACAGAAAACUACCCAAAUAC | 26 | GCCAGUAUUUGGGUAGUUUUCUGU | 426 |
| | UGGC | | GGU | |
| LPA-261 | AGAAAACUACCCAAAUGCUAG | 27 | CAAGCUAGCAUUUGGGUAGUUUUC | 427 |
| | CUTG | | UGU | |
| LPA-263 | AAAACUACCCAAAUGCUGGAU | 28 | AUCAAUCCAGCAUUUGGGUAGUUU | 428 |
| | UGAT | | UCU | |
| LPA-269 | ACCCAAAUGCUGGCUUGAUAA | 29 | UUCAUUAUCAAGCCAGCAUUUGGG | 429 |
| | UGAA | | UAG | |
| LPA-270 | CCCAAAUGCUGGCUUGAUCAU | 30 | GUUCAUGAUCAAGCCAGCAUUUGG | 430 |
| | GAAC | | GUA | |
| LPA-291 | GAACUACUGCAGGAAUCCAAA | 31 | AGCAUUUGGAUUCCUGCAGUAGUU | 431 |
| | UGCT | | CAU | |
| LPA-295 | UACUGCAGGAAUCCAGAUGAU | 32 | CCACAUCAUCUGGAUUCCUGCAGU | 432 |
| | GUGG | | AGU | |
| LPA-296 | ACUGCAGGAAUCCAGAUGCAG | 33 | GCCACUGCAUCUGGAUUCCUGCAG | 433 |
| | UGGC | | UAG | |
| LPA-298 | UGCAGGAAUCCAGAUGCUGAG | 34 | CUGCCUCAGCAUCUGGAUUCCUGC | 434 |
| | GCAG | | AGU | |
| LPA-355 | AGGUGGGAGUACUGCAACCAG | 35 | GCGUCUGGUUGCAGUACUCCCACC | 435 |
| | ACGC | | UGA | |
| LPA-380 | AAUGCUCAGACGCAGAAGGAA | 36 | GCAGUUCCUUCUGCGUCUGAGCAU | 436 |
| | CUGC | | UGC | |
| LPA-417 | GACUGUUACCCCGGUUCCAAG | 37 | UAGGCUUGGAACCGGGGUAACAGU | 437 |
| | CCTA | | CGG | |
| LPA-418 | ACUGUUACCCCGGUUCCAAAC | 38 | CUAGGUUUGGAACCGGGGUAACAG | 438 |
| | CUAG | | UCG | |
| LPA-419 | CUGUUACCCCGGUUCCAAGAC | 39 | UCUAGUCUUGGAACCGGGGUAACA | 439 |
| | UAGA | | GUC | |
| LPA-420 | UGUUACCCCGGUUCCAAGCAU | 40 | CUCUAUGCUUGGAACCGGGGUAAC | 440 |
| | AGAG | | AGU | |
| LPA-421 | GUUACCCCGGUUCCAAGCCAA | 41 | CCUCUUGGCUUGGAACCGGGGUAA | 441 |
| | GAGG | | CAG | |
| LPA-422 | UUACCCCGGUUCCAAGCCUAG | 42 | GCCUCUAGGCUUGGAACCGGGGUA | 442 |
| | AGGC | | ACA | |
| LPA-423 | UACCCCGGUUCCAAGCCUAAA | 43 | AGCCUUUAGGCUUGGAACCGGGGU | 443 |
| | GGCT | | AAC | |
| LPA-492 | GUGCUACCAUGGUAAUGGAAA | 44 | ACUCUUUCCAUUACCAUGGUAGCA | 444 |
| | GAGT | | CUC | |
| LPA-493 | UGCUACCAUGGUAAUGGACAG | 45 | AACUCUGUCCAUUACCAUGGUAGC | 445 |
| | AGTT | | ACU | |
| LPA-494 | GCUACCAUGGUAAUGGACAAA | 46 | UAACUUUGUCCAUUACCAUGGUAG | 446 |
| | GUTA | | CAC | |
| LPA-495 | CUACCAUGGUAAUGGACAGAG | 47 | AUAACUCUGUCCAUUACCAUGGUA | 447 |
| | UUAT | | GCA | |
| LPA-496 | UACCAUGGUAAUGGACAGAAU | 48 | GAUAAUUCUGUCCAUUACCAUGGU | 448 |
| | UATC | | AGC | |
| LPA-497 | ACCAUGGUAAUGGACAGAGAU | 49 | CGAUAUCUCUGUCCAUUACCAUGG | 449 |
| | AUCG | | UAG | |
| LPA-498 | CCAUGGUAAUGGACAGAGUAA | 50 | UCGAUUACUCUGUCCAUUACCAUG | 450 |
| | UCGA | | GUA | |
| LPA-499 | CAUGGUAAUGGACAGAGUUAU | 51 | CUCGAUAACUCUGUCCAUUACCAU | 451 |
| | CGAG | | GGU | |
| LPA-500 | AUGGUAAUGGACAGAGUUAAC | 52 | CCUCGUUAACUCUGUCCAUUACCA | 452 |
| | GAGG | | UGG | |
| LPA-501 | UGGUAAUGGACAGAGUUAUAG | 53 | GCCUCUAUAACUCUGUCCAUUACC | 453 |
| | AGGC | | AUG | |
| LPA-502 | GGUAAUGGACAGAGUUAUCAA | 54 | UGCCUUGAUAACUCUGUCCAUUAC | 454 |
| | GGCA | | CAU | |
| LPA-503 | GUAAUGGACAGAGUUAUCGAG | 55 | GUGCCUCGAUAACUCUGUCCAUUA | 455 |
| | GCAC | | CCA | |
| LPA-523 | GGCACAUACUCCACCACUGAC | 56 | CUGUGUCAGUGGUGGAGUAUGUGC | 456 |
| | ACAG | | CUC | |
| LPA-563 | CUUGGUCAUCUAUGACACCAC | 57 | GAGUGUGGUGUCAUAGAUGACCAA | 457 |
| | ACTC | | GCU | |
| LPA-567 | GUCAUCUAUGACACCACACAC | 58 | AUGCGUGUGUGGUGUCAUAGAUGA | 458 |
| | GCAT | | CCA | |
| LPA-568 | UCAUCUAUGACACCACACUAG | 59 | UAUGCUAGUGUGGUGUCAUAGAUG | 459 |
| | CATA | | ACC | |
| LPA-569 | CAUCUAUGACACCACACUCAC | 60 | CUAUGUGAGUGUGGUGUCAUAGAU | 460 |
| | AUAG | | GAC | |
| LPA-1208 | GCACAUACUCCACCACUGUAA | 61 | CCAGUUACAGUGGUGGAGUAUGUG | 461 |
| | CUGG | | CCU | |
| LPA-2715 | AGCCCCUUAUUGUUAUACGAG | 62 | AUCCCUCGUAUAACAAUAAGGGGC | 462 |
| | GGAT | | UGC | |
| LPA-2716 | GCCCCUUAUUGUUAUACGAAG | 63 | GAUCCUUCGUAUAACAAUAAGGGG | 463 |
| | GATC | | CUG | |
| LPA-2827 | CCAAGCCUAGAGGCUCCUUAU | 64 | GUUCAUAAGGAGCCUCUAGGCUUG | 464 |
| | GAAC | | GAA | |
| LPA-2837 | AGGCUCCUUCUGAACAAGCAC | 65 | GUUGGUGCUUGUUCAGAAGGAGCC | 465 |
| | CAAC | | UCU | |
| LPA-2900 | AUGGACAGAGUUAUCAAGGAA | 66 | UAUGUUCCUUGAUAACUCUGUCCA | 466 |
| | CATA | | UUU | |
| LPA-2901 | UGGACAGAGUUAUCAAGGCAC | 67 | GUAUGUGCCUUGAUAACUCUGUCC | 467 |
| | AUAC | | AUU | |
| LPA-2902 | GGACAGAGUUAUCAAGGCAAA | 68 | AGUAUUUGCCUUGAUAACUCUGUC | 468 |
| | UACT | | CAU | |
| LPA-2903 | GACAGAGUUAUCAAGGCACAU | 69 | AAGUAUGUGCCUUGAUAACUCUGU | 469 |
| | ACTT | | CCA | |
| LPA-2904 | ACAGAGUUAUCAAGGCACAAA | 70 | GAAGUUUGUGCCUUGAUAACUCUG | 470 |
| | CUTC | | UCC | |
| LPA-2905 | CAGAGUUAUCAAGGCACAUAC | 71 | UGAAGUAUGUGCCUUGAUAACUCU | 471 |
| | UUCA | | GUC | |
| LPA-3004 | UACCCAAAUGCUGGCUUGAAC | 72 | UCUUGUUCAAGCCAGCAUUUGGGU | 472 |
| | AAGA | | AGU | |
| LPA-3007 | CCAAAUGCUGGCUUGAUCAAG | 73 | AGUUCUUGAUCAAGCCAGCAUUUG | 473 |
| | AACT | | GGU | |
| LPA-3023 | UCAAGAACUACUGCCGAAAAC | 74 | UCUGGUUUUCGGCAGUAGUUCUUG | 474 |
| | CAGA | | AUC | |
| LPA-3024 | CAAGAACUACUGCCGAAAUAC | 75 | AUCUGUAUUUCGGCAGUAGUUCUU | 475 |
| | AGAT | | GAU | |
| LPA-3025 | AAGAACUACUGCCGAAAUCAA | 76 | GAUCUUGAUUUCGGCAGUAGUUCU | 476 |
| | GATC | | UGA | |
| LPA-3027 | GAACUACUGCCGAAAUCCAAA | 77 | AGGAUUUGGAUUUCGGCAGUAGUU | 477 |
| | UCCT | | CUU | |
| LPA-3030 | CUACUGCCGAAAUCCAGAUAC | 78 | CACAGUAUCUGGAUUUCGGCAGUA | 478 |
| | UGTG | | GUU | |
| LPA-3051 | UGUGGCAGCCCCUUGGUGUAA | 79 | UGUAUUACACCAAGGGGCUGCCAC | 479 |
| | UACA | | AGG | |
| LPA-3052 | GUGGCAGCCCCUUGGUGUUAU | 80 | UUGUAUAACACCAAGGGGCUGCCA | 480 |
| | ACAA | | CAG | |
| LPA-3053 | UGGCAGCCCCUUGGUGUUAAA | 81 | GUUGUUUAACACCAAGGGGCUGCC | 481 |
| | CAAC | | ACA | |
| LPA-3054 | GGCAGCCCCUUGGUGUUAUAC | 82 | UGUUGUAUAACACCAAGGGGCUGC | 482 |
| | AACA | | CAC | |
| LPA-3055 | GCAGCCCCUUGGUGUUAUAAA | 83 | CUGUUUUAUAACACCAAGGGGCUG | 483 |
| | ACAG | | CCA | |
| LPA-3056 | CAGCCCCUUGGUGUUAUACAA | 84 | UCUGUUGUAUAACACCAAGGGGCU | 484 |
| | CAGA | | GCC | |
| LPA-3057 | AGCCCCUUGGUGUUAUACAAC | 85 | AUCUGUUGUAUAACACCAAGGGGC | 485 |
| | AGAT | | UGC | |
| LPA-3058 | GCCCCUUGGUGUUAUACAAAA | 86 | GAUCUUUUGUAUAACACCAAGGGG | 486 |
| | GATC | | CUG | |
| LPA-3059 | CCCCUUGGUGUUAUACAACAG | 87 | GGAUCUGUUGUAUAACACCAAGGG | 487 |
| | AUCC | | GCU | |
| LPA-3092 | GGUGGGAGUACUGCAACCUAA | 88 | CGUGUUAGGUUGCAGUACUCCCAC | 488 |
| | CACG | | CUG | |
| LPA-3093 | GUGGGAGUACUGCAACCUGAC | 89 | UCGUGUCAGGUUGCAGUACUCCCA | 489 |
| | ACGA | | CCU | |
| LPA-3096 | GGAGUACUGCAACCUGACAAG | 90 | GCAUCUUGUCAGGUUGCAGUACUC | 490 |
| | AUGC | | CCA | |
| LPA-3097 | GAGUACUGCAACCUGACACAA | 91 | AGCAUUGUGUCAGGUUGCAGUACU | 491 |
| | UGCT | | CCC | |
| LPA-3099 | GUACUGCAACCUGACACGAAG | 92 | UGAGCUUCGUGUCAGGUUGCAGUA | 492 |
| | CUCA | | CUC | |
| LPA-3100 | UACUGCAACCUGACACGAUAC | 93 | CUGAGUAUCGUGUCAGGUUGCAGU | 493 |
| | UCAG | | ACU | |
| LPA-3101 | ACUGCAACCUGACACGAUGAU | 94 | UCUGAUCAUCGUGUCAGGUUGCAG | 494 |
| | CAGA | | UAC | |
| LPA-3102 | CUGCAACCUGACACGAUGCAC | 95 | AUCUGUGCAUCGUGUCAGGUUGCA | 495 |
| | AGAT | | GUA | |
| LPA-3103 | UGCAACCUGACACGAUGCUAA | 96 | CAUCUUAGCAUCGUGUCAGGUUGC | 496 |
| | GATG | | AGU | |
| LPA-3105 | CAACCUGACACGAUGCUCAAA | 97 | UGCAUUUGAGCAUCGUGUCAGGUU | 497 |
| | UGCA | | GCA | |
| LPA-3107 | ACCUGACACGAUGCUCAGAAG | 98 | UCUGCUUCUGAGCAUCGUGUCAGG | 498 |
| | CAGA | | UUG | |
| LPA-3108 | CCUGACACGAUGCUCAGAUAC | 99 | UUCUGUAUCUGAGCAUCGUGUCAG | 499 |
| | AGAA | | GUU | |
| LPA-3109 | CUGACACGAUGCUCAGAUGAA | 100 | AUUCUUCAUCUGAGCAUCGUGUCA | 500 |
| | GAAT | | GGU | |
| LPA-3110 | UGACACGAUGCUCAGAUGCAG | 101 | CAUUCUGCAUCUGAGCAUCGUGUC | 501 |
| | AATG | | AGG | |
| LPA-3111 | GACACGAUGCUCAGAUGCAAA | 102 | CCAUUUUGCAUCUGAGCAUCGUGU | 502 |
| | AUGG | | CAG | |
| LPA-3112 | ACACGAUGCUCAGAUGCAGAA | 103 | UCCAUUCUGCAUCUGAGCAUCGUG | 503 |
| | UGGA | | UCA | |
| LPA-3113 | CACGAUGCUCAGAUGCAGAAU | 104 | GUCCAUUCUGCAUCUGAGCAUCGU | 504 |
| | GGAC | | GUC | |
| LPA-3229 | UGCUACUACCAUUAUGGACAG | 105 | AACUCUGUCCAUAAUGGUAGUAGC | 505 |
| | AGTT | | AGU | |
| LPA-3230 | GCUACUACCAUUAUGGACAAA | 106 | UAACUUUGUCCAUAAUGGUAGUAG | 506 |
| | GUTA | | CAG | |
| LPA-3231 | CUACUACCAUUAUGGACAGAG | 107 | GUAACUCUGUCCAUAAUGGUAGUA | 507 |
| | UUAC | | GCA | |
| LPA-3232 | UACUACCAUUAUGGACAGAAU | 108 | GGUAAUUCUGUCCAUAAUGGUAGU | 508 |
| | UACC | | AGC | |
| LPA-3233 | ACUACCAUUAUGGACAGAGAU | 109 | CGGUAUCUCUGUCCAUAAUGGUAG | 509 |
| | ACCG | | UAG | |
| LPA-3234 | CUACCAUUAUGGACAGAGUAA | 110 | UCGGUUACUCUGUCCAUAAUGGUA | 510 |
| | CCGA | | GUA | |
| LPA-3235 | UACCAUUAUGGACAGAGUUAC | 111 | CUCGGUAACUCUGUCCAUAAUGGU | 511 |
| | CGAG | | AGU | |
| LPA-3236 | ACCAUUAUGGACAGAGUUAAC | 112 | CCUCGUUAACUCUGUCCAUAAUGG | 512 |
| | GAGG | | UAG | |
| LPA-3257 | GAGGCACAUACUCCACCACAG | 113 | GUGACUGUGGUGGAGUAUGUGCCU | 513 |
| | UCAC | | CGG | |
| LPA-3267 | CUCCACCACUGUCACAGGAAG | 114 | AGUUCUUCCUGUGACAGUGGUGGA | 514 |
| | AACT | | GUA | |
| LPA-3280 | ACAGGAAGAACUUGCCAAGAU | 115 | ACCAAUCUUGGCAAGUUCUUCCUG | 515 |
| | UGGT | | UGA | |
| LPA-3281 | CAGGAAGAACUUGCCAAGCAU | 116 | GACCAUGCUUGGCAAGUUCUUCCU | 516 |
| | GGTC | | GUG | |
| LPA-3282 | AGGAAGAACUUGCCAAGCUAG | 117 | UGACCUAGCUUGGCAAGUUCUUCC | 517 |
| | GUCA | | UGU | |
| LPA-3283 | GGAAGAACUUGCCAAGCUUAG | 118 | AUGACUAAGCUUGGCAAGUUCUUC | 518 |
| | UCAT | | CUG | |
| LPA-3284 | GAAGAACUUGCCAAGCUUGAU | 119 | GAUGAUCAAGCUUGGCAAGUUCUU | 519 |
| | CATC | | CCU | |
| LPA-3285 | AAGAACUUGCCAAGCUUGGAC | 120 | AGAUGUCCAAGCUUGGCAAGUUCU | 520 |
| | AUCT | | UCC | |
| LPA-3286 | AGAACUUGCCAAGCUUGGUAA | 121 | UAGAUUACCAAGCUUGGCAAGUUC | 521 |
| | UCTA | | UUC | |
| LPA-3287 | GAACUUGCCAAGCUUGGUCAU | 122 | AUAGAUGACCAAGCUUGGCAAGUU | 522 |
| | CUAT | | CUU | |
| LPA-3288 | AACUUGCCAAGCUUGGUCAAC | 123 | CAUAGUUGACCAAGCUUGGCAAGU | 523 |
| | UATG | | UCU | |
| LPA-3289 | ACUUGCCAAGCUUGGUCAUAU | 124 | UCAUAUAUGACCAAGCUUGGCAAG | 524 |
| | AUGA | | UUC | |
| LPA-3290 | CUUGCCAAGCUUGGUCAUCAA | 125 | GUCAUUGAUGACCAAGCUUGGCAA | 525 |
| | UGAC | | GUU | |
| LPA-3291 | UUGCCAAGCUUGGUCAUCUAU | 126 | UGUCAUAGAUGACCAAGCUUGGCA | 526 |
| | GACA | | AGU | |
| LPA-3292 | UGCCAAGCUUGGUCAUCUAAG | 127 | GUGUCUUAGAUGACCAAGCUUGGC | 527 |
| | ACAC | | AAG | |
| LPA-3298 | GCUUGGUCAUCUAUGACACAA | 128 | GGUGUUGUGUCAUAGAUGACCAAG | 528 |
| | CACC | | CUU | |
| LPA-3300 | UUGGUCAUCUAUGACACCAAA | 129 | CUGGUUUGGUGUCAUAGAUGACCA | 529 |
| | CCAG | | AGC | |
| LPA-3301 | UGGUCAUCUAUGACACCACAC | 130 | GCUGGUGUGGUGUCAUAGAUGACC | 530 |
| | CAGC | | AAG | |
| LPA-3303 | GUCAUCUAUGACACCACACAA | 131 | AUGCUUGUGUGGUGUCAUAGAUGA | 531 |
| | GCAT | | CCA | |
| LPA-3305 | CAUCUAUGACACCACACCAAC | 132 | CUAUGUUGGUGUGGUGUCAUAGAU | 532 |
| | AUAG | | GAC | |
| LPA-3306 | AUCUAUGACACCACACCAGAA | 133 | ACUAUUCUGGUGUGGUGUCAUAGA | 533 |
| | UAGT | | UGA | |
| LPA-3308 | CUAUGACACCACACCAGCAAA | 134 | CGACUUUGCUGGUGUGGUGUCAUA | 534 |
| | GUCG | | GAU | |
| LPA-3329 | GUCGGACCCCAGAAAACUAAC | 135 | UUUGGUUAGUUUUCUGGGGUCCGA | 535 |
| | CAAA | | CUA | |
| LPA-3330 | UCGGACCCCAGAAAACUACAC | 136 | AUUUGUGUAGUUUUCUGGGGUCCG | 536 |
| | AAAT | | ACU | |
| LPA-3340 | GAAAACUACCCAAAUGCUGAC | 137 | UCAGGUCAGCAUUUGGGUAGUUUU | 537 |
| | CUGA | | CUG | |
| LPA-3391 | GCUGAGAUUCGCCCUUGGUAU | 138 | UGUAAUACCAAGGGCGAAUCUCAG | 538 |
| | UACA | | CAU | |
| LPA-3392 | CUGAGAUUCGCCCUUGGUGAU | 139 | GUGUAUCACCAAGGGCGAAUCUCA | 539 |
| | ACAC | | GCA | |
| LPA-3394 | GAGAUUCGCCCUUGGUGUUAC | 140 | UGGUGUAACACCAAGGGCGAAUCU | 540 |
| | ACCA | | CAG | |
| LPA-3395 | AGAUUCGCCCUUGGUGUUAAA | 141 | AUGGUUUAACACCAAGGGCGAAUC | 541 |
| | CCAT | | UCA | |
| LPA-3398 | UUCGCCCUUGGUGUUACACAA | 142 | UCCAUUGUGUAACACCAAGGGCGA | 542 |
| | UGGA | | AUC | |
| LPA-3404 | CUUGGUGUUACACCAUGGAAC | 143 | CUGGGUUCCAUGGUGUAACACCAA | 543 |
| | CCAG | | GGG | |
| LPA-3405 | UUGGUGUUACACCAUGGAUAC | 144 | ACUGGUAUCCAUGGUGUAACACCA | 544 |
| | CAGT | | AGG | |
| LPA-3406 | UGGUGUUACACCAUGGAUCAC | 145 | CACUGUGAUCCAUGGUGUAACACC | 545 |
| | AGTG | | AAG | |
| LPA-3407 | GGUGUUACACCAUGGAUCCAA | 146 | ACACUUGGAUCCAUGGUGUAACAC | 546 |
| | GUGT | | CAA | |
| LPA-3409 | UGUUACACCAUGGAUCCCAAU | 147 | UGACAUUGGGAUCCAUGGUGUAAC | 547 |
| | GUCA | | ACC | |
| LPA-3472 | GAAUCAAGUGUCCUUGCAAAU | 148 | UGAGAUUUGCAAGGACACUUGAUU | 548 |
| | CUCA | | CUG | |
| LPA-3473 | AAUCAAGUGUCCUUGCAACAC | 149 | GUGAGUGUUGCAAGGACACUUGAU | 549 |
| | UCAC | | UCU | |
| LPA-3474 | AUCAAGUGUCCUUGCAACUAU | 150 | CGUGAUAGUUGCAAGGACACUUGA | 550 |
| | CACG | | UUC | |
| LPA-3584 | AUGGACAGAGUUAUCGAGGAU | 151 | AAUGAUCCUCGAUAACUCUGUCCA | 551 |
| | CATT | | UCA | |
| LPA-3585 | UGGACAGAGUUAUCGAGGCAC | 152 | GAAUGUGCCUCGAUAACUCUGUCC | 552 |
| | AUTC | | AUC | |
| LPA-3655 | ACACCACACUGGCAUCAGAAG | 153 | UUGUCUUCUGAUGCCAGUGUGGUG | 553 |
| | ACAA | | UCA | |
| LPA-3747 | UUGGUGUUAUACCAUGGAUAC | 154 | AUUGGUAUCCAUGGUAUAACACCA | 554 |
| | CAAT | | AGG | |
| LPA-3748 | UGGUGUUAUACCAUGGAUCAC | 155 | CAUUGUGAUCCAUGGUAUAACACC | 555 |
| | AATG | | AAG | |
| LPA-3749 | GGUGUUAUACCAUGGAUCCAA | 156 | ACAUUUGGAUCCAUGGUAUAACAC | 556 |
| | AUGT | | CAA | |
| LPA-3750 | GUGUUAUACCAUGGAUCCCAA | 157 | GACAUUGGGAUCCAUGGUAUAACA | 557 |
| | UGTC | | CCA | |
| LPA-3773 | UCAGAUGGGAGUACUGCAAAC | 158 | GUCAGUUUGCAGUACUCCCAUCUG | 558 |
| | UGAC | | ACA | |
| LPA-3776 | GAUGGGAGUACUGCAACCUAA | 159 | UGUGUUAGGUUGCAGUACUCCCAU | 559 |
| | CACA | | CUG | |
| LPA-3777 | AUGGGAGUACUGCAACCUGAC | 160 | UUGUGUCAGGUUGCAGUACUCCCA | 560 |
| | ACAA | | UCU | |
| LPA-3778 | UGGGAGUACUGCAACCUGAAA | 161 | AUUGUUUCAGGUUGCAGUACUCCC | 561 |
| | CAAT | | AUC | |
| LPA-3779 | GGGAGUACUGCAACCUGACAC | 162 | CAUUGUGUCAGGUUGCAGUACUCC | 562 |
| | AATG | | CAU | |
| LPA-3840 | GGCUGUUUCUGAACAAGCAAC | 163 | CGUUGUUGCUUGUUCAGAAACAGC | 563 |
| | AACG | | CGU | |
| LPA-3844 | GUUUCUGAACAAGCACCAAAG | 164 | GCUCCUUUGGUGCUUGUUCAGAAA | 564 |
| | GAGC | | CAG | |
| LPA-3927 | CUCCACCACUGUUACAGGAAG | 165 | UGUCCUUCCUGUAACAGUGGUGGA | 565 |
| | GACA | | GAA | |
| LPA-3928 | UCCACCACUGUUACAGGAAAG | 166 | AUGUCUUUCCUGUAACAGUGGUGG | 566 |
| | ACAT | | AGA | |
| LPA-3929 | CCACCACUGUUACAGGAAGAA | 167 | CAUGUUCUUCCUGUAACAGUGGUG | 567 |
| | CATG | | GAG | |
| LPA-3972 | GACACCACACUGGCAUCAGAG | 168 | GGUUCUCUGAUGCCAGUGUGGUGU | 568 |
| | AACC | | CAU | |
| LPA-3973 | ACACCACACUGGCAUCAGAAA | 169 | UGGUUUUCUGAUGCCAGUGUGGUG | 569 |
| | ACCA | | UCA | |
| LPA-3999 | AGAAUACUACCCAAAUGGUAG | 170 | CAGGCUACCAUUUGGGUAGUAUUC | 570 |
| | CCTG | | UGU | |
| LPA-4000 | GAAUACUACCCAAAUGGUGAC | 171 | UCAGGUCACCAUUUGGGUAGUAUU | 571 |
| | CUGA | | CUG | |
| LPA-4001 | AAUACUACCCAAAUGGUGGAC | 172 | GUCAGUCCACCAUUUGGGUAGUAU | 572 |
| | UGAC | | UCU | |
| LPA-4185 | UCCUUCUGAAGAAGCACCAAC | 173 | UUCAGUUGGUGCUUCUUCAGAAGG | 573 |
| | UGAA | | AAG | |
| LPA-4186 | CCUUCUGAAGAAGCACCAAAU | 174 | UUUCAUUUGGUGCUUCUUCAGAAG | 574 |
| | GAAA | | GAA | |
| LPA-4187 | CUUCUGAAGAAGCACCAACAG | 175 | UUUUCUGUUGGUGCUUCUUCAGAA | 575 |
| | AAAA | | GGA | |
| LPA-4188 | UUCUGAAGAAGCACCAACUAA | 176 | GUUUUUAGUUGGUGCUUCUUCAGA | 576 |
| | AAAC | | AGG | |
| LPA-4189 | UCUGAAGAAGCACCAACUGAA | 177 | UGUUUUCAGUUGGUGCUUCUUCAG | 577 |
| | AACA | | AAG | |
| LPA-4190 | CUGAAGAAGCACCAACUGAAA | 178 | CUGUUUUCAGUUGGUGCUUCUUCA | 578 |
| | ACAG | | GAA | |
| LPA-4191 | UGAAGAAGCACCAACUGAAAA | 179 | GCUGUUUUCAGUUGGUGCUUCUUC | 579 |
| | CAGC | | AGA | |
| LPA-4192 | GAAGAAGCACCAACUGAAAAC | 180 | UGCUGUUUUCAGUUGGUGCUUCUU | 580 |
| | AGCA | | CAG | |
| LPA-4193 | AAGAAGCACCAACUGAAAAAA | 181 | GUGCUUUUUUCAGUUGGUGCUUCU | 581 |
| | GCAC | | UCA | |
| LPA-4194 | AGAAGCACCAACUGAAAACAG | 182 | AGUGCUGUUUUCAGUUGGUGCUUC | 582 |
| | CACT | | UUC | |
| LPA-4195 | GAAGCACCAACUGAAAACAAC | 183 | CAGUGUUGUUUUCAGUUGGUGCUU | 583 |
| | ACTG | | CUU | |
| LPA-4196 | AAGCACCAACUGAAAACAGAA | 184 | CCAGUUCUGUUUUCAGUUGGUGCU | 584 |
| | CUGG | | UCU | |
| LPA-4239 | AGGUGAUGGACAGAGUUAUAG | 185 | GCCUCUAUAACUCUGUCCAUCACC | 585 |
| | AGGC | | UCG | |
| LPA-4269 | CUCCACCACUAUCACAGGAAG | 186 | UGUUCUUCCUGUGAUAGUGGUGGA | 586 |
| | AACA | | GAG | |
| LPA-4270 | UCCACCACUAUCACAGGAAAA | 187 | AUGUUUUUCCUGUGAUAGUGGUGG | 587 |
| | ACAT | | AGA | |
| LPA-4271 | CCACCACUAUCACAGGAAGAA | 188 | CAUGUUCUUCCUGUGAUAGUGGUG | 588 |
| | CATG | | GAG | |
| LPA-4272 | CACCACUAUCACAGGAAGAAC | 189 | ACAUGUUCUUCCUGUGAUAGUGGU | 589 |
| | AUGT | | GGA | |
| LPA-4273 | ACCACUAUCACAGGAAGAAAA | 190 | GACAUUUUCUUCCUGUGAUAGUGG | 590 |
| | UGTC | | UGG | |
| LPA-4274 | CCACUAUCACAGGAAGAACAU | 191 | UGACAUGUUCUUCCUGUGAUAGUG | 591 |
| | GUCA | | GUG | |
| LPA-4275 | CACUAUCACAGGAAGAACAAG | 192 | CUGACUUGUUCUUCCUGUGAUAGU | 592 |
| | UCAG | | GGU | |
| LPA-4276 | ACUAUCACAGGAAGAACAUAU | 193 | ACUGAUAUGUUCUUCCUGUGAUAG | 593 |
| | CAGT | | UGG | |
| LPA-4277 | CUAUCACAGGAAGAACAUGAC | 194 | GACUGUCAUGUUCUUCCUGUGAUA | 594 |
| | AGTC | | GUG | |
| LPA-4278 | UAUCACAGGAAGAACAUGUAA | 195 | AGACUUACAUGUUCUUCCUGUGAU | 595 |
| | GUCT | | AGU | |
| LPA-4279 | AUCACAGGAAGAACAUGUCAG | 196 | AAGACUGACAUGUUCUUCCUGUGA | 596 |
| | UCTT | | UAG | |
| LPA-4280 | UCACAGGAAGAACAUGUCAAU | 197 | CAAGAUUGACAUGUUCUUCCUGUG | 597 |
| | CUTG | | AUA | |
| LPA-4281 | CACAGGAAGAACAUGUCAGAC | 198 | CCAAGUCUGACAUGUUCUUCCUGU | 598 |
| | UUGG | | GAU | |
| LPA-4282 | ACAGGAAGAACAUGUCAGUAU | 199 | ACCAAUACUGACAUGUUCUUCCUG | 599 |
| | UGGT | | UGA | |
| LPA-4285 | GGAAGAACAUGUCAGUCUUAG | 200 | ACGACUAAGACUGACAUGUUCUUC | 600 |
| | UCGT | | CUG | |
| LPA-4286 | GAAGAACAUGUCAGUCUUGAU | 201 | GACGAUCAAGACUGACAUGUUCUU | 601 |
| | CGTC | | CCU | |
| LPA-4287 | AAGAACAUGUCAGUCUUGGAC | 202 | AGACGUCCAAGACUGACAUGUUCU | 602 |
| | GUCT | | UCC | |
| LPA-4288 | AGAACAUGUCAGUCUUGGUAG | 203 | UAGACUACCAAGACUGACAUGUUC | 603 |
| | UCTA | | UUC | |
| LPA-4325 | GGCAUCGGAGGAUCCCAUUAU | 204 | UAGUAUAAUGGGAUCCUCCGAUGC | 604 |
| | ACTA | | CAA | |
| LPA-4346 | ACUAUCCAAAUGCUGGCCUAA | 205 | CUGGUUAGGCCAGCAUUUGGAUAG | 605 |
| | CCAG | | UAU | |
| LPA-4517 | GCACAGAGGCUCCUUCUGAAC | 206 | GCUUGUUCAGAAGGAGCCUCUGUG | 606 |
| | AAGC | | CUU | |
| LPA-4527 | UCCUUCUGAACAAGCACCAAC | 207 | CUCAGUUGGUGCUUGUUCAGAAGG | 607 |
| | UGAG | | AGC | |
| LPA-4528 | CCUUCUGAACAAGCACCACAU | 208 | UCUCAUGUGGUGCUUGUUCAGAAG | 608 |
| | GAGA | | GAG | |
| LPA-4529 | CUUCUGAACAAGCACCACCAG | 209 | UUCUCUGGUGGUGCUUGUUCAGAA | 609 |
| | AGAA | | GGA | |
| LPA-4530 | UUCUGAACAAGCACCACCUAA | 210 | UUUCUUAGGUGGUGCUUGUUCAGA | 610 |
| | GAAA | | AGG | |
| LPA-4531 | UCUGAACAAGCACCACCUGAG | 211 | UUUUCUCAGGUGGUGCUUGUUCAG | 611 |
| | AAAA | | AAG | |
| LPA-4532 | CUGAACAAGCACCACCUGAAA | 212 | CUUUUUUCAGGUGGUGCUUGUUCA | 612 |
| | AAAG | | GAA | |
| LPA-4533 | UGAACAAGCACCACCUGAGAA | 213 | GCUUUUCUCAGGUGGUGCUUGUUC | 613 |
| | AAGC | | AGA | |
| LPA-4534 | GAACAAGCACCACCUGAGAAA | 214 | GGCUUUUCUCAGGUGGUGCUUGUU | 614 |
| | AGCC | | CAG | |
| LPA-4535 | AACAAGCACCACCUGAGAAAA | 215 | GGGCUUUUCUCAGGUGGUGCUUGU | 615 |
| | GCCC | | UCA | |
| LPA-4537 | CAAGCACCACCUGAGAAAAAC | 216 | CAGGGUUUUUCUCAGGUGGUGCUU | 616 |
| | CCTG | | GUU | |
| LPA-4538 | AAGCACCACCUGAGAAAAGAC | 217 | ACAGGUCUUUUCUCAGGUGGUGCU | 617 |
| | CUGT | | UGU | |
| LPA-4539 | AGCACCACCUGAGAAAAGCAC | 218 | CACAGUGCUUUUCUCAGGUGGUGC | 618 |
| | UGTG | | UUG | |
| LPA-4547 | CUGAGAAAAGCCCUGUGGUAC | 219 | UCCUGUACCACAGGGCUUUUCUCA | 619 |
| | AGGA | | GGU | |
| LPA-4556 | GCCCUGUGGUCCAGGAUUGAU | 220 | UGGUAUCAAUCCUGGACCACAGGG | 620 |
| | ACCA | | CUU | |
| LPA-4559 | CUGUGGUCCAGGAUUGCUAAC | 221 | CCAUGUUAGCAAUCCUGGACCACA | 621 |
| | AUGG | | GGG | |
| LPA-4611 | CUCCACCACUGUCACAGGAAG | 222 | GGUCCUUCCUGUGACAGUGGUGGA | 622 |
| | GACC | | GGA | |
| LPA-4612 | UCCACCACUGUCACAGGAAAG | 223 | AGGUCUUUCCUGUGACAGUGGUGG | 623 |
| | ACCT | | AGG | |
| LPA-4642 | UCUUGGUCAUCUAUGAUACAA | 224 | AGUGUUGUAUCAUAGAUGACCAAG | 624 |
| | CACT | | AUU | |
| LPA-4643 | CUUGGUCAUCUAUGAUACCAC | 225 | CAGUGUGGUAUCAUAGAUGACCAA | 625 |
| | ACTG | | GAU | |
| LPA-4644 | UUGGUCAUCUAUGAUACCAAA | 226 | CCAGUUUGGUAUCAUAGAUGACCA | 626 |
| | CUGG | | AGA | |
| LPA-4645 | UGGUCAUCUAUGAUACCACAC | 227 | GCCAGUGUGGUAUCAUAGAUGACC | 627 |
| | UGGC | | AAG | |
| LPA-4646 | GGUCAUCUAUGAUACCACAAU | 228 | UGCCAUUGUGGUAUCAUAGAUGAC | 628 |
| | GGCA | | CAA | |
| LPA-4647 | GUCAUCUAUGAUACCACACAG | 229 | AUGCCUGUGUGGUAUCAUAGAUGA | 629 |
| | GCAT | | CCA | |
| LPA-4648 | UCAUCUAUGAUACCACACUAG | 230 | GAUGCUAGUGUGGUAUCAUAGAUG | 630 |
| | CATC | | ACC | |
| LPA-4649 | CAUCUAUGAUACCACACUGAC | 231 | UGAUGUCAGUGUGGUAUCAUAGAU | 631 |
| | AUCA | | GAC | |
| LPA-4650 | AUCUAUGAUACCACACUGGAA | 232 | CUGAUUCCAGUGUGGUAUCAUAGA | 632 |
| | UCAG | | UGA | |
| LPA-4651 | UCUAUGAUACCACACUGGCAU | 233 | UCUGAUGCCAGUGUGGUAUCAUAG | 633 |
| | CAGA | | AUG | |
| LPA-4652 | CUAUGAUACCACACUGGCAAC | 234 | CUCUGUUGCCAGUGUGGUAUCAUA | 634 |
| | AGAG | | GAU | |
| LPA-4655 | UGAUACCACACUGGCAUCAAA | 235 | GUCCUUUGAUGCCAGUGUGGUAUC | 635 |
| | GGAC | | AUA | |
| LPA-4657 | AUACCACACUGGCAUCAGAAG | 236 | GGGUCUUCUGAUGCCAGUGUGGUA | 636 |
| | ACCC | | UCA | |
| LPA-4673 | AGAGGACCCCAGAAAACUAAC | 237 | UUUGGUUAGUUUUCUGGGGUCCUC | 637 |
| | CAAA | | UGA | |
| LPA-4674 | GAGGACCCCAGAAAACUACAC | 238 | AUUUGUGUAGUUUUCUGGGGUCCU | 638 |
| | AAAT | | CUG | |
| LPA-4712 | AGAACUACUGCAGGAAUCCAG | 239 | GAAUCUGGAUUCCUGCAGUAGUUC | 639 |
| | AUTC | | UCG | |
| LPA-4715 | ACUACUGCAGGAAUCCAGAAU | 240 | CCAGAUUCUGGAUUCCUGCAGUAG | 640 |
| | CUGG | | UUC | |
| LPA-4717 | UACUGCAGGAAUCCAGAUUAU | 241 | UCCCAUAAUCUGGAUUCCUGCAGU | 641 |
| | GGGA | | AGU | |
| LPA-4718 | ACUGCAGGAAUCCAGAUUCAG | 242 | UUCCCUGAAUCUGGAUUCCUGCAG | 642 |
| | GGAA | | UAG | |
| LPA-4719 | CUGCAGGAAUCCAGAUUCUAG | 243 | UUUCCUAGAAUCUGGAUUCCUGCA | 643 |
| | GAAA | | GUA | |
| LPA-4720 | UGCAGGAAUCCAGAUUCUGAG | 244 | GUUUCUCAGAAUCUGGAUUCCUGC | 644 |
| | AAAC | | AGU | |
| LPA-4721 | GCAGGAAUCCAGAUUCUGGAA | 245 | UGUUUUCCAGAAUCUGGAUUCCUG | 645 |
| | AACA | | CAG | |
| LPA-4724 | GGAAUCCAGAUUCUGGGAAAC | 246 | GGUUGUUUCCCAGAAUCUGGAUUC | 646 |
| | AACC | | CUG | |
| LPA-4738 | GGGAAACAACCCUGGUGUUAC | 247 | UUGUGUAACACCAGGGUUGUUUCC | 647 |
| | ACAA | | CAG | |
| LPA-4739 | GGAAACAACCCUGGUGUUAAA | 248 | GUUGUUUAACACCAGGGUUGUUUC | 648 |
| | CAAC | | CCA | |
| LPA-4771 | UGUGUGAGGUGGGAGUACUAC | 249 | GAUUGUAGUACUCCCACCUCACAC | 649 |
| | AATC | | ACG | |
| LPA-4772 | GUGUGAGGUGGGAGUACUGAA | 250 | AGAUUUCAGUACUCCCACCUCACA | 650 |
| | AUCT | | CAC | |
| LPA-4774 | GUGAGGUGGGAGUACUGCAAU | 251 | UCAGAUUGCAGUACUCCCACCUCA | 651 |
| | CUGA | | CAC | |
| LPA-4775 | UGAGGUGGGAGUACUGCAAAC | 252 | GUCAGUUUGCAGUACUCCCACCUC | 652 |
| | UGAC | | ACA | |
| LPA-4795 | CUGACACAAUGCUCAGAAAAA | 253 | AUUCUUUUUCUGAGCAUUGUGUCA | 653 |
| | GAAT | | GAU | |
| LPA-4796 | UGACACAAUGCUCAGAAACAG | 254 | GAUUCUGUUUCUGAGCAUUGUGUC | 654 |
| | AATC | | AGA | |
| LPA-4797 | GACACAAUGCUCAGAAACAAA | 255 | UGAUUUUGUUUCUGAGCAUUGUGU | 655 |
| | AUCA | | CAG | |
| LPA-4798 | ACACAAUGCUCAGAAACAGAA | 256 | CUGAUUCUGUUUCUGAGCAUUGUG | 656 |
| | UCAG | | UCA | |
| LPA-4799 | CACAAUGCUCAGAAACAGAAU | 257 | CCUGAUUCUGUUUCUGAGCAUUGU | 657 |
| | CAGG | | GUC | |
| LPA-4800 | ACAAUGCUCAGAAACAGAAAC | 258 | ACCUGUUUCUGUUUCUGAGCAUUG | 658 |
| | AGGT | | UGU | |
| LPA-4801 | CAAUGCUCAGAAACAGAAUAA | 259 | CACCUUAUUCUGUUUCUGAGCAUU | 659 |
| | GGTG | | GUG | |
| LPA-4802 | AAUGCUCAGAAACAGAAUCAG | 260 | ACACCUGAUUCUGUUUCUGAGCAU | 660 |
| | GUGT | | UGU | |
| LPA-4803 | AUGCUCAGAAACAGAAUCAAG | 261 | GACACUUGAUUCUGUUUCUGAGCA | 661 |
| | UGTC | | UUG | |
| LPA-4804 | UGCUCAGAAACAGAAUCAGAU | 262 | GGACAUCUGAUUCUGUUUCUGAGC | 662 |
| | GUCC | | AUU | |
| LPA-4806 | CUCAGAAACAGAAUCAGGUAU | 263 | UAGGAUACCUGAUUCUGUUUCUGA | 663 |
| | CCTA | | GCA | |
| LPA-4808 | CAGAAACAGAAUCAGGUGUAC | 264 | UCUAGUACACCUGAUUCUGUUUCU | 664 |
| | UAGA | | GAG | |
| LPA-4809 | AGAAACAGAAUCAGGUGUCAU | 265 | CUCUAUGACACCUGAUUCUGUUUC | 665 |
| | AGAG | | UGA | |
| LPA-4810 | GAAACAGAAUCAGGUGUCCAA | 266 | UCUCUUGGACACCUGAUUCUGUUU | 666 |
| | GAGA | | CUG | |
| LPA-4811 | AAACAGAAUCAGGUGUCCUAG | 267 | GUCUCUAGGACACCUGAUUCUGUU | 667 |
| | AGAC | | UCU | |
| LPA-4812 | AACAGAAUCAGGUGUCCUAAA | 268 | AGUCUUUAGGACACCUGAUUCUGU | 668 |
| | GACT | | UUC | |
| LPA-4814 | CAGAAUCAGGUGUCCUAGAAA | 269 | GGAGUUUCUAGGACACCUGAUUCU | 669 |
| | CUCC | | GUU | |
| LPA-4816 | GAAUCAGGUGUCCUAGAGAAU | 270 | UGGGAUUCUCUAGGACACCUGAUU | 670 |
| | CCCA | | CUG | |
| LPA-4818 | AUCAGGUGUCCUAGAGACUAC | 271 | AGUGGUAGUCUCUAGGACACCUGA | 671 |
| | CACT | | UUC | |
| LPA-4822 | GGUGUCCUAGAGACUCCCAAU | 272 | CAACAUUGGGAGUCUCUAGGACAC | 672 |
| | GUTG | | CUG | |
| LPA-4827 | CCUAGAGACUCCCACUGUUAU | 273 | UGGAAUAACAGUGGGAGUCUCUAG | 673 |
| | UCCA | | GAC | |
| LPA-4828 | CUAGAGACUCCCACUGUUGAU | 274 | CUGGAUCAACAGUGGGAGUCUCUA | 674 |
| | CCAG | | GGA | |
| LPA-4829 | UAGAGACUCCCACUGUUGUAC | 275 | ACUGGUACAACAGUGGGAGUCUCU | 675 |
| | CAGT | | AGG | |
| LPA-4830 | AGAGACUCCCACUGUUGUUAC | 276 | AACUGUAACAACAGUGGGAGUCUC | 676 |
| | AGTT | | UAG | |
| LPA-4831 | GAGACUCCCACUGUUGUUCAA | 277 | GAACUUGAACAACAGUGGGAGUCU | 677 |
| | GUTC | | CUA | |
| LPA-4832 | AGACUCCCACUGUUGUUCCAG | 278 | GGAACUGGAACAACAGUGGGAGUC | 678 |
| | UUCC | | UCU | |
| LPA-4867 | GCUCAUUCUGAAGCAGCACAA | 279 | CAGUUUGUGCUGCUUCAGAAUGAG | 679 |
| | ACTG | | CCU | |
| LPA-4868 | CUCAUUCUGAAGCAGCACCAA | 280 | UCAGUUGGUGCUGCUUCAGAAUGA | 680 |
| | CUGA | | GCC | |
| LPA-4869 | UCAUUCUGAAGCAGCACCAAC | 281 | CUCAGUUGGUGCUGCUUCAGAAUG | 681 |
| | UGAG | | AGC | |
| LPA-4870 | CAUUCUGAAGCAGCACCAAAU | 282 | GCUCAUUUGGUGCUGCUUCAGAAU | 682 |
| | GAGC | | GAG | |
| LPA-4871 | AUUCUGAAGCAGCACCAACAG | 283 | UGCUCUGUUGGUGCUGCUUCAGAA | 683 |
| | AGCA | | UGA | |
| LPA-4872 | UUCUGAAGCAGCACCAACUAA | 284 | UUGCUUAGUUGGUGCUGCUUCAGA | 684 |
| | GCAA | | AUG | |
| LPA-4873 | UCUGAAGCAGCACCAACUGAG | 285 | UUUGCUCAGUUGGUGCUGCUUCAG | 685 |
| | CAAA | | AAU | |
| LPA-4874 | CUGAAGCAGCACCAACUGAAC | 286 | GUUUGUUCAGUUGGUGCUGCUUCA | 686 |
| | AAAC | | GAA | |
| LPA-4875 | UGAAGCAGCACCAACUGAGAA | 287 | GGUUUUCUCAGUUGGUGCUGCUUC | 687 |
| | AACC | | AGA | |
| LPA-4876 | GAAGCAGCACCAACUGAGCAA | 288 | GGGUUUGCUCAGUUGGUGCUGCUU | 688 |
| | ACCC | | CAG | |
| LPA-4877 | AAGCAGCACCAACUGAGCAAA | 289 | GGGGUUUGCUCAGUUGGUGCUGCU | 689 |
| | CCCC | | UCA | |
| LPA-4912 | CAGUGCUACCAUGGUAAUGAC | 290 | UCUGGUCAUUACCAUGGUAGCACU | 690 |
| | CAGA | | GCC | |
| LPA-4913 | AGUGCUACCAUGGUAAUGGAC | 291 | CUCUGUCCAUUACCAUGGUAGCAC | 691 |
| | AGAG | | UGC | |
| LPA-4948 | ACAUUCUCCACCACUGUCAAA | 292 | UUCCUUUGACAGUGGUGGAGAAUG | 692 |
| | GGAA | | UGC | |
| LPA-4959 | CACUGUCACAGGAAGGACAAG | 293 | UUGACUUGUCCUUCCUGUGACAGU | 693 |
| | UCAA | | GGU | |
| LPA-4960 | ACUGUCACAGGAAGGACAUAU | 294 | AUUGAUAUGUCCUUCCUGUGACAG | 694 |
| | CAAT | | UGG | |
| LPA-4961 | CUGUCACAGGAAGGACAUGAC | 295 | GAUUGUCAUGUCCUUCCUGUGACA | 695 |
| | AATC | | GUG | |
| LPA-4962 | UGUCACAGGAAGGACAUGUAA | 296 | AGAUUUACAUGUCCUUCCUGUGAC | 696 |
| | AUCT | | AGU | |
| LPA-4963 | GUCACAGGAAGGACAUGUCAA | 297 | AAGAUUGACAUGUCCUUCCUGUGA | 697 |
| | UCTT | | CAG | |
| LPA-4964 | UCACAGGAAGGACAUGUCAAU | 298 | CAAGAUUGACAUGUCCUUCCUGUG | 698 |
| | CUTG | | ACA | |
| LPA-4966 | ACAGGAAGGACAUGUCAAUAU | 299 | ACCAAUAUUGACAUGUCCUUCCUG | 699 |
| | UGGT | | UGA | |
| LPA-4967 | CAGGAAGGACAUGUCAAUCAU | 300 | GACCAUGAUUGACAUGUCCUUCCU | 700 |
| | GGTC | | GUG | |
| LPA-4968 | AGGAAGGACAUGUCAAUCUAG | 301 | UGACCUAGAUUGACAUGUCCUUCC | 701 |
| | GUCA | | UGU | |
| LPA-4969 | GGAAGGACAUGUCAAUCUUAG | 302 | AUGACUAAGAUUGACAUGUCCUUC | 702 |
| | UCAT | | CUG | |
| LPA-4970 | GAAGGACAUGUCAAUCUUGAU | 303 | GAUGAUCAAGAUUGACAUGUCCUU | 703 |
| | CATC | | CCU | |
| LPA-4971 | AAGGACAUGUCAAUCUUGGAC | 304 | GGAUGUCCAAGAUUGACAUGUCCU | 704 |
| | AUCC | | UCC | |
| LPA-4972 | AGGACAUGUCAAUCUUGGUAA | 305 | UGGAUUACCAAGAUUGACAUGUCC | 705 |
| | UCCA | | UUC | |
| LPA-4973 | GGACAUGUCAAUCUUGGUCAU | 306 | AUGGAUGACCAAGAUUGACAUGUC | 706 |
| | CCAT | | CUU | |
| LPA-4974 | GACAUGUCAAUCUUGGUCAAC | 307 | CAUGGUUGACCAAGAUUGACAUGU | 707 |
| | CATG | | CCU | |
| LPA-4975 | ACAUGUCAAUCUUGGUCAUAC | 308 | UCAUGUAUGACCAAGAUUGACAUG | 708 |
| | AUGA | | UCC | |
| LPA-4976 | CAUGUCAAUCUUGGUCAUCAA | 309 | GUCAUUGAUGACCAAGAUUGACAU | 709 |
| | UGAC | | GUC | |
| LPA-4977 | AUGUCAAUCUUGGUCAUCCAU | 310 | UGUCAUGGAUGACCAAGAUUGACA | 710 |
| | GACA | | UGU | |
| LPA-4978 | UGUCAAUCUUGGUCAUCCAAG | 311 | GUGUCUUGGAUGACCAAGAUUGAC | 711 |
| | ACAC | | AUG | |
| LPA-4979 | GUCAAUCUUGGUCAUCCAUAA | 312 | GGUGUUAUGGAUGACCAAGAUUGA | 712 |
| | CACC | | CAU | |
| LPA-4980 | UCAAUCUUGGUCAUCCAUGAC | 313 | UGGUGUCAUGGAUGACCAAGAUUG | 713 |
| | ACCA | | ACA | |
| LPA-4981 | CAAUCUUGGUCAUCCAUGAAA | 314 | GUGGUUUCAUGGAUGACCAAGAUU | 714 |
| | CCAC | | GAC | |
| LPA-4982 | AAUCUUGGUCAUCCAUGACAC | 315 | UGUGGUGUCAUGGAUGACCAAGAU | 715 |
| | CACA | | UGA | |
| LPA-4983 | AUCUUGGUCAUCCAUGACAAC | 316 | GUGUGUUGUCAUGGAUGACCAAGA | 716 |
| | ACAC | | UUG | |
| LPA-5048 | UGACAAUGAACUACUGCAGAA | 317 | GGAUUUCUGCAGUAGUUCAUUGUC | 717 |
| | AUCC | | AGG | |
| LPA-5049 | GACAAUGAACUACUGCAGGAA | 318 | UGGAUUCCUGCAGUAGUUCAUUGU | 718 |
| | UCCA | | CAG | |
| LPA-5050 | ACAAUGAACUACUGCAGGAAU | 319 | CUGGAUUCCUGCAGUAGUUCAUUG | 719 |
| | CCAG | | UCA | |
| LPA-5051 | CAAUGAACUACUGCAGGAAAC | 320 | UCUGGUUUCCUGCAGUAGUUCAUU | 720 |
| | CAGA | | GUC | |
| LPA-5052 | AAUGAACUACUGCAGGAAUAC | 321 | AUCUGUAUUCCUGCAGUAGUUCAU | 721 |
| | AGAT | | UGU | |
| LPA-5053 | AUGAACUACUGCAGGAAUCAA | 322 | CAUCUUGAUUCCUGCAGUAGUUCA | 722 |
| | GATG | | UUG | |
| LPA-5054 | UGAACUACUGCAGGAAUCCAG | 323 | GCAUCUGGAUUCCUGCAGUAGUUC | 723 |
| | AUGC | | AUU | |
| LPA-5058 | CUACUGCAGGAAUCCAGAUAC | 324 | AUCGGUAUCUGGAUUCCUGCAGUA | 724 |
| | CGAT | | GUU | |
| LPA-5084 | CAGGCCCUUGGUGUUUUACAA | 325 | UCCAUUGUAAAACACCAAGGGCCU | 725 |
| | UGGA | | GUA | |
| LPA-5090 | CUUGGUGUUUUACCAUGGAAC | 326 | CUGGGUUCCAUGGUAAAACACCAA | 726 |
| | CCAG | | GGG | |
| LPA-5091 | UUGGUGUUUUACCAUGGACAC | 327 | GCUGGUGUCCAUGGUAAAACACCA | 727 |
| | CAGC | | AGG | |
| LPA-5092 | UGGUGUUUUACCAUGGACCAC | 328 | UGCUGUGGUCCAUGGUAAAACACC | 728 |
| | AGCA | | AAG | |
| LPA-5093 | GGUGUUUUACCAUGGACCCAA | 329 | AUGCUUGGGUCCAUGGUAAAACAC | 729 |
| | GCAT | | CAA | |
| LPA-5094 | GUGUUUUACCAUGGACCCCAG | 330 | GAUGCUGGGGUCCAUGGUAAAACA | 730 |
| | CATC | | CCA | |
| LPA-5096 | GUUUUACCAUGGACCCCAGAA | 331 | CUGAUUCUGGGGUCCAUGGUAAAA | 731 |
| | UCAG | | CAC | |
| LPA-5124 | GGAGUACUGCAACCUGACGAG | 332 | GCAUCUCGUCAGGUUGCAGUACUC | 732 |
| | AUGC | | CCA | |
| LPA-5125 | GAGUACUGCAACCUGACGCAA | 333 | AGCAUUGCGUCAGGUUGCAGUACU | 733 |
| | UGCT | | CCC | |
| LPA-5127 | GUACUGCAACCUGACGCGAAG | 334 | UGAGCUUCGCGUCAGGUUGCAGUA | 734 |
| | CUCA | | CUC | |
| LPA-5128 | UACUGCAACCUGACGCGAUAC | 335 | CUGAGUAUCGCGUCAGGUUGCAGU | 735 |
| | UCAG | | ACU | |
| LPA-5131 | UGCAACCUGACGCGAUGCUAA | 336 | UGUCUUAGCAUCGCGUCAGGUUGC | 736 |
| | GACA | | AGU | |
| LPA-5136 | CCUGACGCGAUGCUCAGACAC | 337 | UUCUGUGUCUGAGCAUCGCGUCAG | 737 |
| | AGAA | | GUU | |
| LPA-5137 | CUGACGCGAUGCUCAGACAAA | 338 | CUUCUUUGUCUGAGCAUCGCGUCA | 738 |
| | GAAG | | GGU | |
| LPA-5144 | GAUGCUCAGACACAGAAGGAA | 339 | ACAGUUCCUUCUGUGUCUGAGCAU | 739 |
| | CUGT | | CGC | |
| LPA-5145 | AUGCUCAGACACAGAAGGGAC | 340 | CACAGUCCCUUCUGUGUCUGAGCA | 740 |
| | UGTG | | UCG | |
| LPA-5151 | AGACACAGAAGGGACUGUGAU | 341 | AGCGAUCACAGUCCCUUCUGUGUC | 741 |
| | CGCT | | UGA | |
| LPA-5467 | GCAUCCUCUUCAUUUGAUUAU | 342 | UCCCAUAAUCAAAUGAAGAGGAUG | 742 |
| | GGGA | | CAC | |
| LPA-5468 | CAUCCUCUUCAUUUGAUUGAG | 343 | UUCCCUCAAUCAAAUGAAGAGGAU | 743 |
| | GGAA | | GCA | |
| LPA-5469 | AUCCUCUUCAUUUGAUUGUAG | 344 | CUUCCUACAAUCAAAUGAAGAGGA | 744 |
| | GAAG | | UGC | |
| LPA-5470 | UCCUCUUCAUUUGAUUGUGAG | 345 | GCUUCUCACAAUCAAAUGAAGAGG | 745 |
| | AAGC | | AUG | |
| LPA-5471 | CCUCUUCAUUUGAUUGUGGAA | 346 | GGCUUUCCACAAUCAAAUGAAGAG | 746 |
| | AGCC | | GAU | |
| LPA-5474 | CUUCAUUUGAUUGUGGGAAAC | 347 | UGAGGUUUCCCACAAUCAAAUGAA | 747 |
| | CUCA | | GAG | |
| LPA-5475 | UUCAUUUGAUUGUGGGAAGAC | 348 | UUGAGUCUUCCCACAAUCAAAUGA | 748 |
| | UCAA | | AGA | |
| LPA-5476 | UCAUUUGAUUGUGGGAAGCAU | 349 | CUUGAUGCUUCCCACAAUCAAAUG | 749 |
| | CAAG | | AAG | |
| LPA-5477 | CAUUUGAUUGUGGGAAGCCAC | 350 | ACUUGUGGCUUCCCACAAUCAAAU | 750 |
| | AAGT | | GAA | |
| LPA-5478 | AUUUGAUUGUGGGAAGCCUAA | 351 | CACUUUAGGCUUCCCACAAUCAAA | 751 |
| | AGTG | | UGA | |
| LPA-5486 | GUGGGAAGCCUCAAGUGGAAC | 352 | UUCGGUUCCACUUGAGGCUUCCCA | 752 |
| | CGAA | | CAA | |
| LPA-5509 | AAGAAAUGUCCUGGAAGCAAU | 353 | CUACAUUGCUUCCAGGACAUUUCU | 753 |
| | GUAG | | UCG | |
| LPA-5510 | AGAAAUGUCCUGGAAGCAUAG | 354 | CCUACUAUGCUUCCAGGACAUUUC | 754 |
| | UAGG | | UUC | |
| LPA-5511 | GAAAUGUCCUGGAAGCAUUAU | 355 | CCCUAUAAUGCUUCCAGGACAUUU | 755 |
| | AGGG | | CUU | |
| LPA-5513 | AAUGUCCUGGAAGCAUUGUAG | 356 | CCCCCUACAAUGCUUCCAGGACAU | 756 |
| | GGGG | | UUC | |
| LPA-5514 | AUGUCCUGGAAGCAUUGUAAG | 357 | CCCCCUUACAAUGCUUCCAGGACA | 757 |
| | GGGG | | UUU | |
| LPA-5581 | AGAACAAGGUUUGGAAAGCAC | 358 | AGAAGUGCUUUCCAAACCUUGUUC | 758 |
| | UUCT | | UGA | |
| LPA-5582 | GAACAAGGUUUGGAAAGCAAU | 359 | CAGAAUUGCUUUCCAAACCUUGUU | 759 |
| | UCTG | | CUG | |
| LPA-5583 | AACAAGGUUUGGAAAGCACAU | 360 | ACAGAUGUGCUUUCCAAACCUUGU | 760 |
| | CUGT | | UCU | |
| LPA-5584 | ACAAGGUUUGGAAAGCACUAC | 361 | CACAGUAGUGCUUUCCAAACCUUG | 761 |
| | UGTG | | UUC | |
| LPA-5585 | CAAGGUUUGGAAAGCACUUAU | 362 | CCACAUAAGUGCUUUCCAAACCUU | 762 |
| | GUGG | | GUU | |
| LPA-5586 | AAGGUUUGGAAAGCACUUCAG | 363 | UCCACUGAAGUGCUUUCCAAACCU | 763 |
| | UGGA | | UGU | |
| LPA-5587 | AGGUUUGGAAAGCACUUCUAU | 364 | CUCCAUAGAAGUGCUUUCCAAACC | 764 |
| | GGAG | | UUG | |
| LPA-5592 | UGGAAAGCACUUCUGUGGAAG | 365 | GGUGCUUCCACAGAAGUGCUUUCC | 765 |
| | CACC | | AAA | |
| LPA-5606 | GUGGAGGCACCUUAAUAUCAC | 366 | UCUGGUGAUAUUAAGGUGCCUCCA | 766 |
| | CAGA | | CAG | |
| LPA-5616 | CUUAAUAUCCCCAGAGUGGAU | 367 | CAGCAUCCACUCUGGGGAUAUUAA | 767 |
| | GCTG | | GGU | |
| LPA-5618 | UAAUAUCCCCAGAGUGGGUAC | 368 | GUCAGUACCCACUCUGGGGAUAUU | 768 |
| | UGAC | | AAG | |
| LPA-5628 | AGAGUGGGUGCUGACUGCUAC | 369 | GUGAGUAGCAGUCAGCACCCACUC | 769 |
| | UCAC | | UGG | |
| LPA-5685 | CAAGGUCAUCCUGGGUGCAAA | 370 | UUGGUUUGCACCCAGGAUGACCUU | 770 |
| | CCAA | | GUA | |
| LPA-5694 | CCUGGGUGCACACCAAGAAAU | 371 | GUUCAUUUCUUGGUGUGCACCCAG | 771 |
| | GAAC | | GAU | |
| LPA-5699 | GUGCACACCAAGAAGUGAAAC | 372 | UCGAGUUUCACUUCUUGGUGUGCA | 772 |
| | UCGA | | CCC | |
| LPA-5775 | AGCAGAUAUUGCCUUGCUAAA | 373 | UAGCUUUAGCAAGGCAAUAUCUGC | 773 |
| | GCTA | | UUG | |
| LPA-5776 | GCAGAUAUUGCCUUGCUAAAG | 374 | UUAGCUUUAGCAAGGCAAUAUCUG | 774 |
| | CUAA | | CUU | |
| LPA-5777 | CAGAUAUUGCCUUGCUAAAAC | 375 | CUUAGUUUUAGCAAGGCAAUAUCU | 775 |
| | UAAG | | GCU | |
| LPA-5778 | AGAUAUUGCCUUGCUAAAGAU | 376 | GCUUAUCUUUAGCAAGGCAAUAUC | 776 |
| | AAGC | | UGC | |
| LPA-5779 | GAUAUUGCCUUGCUAAAGCAA | 377 | UGCUUUGCUUUAGCAAGGCAAUAU | 777 |
| | AGCA | | CUG | |
| LPA-5780 | AUAUUGCCUUGCUAAAGCUAA | 378 | CUGCUUAGCUUUAGCAAGGCAAUA | 778 |
| | GCAG | | UCU | |
| LPA-5781 | UAUUGCCUUGCUAAAGCUAAG | 379 | CCUGCUUAGCUUUAGCAAGGCAAU | 779 |
| | CAGG | | AUC | |
| LPA-5813 | UCAUCACUGACAAAGUAAUAC | 380 | GCUGGUAUUACUUUGUCAGUGAUG | 780 |
| | CAGC | | ACG | |
| LPA-5873 | GGACUGAAUGUUACAUCACAG | 381 | CAGCCUGUGAUGUAACAUUCAGUC | 781 |
| | GCTG | | CUG | |
| LPA-5874 | GACUGAAUGUUACAUCACUAG | 382 | CCAGCUAGUGAUGUAACAUUCAGU | 782 |
| | CUGG | | CCU | |
| LPA-5875 | ACUGAAUGUUACAUCACUGAC | 383 | CCCAGUCAGUGAUGUAACAUUCAG | 783 |
| | UGGG | | UCC | |
| LPA-5876 | CUGAAUGUUACAUCACUGGAU | 384 | CCCCAUCCAGUGAUGUAACAUUCA | 784 |
| | GGGG | | GUC | |
| LPA-5877 | UGAAUGUUACAUCACUGGCAG | 385 | UCCCCUGCCAGUGAUGUAACAUUC | 785 |
| | GGGA | | AGU | |
| LPA-5879 | AAUGUUACAUCACUGGCUGAG | 386 | UCUCCUCAGCCAGUGAUGUAACAU | 786 |
| | GAGA | | UCA | |
| LPA-5902 | GAAACCCAAGGUACCUUUGAG | 387 | CAGUCUCAAAGGUACCUUGGGUUU | 787 |
| | ACTG | | CUC | |
| LPA-0190-M1 | UCCACCACUGUCACAGGAAAG | 388 | UUUCCUGUGACAGUGGUGGAGG | 788 |
| | CAGCCGAAAGGCUGC | | | |
| LPA-0501-M1 | UGGUAAUGGACAGAGUUAUAG | 389 | UAUAACUCUGUCCAUUACCAGG | 789 |
| | CAGCCGAAAGGCUGC | | | |
| LPA-3100-M1 | UACUGCAACCUGACACGAUAG | 390 | UAUCGUGUCAGGUUGCAGUAGG | 790 |
| | CAGCCGAAAGGCUGC | | | |
| LPA-3286-M1 | AGAACUUGCCAAGCUUGGUAG | 391 | UACCAAGCUUGGCAAGUUCUGG | 791 |
| | CAGCCGAAAGGCUGC | | | |
| LPA-3288-M1 | AACUUGCCAAGCUUGGUCAAG | 392 | UUGACCAAGCUUGGCAAGUUGG | 792 |
| | CAGCCGAAAGGCUGC | | | |
| LPA-3291-M1 | UUGCCAAGCUUGGUCAUCUAG | 393 | UAGAUGACCAAGCUUGGCAAGG | 793 |
| | CAGCCGAAAGGCUGC | | | |
| LPA-3584-M1 | AUGGACAGAGUUAUCGAGGAG | 394 | UCCUCGAUAACUCUGUCCAUGG | 794 |
| | CAGCCGAAAGGCUGC | | | |
| LPA-3585-M1 | UGGACAGAGUUAUCGAGGCAG | 395 | UGCCUCGAUAACUCUGUCCAGG | 795 |
| | CAGCCGAAAGGCUGC | | | |
| LPA-4645-M1 | UGGUCAUCUAUGAUACCACAG | 396 | UGUGGUAUCAUAGAUGACCAGG | 796 |
| | CAGCCGAAAGGCUGC | | | |
| LPA-4717-M1 | UACUGCAGGAAUCCAGAUUAG | 397 | UAAUCUGGAUUCCUGCAGUAGG | 797 |
| | CAGCCGAAAGGCUGC | | | |
| LPA-5510-M1 | AGAAAUGUCCUGGAAGCAUAG | 398 | UAUGCUUCCAGGACAUUUCUGG | 798 |
| | CAGCCGAAAGGCUGC | | | |
| LPA-3750-M1 | GACAACAGAAUAUUAUCCAAG | 399 | UUGGAUAAUAUUCUGUUGUCGG | 799 |
| | CAGCCGAAAGGCUGC | | | |
| LPA-2900-M2 | AUGGACAGAGUUAUCAAGGAG | 400 | UCCUUGAUAACUCUGUCCAUGG | 800 |
| | CAGCCGAAAGGCUGC | | | |
| LPA-3675-M2 | GACAACAGAAUAUUAUCCAAG | 401 | UUGGAUAAUAUUCUGUUGUCGG | 801 |
| | CAGCCGAAAGGCUGC | | | |
| LPA-2900-M3 | AUGGACAGAGUUAUCAAGGAG | 402 | UCCUUGAUAACUCUGUCCAUGG | 802 |
| | CAGCCGAAAGGCUGC | | | |
| LPA-3675-M3 | GACAACAGAAUAUUAUCCAAG | 403 | UUGGAUAAUAUUCUGUUGUCGG | 803 |
| | CAGCCGAAAGGCUGC | | | |

Human (Hs): NM_005577.3 (SEQ ID NO: 1)
CTGGGATTGG GACACACTTT CTGGGCACTG CTGGCCAGTC CCAAAATGGA ACATAAGGAA
GTGGTTCTTC TACTTCTTTT ATTTCTGAAA TCAGCAGCAC CTGAGCAAAG CCATGTGGTC
CAGGATTGCT ACCATGGTGA TGGACAGAGT TATCGAGGCA CGTACTCCAC CACTGTCACA
GGAAGGACCT GCCAAGCTTG GTCATCTATG ACACCACATC AACATAATAG GACCACAGAA
AACTACCCAA ATGCTGGCTT GATCATGAAC TACTGCAGGA ATCCAGATGC TGTGGCAGCT
CCTTATTGTT ATACGAGGGA TCCCGGTGTC AGGTGGGAGT ACTGCAACCT GACGCAATGC
TCAGACGCAG AAGGGACTGC CGTCGCGCCT CCGACTGTTA CCCCGGTTCC AAGCCTAGAG
GCTCCTTCCG AACAAGCACC GACTGAGCAA AGGCCTGGGG TGCAGGAGTG CTACCATGGT
AATGGACAGA GTTATCGAGG CACATACTCC ACCACTGTCA CAGGAAGAAC CTGCCAAGCT
TGGTCATCTA TGACACCACA CTCGCATAGT CGGACCCCAG AATACTACCC AAATGCTGGC
TTGATCATGA ACTACTGCAG GAATCCAGAT GCTGTGGCAG CTCCTTATTG TTATACGAGG
GATCCCGGTG TCAGGTGGGA GTACTGCAAC CTGACGCAAT GCTCAGACGC AGAAGGGACT
GCCGTCGCGC CTCCGACTGT TACCCCGGTT CCAAGCCTAG AGGCTCCTTC CGAACAAGCA
CCGACTGAGC AAAGGCCTGG GGTGCAGGAG TGCTACCATG GTAATGGACA GAGTTATCGA
GGCACATACT CCACCACTGT CACAGGAAGA ACCTGCCAAG CTTGGTCATC TATGACACCA
CACTCGCATA GTCGGACCCC AGAATACTAC CCAAATGCTG GCTTGATCAT GAACTACTGC
AGGAATCCAG ATGCTGTGGC AGCTCCTTAT TGTTATACGA GGGATCCCGG TGTCAGGTGG
GAGTACTGCA ACCTGACGCA ATGCTCAGAC GCAGAAGGGA CTGCCGTCGC GCCTCCGACT
GTTACCCCGG TTCCAAGCCT AGAGGCTCCT TCCGAACAAG CACCGACTGA GCAGAGGCCT
GGGGTGCAGG AGTGCTACCA CGGTAATGGA CAGAGTTATC GAGGCACATA CTCCACCACT
GTCACTGGAA GAACCTGCCA AGCTTGGTCA TCTATGACAC CACACTCGCA TAGTCGGACC
CCAGAATACT ACCCAAATGC TGGCTTGATC ATGAACTACT GCAGGAATCC AGATGCTGTG
GCAGCTCCTT ATTGTTATAC GAGGGATCCC GGTGTCAGGT GGGAGTACTG CAACCTGACG
CAATGCTCAG ACGCAGAAGG GACTGCCGTC GCGCCTCCGA CTGTTACCCC GGTTCCAAGC
CTAGAGGCTC CTTCCGAACA AGCACCGACT GAGCAAAGGC CTGGGGTGCA GGAGTGCTAC
CATGGTAATG GACAGAGTTA TCGAGGCACA TACTCCACCA CTGTCACAGG AAGAACCTGC
CAAGCTTGGT CATCTATGAC ACCACACTCG CATAGTCGGA CCCCAGAATA CTACCCAAAT
GCTGGCTTGA TCATGAACTA CTGCAGGAAT CCAGATGCTG TGGCAGCTCC TTATTGTTAT
ACGAGGGATC CCGGTGTCAG GTGGGAGTAC TGCAACCTGA CGCAATGCTC AGACGCAGAA
GGGACTGCCG TCGCGCCTCC GACTGTTACC CCGGTTCCAA GCCTAGAGGC TCCTTCCGAA
CAAGCACCGA CTGAGCAAAG GCCTGGGGTG CAGGAGTGCT ACCATGGTAA TGGACAGAGT
TATCGAGGCA CATACTCCAC CACTGTCACA GGAAGAACCT GCCAAGCTTG GTCATCTATG
ACACCACACT CGCATAGTCG GACCCCAGAA TACTACCCAA ATGCTGGCTT GATCATGAAC
TACTGCAGGA ATCCAGATGC TGTGGCAGCT CCTTATTGTT ATACGAGGGA TCCCGGTGTC
AGGTGGGAGT ACTGCAACCT GACGCAATGC TCAGACGCAG AAGGGACTGC CGTCGCGCCT
CCGACTGTTA CCCCGGTTCC AAGCCTAGAG GCTCCTTCCG AACAAGCACC GACTGAGCAA
AGGCCTGGGG TGCAGGAGTG CTACCATGGT AATGGACAGA GTTATCGAGG CACATACTCC
ACCACTGTCA CAGGAAGAAC CTGCCAAGCT TGGTCATCTA TGACACCACA CTCGCATAGT
CGGACCCCAG AATACTACCC AAATGCTGGC TTGATCATGA ACTACTGCAG GAATCCAGAT
GCTGTGGCAG CTCCTTATTG TTATACGAGG GATCCCGGTG TCAGGTGGGA GTACTGCAAC
CTGACGCAAT GCTCAGACGC AGAAGGGACT GCCGTCGCGC CTCCGACTGT TACCCCGGTT
CCAAGCCTAG AGGCTCCTTC CGAACAAGCA CCGACTGAGC AGAGGCCTGG GGTGCAGGAG
TGCTACCACG GTAATGGACA GAGTTATCGA GGCACATACT CCACCACTGT CACTGGAAGA
ACCTGCCAAG CTTGGTCATC TATGACACCA CACTCGCATA GTCGGACCCC AGAATACTAC
CCAAATGCTG GCTTGATCAT GAACTACTGC AGGAATCCAG ATCCTGTGGC AGCCCCTTAT
TGTTATACGA GGGATCCCAG TGTCAGGTGG GAGTACTGCA ACCTGACACA ATGCTCAGAC
GCAGAAGGGA CTGCCGTCGC GCCTCCAACT ATTACCCCGA TTCCAAGCCT AGAGGCTCCT
TCTGAACAAG CACCAACTGA GCAAAGGCCT GGGGTGCAGG AGTGCTACCA CGGAAATGGA
CAGAGTTATC AAGGCACATA CTTCATTACT GTCACAGGAA GAACCTGCCA AGCTTGGTCA
TCTATGACAC CACACTCGCA TAGTCGGACC CCAGCATACT ACCCAAATGC TGGCTTGATC
AAGAACTACT GCCGAAATCC AGATCCTGTG GCAGCCCCTT GGTGTTATAC AACAGATCCC
AGTGTCAGGT GGGAGTACTG CAACCTGACA CGATGCTCAG ATGCAGAATG GACTGCCTTC
GTCCCTCCGA ATGTTATTCT GGCTCCAAGC CTAGAGGCTT TTTTTGAACA AGCACTGACT
GAGGAAACCC CCGGGGTACA GGACTGCTAC TACCATTATG GACAGAGTTA CCGAGGCACA
TACTCCACCA CTGTCACAGG AAGAACTTGC CAAGCTTGGT CATCTATGAC ACCACACCAG
CATAGTCGGA CCCCAGAAAA CTACCCAAAT GCTGGCCTGA CCAGGAACTA CTGCAGGAAT
CCAGATGCTG AGATTCGCCC TTGGTGTTAC ACCATGGATC CCAGTGTCAG GTGGGAGTAC
TGCAACCTGA CACAATGCCT GGTGACAGAA TCAAGTGTCC TTGCAACTCT CACGGTGGTC
CCAGATCCAA GCACAGAGGC TTCTTCTGAA GAAGCACCAA CGGAGCAAAG CCCCGGGGTC
CAGGATTGCT ACCATGGTGA TGGACAGAGT TATCGAGGCT CATTCTCTAC CACTGTCACA
GGAAGGACAT GTCAGTCTTG GTCCTCTATG ACACCACACT GGCATCAGAG GACAACAGAA
TATTATCCAA ATGGTGGCCT GACCAGGAAC TACTGCAGGA ATCCAGATGC TGAGATTAGT
CCTTGGTGTT ATACCATGGA TCCCAATGTC AGATGGGAGT ACTGCAACCT GACACAATGT
CCAGTGACAG AATCAAGTGT CCTTGCGACG TCCACGGCTG TTTCTGAACA AGCACCAACG
GAGCAAAGCC CCACAGTCCA GGACTGCTAC CATGGTGATG GACAGAGTTA TCGAGGCTCA
TTCTCCACCA CTGTTACAGG AAGGACATGT CAGTCTTGGT CCTCTATGAC ACCACACTGG
CATCAGAGAA CCACAGAATA CTACCCAAAT GGTGGCCTGA CCAGGAACTA CTGCAGGAAT
CCAGATGCTG AGATTCGCCC TTGGTGTTAT ACCATGGATC CCAGTGTCAG ATGGGAGTAC
TGCAACCTGA CGCAATGTCC AGTGATGGAA TCAACTCTCC TCACAACTCC CACGGTGGTC
CCAGTTCCAA GCACAGAGCT TCCTTCTGAA GAAGCACCAA CTGAAAACAG CACTGGGGTC
CAGGACTGCT ACCGAGGTGA TGGACAGAGT TATCGAGGCA CACTCTCCAC CACTATCACA
GGAAGAACAT GTCAGTCTTG GTCGTCTATG ACACCACATT GGCATCGGAG GATCCCATTA
TACTATCCAA ATGCTGGCCT GACCAGGAAC TACTGCAGGA ATCCAGATGC TGAGATTCGC
CCTTGGTGTT ACACCATGGA TCCCAGTGTC AGGTGGGAGT ACTGCAACCT GACACGATGT
CCAGTGACAG AATCGAGTGT CCTCACAACT CCCACAGTGG CCCCGGTTCC AAGCACAGAG
GCTCCTTCTG AACAAGCACC ACCTGAGAAA AGCCCTGTGG TCCAGGATTG CTACCATGGT
GATGGACGGA GTTATCGAGG CATATCCTCC ACCACTGTCA CAGGAAGGAC CTGTCAATCT
TGGTCATCTA TGATACCACA CTGGCATCAG AGGACCCCAG AAAACTACCC AAATGCTGGC
CTGACCGAGA ACTACTGCAG GAATCCAGAT TCTGGGAAAC AACCCTGGTG TTACACAACC
GATCCGTGTG TGAGGTGGGA GTACTGCAAT CTGACACAAT GCTCAGAAAC AGAATCAGGT
GTCCTAGAGA CTCCCACTGT TGTTCCAGTT CCAAGCATGG AGGCTCATTC TGAAGCAGCA
CCAACTGAGC AAACCCCTGT GGTCCGGCAG TGCTACCATG GTAATGGCCA GAGTTATCGA
GGCACATTCT CCACCACTGT CACAGGAAGG ACATGTCAAT CTTGGTCATC CATGACACCA
CACCGGCATC AGAGGACCCC AGAAAACTAC CCAAATGATG GCCTGACAAT GAACTACTGC
AGGAATCCAG ATGCCGATAC AGGCCCTTGG TGTTTTACCA TGGACCCCAG CATCAGGTGG
GAGTACTGCA ACCTGACGCG ATGCTCAGAC ACAGAAGGGA CTGTGGTCGC TCCTCCGACT
GTCATCCAGG TTCCAAGCCT AGGGCCTCCT TCTGAACAAG ACTGTATGTT TGGGAATGGG
AAAGGATACC GGGGCAAGAA GGCAACCACT GTTACTGGGA CGCCATGCCA GGAATGGGCT
GCCCAGGAGC CCCATAGACA CAGCACGTTC ATTCCAGGGA CAAATAAATG GGCAGGTCTG
GAAAAAAATT ACTGCCGTAA CCCTGATGGT GACATCAATG GTCCCTGGTG CTACACAATG
AATCCAAGAA AACTTTTTGA CTACTGTGAT ATCCCTCTCT GTGCATCCTC TTCATTTGAT
TGTGGGAAGC CTCAAGTGGA GCCGAAGAAA TGTCCTGGAA GCATTGTAGG GGGGTGTGTG
GCCCACCCAC ATTCCTGGCC CTGGCAAGTC AGTCTCAGAA CAAGGTTTGG AAAGCACTTC
TGTGGAGGCA CCTTAATATC CCCAGAGTGG GTGCTGACTG CTGCTCACTG CTTGAAGAAG
TCCTCAAGGC CTTCATCCTA CAAGGTCATC CTGGGTGCAC ACCAAGAAGT GAACCTCGAA
TCTCATGTTC AGGAAATAGA AGTGTCTAGG CTGTTCTTGG AGCCCACACA AGCAGATATT
GCCTTGCTAA AGCTAAGCAG GCCTGCCGTC ATCACTGACA AAGTAATGCC AGCTTGTCTG
CCATCCCCAG ACTACATGGT CACCGCCAGG ACTGAATGTT ACATCACTGG CTGGGGAGAA
ACCCAAGGTA CCTTTGGGAC TGGCCTTCTC AAGGAAGCCC AGCTCCTTGT TATTGAGAAT
GAAGTGTGCA ATCACTATAA GTATATTTGT GCTGAGCATT TGGCCAGAGG CACTGACAGT
TGCCAGGGTG ACAGTGGAGG GCCTCTGGTT TGCTTCGAGA AGGACAAATA CATTTTACAA
GGAGTCACTT CTTGGGGTCT TGGCTGTGCA CGCCCCAATA AGCCTGGTGT CTATGCTCGT
GTTTCAAGGT TTGTTACTTG GATTGAGGGA ATGATGAGAA ATAATTAATT GGACGGGAGA
CAGAGTGAAG CATCAACCTA CTTAGAAGCT GAAACGTGGG TAAGGATTTA GCATGCTGGA
AATAATAGAC AGCAATCAAA CGAAGACACT GTTCCCAGCT ACCAGCTATG CCAAACCTTG
GCATTTTTGG TATTTTTGTG TATAAGCTTT TAAGGTCTGA CTGACAAATT CTGTATTAAG
GTGTCATAGC TATGACATTT GTTAAAAATA AACTCTGCAC TTATTTTGAT TTGA
Cynomolgus monkey (Mf): XM_015448517. 1 (SEQ ID NO: 2)
GATGCTGCAT ACTTAATGTC GAAAGGTTGC TTCATCCAAG AGCCTGGAGT TTTCAGAGAC
ACTGTCCTGA AACTATGTCC TGAAACTATG TCATTGAAAC TGAAACATTG TCCTGAAGCT
GGTATTGGGC AATACCAGCG CCTGCAGGCA ACAGCTCGGA TGCACTTAAG ATTTAAATAT
TACCCACAGA AGTTCTGGCT TGTCTGGGAA AACCTTTTGC TAAACAGAAG AGCAACATTT
TTTTTTTTTT CTTTTCTGGA ATTTGTAAAC AGCATTTATT CTCAGCCTTA CCTTCCAAAC
GTTGCACTTG GAACATTGCT GGGCCCCGTG GAAACAGAAG CGAACGTCAG CCAGGCCGGC
AGGGGGCGGC AGACCCCACA CTTCGCCGGG CGCCCTCACC TCCCTGGGAG GGAGTGTGCA
GCTGCCAAAA TCTTCGGCGG GGTTCAGTCC AAGCGACTTC AGCCAGCAGA TGGTCATTCT
CCTGTGACCG TGTGTACTAC AGACTGTTTC AAAACCGGGC AGGCAATTAA CAATGGGAAT
TCTGCCATCA TCGCTGACAA AGTCATCCCA GTTTGTCTGC CATCCCCAAA TTATGTGGTC
GCCAACCAGA CTGAATGTTA TGTCACTGGC TGGGGAGAAA CCCAAGCACT ACCTGAGCAA
AGCCATGTGG TCCAGGATTG CTACCATGGT GATGGACAGA GTTATCAAGG CACATCCTCC
ACCACTGTCA CAGGAAGGAC CTGCCAAGCT TGGTCATCTA TGGAACCACA TCAGCATAAT
AGAACCACAG AAAACTACCC AAATGCTGGC TTGATCAGGA ACTACTGCAG GAATCCAGAT
CCTGTGGCAG CCCCTTATTG TTATACGATG GATCCCAATG TCAGGTGGGA GTACTGCAAC
CTGACACAAT GCTCAGACGC AGAAGGGACT GCCGTCGCAC CTCCGAATGT CACCCCGGTT
CCAAGCCTAG AGGCTCCTTC CGAACAAGCA CCGACTGAGC AAAGGCCTGG GGTGCAGGAG
TGCTACCACG GTAATGGACA GAGTTATCGA GGCACATACT TCACCACTGT GACAGGAAGA
ACCTGCCAAG CTTGGTCATC TATGACACCG CACTCTCATA GTCGGACCCC GGAAAACTAC
CCAAATGGTG GCTTGATCAG GAACTACTGC AGGAATCCAG ATCCTGTGGC AGCCCCTTAT
TGTTATACCA TGGATCCCAA TGTCAGGTGG GAGTACTGCA ACCTGACACA ATGCTCAGAC
GCAGAAGGGA TTGCCGTCAC ACCTCTGACT GTTACCCCGG TTCCAAGCCT AGAGGCTCCT
TCCAAGCAAG CACCAACTGA GCAAAGGCCT GGTGTCCAGG AGTGCTACCA CGGTAATGGA
CAGAGTTATC GAGGCACATA CTTCACCACT GTGACAGGAA GAACCTGCCA AGCTTGGTCA
TCTATGACAC CACATTCTCA TAGTCGTACC CCAGAAAACT ACCCAAATGG TGGCTTGATC
AGGAACTACT GCAGGAATCC AGATCCTGTG GCAGCCCCTT ATTGTTATAC CATGGATCCC
AATGTCAGGT GGGAGTACTG CAACCTGACA CAATGCTCAG ACGCAGAAGG GACTGCCGTC
GCACCTCCGA CTGTCACCCC GGTTCCAAGC CTAGAGGCTC CTTCCGAACA AGCACCGACT
GAGCAAAGGC CTGGGGTGCA GGAGTGCTAC CACGGTAATG GACAGAGTTA TCGAGGCACA
TACTTCACCA CTGTGACAGG AAGAACCTGC CAAGCTTGGT CATCTATGAC ACCGCACTCT
CATAGTCGGA CCCCGGAAAA CTACCCAAAT GGTGGCTTGA TCAGGAACTA CTGCAGGAAT
CCAGATCCTG TGGCAGCCCC TTATTGTTAT ACCATGGATC CCAATGTCAG GTGGGAGTAC
TGCAACCTGA CACAATGCTC AGACGCAGAA GGGACTGCCG TCGCACCTCC GAATGTCACC
CCGGTTCCAA GCCTAGAGGC TCCTTCTGAG CAAGCACCAA CTGAGCAAAG GCTTGGGGTG
CAGGAGTGCT ACCACGGTAA TGGACAGAGT TATCGAGGCA CATACTTCAC CACTGTGACA
GGAAGAACCT GCCAAGCTTG GTCATCTATG ACACCACACT CTCATAGTCG GACCCCAGAA
AACTACCCAA ATGCTGGCTT GGTCAAGAAC TACTGCCGAA ATCCAGATCC TGTGGCAGCC
CCTTGGTGTT ATACAACGGA TCCCAGTGTC AGGTGGGAGT ACTGCAACCT GACACGATGC
TCAGATGCAG AAGGGACTGC TGTTGTGCCT CCAAATATTA TTCCGGTTCC AAGCCTAGAG
GCTTTTCTTG AACAAGAACC GACTGAGGAA ACCCCCGGGG TACAGGAGTG CTACTACCAT
TATGGACAGA GTTATAGAGG CACATACTCC ACCACTGTTA CAGGAAGAAC TTGCCAAGCT
TGGTCATCTA TGACACCACA CCAGCATAGT CGGACCCCAA AAAACTATCC AAATGCTGGC
CTGACCAGGA ACTACTGCAG GAATCCAGAT GCTGAGATTC GCCCTTGGTG TTATACCATG
GATCCCAGTG TCAGGTGGGA GTACTGCAAC CTGACACAAT GTCTGGTGAC AGAATCAAGT
GTCCTTGAAA CTCTCACAGT GGTCCCAGAT CCAAGCACAC AGGCTTCTTC TGAAGAAGCA
CCAACGGAGC AAAGTCCCGA GGTCCAGGAC TGCTACCATG GTGATGGACA GAGTTATCGA
GGCTCATTCT CCACCACTGT CACAGGAAGG ACATGTCAGT CTTGGTCCTC TATGACACCA
CACTGGCATC AGAGGACAAC AGAATATTAT CCAGATGGTG GCCTGACCAG GAACTACTGC
AGGAATCCAG ATGCTGAGAT TCGCCCTTGG TGTTATACCA TGGATCCCAG TGTCAGGTGG
GAGTACTGCA ACCTGACACA ATGTCCAGTG ACAGAATCAA GTGTCCTCGC AACGTCCATG
GCTGTTTCTG AACAAGCACC AATGGAGCAA AGCCCCGGGG TCCAGGACTG CTACCATGGT
GATGGACAGA GTTATCGAGG TTCATTCTCC ACCACTGTCA CAGGAAGGAC ATGTCAGTCT
TGGTCCTCTA TGACACCACA CTGGCATCAG AGGACCATAG AATACTACCC AAATGGTGGC
CTGACCAAGA ACTACTGCAG GAATCCAGAT GCTGAGATTC GCCCTTGGTG TTATACCATG
GATCCCAGAG TCAGATGGGA GTACTGCAAC CTGACACAAT GTGTGGTGAT GGAATCAAGT
GTCCTTGCAA CTCCCATGGT GGTCCCAGTT CCAAGCAGAG AGGTTCCTTC TGAAGAAGCA
CCAACTGAAA ACAGCCCTGG GGTCCAGGAC TGCTACCAAG GTGATGGACA GAGTTATCGA
GGCACATTCT CCACCACTAT CACAGGAAGA ACATGTCAGT CTTGGTTGTC TATGACACCA
CATCGGCATC GGAGGATCCC ATTACGCTAT CCAAATGCTG GCCTGACCAG GAACTATTGC
AGAAATCCAG ATGCTGAGAT TCGCCCTTGG TGTTACACCA TGGATCCCAG TGTCAGGTGG
GAGTACTGCA ACCTGACACA ATGTCCAGTG ACAGAATCAA GTGTCCTCAC AACTCCCACG
GTGGTCCCGG TTCCAAGCAC AGAGGCTCCT TCTGAACAAG CACCACCTGA GAAAAGCCCT
GTGGTCCAGG ATTGCTACCA TGGTGATGGA CAGAGTTATC GAGGCACATC CTCCACCACT
GTCACAGGAA GGAACTGTCA GTCTTGGTCA TCTATGATAC CACACTGGCA TCAGAGGACC
CCAGAAAACT ACCCAAATGC TGGCCTGACC AGGAACTACT GCAGGAATCC AGATTCTGGG
AAACAACCCT GGTGTTACAC GACTGATCCA TGTGTGAGGT GGGAGTACTG CAACCTGACA
CAATGCTCAG AAACAGAATC AGGTGTCCTA GAGACTCCCA CTGTTGTTCC GGTTCCAAGC
ATGGAAGCTC ATTCTGAAGC AGCACCAACT GAGCAAACTC CTGTGGTCCA GCAGTGCTAC
CATGGTAATG GACAGAGTTA TCGAGGCACA TTCTCCACCA CTGTCACAGG AAGGACATGT
CAATCTTGGT CATCCATGAC ACCACACCAG CATAAGAGGA CCCCGGAAAA CCACCCAAAT
GATGACTTGA CAATGAACTA CTGCAGGAAT CCAGATGCTG ACACAGGCCC TTGGTGTTTT
ACCATGGACC CCAGCGTCAG GCGGGAGTAC TGCAACCTGA CGCGATGCTC AGACACAGAA
GGGACTGTGG TCACACCTCC GACTGTTATC CCGGTTCCAA GCCTAGAGGC TCCTTCTGAA
CAAGCATCCT CTTCATTTGA TTGTGGGAAG CCTCAAGTGG AGCCAAAGAA ATGTCCTGGA
AGCATTGTAG GTGGGTGTGT GGCCCACCCA CATTCCTGGC CCTGGCAAGT CAGTCTTAGA
ACAAGGTTTG GAAAGCACTT CTGTGGAGGC ACCTTAATAT CCCCAGAGTG GGTGCTGACT
GCTGCTTGCT GCTTGGAGAC GTTCTCAAGG CCTTCCTTCT ACAAGGTCAT CCTGGGTGCA
CACCAAGAAG TGAATCTCGA ATCTCACGTT CAAGAAATAG AAGTGTCTAG GTTGTTCTTG
GAGCCCATAG GAGCAGATAT TGCCTTGCTA AAGCTAAGCA GGCCTGCCAT CATCACTGAC
AAAGTAATCC CAGCCTGTCT GCCGTCTCCA AATTACGTGA TCACCGTCTG GACTGAATGT
TACATCACTG GCTGGGGAGA AACCCAAGGT ACCTTTGGGG CTGGCCTTCT CAAGGAAGCC
CAGCTTCATG TGATTGAGAA TACAGTGTGC AATCACTACG AGTTTCTGAA TGGAAGAGTC
AAATCCACCG AGCTCTGTGC TGGGCATTTG GCCGGAGGCA CTGACAGATG CCAGGGTGAC
AGTGGAGGGC CTGTGGTTTG CTTCGACAAG GACAAATACA TTTTACGAGG AATAACTTCT
TGGGGTCCTG GCTGTGCATG CCCCAATAAG CCTGGTGTCT ATGTTCGTGT TTCAAGCTTT
GTCACTTGGA TTGAGGGAGT GATGAGAAAT AATTAATTGA ACAAGAGACA GAGTGAAGCA
TTGACTCACC TAGAGGCTAG AATGGGGGTA GGGATTTAGC ACGCTGGAAA TAACGGACAG
TAATCAAACG AAGACACTGT CCCCAGCTAC CAACTATGCC AAACCTCAGC ATTTTTGGTA
TTATTGTGTA TAAGCTTTTC CCGTCTGACT GCTGGGTTCT CCAATAAGGT GACATAGCTA
TGCCATTTGT TAAAAATAAA CTCTGTACTT ATTTTGATTT GAGTAAA
Rhesus monkey: XM_028847001.1 (SEQ ID NO: 3)
AGCCTTGCCT TTGAAATGTT CCAGTTGGAA CATTGCTGGG CAGCGTGCAA ACAGGAGCGA
ACGTCAGCCG GGGCGGCAGG GGGCAGCAGA CCCCACACTT TGTCCATGCC TCAGGTGGGA
GGAAGTGTCC GGCTCCAGAA ACCTGCCGCG GGCTTTATCC CAAGCGACTT CAGCCAGCAG
ACGGTTCATG TCCTGAGGCT GCAAAATACG AGTTCTGCCA TCATCGCTGA CAAAGTCATC
CCAGTTTGTC TGCCATCCCC AAATTATGCG ATCGCCAACC AGACTGAATG TTATGTCACT
GGCTGGGGAG AAACCCAAGC ACTACCTGAG CAAAGCCATG TGGTCCAGGA TTGCTACCAT
GGTGATGGAC AGAGTTATCA AGGCACATCC TCCACCACTG TCACAGGAAG GACCTGCCAA
GCTTGGTCAT CTATGGAACC ACATCAGCAT AATAGAACCA CAGAAAACTA CCCAAATGCT
GGCTTGATCA GGAACTACTG CAGGAATCCA GATCCTGTGG CAGCCCCTTA TTGTTATACG
ATGGATCCCA ATGTCAGGTG GGAGTACTGC AACCTGACAC AATGCTCAGA CGCAGAAGGG
ACTGCCGTCG CACCTCCGAA TGTCACCCCG GTTCCAAGCC TAGAGGCTCT TTCCGAACAA
GCACCGACTG AGCAAAGGCC TGGGGTGCAG GAGTGCTACC ACGGTAATGG ACAGAGTTAT
CGAGGCACAT ACTTCACCAC TGTGACAGGA AGAACCTGCC AAGCTTGGTC ATCTATGACA
CCACATTCTC ATAGTCGTAC CCCAGAAAAC TACCCAAATG GTGGCTTGAT CAGGAACTAC
TGCAGGAATC CAGATCCTGT GGCAGCCCCT TATTGTTATA CCATGGATCC CAATGTCAGG
TGGGAGTACT GCAACCTGAC ACAATGCTCA GACGCAGAAG GGACTGCCGT CGCACCTCCG
AATGTCACCC CGGTTCCAAG CCTAGAGGCT CCTTCCGAAC AAGCACCGAC TGAGCAAAGG
CCTGGGGTGC AGGAGTGCTA CCACGGTAAT GGACAGAGTT ATCGAGGCAC ATACTTCACC
ACTGTGACAG GAAGAACCTG CCAAGCTTGG TCATCTATGA CACCACATTC TCATAGTCGT
ACCCCAGAAA ACTACCCAAA TGGTGGCTTG ATCAGGAACT ACTGCAGGAA TCCAGATCCT
GTGGCAGCCC CTTATTGTTA TACCATGGAT CCCAATGTCA GGTGGGAGTA CTGCAACCTG
ACACAATGCT CAGACGCAGA GGGGACTGCC GTCGCACCTC CGACTGTCAC CCCGGTTCCA
AGCCTAGAGG CTCCTTCTGA GCAAGCACCG ACTGAGCAAA GGCCTGGGGT GCAGGAGTGC
TACCACGGTA ATGGACAGAG TTATCGAGGC ACATACTTCA CCACTGTGAC AGGAAGAACC
TGCCAAGCTT GGTCATCTAT GACACCGCAC TCTCATAGTC GGACCCCGGA AAACTACCCA
AATGGTGGCT TGATCAGGAA CTACTGCAGG AATCCAGATC CTGTGGCAGC CCCTTATTGT
TATACGATGG ATCCCAATGT CAGGTGGGAG TACTGCAACC TGACACAATG CTCAGACGCA
GAAGGGACTG CCGTCGCACC TCCGAATGTC ACCCCGGTTC CAAGCCTAGA GGCTCCTTCC
GAACAAGCAC CGACTGAGCA AAGGCCTGGG GTGCAGGAGT GCTACCACGG TAATGGACAG
AGTTATCGAG GCACATACTT CACCACTGTG ACAGGAAGAA CCTGCCAAGC TTGGTCATCT
ATGACACCGC ACTCTCATAG TCGGACCCCG GAAAACTACC CAAATGGTGG CTTGATCAGG
AACTACTGCA GGAATCCAGA TCCTGTGGCA GCCCCTTATT GTTATACGAT GGATCCCAAT
GTCAGGTGGG AGTACTGCAA CCTGACACAA TGCTCAGACG CAGAAGGGAC TGCCGTCGCA
CCTCCGAATG TCACCCCGGT TCCAAGCCTA GAGGCTCCTT CCGAACAAGC ACCAACTGAG
CAAAGGCCTG GGNTGCAGGA GTGCTACCAT GGTAATGGAC AGAGTTATCG AGGCACATAC
TTCACCACTG TGACAGGAAG AACCTGCCAA GCTTGGTCAT CTATGACACC GCACTCTCAT
AGTCGGACCC CGGAAAACTA CCCAAATGGT GGCTTGATCA GGAACTACTG CAGGAATCCA
GATCCTGTGG CAGCCCCTTA TTGTTATACC ATGGATCCCA ATGTCAGGTG GNAGTACTGC
AACCTGACAC AATGCTCAGA CGCAGAAGGG ACTGCCGTCG CACCTCCGAC TGTCACCCCG
GTTCCAAGCC TAGAGGCTCC TTCGAGCAAG GCACCGACTG AGCAAAGGCC TGGGNTGCAG
GAGTGCTACC ACGGTAATGG ACAGAGTTAT CGAGGCACAT ACTTCACCAC TGTGACAGGA
AGAACCTGCC AAGCTTGGTC ATCTATGACA CCGCACTCTC ATAGTCGGAC CCCGGAAAAC
TACCCAAATG GTGGCTTGAT CAGGAACTAC TGCAGGAATC CAGATCCTGT GGCAGCCCCT
TATTGTTATA CGATGGATCC CAATGTCAGG TGGGAGTACT GCAACCTGAC ACAATGCTCA
GACGCAGAAG GGACTGCCGT CGCACCTCCG AATGTCACCC CGGTTCCAAG CCTAGAGGCT
CCTTCCGAAC AAGCACCGAC TGAGCAAAGG CCTGGGGTGC AGGAGTGCTA CCACGGTAAT
GGACAGAGTT ATCGAGGCAC ATACTTCACC ACTGTGACAG GAAGAACCTG CCAAGCTTGG
TCATCTATGA CACCGCACTC TCATAGTCGG ACCCCGGAAA ACTACCCAAA TGGTGGCTTG
ATCAGGAACT ACTGCAGGAA TCCAGATCCT GTGGCAGCCC CTTATTGTTA TACCATGGAT
CCCAATGTCA GGTGGGAGTA CTGCAACCTG ACACAATGCT CAGACGCAGA AGGGACTGCC
GTCGCACCTC CGAATGTCAC CCCGGTTCCA AGCCTAGAGG CTCCTTCTGA GCAAGCACCA
ACTGAGCAAA GGCTTGGGGT GCAGGAGTGC TACCACAGTA ATGGACAGAG TTATCGAGGC
ACATACTTCA CCACTGTGAC AGGAAGAACC TGCCAAGCTT GGTCATCTAT GACACCACAC
TCTCATAGTC GGACCCCAGA AAACTACCCA AATGCTGGCT TGGTCAAGAA CTACTGCCGA
AATCCAGATC CTGTGGCAGC CCCTTGGTGT TATACAACGG ATCCCAGTGT CAGGTGGGAG
TACTGCAACC TGACACGATG CTCAGATGCA GAAGGGACTG CTGTCATGCC TCCAAATATT
ATTCCGGTTC CAAGCCTAGA GGCTTTTCTT GAACAAGAAC CTACTGAGGA AACCCCCGGG
GTACAGGAGT GCTACTACCA TTATGGACAG AGTTATCGAG GCACATACTC CACCACTGTT
ACAGGAAGAA CTTGCCAAGC TTGGTCATCT ATGACACCAC ACCAGCATAG TCGGACCCCA
AAAAACTATC CAAATGCTGG CCTGACCAGG AACTACTGCA GGAATCCAGA TGCTGAGATT
CGCCCTTGGT GTTATACCAT GGATCCCAGT GTCAGGTGGG AGTACTGCAA CCTGACACAA
TGTCTGGTGA CAGAATCAAG TGTCCTTGAA ACTCTCACAG TGGTCCCAGA TCCAAGCACA
CAGGCTTCTT CTGAAGAAGC ACCAACGGAG CAAAGTCCCG AGGTCCAGGA CTGCTACCAT
GGTGATGGAC AGAGTTATCG AGGCTCATTC TCCACCACTG TCACAGGAAG GACATGTCAG
TCTTGGTCCT CTATGACACC ACACTGGCAT CAGAGGACAA CAGAATATTA TCCAGATGGT
GGCCTGACCA GGAACTACTG CAGGAATCCA GATGCTGAGA TTCGCCCTTG GTGTTATACC
ATGGATCCCA GTGTCAGGTG GGAGTACTGC AACCTGACAC AATGTCCAGT GACAGAATCA
AGTGTCCTCG CAACGTCCAT GGCTGTTTCT GAACAAGCAC CAATGGAGCA AAGCCCCGGG
GTCCAGGACT GCTACCATGG TGATGGACAG AGTTATCGAG GTTCATTCTC CACCACTGTC
ACAGGAAGGA CATGTCAGTC TTGGTCCTCT ATGACACCAC ACTGGCATCA GAGGACCATA
GAATACTACC CAAATGGTGG CCTGACCAAG AACTACTGCA GGAATCCAGA TGCTGAGATT
CGCCCTTGGT GTTATACCAT GGATCCCAGA GTCAGATGGG AGTACTGCAA CCTGACACAA
TGTGTGGTGA TGGAATCAAG TGTCCTTGCA ACTCCCATGG TGGTCCCAGT TCCAAGCAGA
GAGGTTCCTT CTGAAGAAGC ACCAACTGAA AACAGCCCTG GGGTCCAGGA CTGCTACCAA
GGTGATGGAC AGAGTTATCG AGGCACATTC TCCACCACTA TCACAGGAAG AACATGTCAG
TCTTGGTTGT CTATGACACC ACATCGGCAT CGGAGGATCC CATTACGCTA TCCAAATGCT
GGCCTGACCA GGAACTATTG CAGAAATCCA GATGCTGAGA TTCGCCCTTG GTGTTACACC
ATGGATCCCA GTGTCAGGTG GGAGTACTGC AACCTGACAC AATGTCCAGT GACAGAATCA
AGTGTCCTCA CAACTCCCAC GGTGGTCCCG GTTCCAAGCA CAGAGGCTCC TTCTGAACAA
GCACCACCTG AGAAAAGCCC TGTGGTCCAG GATTGCTACC ATGGTGATGG ACAGAGTTAT
CGAGGCACAT CCTCCACCAC TGTCACAGGA AGGAACTGTC AATCTTGGTC ATCTATGATA
CCACACTGGC ATCAGAGGAC CCCAGAAAAC TACCCAAATG CTGGCCTGAC CAGGAACTAC
TGCAGGAATC CAGATTCTGG GAAACAACCC TGGTGTTACA CGACTGATCC ATGTGTGAGG
TGGGAGTACT GCAACCTGAC ACAATGCTCA GAAACAGAAT CAGGTGTCCT AGAGACTCCC
ACTGTTGTTC CGGTTCCAAG CATGGAAGCT CATTCTGAAG CAGCACCAAC TGAGCAAACC
CCTGTGGTCC AGCAGTGCTA CCATGGTAAT GGACAGAGTT ATCGAGGCAC ATTCTCCACC
ACTGTCACAG GAAGGACATG TCAATCTTGG TCATCCATGA CACCACACCA GCATAAGAGG
ACCCCGGAAA ACCACCCAAA TGATGACTTG ACAATGAACT ACTGCAGGAA TCCAGATGCT
GACACAGGCC CTTGGTGTTT TACCATGGAC CCCAGCGTCA GGCGGGAGTA CTGCAACCTG
ACGCGATGCT CAGACACAGA AGGGACTGTG GTCACACCTC CGACTGTTAT CCCGGTTCCA
AGCCTAGAGG CTCCTTCTGA ACAAGCATCC TCTTCATTTG ATTGTGGGAA GCCTCAAGTG
GAGCCAAAGA AATGTCCTGG AAGCATTGTA GGTGGGTGTG TGGCCCACCC ACATTCCTGG
CCCTGGCAAG TCAGTCTTAG AACAAGGTTT GGAAAGCACT TCTGTGGAGG CACCTTAATA
TCCCCAGAGT GGGTGCTGAC TGCTGCTTGC TGCTTGGAGA CGTTCTCAAG GCCTTCCTTC
TACAAGGTCA TCCTGGGTGC ACACCAAGAA GTGAATCTCG AATCTCATGT TCAAGAAATA
GAAGTGTCTA GGTTGTTCTT GGAGCCCATA GGAGCAGATA TTGCCTTGCT AAAGCTAAGC
AGGCCTGCCA TCATCACTGA CAAAGTAATC CCAGCCTGTC TGCCGTCTCC AAATTACGTG
ATCACCGCCT GGACTGAATG TTACATCACT GGCTGGGGAG AAACCCAAGG TACCTTTGGG
GCTGGCCTTC TCAAGGAAGC CCAGCTTCAT GTGATTGAGA ATACAGTGTG CAATCACTAC
GAGTTTCTGA ATGGAAGAGT CAAATCCACT GAGCTCTGTG CTGGGCATTT GGCCGGAGGC
ACTGACAGAT GCCAGGGTGA CAATGGAGGG CCTGTGGTTT GCTTCGACAA GGACAAATAC
ATTTTACGAG GAATAACTTC TTGGGGTCCT GGCTGTGCAT GCCCCAATAA GCCTGGTGTC
TATGTTCGTG TTTCAAGCTT TGTCACTTGG ATTGAGGGAG TGATGAGAAA TAATTAATTG
AACAAGAGAC AGAGTGAAGC ATTGACTCAC CTAGAGGCTA GAATGGGGGT AGGGATTTAG
CACGCTGGAA ATAACGGACA GTAATCAAAC GAAGACACTG TCCCCAGCTA CCAACTATGC
CAAACCTCAG CATTTTTGGT ATTATTGTGT ATAAGCTTTT CCTGTCTGAC TGCTGGGTTC
TCCAATAAGG TGACATAGCT ATGCCATTTG TTAAAAATAA ACTCTGTACT TATTTTGATT
TGAGTAAA

TABLE 6
LPA Oligonucleotide Sequences (modified)

| | | SEQ | | SEQ |
|---|---|---|---|---|
| | Sequence | ID | Sequence | ID |
| Oligonucleotide | (Sense Strand) | NO: | (Antisense Strand) | NO: |
| LPA-0190-M1 | [mUs][mC][mC][mA][mC] | 388 | [Me Phosphonate-4O- | 788 |
| | [mC][mA][fC][fU][fG] | | mUs][fUs][fUs][fC][fC] | |
| | [fU][mC][mA][mC][mA] | | [mU][fG][mU][mG][fA][mC] | |
| | [mG][mG][mA][mA][mA] | | [mA][mG][fU][mG][mG][mU] | |
| | [mG][mC][mA][mG][mC][mC] | | [mG][mG][mAs][mGs][mG] | |
| | [mG][ademA- | | | |
| | GalNAc][ademA- | | | |
| | GalNAc][ademA- | | | |
| | GalNAc][mG][mG][mC] | | | |
| | [mU][mG][mC] | | | |
| LPA-0501-M1 | [mUs][mG][mG][mU][mA] | 389 | [Me Phosphonate-4O- | 789 |
| | [mA][mU][fG][fG][fA] | | mUs][fAs][fUs][fA][fA] | |
| | [fC][mA][mG][mA][mG][mU] | | [mC][fU][mC][mU][fG][mU] | |
| | [mU][mA][mU][mA][mG] | | [mC][mC][fA][mU][mU][mA] | |
| | [mC][mA][mG][mC][mC] | | [mC][mC][mAs][mGs][mG] | |
| | [mG][ademA- | | | |
| | GalNAc][ademA- | | | |
| | GalNAc][ademA- | | | |
| | GalNAc][mG][mG][mC] | | | |
| | [mU][mG][mC] | | | |
| LPA-3100-M1 | [mUs][mA][mC][mU][mG] | 390 | [Me Phosphonate-4O- | 790 |
| | [mC][mA][fA][fC][fC] | | mUs][fAs][fUs][fC][fG] | |
| | [fU][mG][mA][mC][mA][mC] | | [mU][fG][mU][mC][fA][mG] | |
| | [mG][mA][mU][mA][mG] | | [mG][mU][fU][mG][mC][mA] | |
| | [mC][mA][mG][mC][mC] | | [mG][mU][mAs][mGs][mG] | |
| | [mG][ademA- | | | |
| | GalNAc][ademA- | | | |
| | GalNAc][ademA- | | | |
| | GalNAc][mG][mG][mC][mU] | | | |
| | [mG][mC] | | | |
| LPA-3286-M1 | [mAs][mG][mA][mA][mC] | 391 | [Me Phosphonate-4O- | 791 |
| | [mU][mU][fG][fC][fC] | | mUs][fAs][fCs][fC][fA] | |
| | [fA][mA][mG][mC][mU][mU] | | [mA][fG][mC][mU][fU][mG] | |
| | [mG][mG][mU][mA][mG] | | [mG][mC][fA][mA][mG][mU] | |
| | [mC][mA][mG][mC][mC] | | [mU][mC][mUs][mGs][mG] | |
| | [mG][ademA- | | | |
| | GalNAc][ademA- | | | |
| | GalNAc][ademA- | | | |
| | GalNAc][mG][mG][mC][mU] | | | |
| | [mG][mC] | | | |
| LPA-3288-M1 | [mAs][mA][mC][mU][mU] | 392 | [Me Phosphonate-4O- | 792 |
| | [mG][mC][fC][fA][fA] | | mUs][fUs][fGs][fA][fC] | |
| | [fG][mC][mU][mU][mG][mG] | | [mC][FA][mA][mG][fC][mU] | |
| | [mU][mC][mA][mA][mG] | | [mU][mG][fG][mC][mA][mA] | |
| | [mC][mA][mG][mC][mC] | | [mG][mU][mUs][mGs][mG] | |
| | [mG][ademA- | | | |
| | GalNAc][ademA- | | | |
| | GalNAc][ademA- | | | |
| | GalNAc][mG][mG][mC][mU] | | | |
| | [mG][mC] | | | |
| LPA-3291-M1 | [mUs][mU][mG][mC][mC] | 393 | [Me Phosphonate-4O- | 793 |
| | [mA][mA][fG][fC][fU] | | mUs][fAs][fGs][fA][fU] | |
| | [fU][mG][mG][mU][mC][mA] | | [mG][fA][mC][mC][fA][mA] | |
| | [mU][mC][mU][mA][mG] | | [mG][mC][fU][mU][mG][mG] | |
| | [mC][mA][mG][mC][mC] | | [mC][mA][mAs][mGs][mG] | |
| | [mG][ademA- | | | |
| | GalNAc][ademA- | | | |
| | GalNAc][ademA- | | | |
| | GalNAc][mG][mG][mC][mU] | | | |
| | [mG][mC] | | | |
| LPA-3584-M1 | [mAs][mU][mG][mG][mA] | 394 | [Me Phosphonate-4O- | 794 |
| | [mC][mA][fG][fA][fG] | | mUs][fCs][fCs][fU][fC] | |
| | [fU][mU][mA][mU][mC][mG] | | [mG][fA][mU][mA][fA][mC] | |
| | [mA][mG][mG][mA][mG] | | [mU][mC][fU][mG][mU][mC] | |
| | [mC][mA][mG][mC][mC] | | [mC][mA][mUs][mGs][mG] | |
| | [mG][ademA- | | | |
| | GalNAc][ademA- | | | |
| | GalNAc][ademA- | | | |
| | GalNAc][mG][mG][mC][mU] | | | |
| | [mG][mC] | | | |
| LPA-3585-M1 | [mUs][mG][mG][mA][mC] | 395 | [Me Phosphonate-4O- | 795 |
| | [mA][mG][fA][fG][fU] | | mUs][fGs][fCs][fC][fU] | |
| | [fU][mA][mU][mC][mG][mA] | | [mC][fG][mA][mU][fA][mA] | |
| | [mG][mG][mC][mA][mG] | | [mC][mU][fC][mU][mG][mU] | |
| | [mC][mA][mG][mC][mC] | | [mC][mC][mAs][mGs][mG] | |
| | [mG][ademA- | | | |
| | GalNAc][ademA- | | | |
| | GalNAc][ademA- | | | |
| | GalNAc][mG][mG][mC][mU] | | | |
| | [mG][mC] | | | |
| LPA-4645-M1 | [mUs][mG][mG][mU][mC] | 396 | [Me Phosphonate-4O- | 796 |
| | [mA][mU][fC][fU][fA] | | mUs][fGs][fUs][fG][fG] | |
| | [fU][mG][mA][mU][mA][mC] | | [mU][fA][mU][mC][fA][mU] | |
| | [mC][mA][mC][mA][mG] | | [mA][mG][fA][mU][mG][mA] | |
| | [mC][mA][mG][mC][mC] | | [mC][mC][mAs][mGs][mG] | |
| | [mG][ademA- | | | |
| | GalNAc][ademA- | | | |
| | GalNAc][ademA- | | | |
| | GalNAc][mG][mG][mC][mU] | | | |
| | [mG][mC] | | | |
| LPA-4717-M1 | [mUs][mA][mC][mU][mG] | 397 | [Me Phosphonate-4O- | 797 |
| | [mC][mA][fG][fG][fA] | | mUs][fAs][fAs][fU][fC] | |
| | [fA][mU][mC][mC][mA][mG] | | [mU][fG][mG][mA][fU][mU] | |
| | [mA][mU][mU][mA][mG] | | [mC][mC][fU][mG][mC][mA] | |
| | [mC][mA][mG][mC][mC] | | [mG][mU][mAs][mGs][mG] | |
| | [mG][ademA- | | | |
| | GalNAc][ademA- | | | |
| | GalNAc][ademA- | | | |
| | GalNAc][mG][mG][mC][mU] | | | |
| | [mG][mC] | | | |
| LPA-5510-M1 | [mAs][mG][mA][mA][mA] | 398 | [Me Phosphonate-4O- | 798 |
| | [mU][mG][fU][fC][fC] | | mUs][fAs][fUs][fG][fC] | |
| | [fU][mG][mG][mA][mA][mG] | | [mU][fU][mC][mC][fA][mG] | |
| | [mC][mA][mU][mA][mG] | | [mG][mA][fC][mA][mU][mU] | |
| | [mC][mA][mG][mC][mC] | | [mU][mC][mUs][mGs][mG] | |
| | [mG][ademA- | | | |
| | GalNAc][ademA- | | | |
| | GalNAc][ademA- | | | |
| | GalNAc][mG][mG][mC][mU] | | | |
| | [mG][mC] | | | |
| LPA-3750-M1 | [mGs][mA][mC][mA][mA] | 399 | [Me Phosphonate-4O- | 799 |
| | [mC][mA][fG][fA][fA] | | mUs][fUs][fGs][fG][fA] | |
| | [fU][mA][mU][mU][mA][mU] | | [mU][fA][mA][mU][fA][mU] | |
| | [mC][mC][mA][mA][mG] | | [mU][mC][fU][mG][mU][mU] | |
| | [mC][mA][mG][mC][mC] | | [mG][mU][mCs][mGs][mG] | |
| | [mG][ademA- | | | |
| | GalNAc][ademA- | | | |
| | GalNAc][ademA- | | | |
| | GalNAc][mG][mG][mC][mU] | | | |
| | [mG][mC] | | | |
| LPA-2900-M2 | [mAs][mU][mG][mG][mA] | 400 | [Me Phosphonate-4O- | 800 |
| | [mC][mA][fG][fA][fG] | | mUs][fCs][fC][fU][fU][mG] | |
| | [fU][mU][mA][mU][mC][mA] | | [fA][mU][mA][fA][mC][mU] | |
| | [mA][mG][mG][mA][mG] | | [mC][fU][mG][mU][mC][mC] | |
| | [mC][mA][mG][mC][mC] | | [mA][mUs][mGs][mG] | |
| | [mG][ademA- | | | |
| | GalNAc][ademA- | | | |
| | GalNAc][ademA- | | | |
| | GalNAc][mG][mG][mC][mU] | | | |
| | [mG][mC] | | | |
| LPA-3675-M2 | [mGs][mA][mC][mA][mA] | 401 | [Me Phosphonate-4O- | 801 |
| | [mC][mA][fG][fA][fA] | | mUs][fUs][fG][fG][fA][mU] | |
| | [fU][mA][mU][mU][mA][mU] | | [fA][mA][mU][fA][mU][mU] | |
| | [mC][mC][mA][mA][mG] | | [mC][fU][mG][mU][mU][mG] | |
| | [mC][mA][mG][mC][mC] | | [mU][mCs][mGs][mG] | |
| | [mG][ademA- | | | |
| | GalNAc][ademA- | | | |
| | GalNAc][ademA- | | | |
| | GalNAc][mG][mG][mC][mU] | | | |
| | [mG][mC] | | | |
| LPA-2900-M3 | [mAs][mU][mG][mG][mA] | 402 | [Me Phosphonate-4O- | 802 |
| | [mC][mA][fG][fA][fG] | | mUs][fCs][fC][mU][fU][mG] | |
| | [fU][mU][mA][mU][mC][mA] | | [fA][mU][mA][fA][mC][mU] | |
| | [mA][mG][mG][mA][mG] | | [mC][fU][mG][mU][mC][mC] | |
| | [mC][mA][mG][mC][mC] | | [mA][mUs][mGs][mG] | |
| | [mG][ademA- | | | |
| | GalNAc][ademA- | | | |
| | GalNAc][ademA- | | | |
| | GalNAc][mG][mG][mC][mU] | | | |
| | [mG][mC] | | | |
| LPA-3675-M3 | [mGs][mA][mC][mA][mA] | 403 | [Me Phosphonate-4O- | 803 |
| | [mC][mA][fG][fA][fA] | | mUs][fUs][fG][mG][fA][mU] | |
| | [fU][mA][mU][mU][mA][mU] | | [fA][mA][mU][fA][mU][mU] | |
| | [mC][mC][mA][mA][mG] | | [mC][fU][mG][mU][mU][mG] | |
| | [mC][mA][mG][mC][mC] | | [mU][mCs][mGs][mG] | |
| | [mG][ademA- | | | |
| | GalNAc][ademA- | | | |
| | GalNAc][ademA- | | | |
| | GalNAc][mG][mG][mC][mU] | | | |
| | [mG][mC] | | | |

### Modifications in Table 6:

mC, mA, mG, mU=2′-OMe ribonucleosides; fA, fC, fG, fU=2′-F ribonucleosides; s=phosphorothioate; MePhosphonate-40-mUs=

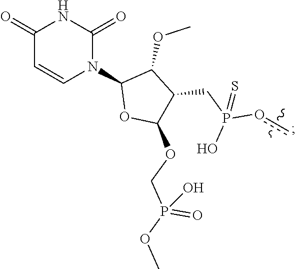

ademA-GalNAc=GalNAc attached to an adenine nucleotide:

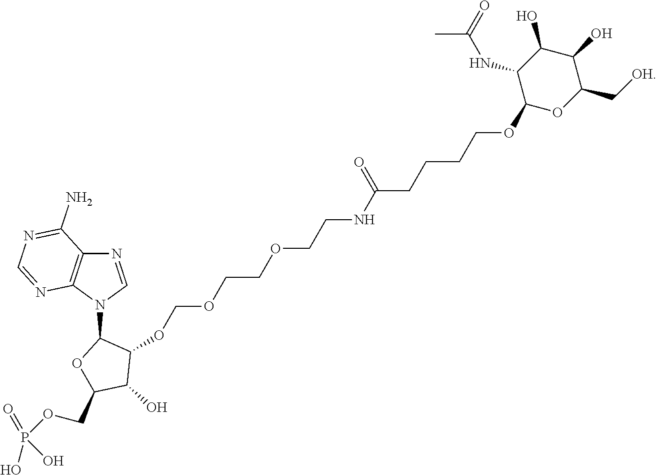 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O]

1. An RNAi oligonucleotide for reducing LPA expression, the oligonucleotide comprising a sense strand and an antisense strand, wherein the sense strand and the antisense strand form a duplex region, wherein the antisense strand comprises a region of complementarity to a LPA mRNA target sequence of any one of SEQ ID NOs: 4-387, and wherein the region of complementarity is at least 15 contiguous nucleotides in length.

2. The RNAi oligonucleotide of claim 1, wherein the sense strand is 15 to 50 nucleotides in length.

3. (canceled)

4. The RNAi oligonucleotide of claim 1, wherein the antisense strand is 15 to 30 nucleotides in length, optionally wherein the antisense strand is 22 nucleotides in length and wherein antisense strand and the sense strand form a duplex region of at least 19 nucleotides in length, optionally at least 20 nucleotides in length.

5.-6. (canceled)

7. The RNAi oligonucleotide of claim 1, wherein the 3′ end of the sense strand comprises a stem-loop set forth as S1-L-S2, wherein S1 is complementary to S2, and wherein L forms a loop between S1 and S2 of 3-5 nucleotides in length.

8.-14. (canceled)

15. The RNAi oligonucleotide of claim 1 wherein the sense strand is of 36 nucleotides in length and the antisense strand is of 22 nucleotides in length, wherein the sense strand and the antisense strand form a duplex region of at least 19 nucleotides in length, optionally 20 nucleotides in length, wherein the 3′ end of the sense strand comprises a stem-loop set forth as S1-L-S2, wherein S1 is complementary to S2, and wherein L forms a loop between S1 and S2 of 3-5 nucleotides in length, wherein the region of complementarity is 19 contiguous nucleotides in length, optionally 20 nucleotides in length.

16. The RNAi oligonucleotide of claim 15, wherein L is a triloop or a tetraloop, optionally wherein the tetraloop comprises the sequence 5′-GAAA-3′.

17.-18. (canceled)

19. The RNAi oligonucleotide of claim 15, wherein the S1 and S2 are 1-10 nucleotides in length and have the same length, optionally wherein the stem-loop comprises the sequence 5′-GCAGCCGAAAGGCUGC-3′ (SEQ ID NO: 190).

20.-22. (canceled)

23. The RNAi oligonucleotide of claim 1, wherein the antisense strand comprises a 3′ overhang sequence of one or more nucleotides in length, optionally wherein the 3′ overhang sequence is 2 nucleotides in length, optionally wherein the 3′ overhang sequence is GG.

24. (canceled)

25. The RNAi oligonucleotide of claim 1, wherein the oligonucleotide comprises at least one modified nucleotide.

26. The RNAi oligonucleotide of claim 25, wherein the modified nucleotide comprises a 2′-modification, optionally wherein the 2′-modification is a modification selected from 2′-aminoethyl, 2′-fluoro, 2′-O-methyl, 2′-O-methoxyethyl, and 2′-deoxy-2′-fluoro-β-d-arabinonucleic acid.

27. (canceled)

28. The RNAi oligonucleotide of claim 1, wherein all nucleotides comprising the oligonucleotide are modified, optionally wherein the modification is a 2′-modification selected from 2′-fluoro and 2′-O-methyl.

29. The RNAi oligonucleotide of claim 1, wherein the oligonucleotide comprises at least one modified internucleotide linkage, optionally wherein the at least one modified internucleotide linkage is a phosphorothioate linkage.

30. (canceled)

31. The RNAi oligonucleotide of claim 1, wherein the 4′-carbon of the sugar of the 5′-nucleotide of the antisense strand comprises a phosphate analog, optionally wherein the phosphate analog is oxymethylphosphonate, vinylphosphonate or malonyl phosphonate, optionally wherein the phosphate analog is a 4′-phosphate analog comprising 5′-methoxyphosphonate-4′-oxy.

32. (canceled)

33. The RNAi oligonucleotide of claim 1, wherein at least one nucleotide of the oligonucleotide is conjugated to one or more targeting ligands.

34. (canceled)

35. The RNAi oligonucleotide of claim 33, wherein each targeting ligand comprises a N-acetylgalactosamine (GalNAc) moiety, optionally wherein the GalNAc moiety is a monovalent GalNAc moiety, a bivalent GalNAc moiety, a trivalent GalNAc moiety or a tetravalent GalNAc moiety.

36. (canceled)

37. The RNAi oligonucleotide of claim 15, wherein up to 4 nucleotides of L of the stem-loop are each conjugated to a monovalent GalNAc moiety.

38. The RNAi oligonucleotide of claim 1, wherein the sense strand comprises a nucleotide sequence of any one of SEQ ID NOs: 388, 389, 390, 391, 392, 393, 394, 395, 396, 397, 398, 399, 400, 401, 402, and 403.

39. The RNAi oligonucleotide of claim 1, wherein the antisense strand comprises a nucleotide sequence of any one of SEQ ID NOs: 788, 789, 790, 791, 792, 793, 794, 795, 796, 797, 798, 799, 800, 801, 802, and 803.

40. The RNAi oligonucleotide of claim 1, wherein the sense strand and antisense strands comprise nucleotide sequences selected from the group consisting of:
(a) SEQ ID NOs: 393 and 793, respectively;
(b) SEQ ID NOs: 388 and 788, respectively;
(c) SEQ ID NOs: 389 and 789, respectively;
(d) SEQ ID NOs: 390 and 790, respectively;
(e) SEQ ID NOs: 391 and 791, respectively;
(f) SEQ ID NOs: 392 and 792, respectively;
(g) SEQ ID NOs: 394 and 794, respectively;
(h) SEQ ID NOs: 395 and 795, respectively;
(i) SEQ ID NOs: 396 and 796, respectively;
(j) SEQ ID NOs: 397 and 797, respectively;
(k) SEQ ID NOs: 398 and 798, respectively;
(l) SEQ ID NOs: 399 and 799, respectively;
(m) SEQ ID NOs: 400 and 800, respectively;
(n) SEQ ID NOs: 401 and 801, respectively;
(o) SEQ ID NOs: 402 and 802, respectively; and
(p) SEQ ID NOs: 403 and 803, respectively.

41.-60. (canceled)

61. A method for treating a subject having a disease, disorder or condition associated with LPA expression, the method comprising administering to the subject a therapeutically effective amount of the RNAi oligonucleotide of claim 1, thereby treating the subject.

62. A pharmaceutical composition comprising the RNAi oligonucleotide of claim 1, and a pharmaceutically acceptable carrier, delivery agent or excipient.

63. (canceled)

64. A method for reducing LPA expression in a cell, a population of cells or a subject, the method comprising the step of:
i. contacting the cell or the population of cells with the RNAi oligonucleotide of claim 1; or
ii. administering to the subject the RNAi oligonucleotide of claim 1.

65.-92. (canceled)


SEQUENCE LISTING (1197 sequences)

SEQ ID NO: 1 - DNA, 6414 bp, Homo sapiens
ctgggattgggacacactttctgggcactgctggccagtcccaaaatggaacataaggaagtggttcttctacttcttttatttctgaaatcagcagcacctgagcaaagccatgtggtccaggattgctaccatggtgatggacagagttatcgaggcacgtactccaccactgtcacaggaaggacctgccaagcttggtcatctatgacaccacatcaacataataggaccacagaaaactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcagaggcctggggtgcaggagtgctaccacggtaatggacagagttatcgaggcacatactccaccactgtcactggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcagaggcctggggtgcaggagtgctaccacggtaatggacagagttatcgaggcacatactccaccactgtcactggaagaacctgccaagcttggtcatctatgacaccacactcgcatagtcggaccccagaatactacccaaatgctggcttgatcatgaactactgcaggaatccagatcctgtggcagccccttattgttatacgagggatcccagtgtcaggtgggagtactgcaacctgacacaatgctcagacgcagaagggactgccgtcgcgcctccaactattaccccgattccaagcctagaggctccttctgaacaagcaccaactgagcaaaggcctggggtgcaggagtgctaccacggaaatggacagagttatcaaggcacatacttcattactgtcacaggaagaacctgccaagcttggtcatctatgacaccacactcgcatagtcggaccccagcatactacccaaatgctggcttgatcaagaactactgccgaaatccagatcctgtggcagccccttggtgttatacaacagatcccagtgtcaggtgggagtactgcaacctgacacgatgctcagatgcagaatggactgccttcgtccctccgaatgttattctggctccaagcctagaggctttttttgaacaagcactgactgaggaaacccccggggtacaggactgctactaccattatggacagagttaccgaggcacatactccaccactgtcacaggaagaacttgccaagcttggtcatctatgacaccacaccagcatagtcggaccccagaaaactacccaaatgctggcctgaccaggaactactgcaggaatccagatgctgagattcgcccttggtgttacaccatggatcccagtgtcaggtgggagtactgcaacctgacacaatgcctggtgacagaatcaagtgtccttgcaactctcacggtggtcccagatccaagcacagaggcttcttctgaagaagcaccaacggagcaaagccccggggtccaggattgctaccatggtgatggacagagttatcgaggctcattctctaccactgtcacaggaaggacatgtcagtcttggtcctctatgacaccacactggcatcagaggacaacagaatattatccaaatggtggcctgaccaggaactactgcaggaatccagatgctgagattagtccttggtgttataccatggatcccaatgtcagatgggagtactgcaacctgacacaatgtccagtgacagaatcaagtgtccttgcgacgtccacggctgtttctgaacaagcaccaacggagcaaagccccacagtccaggactgctaccatggtgatggacagagttatcgaggctcattctccaccactgttacaggaaggacatgtcagtcttggtcctctatgacaccacactggcatcagagaaccacagaatactacccaaatggtggcctgaccaggaactactgcaggaatccagatgctgagattcgcccttggtgttataccatggatcccagtgtcagatgggagtactgcaacctgacgcaatgtccagtgatggaatcaactctcctcacaactcccacggtggtcccagttccaagcacagagcttccttctgaagaagcaccaactgaaaacagcactggggtccaggactgctaccgaggtgatggacagagttatcgaggcacactctccaccactatcacaggaagaacatgtcagtcttggtcgtctatgacaccacattggcatcggaggatcccattatactatccaaatgctggcctgaccaggaactactgcaggaatccagatgctgagattcgcccttggtgttacaccatggatcccagtgtcaggtgggagtactgcaacctgacacgatgtccagtgacagaatcgagtgtcctcacaactcccacagtggccccggttccaagcacagaggctccttctgaacaagcaccacctgagaaaagccctgtggtccaggattgctaccatggtgatggacggagttatcgaggcatatcctccaccactgtcacaggaaggacctgtcaatcttggtcatctatgataccacactggcatcagaggaccccagaaaactacccaaatgctggcctgaccgagaactactgcaggaatccagattctgggaaacaaccctggtgttacacaaccgatccgtgtgtgaggtgggagtactgcaatctgacacaatgctcagaaacagaatcaggtgtcctagagactcccactgttgttccagttccaagcatggaggctcattctgaagcagcaccaactgagcaaacccctgtggtccggcagtgctaccatggtaatggccagagttatcgaggcacattctccaccactgtcacaggaaggacatgtcaatcttggtcatccatgacaccacaccggcatcagaggaccccagaaaactacccaaatgatggcctgacaatgaactactgcaggaatccagatgccgatacaggcccttggtgttttaccatggaccccagcatcaggtgggagtactgcaacctgacgcgatgctcagacacagaagggactgtggtcgctcctccgactgtcatccaggttccaagcctagggcctccttctgaacaagactgtatgtttgggaatgggaaaggataccggggcaagaaggcaaccactgttactgggacgccatgccaggaatgggctgcccaggagccccatagacacagcacgttcattccagggacaaataaatgggcaggtctggaaaaaaattactgccgtaaccctgatggtgacatcaatggtccctggtgctacacaatgaatccaagaaaactttttgactactgtgatatccctctctgtgcatcctcttcatttgattgtgggaagcctcaagtggagccgaagaaatgtcctggaagcattgtaggggggtgtgtggcccacccacattcctggccctggcaagtcagtctcagaacaaggtttggaaagcacttctgtggaggcaccttaatatccccagagtgggtgctgactgctgctcactgcttgaagaagtcctcaaggccttcatcctacaaggtcatcctgggtgcacaccaagaagtgaacctcgaatctcatgttcaggaaatagaagtgtctaggctgttcttggagcccacacaagcagatattgccttgctaaagctaagcaggcctgccgtcatcactgacaaagtaatgccagcttgtctgccatccccagactacatggtcaccgccaggactgaatgttacatcactggctggggagaaacccaaggtacctttgggactggccttctcaaggaagcccagctccttgttattgagaatgaagtgtgcaatcactataagtatatttgtgctgagcatttggccagaggcactgacagttgccagggtgacagtggagggcctctggtttgcttcgagaaggacaaatacattttacaaggagtcacttcttggggtcttggctgtgcacgccccaataagcctggtgtctatgctcgtgtttcaaggtttgttacttggattgagggaatgatgagaaataattaattggacgggagacagagtgaagcatcaacctacttagaagctgaaacgtgggtaaggatttagcatgctggaaataatagacagcaatcaaacgaagacactgttcccagctaccagctatgccaaaccttggcatttttggtatttttgtgtataagcttttaaggtctgactgacaaattctgtattaaggtgtcatagctatgacatttgttaaaaataaactctgcacttattttgatttga

SEQ ID NO: 2 - DNA, 5387 bp, Macaca fascicularis
gatgctgcatacttaatgtcgaaaggttgcttcatccaagagcctggagttttcagagacactgtcctgaaactatgtcctgaaactatgtcattgaaactgaaacattgtcctgaagctggtattgggcaataccagcgcctgcaggcaacagctcggatgcacttaagatttaaatattacccacagaagttctggcttgtctgggaaaaccttttgctaaacagaagagcaacatttttttttttttcttttctggaatttgtaaacagcatttattctcagccttaccttccaaacgttgcacttggaacattgctgggccccgtggaaacagaagcgaacgtcagccaggccggcagggggcggcagaccccacacttcgccgggcgccctcacctccctgggagggagtgtgcagctgccaaaatcttcggcggggttcagtccaagcgacttcagccagcagatggtcattctcctgtgaccgtgtgtactacagactgtttcaaaaccgggcaggcaattaacaatgggaattctgccatcatcgctgacaaagtcatcccagtttgtctgccatccccaaattatgtggtcgccaaccagactgaatgttatgtcactggctggggagaaacccaagcactacctgagcaaagccatgtggtccaggattgctaccatggtgatggacagagttatcaaggcacatcctccaccactgtcacaggaaggacctgccaagcttggtcatctatggaaccacatcagcataatagaaccacagaaaactacccaaatgctggcttgatcaggaactactgcaggaatccagatcctgtggcagccccttattgttatacgatggatcccaatgtcaggtgggagtactgcaacctgacacaatgctcagacgcagaagggactgccgtcgcacctccgaatgtcaccccggttccaagcctagaggctccttccgaacaagcaccgactgagcaaaggcctggggtgcaggagtgctaccacggtaatggacagagttatcgaggcacatacttcaccactgtgacaggaagaacctgccaagcttggtcatctatgacaccgcactctcatagtcggaccccggaaaactacccaaatggtggcttgatcaggaactactgcaggaatccagatcctgtggcagccccttattgttataccatggatcccaatgtcaggtgggagtactgcaacctgacacaatgctcagacgcagaagggattgccgtcacacctctgactgttaccccggttccaagcctagaggctccttccaagcaagcaccaactgagcaaaggcctggtgtccaggagtgctaccacggtaatggacagagttatcgaggcacatacttcaccactgtgacaggaagaacctgccaagcttggtcatctatgacaccacattctcatagtcgtaccccagaaaactacccaaatggtggcttgatcaggaactactgcaggaatccagatcctgtggcagccccttattgttataccatggatcccaatgtcaggtgggagtactgcaacctgacacaatgctcagacgcagaagggactgccgtcgcacctccgactgtcaccccggttccaagcctagaggctccttccgaacaagcaccgactgagcaaaggcctggggtgcaggagtgctaccacggtaatggacagagttatcgaggcacatacttcaccactgtgacaggaagaacctgccaagcttggtcatctatgacaccgcactctcatagtcggaccccggaaaactacccaaatggtggcttgatcaggaactactgcaggaatccagatcctgtggcagccccttattgttataccatggatcccaatgtcaggtgggagtactgcaacctgacacaatgctcagacgcagaagggactgccgtcgcacctccgaatgtcaccccggttccaagcctagaggctccttctgagcaagcaccaactgagcaaaggcttggggtgcaggagtgctaccacggtaatggacagagttatcgaggcacatacttcaccactgtgacaggaagaacctgccaagcttggtcatctatgacaccacactctcatagtcggaccccagaaaactacccaaatgctggcttggtcaagaactactgccgaaatccagatcctgtggcagccccttggtgttatacaacggatcccagtgtcaggtgggagtactgcaacctgacacgatgctcagatgcagaagggactgctgttgtgcctccaaatattattccggttccaagcctagaggcttttcttgaacaagaaccgactgaggaaacccccggggtacaggagtgctactaccattatggacagagttatagaggcacatactccaccactgttacaggaagaacttgccaagcttggtcatctatgacaccacaccagcatagtcggaccccaaaaaactatccaaatgctggcctgaccaggaactactgcaggaatccagatgctgagattcgcccttggtgttataccatggatcccagtgtcaggtgggagtactgcaacctgacacaatgtctggtgacagaatcaagtgtccttgaaactctcacagtggtcccagatccaagcacacaggcttcttctgaagaagcaccaacggagcaaagtcccgaggtccaggactgctaccatggtgatggacagagttatcgaggctcattctccaccactgtcacaggaaggacatgtcagtcttggtcctctatgacaccacactggcatcagaggacaacagaatattatccagatggtggcctgaccaggaactactgcaggaatccagatgctgagattcgcccttggtgttataccatggatcccagtgtcaggtgggagtactgcaacctgacacaatgtccagtgacagaatcaagtgtcctcgcaacgtccatggctgtttctgaacaagcaccaatggagcaaagccccggggtccaggactgctaccatggtgatggacagagttatcgaggttcattctccaccactgtcacaggaaggacatgtcagtcttggtcctctatgacaccacactggcatcagaggaccatagaatactacccaaatggtggcctgaccaagaactactgcaggaatccagatgctgagattcgcccttggtgttataccatggatcccagagtcagatgggagtactgcaacctgacacaatgtgtggtgatggaatcaagtgtccttgcaactcccatggtggtcccagttccaagcagagaggttccttctgaagaagcaccaactgaaaacagccctggggtccaggactgctaccaaggtgatggacagagttatcgaggcacattctccaccactatcacaggaagaacatgtcagtcttggttgtctatgacaccacatcggcatcggaggatcccattacgctatccaaatgctggcctgaccaggaactattgcagaaatccagatgctgagattcgcccttggtgttacaccatggatcccagtgtcaggtgggagtactgcaacctgacacaatgtccagtgacagaatcaagtgtcctcacaactcccacggtggtcccggttccaagcacagaggctccttctgaacaagcaccacctgagaaaagccctgtggtccaggattgctaccatggtgatggacagagttatcgaggcacatcctccaccactgtcacaggaaggaactgtcagtcttggtcatctatgataccacactggcatcagaggaccccagaaaactacccaaatgctggcctgaccaggaactactgcaggaatccagattctgggaaacaaccctggtgttacacgactgatccatgtgtgaggtgggagtactgcaacctgacacaatgctcagaaacagaatcaggtgtcctagagactcccactgttgttccggttccaagcatggaagctcattctgaagcagcaccaactgagcaaactcctgtggtccagcagtgctaccatggtaatggacagagttatcgaggcacattctccaccactgtcacaggaaggacatgtcaatcttggtcatccatgacaccacaccagcataagaggaccccggaaaaccacccaaatgatgacttgacaatgaactactgcaggaatccagatgctgacacaggcccttggtgttttaccatggaccccagcgtcaggcgggagtactgcaacctgacgcgatgctcagacacagaagggactgtggtcacacctccgactgttatcccggttccaagcctagaggctccttctgaacaagcatcctcttcatttgattgtgggaagcctcaagtggagccaaagaaatgtcctggaagcattgtaggtgggtgtgtggcccacccacattcctggccctggcaagtcagtcttagaacaaggtttggaaagcacttctgtggaggcaccttaatatccccagagtgggtgctgactgctgcttgctgcttggagacgttctcaaggccttccttctacaaggtcatcctgggtgcacaccaagaagtgaatctcgaatctcacgttcaagaaatagaagtgtctaggttgttcttggagcccataggagcagatattgccttgctaaagctaagcaggcctgccatcatcactgacaaagtaatcccagcctgtctgccgtctccaaattacgtgatcaccgtctggactgaatgttacatcactggctggggagaaacccaaggtacctttggggctggccttctcaaggaagcccagcttcatgtgattgagaatacagtgtgcaatcactacgagtttctgaatggaagagtcaaatccaccgagctctgtgctgggcatttggccggaggcactgacagatgccagggtgacagtggagggcctgtggtttgcttcgacaaggacaaatacattttacgaggaataacttcttggggtcctggctgtgcatgccccaataagcctggtgtctatgttcgtgtttcaagctttgtcacttggattgagggagtgatgagaaataattaattgaacaagagacagagtgaagcattgactcacctagaggctagaatgggggtagggatttagcacgctggaaataacggacagtaatcaaacgaagacactgtccccagctaccaactatgccaaacctcagcatttttggtattattgtgtataagcttttcccgtctgactgctgggttctccaataaggtgacatagctatgccatttgttaaaaataaactctgtacttattttgatttgagtaaa

SEQ ID NO: 3 - DNA, 6428 bp, Macaca mulatta
  misc_feature (2053)..(2053): n is a, c, g, or t
  misc_feature (2272)..(2272): n is a, c, g, or t
  misc_feature (2395)..(2395): n is a, c, g, or t
agccttgcctttgaaatgttccagttggaacattgctgggcagcgtgcaaacaggagcgaacgtcagccggggcggcagggggcagcagaccccacactttgtccatgcctcaggtgggaggaagtgtccggctccagaaacctgccgcgggctttatcccaagcgacttcagccagcagacggttcatgtcctgaggctgcaaaatacgagttctgccatcatcgctgacaaagtcatcccagtttgtctgccatccccaaattatgcgatcgccaaccagactgaatgttatgtcactggctggggagaaacccaagcactacctgagcaaagccatgtggtccaggattgctaccatggtgatggacagagttatcaaggcacatcctccaccactgtcacaggaaggacctgccaagcttggtcatctatggaaccacatcagcataatagaaccacagaaaactacccaaatgctggcttgatcaggaactactgcaggaatccagatcctgtggcagccccttattgttatacgatggatcccaatgtcaggtgggagtactgcaacctgacacaatgctcagacgcagaagggactgccgtcgcacctccgaatgtcaccccggttccaagcctagaggctctttccgaacaagcaccgactgagcaaaggcctggggtgcaggagtgctaccacggtaatggacagagttatcgaggcacatacttcaccactgtgacaggaagaacctgccaagcttggtcatctatgacaccacattctcatagtcgtaccccagaaaactacccaaatggtggcttgatcaggaactactgcaggaatccagatcctgtggcagccccttattgttataccatggatcccaatgtcaggtgggagtactgcaacctgacacaatgctcagacgcagaagggactgccgtcgcacctccgaatgtcaccccggttccaagcctagaggctccttccgaacaagcaccgactgagcaaaggcctggggtgcaggagtgctaccacggtaatggacagagttatcgaggcacatacttcaccactgtgacaggaagaacctgccaagcttggtcatctatgacaccacattctcatagtcgtaccccagaaaactacccaaatggtggcttgatcaggaactactgcaggaatccagatcctgtggcagccccttattgttataccatggatcccaatgtcaggtgggagtactgcaacctgacacaatgctcagacgcagaggggactgccgtcgcacctccgactgtcaccccggttccaagcctagaggctccttctgagcaagcaccgactgagcaaaggcctggggtgcaggagtgctaccacggtaatggacagagttatcgaggcacatacttcaccactgtgacaggaagaacctgccaagcttggtcatctatgacaccgcactctcatagtcggaccccggaaaactacccaaatggtggcttgatcaggaactactgcaggaatccagatcctgtggcagccccttattgttatacgatggatcccaatgtcaggtgggagtactgcaacctgacacaatgctcagacgcagaagggactgccgtcgcacctccgaatgtcaccccggttccaagcctagaggctccttccgaacaagcaccgactgagcaaaggcctggggtgcaggagtgctaccacggtaatggacagagttatcgaggcacatacttcaccactgtgacaggaagaacctgccaagcttggtcatctatgacaccgcactctcatagtcggaccccggaaaactacccaaatggtggcttgatcaggaactactgcaggaatccagatcctgtggcagccccttattgttatacgatggatcccaatgtcaggtgggagtactgcaacctgacacaatgctcagacgcagaagggactgccgtcgcacctccgaatgtcaccccggttccaagcctagaggctccttccgaacaagcaccaactgagcaaaggcctgggntgcaggagtgctaccatggtaatggacagagttatcgaggcacatacttcaccactgtgacaggaagaacctgccaagcttggtcatctatgacaccgcactctcatagtcggaccccggaaaactacccaaatggtggcttgatcaggaactactgcaggaatccagatcctgtggcagccccttattgttataccatggatcccaatgtcaggtggnagtactgcaacctgacacaatgctcagacgcagaagggactgccgtcgcacctccgactgtcaccccggttccaagcctagaggctccttcgagcaaggcaccgactgagcaaaggcctgggntgcaggagtgctaccacggtaatggacagagttatcgaggcacatacttcaccactgtgacaggaagaacctgccaagcttggtcatctatgacaccgcactctcatagtcggaccccggaaaactacccaaatggtggcttgatcaggaactactgcaggaatccagatcctgtggcagccccttattgttatacgatggatcccaatgtcaggtgggagtactgcaacctgacacaatgctcagacgcagaagggactgccgtcgcacctccgaatgtcaccccggttccaagcctagaggctccttccgaacaagcaccgactgagcaaaggcctggggtgcaggagtgctaccacggtaatggacagagttatcgaggcacatacttcaccactgtgacaggaagaacctgccaagcttggtcatctatgacaccgcactctcatagtcggaccccggaaaactacccaaatggtggcttgatcaggaactactgcaggaatccagatcctgtggcagccccttattgttataccatggatcccaatgtcaggtgggagtactgcaacctgacacaatgctcagacgcagaagggactgccgtcgcacctccgaatgtcaccccggttccaagcctagaggctccttctgagcaagcaccaactgagcaaaggcttggggtgcaggagtgctaccacagtaatggacagagttatcgaggcacatacttcaccactgtgacaggaagaacctgccaagcttggtcatctatgacaccacactctcatagtcggaccccagaaaactacccaaatgctggcttggtcaagaactactgccgaaatccagatcctgtggcagccccttggtgttatacaacggatcccagtgtcaggtgggagtactgcaacctgacacgatgctcagatgcagaagggactgctgtcatgcctccaaatattattccggttccaagcctagaggcttttcttgaacaagaacctactgaggaaacccccggggtacaggagtgctactaccattatggacagagttatcgaggcacatactccaccactgttacaggaagaacttgccaagcttggtcatctatgacaccacaccagcatagtcggaccccaaaaaactatccaaatgctggcctgaccaggaactactgcaggaatccagatgctgagattcgcccttggtgttataccatggatcccagtgtcaggtgggagtactgcaacctgacacaatgtctggtgacagaatcaagtgtccttgaaactctcacagtggtcccagatccaagcacacaggcttcttctgaagaagcaccaacggagcaaagtcccgaggtccaggactgctaccatggtgatggacagagttatcgaggctcattctccaccactgtcacaggaaggacatgtcagtcttggtcctctatgacaccacactggcatcagaggacaacagaatattatccagatggtggcctgaccaggaactactgcaggaatccagatgctgagattcgcccttggtgttataccatggatcccagtgtcaggtgggagtactgcaacctgacacaatgtccagtgacagaatcaagtgtcctcgcaacgtccatggctgtttctgaacaagcaccaatggagcaaagccccggggtccaggactgctaccatggtgatggacagagttatcgaggttcattctccaccactgtcacaggaaggacatgtcagtcttggtcctctatgacaccacactggcatcagaggaccatagaatactacccaaatggtggcctgaccaagaactactgcaggaatccagatgctgagattcgcccttggtgttataccatggatcccagagtcagatgggagtactgcaacctgacacaatgtgtggtgatggaatcaagtgtccttgcaactcccatggtggtcccagttccaagcagagaggttccttctgaagaagcaccaactgaaaacagccctggggtccaggactgctaccaaggtgatggacagagttatcgaggcacattctccaccactatcacaggaagaacatgtcagtcttggttgtctatgacaccacatcggcatcggaggatcccattacgctatccaaatgctggcctgaccaggaactattgcagaaatccagatgctgagattcgcccttggtgttacaccatggatcccagtgtcaggtgggagtactgcaacctgacacaatgtccagtgacagaatcaagtgtcctcacaactcccacggtggtcccggttccaagcacagaggctccttctgaacaagcaccacctgagaaaagccctgtggtccaggattgctaccatggtgatggacagagttatcgaggcacatcctccaccactgtcacaggaaggaactgtcaatcttggtcatctatgataccacactggcatcagaggaccccagaaaactacccaaatgctggcctgaccaggaactactgcaggaatccagattctgggaaacaaccctggtgttacacgactgatccatgtgtgaggtgggagtactgcaacctgacacaatgctcagaaacagaatcaggtgtcctagagactcccactgttgttccggttccaagcatggaagctcattctgaagcagcaccaactgagcaaacccctgtggtccagcagtgctaccatggtaatggacagagttatcgaggcacattctccaccactgtcacaggaaggacatgtcaatcttggtcatccatgacaccacaccagcataagaggaccccggaaaaccacccaaatgatgacttgacaatgaactactgcaggaatccagatgctgacacaggcccttggtgttttaccatggaccccagcgtcaggcgggagtactgcaacctgacgcgatgctcagacacagaagggactgtggtcacacctccgactgttatcccggttccaagcctagaggctccttctgaacaagcatcctcttcatttgattgtgggaagcctcaagtggagccaaagaaatgtcctggaagcattgtaggtgggtgtgtggcccacccacattcctggccctggcaagtcagtcttagaacaaggtttggaaagcacttctgtggaggcaccttaatatccccagagtgggtgctgactgctgcttgctgcttggagacgttctcaaggccttccttctacaaggtcatcctgggtgcacaccaagaagtgaatctcgaatctcatgttcaagaaatagaagtgtctaggttgttcttggagcccataggagcagatattgccttgctaaagctaagcaggcctgccatcatcactgacaaagtaatcccagcctgtctgccgtctccaaattacgtgatcaccgcctggactgaatgttacatcactggctggggagaaacccaaggtacctttggggctggccttctcaaggaagcccagcttcatgtgattgagaatacagtgtgcaatcactacgagtttctgaatggaagagtcaaatccactgagctctgtgctgggcatttggccggaggcactgacagatgccagggtgacaatggagggcctgtggtttgcttcgacaaggacaaatacattttacgaggaataacttcttggggtcctggctgtgcatgccccaataagcctggtgtctatgttcgtgtttcaagctttgtcacttggattgagggagtgatgagaaataattaattgaacaagagacagagtgaagcattgactcacctagaggctagaatgggggtagggatttagcacgctggaaataacggacagtaatcaaacgaagacactgtccccagctaccaactatgccaaacctcagcatttttggtattattgtgtataagcttttcctgtctgactgctgggttctccaataaggtgacatagctatgccatttgttaaaaataaactctgtacttattttgatttgagtaaa

SEQ ID NO: 4 - RNA, 25 nt, Artificial Sequence
  Synthetic
cugagcaaagccaugugguacagga

SEQ ID NO: 5 - DNA, 25 bp, Artificial Sequence
  Synthetic
agcaaagccaugugguccaagautg

SEQ ID NO: 6 - RNA, 25 nt, Artificial Sequence
  Synthetic
aagccaugugguccaggauagcuac

SEQ ID NO: 7 - RNA, 25 nt, Artificial Sequence
  Synthetic
agccaugugguccaggauuacuacc

SEQ ID NO: 8 - RNA, 25 nt, Artificial Sequence
  Synthetic
gccaugugguccaggauugauacca

SEQ ID NO: 9 - DNA, 25 bp, Artificial Sequence
  Synthetic
ccaugugguccaggauugcaaccat

SEQ ID NO: 10 - DNA, 25 bp, Artificial Sequence
  Synthetic
caugugguccaggauugcuaccatg

SEQ ID NO: 11 - RNA, 25 nt, Artificial Sequence
  Synthetic
augugguccaggauugcuaacaugg

SEQ ID NO: 12 - DNA, 25 bp, Artificial Sequence
  Synthetic
ugugguccaggauugcuacaauggt

SEQ ID NO: 13 - RNA, 25 nt, Artificial Sequence
  Synthetic
ggugauggacagaguuaucaaggca

SEQ ID NO: 14 - DNA, 25 bp, Artificial Sequence
  Synthetic
uccaccacugucacaggaaagacct

SEQ ID NO: 15 - DNA, 25 bp, Artificial Sequence
  Synthetic
ccaccacugucacaggaagaacctg

SEQ ID NO: 16 - RNA, 25 nt, Artificial Sequence
  Synthetic
cugucacaggaaggaccugacaagc

SEQ ID NO: 17 - DNA, 25 bp, Artificial Sequence
  Synthetic
ggaaggaccugccaagcuuagucat

SEQ ID NO: 18 - DNA, 25 bp, Artificial Sequence
  Synthetic
gaaggaccugccaagcuugaucatc

SEQ ID NO: 19 - DNA, 25 bp, Artificial Sequence
  Synthetic
aggaccugccaagcuugguaaucta

SEQ ID NO: 20 - DNA, 25 bp, Artificial Sequence
  Synthetic
ggaccugccaagcuuggucaucuat

SEQ ID NO: 21 - DNA, 25 bp, Artificial Sequence
  Synthetic
gaccugccaagcuuggucaacuatg

SEQ ID NO: 22 - RNA, 25 nt, Artificial Sequence
  Synthetic
accugccaagcuuggucauauauga

SEQ ID NO: 23 - RNA, 25 nt, Artificial Sequence
  Synthetic
ccugccaagcuuggucaucaaugac

SEQ ID NO: 24 - DNA, 25 bp, Artificial Sequence
  Synthetic
agcuuggucaucuaugacaacacat

SEQ ID NO: 25 - DNA, 25 bp, Artificial Sequence
  Synthetic
gucaucuaugacaccacauaaacat

SEQ ID NO: 26 - RNA, 25 nt, Artificial Sequence
  Synthetic
cacagaaaacuacccaaauacuggc

SEQ ID NO: 27 - DNA, 25 bp, Artificial Sequence
  Synthetic
agaaaacuacccaaaugcuagcutg

SEQ ID NO: 28 - DNA, 25 bp, Artificial Sequence
  Synthetic
aaaacuacccaaaugcuggauugat

SEQ ID NO: 29 - RNA, 25 nt, Artificial Sequence
  Synthetic
acccaaaugcuggcuugauaaugaa

SEQ ID NO: 30 - RNA, 25 nt, Artificial Sequence
  Synthetic
cccaaaugcuggcuugaucaugaac

SEQ ID NO: 31 - DNA, 25 bp, Artificial Sequence
  Synthetic
gaacuacugcaggaauccaaaugct

SEQ ID NO: 32 - RNA, 25 nt, Artificial Sequence
  Synthetic
uacugcaggaauccagaugaugugg

SEQ ID NO: 33 - RNA, 25 nt, Artificial Sequence
  Synthetic
acugcaggaauccagaugcaguggc

SEQ ID NO: 34 - RNA, 25 nt, Artificial Sequence
  Synthetic
ugcaggaauccagaugcugaggcag

SEQ ID NO: 35 - RNA, 25 nt, Artificial Sequence
  Synthetic
aggugggaguacugcaaccagacgc

SEQ ID NO: 36 - RNA, 25 nt, Artificial Sequence
  Synthetic
aaugcucagacgcagaaggaacugc

SEQ ID NO: 37 - DNA, 25 bp, Artificial Sequence
  Synthetic
gacuguuaccccgguuccaagccta

SEQ ID NO: 38 - RNA, 25 nt, Artificial Sequence
  Synthetic
acuguuaccccgguuccaaaccuag

SEQ ID NO: 39 - RNA, 25 nt, Artificial Sequence
  Synthetic
cuguuaccccgguuccaagacuaga

SEQ ID NO: 40 - RNA, 25 nt, Artificial Sequence
  Synthetic
uguuaccccgguuccaagcauagag

SEQ ID NO: 41 - RNA, 25 nt, Artificial Sequence
  Synthetic
guuaccccgguuccaagccaagagg

SEQ ID NO: 42 - RNA, 25 nt, Artificial Sequence
  Synthetic
uuaccccgguuccaagccuagaggc

SEQ ID NO: 43 - DNA, 25 bp, Artificial Sequence
  Synthetic
uaccccgguuccaagccuaaaggct

SEQ ID NO: 44 - DNA, 25 bp, Artificial Sequence
  Synthetic
gugcuaccaugguaauggaaagagt

SEQ ID NO: 45 - DNA, 25 bp, Artificial Sequence
  Synthetic
ugcuaccaugguaauggacagagtt

SEQ ID NO: 46 - DNA, 25 bp, Artificial Sequence
  Synthetic
gcuaccaugguaauggacaaaguta

SEQ ID NO: 47 - DNA, 25 bp, Artificial Sequence
  Synthetic
cuaccaugguaauggacagaguuat

SEQ ID NO: 48 - DNA, 25 bp, Artificial Sequence
  Synthetic
uaccaugguaauggacagaauuatc

SEQ ID NO: 49 - RNA, 25 nt, Artificial Sequence
  Synthetic
accaugguaauggacagagauaucg

SEQ ID NO: 50 - RNA, 25 nt, Artificial Sequence
  Synthetic
ccaugguaauggacagaguaaucga

SEQ ID NO: 51 - RNA, 25 nt, Artificial Sequence
  Synthetic
caugguaauggacagaguuaucgag

SEQ ID NO: 52 - RNA, 25 nt, Artificial Sequence
  Synthetic
augguaauggacagaguuaacgagg

SEQ ID NO: 53 - RNA, 25 nt, Artificial Sequence
  Synthetic
ugguaauggacagaguuauagaggc

SEQ ID NO: 54 - RNA, 25 nt, Artificial Sequence
  Synthetic
gguaauggacagaguuaucaaggca

SEQ ID NO: 55 - RNA, 25 nt, Artificial Sequence
  Synthetic
guaauggacagaguuaucgaggcac

SEQ ID NO: 56 - RNA, 25 nt, Artificial Sequence
  Synthetic
ggcacauacuccaccacugacacag

SEQ ID NO: 57 - DNA, 25 bp, Artificial Sequence
  Synthetic
cuuggucaucuaugacaccacactc

SEQ ID NO: 58 - DNA, 25 bp, Artificial Sequence
  Synthetic
gucaucuaugacaccacacacgcat

SEQ ID NO: 59 - DNA, 25 bp, Artificial Sequence
  Synthetic
ucaucuaugacaccacacuagcata

SEQ ID NO: 60 - RNA, 25 nt, Artificial Sequence
  Synthetic
caucuaugacaccacacucacauag

SEQ ID NO: 61 - RNA, 25 nt, Artificial Sequence
  Synthetic
gcacauacuccaccacuguaacugg

SEQ ID NO: 62 - DNA, 25 bp, Artificial Sequence
  Synthetic
agccccuuauuguuauacgagggat

SEQ ID NO: 63 - DNA, 25 bp, Artificial Sequence
  Synthetic
gccccuuauuguuauacgaaggatc

SEQ ID NO: 64 - RNA, 25 nt, Artificial Sequence
  Synthetic
ccaagccuagaggcuccuuaugaac

SEQ ID NO: 65 - RNA, 25 nt, Artificial Sequence
  Synthetic
aggcuccuucugaacaagcaccaac

SEQ ID NO: 66 - DNA, 25 bp, Artificial Sequence
  Synthetic
auggacagaguuaucaaggaacata

SEQ ID NO: 67 - RNA, 25 nt, Artificial Sequence
  Synthetic
uggacagaguuaucaaggcacauac

SEQ ID NO: 68 - DNA, 25 bp, Artificial Sequence
  Synthetic
ggacagaguuaucaaggcaaauact

SEQ ID NO: 69 - DNA, 25 bp, Artificial Sequence
  Synthetic
gacagaguuaucaaggcacauactt

SEQ ID NO: 70 - DNA, 25 bp, Artificial Sequence
  Synthetic
acagaguuaucaaggcacaaacutc

SEQ ID NO: 71 - RNA, 25 nt, Artificial Sequence
  Synthetic
cagaguuaucaaggcacauacuuca

SEQ ID NO: 72 - RNA, 25 nt, Artificial Sequence
  Synthetic
uacccaaaugcuggcuugaacaaga

SEQ ID NO: 73 - DNA, 25 bp, Artificial Sequence
  Synthetic
ccaaaugcuggcuugaucaagaact

SEQ ID NO: 74 - RNA, 25 nt, Artificial Sequence
  Synthetic
ucaagaacuacugccgaaaaccaga

SEQ ID NO: 75 - DNA, 25 bp, Artificial Sequence
  Synthetic
caagaacuacugccgaaauacagat

SEQ ID NO: 76 - DNA, 25 bp, Artificial Sequence
  Synthetic
aagaacuacugccgaaaucaagatc

SEQ ID NO: 77 - DNA, 25 bp, Artificial Sequence
  Synthetic
gaacuacugccgaaauccaaaucct

SEQ ID NO: 78 - DNA, 25 bp, Artificial Sequence
  Synthetic
cuacugccgaaauccagauacugtg

SEQ ID NO: 79 - RNA, 25 nt, Artificial Sequence
  Synthetic
uguggcagccccuugguguaauaca

SEQ ID NO: 80 - RNA, 25 nt, Artificial Sequence
  Synthetic
guggcagccccuugguguuauacaa

SEQ ID NO: 81 - RNA, 25 nt, Artificial Sequence
  Synthetic
uggcagccccuugguguuaaacaac

SEQ ID NO: 82 - RNA, 25 nt, Artificial Sequence
  Synthetic
ggcagccccuugguguuauacaaca

SEQ ID NO: 83 - RNA, 25 nt, Artificial Sequence
  Synthetic
gcagccccuugguguuauaaaacag

SEQ ID NO: 84 - RNA, 25 nt, Artificial Sequence
  Synthetic
cagccccuugguguuauacaacaga

SEQ ID NO: 85 - DNA, 25 bp, Artificial Sequence
  Synthetic
agccccuugguguuauacaacagat

SEQ ID NO: 86 - DNA, 25 bp, Artificial Sequence
  Synthetic
gccccuugguguuauacaaaagatc

SEQ ID NO: 87 - RNA, 25 nt, Artificial Sequence
  Synthetic
ccccuugguguuauacaacagaucc

SEQ ID NO: 88 - RNA, 25 nt, Artificial Sequence
  Synthetic
ggugggaguacugcaaccuaacacg

SEQ ID NO: 89 - RNA, 25 nt, Artificial Sequence
  Synthetic
gugggaguacugcaaccugacacga

SEQ ID NO: 90 - RNA, 25 nt, Artificial Sequence
  Synthetic
ggaguacugcaaccugacaagaugc

SEQ ID NO: 91 - DNA, 25 bp, Artificial Sequence
  Synthetic
gaguacugcaaccugacacaaugct

SEQ ID NO: 92 - RNA, 25 nt, Artificial Sequence
  Synthetic
guacugcaaccugacacgaagcuca

SEQ ID NO: 93 - RNA, 25 nt, Artificial Sequence
  Synthetic
uacugcaaccugacacgauacucag

SEQ ID NO: 94 - RNA, 25 nt, Artificial Sequence
  Synthetic
acugcaaccugacacgaugaucaga

SEQ ID NO: 95 - DNA, 25 bp, Artificial Sequence
  Synthetic
cugcaaccugacacgaugcacagat

SEQ ID NO: 96 - DNA, 25 bp, Artificial Sequence
  Synthetic
ugcaaccugacacgaugcuaagatg

SEQ ID NO: 97 - RNA, 25 nt, Artificial Sequence
  Synthetic
caaccugacacgaugcucaaaugca

SEQ ID NO: 98 - RNA, 25 nt, Artificial Sequence
  Synthetic
accugacacgaugcucagaagcaga

SEQ ID NO: 99 - RNA, 25 nt, Artificial Sequence
  Synthetic
ccugacacgaugcucagauacagaa

SEQ ID NO: 100 - DNA, 25 bp, Artificial Sequence
  Synthetic
cugacacgaugcucagaugaagaat

SEQ ID NO: 101 - DNA, 25 bp, Artificial Sequence
  Synthetic
ugacacgaugcucagaugcagaatg

SEQ ID NO: 102 - RNA, 25 nt, Artificial Sequence
  Synthetic
gacacgaugcucagaugcaaaaugg

SEQ ID NO: 103 - RNA, 25 nt, Artificial Sequence
  Synthetic
acacgaugcucagaugcagaaugga

SEQ ID NO: 104 - RNA, 25 nt, Artificial Sequence
  Synthetic
cacgaugcucagaugcagaauggac

SEQ ID NO: 105 - DNA, 25 bp, Artificial Sequence
  Synthetic
ugcuacuaccauuauggacagagtt

SEQ ID NO: 106 - DNA, 25 bp, Artificial Sequence
  Synthetic
gcuacuaccauuauggacaaaguta

SEQ ID NO: 107 - RNA, 25 nt, Artificial Sequence
  Synthetic
cuacuaccauuauggacagaguuac

SEQ ID NO: 108 - RNA, 25 nt, Artificial Sequence
  Synthetic
uacuaccauuauggacagaauuacc

SEQ ID NO: 109 - RNA, 25 nt, Artificial Sequence
  Synthetic
acuaccauuauggacagagauaccg

SEQ ID NO: 110 - RNA, 25 nt, Artificial Sequence
  Synthetic
cuaccauuauggacagaguaaccga

SEQ ID NO: 111 - RNA, 25 nt, Artificial Sequence
  Synthetic
uaccauuauggacagaguuaccgag

SEQ ID NO: 112 - RNA, 25 nt, Artificial Sequence
  Synthetic
accauuauggacagaguuaacgagg

SEQ ID NO: 113 - RNA, 25 nt, Artificial Sequence
  Synthetic
gaggcacauacuccaccacagucac

SEQ ID NO: 114 - DNA, 25 bp, Artificial Sequence
  Synthetic
cuccaccacugucacaggaagaact

SEQ ID NO: 115 - DNA, 25 bp, Artificial Sequence
  Synthetic
acaggaagaacuugccaagauuggt

SEQ ID NO: 116 - DNA, 25 bp, Artificial Sequence
  Synthetic
caggaagaacuugccaagcauggtc

SEQ ID NO: 117 - RNA, 25 nt, Artificial Sequence
  Synthetic
aggaagaacuugccaagcuagguca

SEQ ID NO: 118 - DNA, 25 bp, Artificial Sequence
  Synthetic
ggaagaacuugccaagcuuagucat

SEQ ID NO: 119 - DNA, 25 bp, Artificial Sequence
  Synthetic
gaagaacuugccaagcuugaucatc

SEQ ID NO: 120 - DNA, 25 bp, Artificial Sequence
  Synthetic
aagaacuugccaagcuuggacauct

SEQ ID NO: 121 - DNA, 25 bp, Artificial Sequence
  Synthetic
agaacuugccaagcuugguaaucta

SEQ ID NO: 122 - DNA, 25 bp, Artificial Sequence
  Synthetic
gaacuugccaagcuuggucaucuat

SEQ ID NO: 123 - DNA, 25 bp, Artificial Sequence
  Synthetic
aacuugccaagcuuggucaacuatg

SEQ ID NO: 124 - RNA, 25 nt, Artificial Sequence
  Synthetic
acuugccaagcuuggucauauauga

SEQ ID NO: 125 - RNA, 25 nt, Artificial Sequence
  Synthetic
cuugccaagcuuggucaucaaugac

SEQ ID NO: 126 - RNA, 25 nt, Artificial Sequence
  Synthetic
uugccaagcuuggucaucuaugaca

SEQ ID NO: 127 - RNA, 25 nt, Artificial Sequence
  Synthetic
ugccaagcuuggucaucuaagacac

SEQ ID NO: 128 - RNA, 25 nt, Artificial Sequence
  Synthetic
gcuuggucaucuaugacacaacacc

SEQ ID NO: 129 - RNA, 25 nt, Artificial Sequence
  Synthetic
uuggucaucuaugacaccaaaccag

SEQ ID NO: 130 - RNA, 25 nt, Artificial Sequence
  Synthetic
uggucaucuaugacaccacaccagc

SEQ ID NO: 131 - DNA, 25 bp, Artificial Sequence
  Synthetic
gucaucuaugacaccacacaagcat

SEQ ID NO: 132 - RNA, 25 nt, Artificial Sequence
  Synthetic
caucuaugacaccacaccaacauag

SEQ ID NO: 133 - DNA, 25 bp, Artificial Sequence
  Synthetic
aucuaugacaccacaccagaauagt

SEQ ID NO: 134 - DNA, 25 bp, Artificial Sequence
  Synthetic
cuaugacaccacaccagcaaagucg

SEQ ID NO: 135 - RNA, 25 nt, Artificial Sequence
  Synthetic
gucggaccccagaaaacuaaccaaa

SEQ ID NO: 136 - DNA, 25 bp, Artificial Sequence
  Synthetic
ucggaccccagaaaacuacacaaat

SEQ ID NO: 137 - RNA, 25 nt, Artificial Sequence
  Synthetic
gaaaacuacccaaaugcugaccuga

SEQ ID NO: 138 - RNA, 25 nt, Artificial Sequence
  Synthetic
gcugagauucgcccuugguauuaca

SEQ ID NO: 139 - RNA, 25 nt, Artificial Sequence
  Synthetic
cugagauucgcccuuggugauacac

SEQ ID NO: 140 - RNA, 25 nt, Artificial Sequence
  Synthetic
gagauucgcccuugguguuacacca

SEQ ID NO: 141 - DNA, 25 bp, Artificial Sequence
  Synthetic
agauucgcccuugguguuaaaccat

SEQ ID NO: 142 - RNA, 25 nt, Artificial Sequence
  Synthetic
uucgcccuugguguuacacaaugga

SEQ ID NO: 143 - RNA, 25 nt, Artificial Sequence
  Synthetic
cuugguguuacaccauggaacccag

SEQ ID NO: 144 - DNA, 25 bp, Artificial Sequence
  Synthetic
uugguguuacaccauggauaccagt

SEQ ID NO: 145 - DNA, 25 bp, Artificial Sequence
  Synthetic
ugguguuacaccauggaucacagtg

SEQ ID NO: 146 - DNA, 25 bp, Artificial Sequence
  Synthetic
gguguuacaccauggauccaagugt

SEQ ID NO: 147 - RNA, 25 nt, Artificial Sequence
  Synthetic
uguuacaccauggaucccaauguca

SEQ ID NO: 148 - RNA, 25 nt, Artificial Sequence
  Synthetic
gaaucaaguguccuugcaaaucuca

SEQ ID NO: 149 - RNA, 25 nt, Artificial Sequence
  Synthetic
aaucaaguguccuugcaacacucac

SEQ ID NO: 150 - RNA, 25 nt, Artificial Sequence
  Synthetic
aucaaguguccuugcaacuaucacg

SEQ ID NO: 151 - DNA, 25 bp, Artificial Sequence
  Synthetic
auggacagaguuaucgaggaucatt

SEQ ID NO: 152 - DNA, 25 bp, Artificial Sequence
  Synthetic
uggacagaguuaucgaggcacautc

SEQ ID NO: 153 - DNA, 25 bp, Artificial Sequence
  Synthetic
acaccacacuggcaucagaagacaa

SEQ ID NO: 154 - DNA, 25 bp, Artificial Sequence
  Synthetic
uugguguuauaccauggauaccaat

SEQ ID NO: 155 - DNA, 25 bp, Artificial Sequence
  Synthetic
ugguguuauaccauggaucacaatg

SEQ ID NO: 156 - DNA, 25 bp, Artificial Sequence
  Synthetic
gguguuauaccauggauccaaaugt

SEQ ID NO: 157 - DNA, 25 bp, Artificial Sequence
  Synthetic
guguuauaccauggaucccaaugtc

SEQ ID NO: 158 - RNA, 25 nt, Artificial Sequence
  Synthetic
ucagaugggaguacugcaaacugac

SEQ ID NO: 159 - RNA, 25 nt, Artificial Sequence
  Synthetic
gaugggaguacugcaaccuaacaca

SEQ ID NO: 160 - RNA, 25 nt, Artificial Sequence
  Synthetic
augggaguacugcaaccugacacaa

SEQ ID NO: 161 - DNA, 25 bp, Artificial Sequence
  Synthetic
ugggaguacugcaaccugaaacaat

SEQ ID NO: 162 - DNA, 25 bp, Artificial Sequence
  Synthetic
gggaguacugcaaccugacacaatg

SEQ ID NO: 163 - RNA, 25 nt, Artificial Sequence
  Synthetic
ggcuguuucugaacaagcaacaacg

SEQ ID NO: 164 - RNA, 25 nt, Artificial Sequence
  Synthetic
guuucugaacaagcaccaaaggagc

SEQ ID NO: 165 - RNA, 25 nt, Artificial Sequence
  Synthetic
cuccaccacuguuacaggaaggaca

SEQ ID NO: 166 - RNA, 24 nt, Artificial Sequence
  Synthetic
uccaccacuguuacaggaaagaca

SEQ ID NO: 167 - RNA, 24 nt, Artificial Sequence
  Synthetic
ccaccacuguuacaggaagaacag

SEQ ID NO: 168 - RNA, 25 nt, Artificial Sequence
  Synthetic
gacaccacacuggcaucagagaacc

SEQ ID NO: 169 - RNA, 25 nt, Artificial Sequence
  Synthetic
acaccacacuggcaucagaaaacca

SEQ ID NO: 170 - RNA, 24 nt, Artificial Sequence
  Synthetic
agaauacuacccaaaugguagccg

SEQ ID NO: 171 - RNA, 25 nt, Artificial Sequence
  Synthetic
gaauacuacccaaauggugaccuga

SEQ ID NO: 172 - RNA, 25 nt, Artificial Sequence
  Synthetic
aauacuacccaaaugguggacugac

SEQ ID NO: 173 - RNA, 25 nt, Artificial Sequence
  Synthetic
uccuucugaagaagcaccaacugaa

SEQ ID NO: 174 - RNA, 25 nt, Artificial Sequence
  Synthetic
ccuucugaagaagcaccaaaugaaa

SEQ ID NO: 175 - RNA, 25 nt, Artificial Sequence
  Synthetic
cuucugaagaagcaccaacagaaaa

SEQ ID NO: 176 - RNA, 25 nt, Artificial Sequence
  Synthetic
uucugaagaagcaccaacuaaaaac

SEQ ID NO: 177 - RNA, 25 nt, Artificial Sequence
  Synthetic
ucugaagaagcaccaacugaaaaca

SEQ ID NO: 178 - RNA, 25 nt, Artificial Sequence
  Synthetic
cugaagaagcaccaacugaaaacag

SEQ ID NO: 179 - RNA, 25 nt, Artificial Sequence
  Synthetic
ugaagaagcaccaacugaaaacagc

SEQ ID NO: 180 - RNA, 25 nt, Artificial Sequence
  Synthetic
gaagaagcaccaacugaaaacagca

SEQ ID NO: 181 - RNA, 25 nt, Artificial Sequence
  Synthetic
aagaagcaccaacugaaaaaagcac

SEQ ID NO: 182 - RNA, 24 nt, Artificial Sequence
  Synthetic
agaagcaccaacugaaaacagcac

SEQ ID NO: 183 - RNA, 24 nt, Artificial Sequence
  Synthetic
gaagcaccaacugaaaacaacacg

SEQ ID NO: 184 - RNA, 25 nt, Artificial Sequence
  Synthetic
aagcaccaacugaaaacagaacugg

SEQ ID NO: 185 - RNA, 25 nt, Artificial Sequence
  Synthetic
aggugauggacagaguuauagaggc

SEQ ID NO: 186 - RNA, 25 nt, Artificial Sequence
  Synthetic
cuccaccacuaucacaggaagaaca

SEQ ID NO: 187 - RNA, 24 nt, Artificial Sequence
  Synthetic
uccaccacuaucacaggaaaaaca

SEQ ID NO: 188 - RNA, 24 nt, Artificial Sequence
  Synthetic
ccaccacuaucacaggaagaacag

SEQ ID NO: 189 - RNA, 24 nt, Artificial Sequence
  Synthetic
caccacuaucacaggaagaacaug

SEQ ID NO: 190 - RNA, 24 nt, Artificial Sequence
  Synthetic
accacuaucacaggaagaaaaugc

SEQ ID NO: 191 - RNA, 25 nt, Artificial Sequence
  Synthetic
ccacuaucacaggaagaacauguca

SEQ ID NO: 192 - RNA, 25 nt, Artificial Sequence
  Synthetic
cacuaucacaggaagaacaagucag

SEQ ID NO: 193 - RNA, 24 nt, Artificial Sequence
  Synthetic
acuaucacaggaagaacauaucag

SEQ ID NO: 194 - RNA, 24 nt, Artificial Sequence
  Synthetic
cuaucacaggaagaacaugacagc

SEQ ID NO: 195 - RNA, 24 nt, Artificial Sequence
  Synthetic
uaucacaggaagaacauguaaguc

SEQ ID NO: 196 - RNA, 23 nt, Artificial Sequence
  Synthetic
aucacaggaagaacaugucaguc

SEQ ID NO: 197 - RNA, 24 nt, Artificial Sequence
  Synthetic
ucacaggaagaacaugucaaucug

SEQ ID NO: 198 - RNA, 25 nt, Artificial Sequence
  Synthetic
cacaggaagaacaugucagacuugg

SEQ ID NO: 199 - RNA, 24 nt, Artificial Sequence
  Synthetic
acaggaagaacaugucaguauugg

SEQ ID NO: 200 - RNA, 24 nt, Artificial Sequence
  Synthetic
ggaagaacaugucagucuuagucg

SEQ ID NO: 201 - RNA, 24 nt, Artificial Sequence
  Synthetic
gaagaacaugucagucuugaucgc

SEQ ID NO: 202 - RNA, 24 nt, Artificial Sequence
  Synthetic
aagaacaugucagucuuggacguc

SEQ ID NO: 203 - RNA, 24 nt, Artificial Sequence
  Synthetic
agaacaugucagucuugguaguca

SEQ ID NO: 204 - RNA, 24 nt, Artificial Sequence
  Synthetic
ggcaucggaggaucccauuauaca

SEQ ID NO: 205 - RNA, 25 nt, Artificial Sequence
  Synthetic
acuauccaaaugcuggccuaaccag

SEQ ID NO: 206 - RNA, 25 nt, Artificial Sequence
  Synthetic
gcacagaggcuccuucugaacaagc

SEQ ID NO: 207 - RNA, 25 nt, Artificial Sequence
  Synthetic
uccuucugaacaagcaccaacugag

SEQ ID NO: 208 - RNA, 25 nt, Artificial Sequence
  Synthetic
ccuucugaacaagcaccacaugaga

SEQ ID NO: 209 - RNA, 25 nt, Artificial Sequence
  Synthetic
cuucugaacaagcaccaccagagaa

SEQ ID NO: 210 - RNA, 25 nt, Artificial Sequence
  Synthetic
uucugaacaagcaccaccuaagaaa

SEQ ID NO: 211 - RNA, 25 nt, Artificial Sequence
  Synthetic
ucugaacaagcaccaccugagaaaa

SEQ ID NO: 212 - RNA, 25 nt, Artificial Sequence
  Synthetic
cugaacaagcaccaccugaaaaaag

SEQ ID NO: 213 - RNA, 25 nt, Artificial Sequence
  Synthetic
ugaacaagcaccaccugagaaaagc

SEQ ID NO: 214 - RNA, 25 nt, Artificial Sequence
  Synthetic
gaacaagcaccaccugagaaaagcc

SEQ ID NO: 215 - RNA, 25 nt, Artificial Sequence
  Synthetic
aacaagcaccaccugagaaaagccc

SEQ ID NO: 216 - RNA, 24 nt, Artificial Sequence
  Synthetic
caagcaccaccugagaaaaacccg

SEQ ID NO: 217 - RNA, 24 nt, Artificial Sequence
  Synthetic
aagcaccaccugagaaaagaccug

SEQ ID NO: 218 - RNA, 24 nt, Artificial Sequence
  Synthetic
agcaccaccugagaaaagcacugg

SEQ ID NO: 219 - RNA, 25 nt, Artificial Sequence
  Synthetic
cugagaaaagcccugugguacagga

SEQ ID NO: 220 - RNA, 25 nt, Artificial Sequence
  Synthetic
gcccugugguccaggauugauacca

SEQ ID NO: 221 - RNA, 25 nt, Artificial Sequence
  Synthetic
cugugguccaggauugcuaacaugg

SEQ ID NO: 222 - RNA, 25 nt, Artificial Sequence
  Synthetic
cuccaccacugucacaggaaggacc

SEQ ID NO: 223 - RNA, 24 nt, Artificial Sequence
  Synthetic
uccaccacugucacaggaaagacc

SEQ ID NO: 224 - RNA, 24 nt, Artificial Sequence
  Synthetic
ucuuggucaucuaugauacaacac

SEQ ID NO: 225 - RNA, 24 nt, Artificial Sequence
  Synthetic
cuuggucaucuaugauaccacacg

SEQ ID NO: 226 - RNA, 25 nt, Artificial Sequence
  Synthetic
uuggucaucuaugauaccaaacugg

SEQ ID NO: 227 - RNA, 25 nt, Artificial Sequence
  Synthetic
uggucaucuaugauaccacacuggc

SEQ ID NO: 228 - RNA, 25 nt, Artificial Sequence
  Synthetic
ggucaucuaugauaccacaauggca

SEQ ID NO: 229 - RNA, 24 nt, Artificial Sequence
  Synthetic
gucaucuaugauaccacacaggca

SEQ ID NO: 230 - RNA, 24 nt, Artificial Sequence
  Synthetic
ucaucuaugauaccacacuagcac

SEQ ID NO: 231 - RNA, 25 nt, Artificial Sequence
  Synthetic
caucuaugauaccacacugacauca

SEQ ID NO: 232 - RNA, 25 nt, Artificial Sequence
  Synthetic
aucuaugauaccacacuggaaucag

SEQ ID NO: 233 - RNA, 25 nt, Artificial Sequence
  Synthetic
ucuaugauaccacacuggcaucaga

SEQ ID NO: 234 - RNA, 25 nt, Artificial Sequence
  Synthetic
cuaugauaccacacuggcaacagag

SEQ ID NO: 235 - RNA, 25 nt, Artificial Sequence
  Synthetic
ugauaccacacuggcaucaaaggac

SEQ ID NO: 236 - RNA, 25 nt, Artificial Sequence
  Synthetic
auaccacacuggcaucagaagaccc

SEQ ID NO: 237 - RNA, 25 nt, Artificial Sequence
  Synthetic
agaggaccccagaaaacuaaccaaa

SEQ ID NO: 238 - RNA, 24 nt, Artificial Sequence
  Synthetic
gaggaccccagaaaacuacacaaa

SEQ ID NO: 239 - RNA, 24 nt, Artificial Sequence
  Synthetic
agaacuacugcaggaauccagauc

SEQ ID NO: 240 - RNA, 25 nt, Artificial Sequence
  Synthetic
acuacugcaggaauccagaaucugg

SEQ ID NO: 241 - RNA, 25 nt, Artificial Sequence
  Synthetic
uacugcaggaauccagauuauggga

SEQ ID NO: 242 - RNA, 25 nt, Artificial Sequence
  Synthetic
acugcaggaauccagauucagggaa

SEQ ID NO: 243 - RNA, 25 nt, Artificial Sequence
  Synthetic
cugcaggaauccagauucuaggaaa

SEQ ID NO: 244 - RNA, 25 nt, Artificial Sequence
  Synthetic
ugcaggaauccagauucugagaaac

SEQ ID NO: 245 - RNA, 25 nt, Artificial Sequence
  Synthetic
gcaggaauccagauucuggaaaaca

SEQ ID NO: 246 - RNA, 25 nt, Artificial Sequence
  Synthetic
ggaauccagauucugggaaacaacc

SEQ ID NO: 247 - RNA, 25 nt, Artificial Sequence
  Synthetic
gggaaacaacccugguguuacacaa

SEQ ID NO: 248 - RNA, 25 nt, Artificial Sequence
  Synthetic
ggaaacaacccugguguuaaacaac

SEQ ID NO: 249 - RNA, 24 nt, Artificial Sequence
  Synthetic
ugugugaggugggaguacuacaac

SEQ ID NO: 250 - RNA, 24 nt, Artificial Sequence
  Synthetic
gugugaggugggaguacugaaauc

SEQ ID NO: 251 - RNA, 25 nt, Artificial Sequence
  Synthetic
gugaggugggaguacugcaaucuga

SEQ ID NO: 252 - RNA, 25 nt, Artificial Sequence
  Synthetic
ugaggugggaguacugcaaacugac

SEQ ID NO: 253 - RNA, 24 nt, Artificial Sequence
  Synthetic
cugacacaaugcucagaaaaagaa

SEQ ID NO: 254 - RNA, 24 nt, Artificial Sequence
  Synthetic
ugacacaaugcucagaaacagaac

SEQ ID NO: 255 - RNA, 25 nt, Artificial Sequence
  Synthetic
gacacaaugcucagaaacaaaauca

SEQ ID NO: 256 - RNA, 25 nt, Artificial Sequence
  Synthetic
acacaaugcucagaaacagaaucag

SEQ ID NO: 257 - RNA, 25 nt, Artificial Sequence
  Synthetic
cacaaugcucagaaacagaaucagg

SEQ ID NO: 258 - RNA, 24 nt, Artificial Sequence
  Synthetic
acaaugcucagaaacagaaacagg

SEQ ID NO: 259 - RNA, 24 nt, Artificial Sequence
  Synthetic
caaugcucagaaacagaauaaggg

SEQ ID NO: 260 - RNA, 24 nt, Artificial Sequence
  Synthetic
aaugcucagaaacagaaucaggug

SEQ ID NO: 261 - RNA, 24 nt, Artificial Sequence
  Synthetic
augcucagaaacagaaucaagugc

SEQ ID NO: 262 - RNA, 25 nt, Artificial Sequence
  Synthetic
ugcucagaaacagaaucagaugucc

SEQ ID NO: 263 - RNA, 24 nt, Artificial Sequence
  Synthetic
cucagaaacagaaucagguaucca

SEQ ID NO: 264 - RNA, 25 nt, Artificial Sequence
  Synthetic
cagaaacagaaucagguguacuaga

SEQ ID NO: 265 - RNA, 25 nt, Artificial Sequence
  Synthetic
agaaacagaaucaggugucauagag

SEQ ID NO: 266 - RNA, 25 nt, Artificial Sequence
  Synthetic
gaaacagaaucagguguccaagaga

SEQ ID NO: 267 - RNA, 25 nt, Artificial Sequence
  Synthetic
aaacagaaucagguguccuagagac

SEQ ID NO: 268 - RNA, 24 nt, Artificial Sequence
  Synthetic
aacagaaucagguguccuaaagac

SEQ ID NO: 269 - RNA, 25 nt, Artificial Sequence
  Synthetic
cagaaucagguguccuagaaacucc

SEQ ID NO: 270 - RNA, 25 nt, Artificial Sequence
  Synthetic
gaaucagguguccuagagaauccca

SEQ ID NO: 271 - RNA, 24 nt, Artificial Sequence
  Synthetic
aucagguguccuagagacuaccac

SEQ ID NO: 272 - RNA, 24 nt, Artificial Sequence
  Synthetic
gguguccuagagacucccaaugug

SEQ ID NO: 273 - RNA, 25 nt, Artificial Sequence
  Synthetic
ccuagagacucccacuguuauucca

SEQ ID NO: 274 - RNA, 25 nt, Artificial Sequence
  Synthetic
cuagagacucccacuguugauccag

SEQ ID NO: 275 - RNA, 24 nt, Artificial Sequence
  Synthetic
uagagacucccacuguuguaccag

SEQ ID NO: 276 - RNA, 23 nt, Artificial Sequence
  Synthetic
agagacucccacuguuguuacag

SEQ ID NO: 277 - RNA, 24 nt, Artificial Sequence
  Synthetic
gagacucccacuguuguucaaguc

SEQ ID NO: 278 - RNA, 25 nt, Artificial Sequence
  Synthetic
agacucccacuguuguuccaguucc

SEQ ID NO: 279 - RNA, 24 nt, Artificial Sequence
  Synthetic
gcucauucugaagcagcacaaacg

SEQ ID NO: 280 - RNA, 25 nt, Artificial Sequence
  Synthetic
cucauucugaagcagcaccaacuga

SEQ ID NO: 281 - RNA, 25 nt, Artificial Sequence
  Synthetic
ucauucugaagcagcaccaacugag

SEQ ID NO: 282 - RNA, 25 nt, Artificial Sequence
  Synthetic
cauucugaagcagcaccaaaugagc

SEQ ID NO: 283 - RNA, 25 nt, Artificial Sequence
  Synthetic
auucugaagcagcaccaacagagca

SEQ ID NO: 284 - RNA, 25 nt, Artificial Sequence
  Synthetic
uucugaagcagcaccaacuaagcaa

SEQ ID NO: 285 - RNA, 25 nt, Artificial Sequence
  Synthetic
ucugaagcagcaccaacugagcaaa

SEQ ID NO: 286 - RNA, 25 nt, Artificial Sequence
  Synthetic
cugaagcagcaccaacugaacaaac

SEQ ID NO: 287 - RNA, 25 nt, Artificial Sequence
  Synthetic
ugaagcagcaccaacugagaaaacc

SEQ ID NO: 288 - RNA, 25 nt, Artificial Sequence
  Synthetic
gaagcagcaccaacugagcaaaccc

SEQ ID NO: 289 - RNA, 25 nt, Artificial Sequence
  Synthetic
aagcagcaccaacugagcaaacccc

SEQ ID NO: 290 - RNA, 25 nt, Artificial Sequence
  Synthetic
cagugcuaccaugguaaugaccaga

SEQ ID NO: 291 - RNA, 25 nt, Artificial Sequence
  Synthetic
agugcuaccaugguaauggacagag

SEQ ID NO: 292 - RNA, 25 nt, Artificial Sequence
  Synthetic
acauucuccaccacugucaaaggaa

SEQ ID NO: 293 - RNA, 25 nt, Artificial Sequence
  Synthetic
cacugucacaggaaggacaagucaa

SEQ ID NO: 294 - RNA, 24 nt, Artificial Sequence
  Synthetic
acugucacaggaaggacauaucaa

SEQ ID NO: 295 - RNA, 24 nt, Artificial Sequence
  Synthetic
cugucacaggaaggacaugacaac

SEQ ID NO: 296 - RNA, 24 nt, Artificial Sequence
  Synthetic
ugucacaggaaggacauguaaauc

SEQ ID NO: 297 - RNA, 23 nt, Artificial Sequence
  Synthetic
gucacaggaaggacaugucaauc

SEQ ID NO: 298 - RNA, 24 nt, Artificial Sequence
  Synthetic
ucacaggaaggacaugucaaucug

SEQ ID NO: 299 - RNA, 24 nt, Artificial Sequence
  Synthetic
acaggaaggacaugucaauauugg

SEQ ID NO: 300 - RNA, 24 nt, Artificial Sequence
  Synthetic
caggaaggacaugucaaucauggc

SEQ ID NO: 301 - RNA, 25 nt, Artificial Sequence
  Synthetic
aggaaggacaugucaaucuagguca

SEQ ID NO: 302 - RNA, 24 nt, Artificial Sequence
  Synthetic
ggaaggacaugucaaucuuaguca

SEQ ID NO: 303 - RNA, 24 nt, Artificial Sequence
  Synthetic
gaaggacaugucaaucuugaucac

SEQ ID NO: 304 - RNA, 25 nt, Artificial Sequence
  Synthetic
aaggacaugucaaucuuggacaucc

SEQ ID NO: 305 - RNA, 25 nt, Artificial Sequence
  Synthetic
aggacaugucaaucuugguaaucca

SEQ ID NO: 306 - RNA, 24 nt, Artificial Sequence
  Synthetic
ggacaugucaaucuuggucaucca

SEQ ID NO: 307 - RNA, 24 nt, Artificial Sequence
  Synthetic
gacaugucaaucuuggucaaccag

SEQ ID NO: 308 - RNA, 25 nt, Artificial Sequence
  Synthetic
acaugucaaucuuggucauacauga

SEQ ID NO: 309 - RNA, 25 nt, Artificial Sequence
  Synthetic
caugucaaucuuggucaucaaugac

SEQ ID NO: 310 - RNA, 25 nt, Artificial Sequence
  Synthetic
augucaaucuuggucauccaugaca

SEQ ID NO: 311 - RNA, 25 nt, Artificial Sequence
  Synthetic
ugucaaucuuggucauccaagacac

SEQ ID NO: 312 - RNA, 25 nt, Artificial Sequence
  Synthetic
gucaaucuuggucauccauaacacc

SEQ ID NO: 313 - RNA, 25 nt, Artificial Sequence
  Synthetic
ucaaucuuggucauccaugacacca

SEQ ID NO: 314 - RNA, 25 nt, Artificial Sequence
  Synthetic
caaucuuggucauccaugaaaccac

SEQ ID NO: 315 - RNA, 25 nt, Artificial Sequence
  Synthetic
aaucuuggucauccaugacaccaca

SEQ ID NO: 316 - RNA, 25 nt, Artificial Sequence
  Synthetic
aucuuggucauccaugacaacacac

SEQ ID NO: 317 - RNA, 25 nt, Artificial Sequence
  Synthetic
ugacaaugaacuacugcagaaaucc

SEQ ID NO: 318 - RNA, 25 nt, Artificial Sequence
  Synthetic
gacaaugaacuacugcaggaaucca

SEQ ID NO: 319 - RNA, 25 nt, Artificial Sequence
  Synthetic
acaaugaacuacugcaggaauccag

SEQ ID NO: 320 - RNA, 25 nt, Artificial Sequence
  Synthetic
caaugaacuacugcaggaaaccaga

SEQ ID NO: 321 - RNA, 24 nt, Artificial Sequence
  Synthetic
aaugaacuacugcaggaauacaga

SEQ ID NO: 322 - RNA, 24 nt, Artificial Sequence
  Synthetic
augaacuacugcaggaaucaagag

SEQ ID NO: 323 - RNA, 25 nt, Artificial Sequence
  Synthetic
ugaacuacugcaggaauccagaugc

SEQ ID NO: 324 - RNA, 24 nt, Artificial Sequence
  Synthetic
cuacugcaggaauccagauaccga

SEQ ID NO: 325 - RNA, 25 nt, Artificial Sequence
  Synthetic
caggcccuugguguuuuacaaugga

SEQ ID NO: 326 - RNA, 25 nt, Artificial Sequence
  Synthetic
cuugguguuuuaccauggaacccag

SEQ ID NO: 327 - RNA, 25 nt, Artificial Sequence
  Synthetic
uugguguuuuaccauggacaccagc

SEQ ID NO: 328 - RNA, 25 nt, Artificial Sequence
  Synthetic
ugguguuuuaccauggaccacagca

SEQ ID NO: 329 - RNA, 24 nt, Artificial Sequence
  Synthetic
gguguuuuaccauggacccaagca

SEQ ID NO: 330 - RNA, 24 nt, Artificial Sequence
  Synthetic
guguuuuaccauggaccccagcac

SEQ ID NO: 331 - RNA, 25 nt, Artificial Sequence
  Synthetic
guuuuaccauggaccccagaaucag

SEQ ID NO: 332 - RNA, 25 nt, Artificial Sequence
  Synthetic
ggaguacugcaaccugacgagaugc

SEQ ID NO: 333 - RNA, 24 nt, Artificial Sequence
  Synthetic
gaguacugcaaccugacgcaaugc

SEQ ID NO: 334 - RNA, 25 nt, Artificial Sequence
  Synthetic
guacugcaaccugacgcgaagcuca

SEQ ID NO: 335 - RNA, 25 nt, Artificial Sequence
  Synthetic
uacugcaaccugacgcgauacucag

SEQ ID NO: 336 - RNA, 25 nt, Artificial Sequence
  Synthetic
ugcaaccugacgcgaugcuaagaca

SEQ ID NO: 337 - RNA, 25 nt, Artificial Sequence
  Synthetic
ccugacgcgaugcucagacacagaa

SEQ ID NO: 338 - RNA, 25 nt, Artificial Sequence
  Synthetic
cugacgcgaugcucagacaaagaag

SEQ ID NO: 339 - RNA, 24 nt, Artificial Sequence
  Synthetic
gaugcucagacacagaaggaacug

SEQ ID NO: 340 - RNA, 24 nt, Artificial Sequence
  Synthetic
augcucagacacagaagggacugg

SEQ ID NO: 341 - RNA, 24 nt, Artificial Sequence
  Synthetic
agacacagaagggacugugaucgc

SEQ ID NO: 342 - RNA, 25 nt, Artificial Sequence
  Synthetic
gcauccucuucauuugauuauggga

SEQ ID NO: 343 - RNA, 25 nt, Artificial Sequence
  Synthetic
cauccucuucauuugauugagggaa

SEQ ID NO: 344 - RNA, 25 nt, Artificial Sequence
  Synthetic
auccucuucauuugauuguaggaag

SEQ ID NO: 345 - RNA, 25 nt, Artificial Sequence
  Synthetic
uccucuucauuugauugugagaagc

SEQ ID NO: 346 - RNA, 25 nt, Artificial Sequence
  Synthetic
ccucuucauuugauuguggaaagcc

SEQ ID NO: 347 - RNA, 25 nt, Artificial Sequence
  Synthetic
cuucauuugauugugggaaaccuca

SEQ ID NO: 348 - RNA, 25 nt, Artificial Sequence
  Synthetic
uucauuugauugugggaagacucaa

SEQ ID NO: 349 - RNA, 25 nt, Artificial Sequence
  Synthetic
ucauuugauugugggaagcaucaag

SEQ ID NO: 350 - RNA, 24 nt, Artificial Sequence
  Synthetic
cauuugauugugggaagccacaag

SEQ ID NO: 351 - RNA, 24 nt, Artificial Sequence
  Synthetic
auuugauugugggaagccuaaagg

SEQ ID NO: 352 - RNA, 25 nt, Artificial Sequence
  Synthetic
gugggaagccucaaguggaaccgaa

SEQ ID NO: 353 - RNA, 25 nt, Artificial Sequence
  Synthetic
aagaaauguccuggaagcaauguag

SEQ ID NO: 354 - RNA, 25 nt, Artificial Sequence
  Synthetic
agaaauguccuggaagcauaguagg

SEQ ID NO: 355 - RNA, 25 nt, Artificial Sequence
  Synthetic
gaaauguccuggaagcauuauaggg

SEQ ID NO: 356 - RNA, 25 nt, Artificial Sequence
  Synthetic
aauguccuggaagcauuguaggggg

SEQ ID NO: 357 - RNA, 25 nt, Artificial Sequence
  Synthetic
auguccuggaagcauuguaaggggg

SEQ ID NO: 358 - RNA, 24 nt, Artificial Sequence
  Synthetic
agaacaagguuuggaaagcacuuc

SEQ ID NO: 359 - RNA, 24 nt, Artificial Sequence
  Synthetic
gaacaagguuuggaaagcaauucg

SEQ ID NO: 360 - RNA, 24 nt, Artificial Sequence
  Synthetic
aacaagguuuggaaagcacaucug

SEQ ID NO: 361 - RNA, 24 nt, Artificial Sequence
  Synthetic
acaagguuuggaaagcacuacugg

SEQ ID NO: 362 - RNA, 25 nt, Artificial Sequence
  Synthetic
caagguuuggaaagcacuuaugugg

SEQ ID NO: 363 - RNA, 25 nt, Artificial Sequence
  Synthetic
aagguuuggaaagcacuucagugga

SEQ ID NO: 364 - RNA, 25 nt, Artificial Sequence
  Synthetic
agguuuggaaagcacuucuauggag

SEQ ID NO: 365 - RNA, 25 nt, Artificial Sequence
  Synthetic
uggaaagcacuucuguggaagcacc

SEQ ID NO: 366 - RNA, 25 nt, Artificial Sequence
  Synthetic
guggaggcaccuuaauaucaccaga

SEQ ID NO: 367 - RNA, 24 nt, Artificial Sequence
  Synthetic
cuuaauauccccagaguggaugcg

SEQ ID NO: 368 - RNA, 25 nt, Artificial Sequence
  Synthetic
uaauauccccagaguggguacugac

SEQ ID NO: 369 - RNA, 25 nt, Artificial Sequence
  Synthetic
agagugggugcugacugcuacucac

SEQ ID NO: 370 - RNA, 25 nt, Artificial Sequence
  Synthetic
caaggucauccugggugcaaaccaa

SEQ ID NO: 371 - RNA, 25 nt, Artificial Sequence
  Synthetic
ccugggugcacaccaagaaaugaac

SEQ ID NO: 372 - RNA, 25 nt, Artificial Sequence
  Synthetic
gugcacaccaagaagugaaacucga

SEQ ID NO: 373 - RNA, 24 nt, Artificial Sequence
  Synthetic
agcagauauugccuugcuaaagca

SEQ ID NO: 374 - RNA, 25 nt, Artificial Sequence
  Synthetic
gcagauauugccuugcuaaagcuaa

SEQ ID NO: 375 - RNA, 25 nt, Artificial Sequence
  Synthetic
cagauauugccuugcuaaaacuaag

SEQ ID NO: 376 - RNA, 25 nt, Artificial Sequence
  Synthetic
agauauugccuugcuaaagauaagc

SEQ ID NO: 377 - RNA, 25 nt, Artificial Sequence
  Synthetic
gauauugccuugcuaaagcaaagca

SEQ ID NO: 378 - RNA, 25 nt, Artificial Sequence
  Synthetic
auauugccuugcuaaagcuaagcag

SEQ ID NO: 379 - RNA, 25 nt, Artificial Sequence
  Synthetic
uauugccuugcuaaagcuaagcagg

SEQ ID NO: 380 - RNA, 25 nt, Artificial Sequence
  Synthetic
ucaucacugacaaaguaauaccagc

SEQ ID NO: 381 - RNA, 24 nt, Artificial Sequence
  Synthetic
ggacugaauguuacaucacaggcg

SEQ ID NO: 382 - RNA, 25 nt, Artificial Sequence
  Synthetic
gacugaauguuacaucacuagcugg

SEQ ID NO: 383 - RNA, 25 nt, Artificial Sequence
  Synthetic
acugaauguuacaucacugacuggg

SEQ ID NO: 384 - RNA, 25 nt, Artificial Sequence
  Synthetic
cugaauguuacaucacuggaugggg

SEQ ID NO: 385 - RNA, 25 nt, Artificial Sequence
  Synthetic
ugaauguuacaucacuggcagggga

SEQ ID NO: 386 - RNA, 25 nt, Artificial Sequence
  Synthetic
aauguuacaucacuggcugaggaga

SEQ ID NO: 387 - RNA, 24 nt, Artificial Sequence
  Synthetic
gaaacccaagguaccuuugagacg

SEQ ID NO: 388 - RNA, 36 nt, Artificial Sequence
  Synthetic
uccaccacugucacaggaaagcagccgaaaggcugc

SEQ ID NO: 389 - RNA, 36 nt, Artificial Sequence
  Synthetic
ugguaauggacagaguuauagcagccgaaaggcugc

SEQ ID NO: 390 - RNA, 36 nt, Artificial Sequence
  Synthetic
uacugcaaccugacacgauagcagccgaaaggcugc

SEQ ID NO: 391 - RNA, 36 nt, Artificial Sequence
  Synthetic
agaacuugccaagcuugguagcagccgaaaggcugc

SEQ ID NO: 392 - RNA, 36 nt, Artificial Sequence
  Synthetic
aacuugccaagcuuggucaagcagccgaaaggcugc

SEQ ID NO: 393 - RNA, 36 nt, Artificial Sequence
  Synthetic
uugccaagcuuggucaucuagcagccgaaaggcugc

SEQ ID NO: 394 - RNA, 36 nt, Artificial Sequence
  Synthetic
auggacagaguuaucgaggagcagccgaaaggcugc

SEQ ID NO: 395 - RNA, 36 nt, Artificial Sequence
  Synthetic
uggacagaguuaucgaggcagcagccgaaaggcugc

SEQ ID NO: 396 - RNA, 36 nt, Artificial Sequence
  Synthetic
uggucaucuaugauaccacagcagccgaaaggcugc

SEQ ID NO: 397 - RNA, 36 nt, Artificial Sequence
  Synthetic
uacugcaggaauccagauuagcagccgaaaggcugc

SEQ ID NO: 398 - RNA, 36 nt, Artificial Sequence
  Synthetic
agaaauguccuggaagcauagcagccgaaaggcugc

SEQ ID NO: 399 - RNA, 36 nt, Artificial Sequence
  Synthetic
gacaacagaauauuauccaagcagccgaaaggcugc

SEQ ID NO: 400 - RNA, 36 nt, Artificial Sequence
  Synthetic
auggacagaguuaucaaggagcagccgaaaggcugc

SEQ ID NO: 401 - RNA, 36 nt, Artificial Sequence; identical to SEQ ID NO: 399
  Synthetic

SEQ ID NO: 402 - RNA, 36 nt, Artificial Sequence; identical to SEQ ID NO: 400
  Synthetic

SEQ ID NO: 403 - RNA, 36 nt, Artificial Sequence; identical to SEQ ID NO: 399
  Synthetic

SEQ ID NO: 404 - RNA, 27 nt, Artificial Sequence
  Synthetic
uccuguaccacauggcuuugcucaggu

SEQ ID NO: 405 - RNA, 27 nt, Artificial Sequence
  Synthetic
caaucuuggaccacauggcuuugcuca

SEQ ID NO: 406 - RNA, 27 nt, Artificial Sequence
  Synthetic
guagcuauccuggaccacauggcuuug

SEQ ID NO: 407 - RNA, 27 nt, Artificial Sequence
  Synthetic
gguaguaauccuggaccacauggcuuu

SEQ ID NO: 408 - RNA, 27 nt, Artificial Sequence
  Synthetic
ugguaucaauccuggaccacauggcuu

SEQ ID NO: 409 - RNA, 27 nt, Artificial Sequence
  Synthetic
augguugcaauccuggaccacauggcu

SEQ ID NO: 410 - RNA, 27 nt, Artificial Sequence
  Synthetic
caugguagcaauccuggaccacauggc

SEQ ID NO: 411 - RNA, 27 nt, Artificial Sequence
  Synthetic
ccauguuagcaauccuggaccacaugg

SEQ ID NO: 412 - RNA, 27 nt, Artificial Sequence
  Synthetic
accauuguagcaauccuggaccacaug

SEQ ID NO: 413 - RNA, 27 nt, Artificial Sequence
  Synthetic
ugccuugauaacucuguccaucaccau

SEQ ID NO: 414 - RNA, 27 nt, Artificial Sequence
  Synthetic
aggucuuuccugugacagugguggagu

SEQ ID NO: 415 - RNA, 27 nt, Artificial Sequence
  Synthetic
cagguucuuccugugacagugguggag

SEQ ID NO: 416 - RNA, 27 nt, Artificial Sequence
  Synthetic
gcuugucagguccuuccugugacagug

SEQ ID NO: 417 - RNA, 27 nt, Artificial Sequence
  Synthetic
augacuaagcuuggcagguccuuccug

SEQ ID NO: 418 - RNA, 27 nt, Artificial Sequence
  Synthetic
gaugaucaagcuuggcagguccuuccu

SEQ ID NO: 419 - RNA, 27 nt, Artificial Sequence
  Synthetic
uagauuaccaagcuuggcagguccuuc

SEQ ID NO: 420 - RNA, 27 nt, Artificial Sequence
  Synthetic
auagaugaccaagcuuggcagguccuu

SEQ ID NO: 421 - RNA, 27 nt, Artificial Sequence
  Synthetic
cauaguugaccaagcuuggcagguccu

SEQ ID NO: 422 - RNA, 27 nt, Artificial Sequence
  Synthetic
ucauauaugaccaagcuuggcaggucc

SEQ ID NO: 423 - RNA, 27 nt, Artificial Sequence
  Synthetic
gucauugaugaccaagcuuggcagguc

SEQ ID NO: 424 - RNA, 27 nt, Artificial Sequence
  Synthetic
auguguugucauagaugaccaagcuug

SEQ ID NO: 425 - RNA, 27 nt, Artificial Sequence
  Synthetic
auguuuauguggugucauagaugacca

SEQ ID NO: 426 - RNA, 27 nt, Artificial Sequence
  Synthetic
gccaguauuuggguaguuuucuguggu

SEQ ID NO: 427 - RNA, 27 nt, Artificial Sequence
  Synthetic
caagcuagcauuuggguaguuuucugu

SEQ ID NO: 428 - RNA, 27 nt, Artificial Sequence
  Synthetic
aucaauccagcauuuggguaguuuucu

SEQ ID NO: 429 - RNA, 27 nt, Artificial Sequence
  Synthetic
uucauuaucaagccagcauuuggguag

SEQ ID NO: 430 - RNA, 27 nt, Artificial Sequence
  Synthetic
guucaugaucaagccagcauuugggua

SEQ ID NO: 431 - RNA, 27 nt, Artificial Sequence
  Synthetic
agcauuuggauuccugcaguaguucau

SEQ ID NO: 432 - RNA, 27 nt, Artificial Sequence
  Synthetic
ccacaucaucuggauuccugcaguagu

SEQ ID NO: 433 - RNA, 27 nt, Artificial Sequence
  Synthetic
gccacugcaucuggauuccugcaguag

SEQ ID NO: 434 - RNA, 27 nt, Artificial Sequence
  Synthetic
cugccucagcaucuggauuccugcagu

SEQ ID NO: 435 - RNA, 27 nt, Artificial Sequence
  Synthetic
gcgucugguugcaguacucccaccuga

SEQ ID NO: 436 - RNA, 27 nt, Artificial Sequence
  Synthetic
gcaguuccuucugcgucugagcauugc

SEQ ID NO: 437 - RNA, 27 nt, Artificial Sequence
  Synthetic
uaggcuuggaaccgggguaacagucgg

SEQ ID NO: 438 - RNA, 27 nt, Artificial Sequence
  Synthetic
cuagguuuggaaccgggguaacagucg

SEQ ID NO: 439 - RNA, 27 nt, Artificial Sequence
  Synthetic
ucuagucuuggaaccgggguaacaguc

SEQ ID NO: 440 - RNA, 27 nt, Artificial Sequence
  Synthetic
cucuaugcuuggaaccgggguaacagu

SEQ ID NO: 441 - RNA, 27 nt, Artificial Sequence
  Synthetic
ccucuuggcuuggaaccgggguaacag

SEQ ID NO: 442 - RNA, 27 nt, Artificial Sequence
  Synthetic
gccucuaggcuuggaaccgggguaaca

SEQ ID NO: 443 - RNA, 27 nt, Artificial Sequence
  Synthetic
agccuuuaggcuuggaaccgggguaac

SEQ ID NO: 444 - RNA, 27 nt, Artificial Sequence
  Synthetic
acucuuuccauuaccaugguagcacuc

SEQ ID NO: 445 - RNA, 27 nt, Artificial Sequence
  Synthetic
aacucuguccauuaccaugguagcacu

SEQ ID NO: 446 - RNA, 27 nt, Artificial Sequence
  Synthetic
uaacuuuguccauuaccaugguagcac

SEQ ID NO: 447 - RNA, 27 nt, Artificial Sequence
  Synthetic
auaacucuguccauuaccaugguagca

SEQ ID NO: 448 - RNA, 27 nt, Artificial Sequence
  Synthetic
gauaauucuguccauuaccaugguagc

SEQ ID NO: 449 - RNA, 27 nt, Artificial Sequence
  Synthetic
cgauaucucuguccauuaccaugguag

SEQ ID NO: 450 - RNA, 27 nt, Artificial Sequence
  Synthetic
ucgauuacucuguccauuaccauggua

SEQ ID NO: 451 - RNA, 27 nt, Artificial Sequence
  Synthetic
cucgauaacucuguccauuaccauggu

SEQ ID NO: 452 - RNA, 27 nt, Artificial Sequence
  Synthetic
ccucguuaacucuguccauuaccaugg

SEQ ID NO: 453 - RNA, 27 nt, Artificial Sequence
  Synthetic
gccucuauaacucuguccauuaccaug

SEQ ID NO: 454 - RNA, 27 nt, Artificial Sequence
  Synthetic
ugccuugauaacucuguccauuaccau

SEQ ID NO: 455 - RNA, 27 nt, Artificial Sequence
  Synthetic
gugccucgauaacucuguccauuacca

SEQ ID NO: 456 - RNA, 27 nt, Artificial Sequence
  Synthetic
cugugucagugguggaguaugugccuc

SEQ ID NO: 457 - RNA, 27 nt, Artificial Sequence
  Synthetic
gaguguggugucauagaugaccaagcu

SEQ ID NO: 458 - RNA, 27 nt, Artificial Sequence
  Synthetic
augcguguguggugucauagaugacca

SEQ ID NO: 459 - RNA, 27 nt, Artificial Sequence
  Synthetic
uaugcuaguguggugucauagaugacc

SEQ ID NO: 460 - RNA, 27 nt, Artificial Sequence
  Synthetic
cuaugugaguguggugucauagaugac

SEQ ID NO: 461 - RNA, 27 nt, Artificial Sequence
  Synthetic
ccaguuacagugguggaguaugugccu

SEQ ID NO: 462 - RNA, 27 nt, Artificial Sequence
  Synthetic
aucccucguauaacaauaaggggcugc

SEQ ID NO: 463 - RNA, 27 nt, Artificial Sequence
  Synthetic
gauccuucguauaacaauaaggggcug

SEQ ID NO: 464 - RNA, 27 nt, Artificial Sequence
  Synthetic
guucauaaggagccucuaggcuuggaa

SEQ ID NO: 465 - RNA, 27 nt, Artificial Sequence
  Synthetic
guuggugcuuguucagaaggagccucu

SEQ ID NO: 466 - RNA, 27 nt, Artificial Sequence
  Synthetic
uauguuccuugauaacucuguccauuu

SEQ ID NO: 467 - RNA, 27 nt, Artificial Sequence
  Synthetic
guaugugccuugauaacucuguccauu

SEQ ID NO: 468 - RNA, 27 nt, Artificial Sequence
  Synthetic
aguauuugccuugauaacucuguccau

SEQ ID NO: 469 - RNA, 27 nt, Artificial Sequence
  Synthetic
aaguaugugccuugauaacucugucca

SEQ ID NO: 470 - RNA, 27 nt, Artificial Sequence
  Synthetic
gaaguuugugccuugauaacucugucc

SEQ ID NO: 471 - RNA, 27 nt, Artificial Sequence
  Synthetic
ugaaguaugugccuugauaacucuguc

SEQ ID NO: 472 - RNA, 27 nt, Artificial Sequence
  Synthetic
ucuuguucaagccagcauuuggguagu

SEQ ID NO: 473 - RNA, 27 nt, Artificial Sequence
  Synthetic
aguucuugaucaagccagcauuugggu

SEQ ID NO: 474 - RNA, 27 nt, Artificial Sequence
  Synthetic
ucugguuuucggcaguaguucuugauc

SEQ ID NO: 475 - RNA, 27 nt, Artificial Sequence
  Synthetic
aucuguauuucggcaguaguucuugau

SEQ ID NO: 476 - RNA, 27 nt, Artificial Sequence
  Synthetic
gaucuugauuucggcaguaguucuuga

SEQ ID NO: 477 - RNA, 27 nt, Artificial Sequence
  Synthetic
aggauuuggauuucggcaguaguucuu

SEQ ID NO: 478 - RNA, 27 nt, Artificial Sequence
  Synthetic
cacaguaucuggauuucggcaguaguu

SEQ ID NO: 479 - RNA, 27 nt, Artificial Sequence
  Synthetic
uguauuacaccaaggggcugccacagg

SEQ ID NO: 480 - RNA, 27 nt, Artificial Sequence
  Synthetic
uuguauaacaccaaggggcugccacag

SEQ ID NO: 481 - RNA, 27 nt, Artificial Sequence
  Synthetic
guuguuuaacaccaaggggcugccaca

SEQ ID NO: 482 - RNA, 27 nt, Artificial Sequence
  Synthetic
uguuguauaacaccaaggggcugccac

SEQ ID NO: 483 - RNA, 27 nt, Artificial Sequence
  Synthetic
cuguuuuauaacaccaaggggcugcca

SEQ ID NO: 484 - RNA, 27 nt, Artificial Sequence
  Synthetic
ucuguuguauaacaccaaggggcugcc

SEQ ID NO: 485 - RNA, 27 nt, Artificial Sequence
  Synthetic
aucuguuguauaacaccaaggggcugc

SEQ ID NO: 486 - RNA, 27 nt, Artificial Sequence
  Synthetic
gaucuuuuguauaacaccaaggggcug

SEQ ID NO: 487 - RNA, 27 nt, Artificial Sequence
  Synthetic
ggaucuguuguauaacaccaaggggcu

SEQ ID NO: 488 - RNA, 27 nt, Artificial Sequence
  Synthetic
cguguuagguugcaguacucccaccug

SEQ ID NO: 489 - RNA, 27 nt, Artificial Sequence
  Synthetic
ucgugucagguugcaguacucccaccu

SEQ ID NO: 490 - RNA, 27 nt, Artificial Sequence
  Synthetic
gcaucuugucagguugcaguacuccca

SEQ ID NO: 491 - RNA, 27 nt, Artificial Sequence
  Synthetic
agcauugugucagguugcaguacuccc

SEQ ID NO: 492 - RNA, 27 nt, Artificial Sequence
  Synthetic
ugagcuucgugucagguugcaguacuc

SEQ ID NO: 493 - RNA, 27 nt, Artificial Sequence
  Synthetic
cugaguaucgugucagguugcaguacu

SEQ ID NO: 494 - RNA, 27 nt, Artificial Sequence
  Synthetic
ucugaucaucgugucagguugcaguac

SEQ ID NO: 495 - RNA, 27 nt, Artificial Sequence
  Synthetic
aucugugcaucgugucagguugcagua

SEQ ID NO: 496 - RNA, 27 nt, Artificial Sequence
  Synthetic
caucuuagcaucgugucagguugcagu

SEQ ID NO: 497 - RNA, 27 nt, Artificial Sequence
  Synthetic
ugcauuugagcaucgugucagguugca

SEQ ID NO: 498 - RNA, 27 nt, Artificial Sequence
  Synthetic
ucugcuucugagcaucgugucagguug

SEQ ID NO: 499 - RNA, 27 nt, Artificial Sequence
  Synthetic
uucuguaucugagcaucgugucagguu

SEQ ID NO: 500 - RNA, 27 nt, Artificial Sequence
  Synthetic
auucuucaucugagcaucgugucaggu

SEQ ID NO: 501 - RNA, 27 nt, Artificial Sequence
  Synthetic
cauucugcaucugagcaucgugucagg

SEQ ID NO: 502 - RNA, 27 nt, Artificial Sequence
  Synthetic
ccauuuugcaucugagcaucgugucag

SEQ ID NO: 503 - RNA, 27 nt, Artificial Sequence
  Synthetic
uccauucugcaucugagcaucguguca

SEQ ID NO: 504 - RNA, 27 nt, Artificial Sequence
  Synthetic
guccauucugcaucugagcaucguguc

SEQ ID NO: 505 - RNA, 27 nt, Artificial Sequence
  Synthetic
aacucuguccauaaugguaguagcagu

SEQ ID NO: 506 - RNA, 27 nt, Artificial Sequence
  Synthetic
uaacuuuguccauaaugguaguagcag

SEQ ID NO: 507 - RNA, 27 nt, Artificial Sequence
  Synthetic
guaacucuguccauaaugguaguagca

SEQ ID NO: 508 - RNA, 27 nt, Artificial Sequence
  Synthetic
gguaauucuguccauaaugguaguagc

SEQ ID NO: 509 - RNA, 27 nt, Artificial Sequence
  Synthetic
cgguaucucuguccauaaugguaguag

SEQ ID NO: 510 - RNA, 27 nt, Artificial Sequence
  Synthetic
ucgguuacucuguccauaaugguagua

SEQ ID NO: 511 - RNA, 27 nt, Artificial Sequence
  Synthetic
cucgguaacucuguccauaaugguagu

SEQ ID NO: 512 - RNA, 27 nt, Artificial Sequence
  Synthetic
ccucguuaacucuguccauaaugguag

SEQ ID NO: 513 - RNA, 27 nt, Artificial Sequence
  Synthetic
gugacugugguggaguaugugccucgg

SEQ ID NO: 514 - RNA, 27 nt, Artificial Sequence
  Synthetic
aguucuuccugugacagugguggagua

SEQ ID NO: 515 - RNA, 27 nt, Artificial Sequence
  Synthetic
accaaucuuggcaaguucuuccuguga

SEQ ID NO: 516 - RNA, 27 nt, Artificial Sequence
  Synthetic
gaccaugcuuggcaaguucuuccugug

SEQ ID NO: 517 - RNA, 27 nt, Artificial Sequence
  Synthetic
ugaccuagcuuggcaaguucuuccugu

SEQ ID NO: 518 - RNA, 27 nt, Artificial Sequence
  Synthetic
augacuaagcuuggcaaguucuuccug

SEQ ID NO: 519 - RNA, 27 nt, Artificial Sequence
  Synthetic
gaugaucaagcuuggcaaguucuuccu

SEQ ID NO: 520 - RNA, 27 nt, Artificial Sequence
  Synthetic
agauguccaagcuuggcaaguucuucc

SEQ ID NO: 521 - RNA, 27 nt, Artificial Sequence
  Synthetic
uagauuaccaagcuuggcaaguucuuc

SEQ ID NO: 522 - RNA, 27 nt, Artificial Sequence
  Synthetic
auagaugaccaagcuuggcaaguucuu

SEQ ID NO: 523 - RNA, 27 nt, Artificial Sequence
  Synthetic
cauaguugaccaagcuuggcaaguucu

SEQ ID NO: 524 - RNA, 27 nt, Artificial Sequence
  Synthetic
ucauauaugaccaagcuuggcaaguuc

SEQ ID NO: 525 - RNA, 27 nt, Artificial Sequence
  Synthetic
gucauugaugaccaagcuuggcaaguu

SEQ ID NO: 526 - RNA, 27 nt, Artificial Sequence
  Synthetic
ugucauagaugaccaagcuuggcaagu

SEQ ID NO: 527 - RNA, 27 nt, Artificial Sequence
  Synthetic
gugucuuagaugaccaagcuuggcaag

SEQ ID NO: 528 - RNA, 27 nt, Artificial Sequence
  Synthetic
gguguugugucauagaugaccaagcuu

SEQ ID NO: 529 - RNA, 27 nt, Artificial Sequence
  Synthetic
cugguuuggugucauagaugaccaagc

SEQ ID NO: 530 - RNA, 27 nt, Artificial Sequence
  Synthetic
gcugguguggugucauagaugaccaag

SEQ ID NO: 531 - RNA, 27 nt, Artificial Sequence
  Synthetic
augcuuguguggugucauagaugacca

SEQ ID NO: 532 - RNA, 27 nt, Artificial Sequence
  Synthetic
cuauguugguguggugucauagaugac

SEQ ID NO: 533 - RNA, 27 nt, Artificial Sequence
  Synthetic
acuauucugguguggugucauagauga

SEQ ID NO: 534 - RNA, 27 nt, Artificial Sequence
  Synthetic
cgacuuugcugguguggugucauagau

SEQ ID NO: 535 - RNA, 27 nt, Artificial Sequence
  Synthetic
uuugguuaguuuucugggguccgacua

SEQ ID NO: 536 - RNA, 27 nt, Artificial Sequence
  Synthetic
auuuguguaguuuucugggguccgacu

SEQ ID NO: 537 - RNA, 27 nt, Artificial Sequence
  Synthetic
ucaggucagcauuuggguaguuuucug

SEQ ID NO: 538 - RNA, 27 nt, Artificial Sequence
  Synthetic
uguaauaccaagggcgaaucucagcau

SEQ ID NO: 539 - RNA, 27 nt, Artificial Sequence
  Synthetic
guguaucaccaagggcgaaucucagca

SEQ ID NO: 540 - RNA, 27 nt, Artificial Sequence
  Synthetic
ugguguaacaccaagggcgaaucucag

SEQ ID NO: 541 - RNA, 27 nt, Artificial Sequence
  Synthetic
augguuuaacaccaagggcgaaucuca

SEQ ID NO: 542 - RNA, 27 nt, Artificial Sequence
  Synthetic
uccauuguguaacaccaagggcgaauc

SEQ ID NO: 543 - RNA, 27 nt, Artificial Sequence
  Synthetic
cuggguuccaugguguaacaccaaggg

SEQ ID NO: 544 - RNA, 27 nt, Artificial Sequence
  Synthetic
acugguauccaugguguaacaccaagg

SEQ ID NO: 545 - RNA, 27 nt, Artificial Sequence
  Synthetic
cacugugauccaugguguaacaccaag

SEQ ID NO: 546 - RNA, 27 nt, Artificial Sequence
  Synthetic
acacuuggauccaugguguaacaccaa

SEQ ID NO: 547 - RNA, 27 nt, Artificial Sequence
  Synthetic
ugacauugggauccaugguguaacacc

SEQ ID NO: 548 - RNA, 27 nt, Artificial Sequence
  Synthetic
ugagauuugcaaggacacuugauucug

SEQ ID NO: 549 - RNA, 27 nt, Artificial Sequence
  Synthetic
gugaguguugcaaggacacuugauucu

SEQ ID NO: 550 - RNA, 27 nt, Artificial Sequence
  Synthetic
cgugauaguugcaaggacacuugauuc

SEQ ID NO: 551 - RNA, 27 nt, Artificial Sequence
  Synthetic
aaugauccucgauaacucuguccauca

SEQ ID NO: 552 - RNA, 27 nt, Artificial Sequence
  Synthetic
gaaugugccucgauaacucuguccauc

SEQ ID NO: 553 - RNA, 27 nt, Artificial Sequence
  Synthetic
uugucuucugaugccagugugguguca

SEQ ID NO: 554 - RNA, 27 nt, Artificial Sequence
  Synthetic
auugguauccaugguauaacaccaagg

SEQ ID NO: 555 - RNA, 27 nt, Artificial Sequence
  Synthetic
cauugugauccaugguauaacaccaag

SEQ ID NO: 556 - RNA, 27 nt, Artificial Sequence
  Synthetic
acauuuggauccaugguauaacaccaa

SEQ ID NO: 557 - RNA, 27 nt, Artificial Sequence
  Synthetic
gacauugggauccaugguauaacacca

SEQ ID NO: 558 - RNA, 27 nt, Artificial Sequence
  Synthetic
gucaguuugcaguacucccaucugaca

SEQ ID NO: 559 - RNA, 27 nt, Artificial Sequence
  Synthetic
uguguuagguugcaguacucccaucug

SEQ ID NO: 560 - RNA, 27 nt, Artificial Sequence
  Synthetic
uugugucagguugcaguacucccaucu

SEQ ID NO: 561 - RNA, 27 nt, Artificial Sequence
  Synthetic
auuguuucagguugcaguacucccauc

SEQ ID NO: 562 - RNA, 27 nt, Artificial Sequence
  Synthetic
cauugugucagguugcaguacucccau

SEQ ID NO: 563 - RNA, 27 nt, Artificial Sequence
  Synthetic
cguuguugcuuguucagaaacagccgu

SEQ ID NO: 564 - RNA, 27 nt, Artificial Sequence
  Synthetic
gcuccuuuggugcuuguucagaaacag

SEQ ID NO: 565 - RNA, 27 nt, Artificial Sequence
  Synthetic
uguccuuccuguaacagugguggagaa

SEQ ID NO: 566 - RNA, 27 nt, Artificial Sequence
  Synthetic
augucuuuccuguaacagugguggaga

SEQ ID NO: 567 - RNA, 27 nt, Artificial Sequence
  Synthetic
cauguucuuccuguaacagugguggag

SEQ ID NO: 568 - RNA, 27 nt, Artificial Sequence
  Synthetic
gguucucugaugccaguguggugucau

SEQ ID NO: 569 - RNA, 27 nt, Artificial Sequence
  Synthetic
ugguuuucugaugccagugugguguca

SEQ ID NO: 570 - RNA, 27 nt, Artificial Sequence
  Synthetic
caggcuaccauuuggguaguauucugu

SEQ ID NO: 571 - RNA, 27 nt, Artificial Sequence
  Synthetic
ucaggucaccauuuggguaguauucug

SEQ ID NO: 572 - RNA, 27 nt, Artificial Sequence
  Synthetic
gucaguccaccauuuggguaguauucu

SEQ ID NO: 573 - RNA, 27 nt, Artificial Sequence
  Synthetic
uucaguuggugcuucuucagaaggaag

SEQ ID NO: 574 - RNA, 27 nt, Artificial Sequence
  Synthetic
uuucauuuggugcuucuucagaaggaa

SEQ ID NO: 575 - RNA, 27 nt, Artificial Sequence
  Synthetic
uuuucuguuggugcuucuucagaagga

SEQ ID NO: 576 - RNA, 27 nt, Artificial Sequence
  Synthetic
guuuuuaguuggugcuucuucagaagg

SEQ ID NO: 577 - RNA, 27 nt, Artificial Sequence
  Synthetic
uguuuucaguuggugcuucuucagaag

SEQ ID NO: 578 - RNA, 27 nt, Artificial Sequence
  Synthetic
cuguuuucaguuggugcuucuucagaa

SEQ ID NO: 579 - RNA, 27 nt, Artificial Sequence
  Synthetic
gcuguuuucaguuggugcuucuucaga

SEQ ID NO: 580 - RNA, 27 nt, Artificial Sequence
  Synthetic
ugcuguuuucaguuggugcuucuucag

SEQ ID NO: 581 - RNA, 27 nt, Artificial Sequence
  Synthetic
gugcuuuuuucaguuggugcuucuuca

SEQ ID NO: 582 - RNA, 27 nt, Artificial Sequence
  Synthetic
agugcuguuuucaguuggugcuucuuc

SEQ ID NO: 583 - RNA, 27 nt, Artificial Sequence
  Synthetic
caguguuguuuucaguuggugcuucuu

SEQ ID NO: 584 - RNA, 27 nt, Artificial Sequence
  Synthetic
ccaguucuguuuucaguuggugcuucu

SEQ ID NO: 585 - RNA, 27 nt, Artificial Sequence
  Synthetic
gccucuauaacucuguccaucaccucg

SEQ ID NO: 586 - RNA, 27 nt, Artificial Sequence
  Synthetic
uguucuuccugugauagugguggagag

SEQ ID NO: 587 - RNA, 27 nt, Artificial Sequence
  Synthetic
auguuuuuccugugauagugguggaga

SEQ ID NO: 588 - RNA, 27 nt, Artificial Sequence
  Synthetic
cauguucuuccugugauagugguggag

SEQ ID NO: 589 - RNA, 27 nt, Artificial Sequence
  Synthetic
acauguucuuccugugauaguggugga

SEQ ID NO: 590 - RNA, 27 nt, Artificial Sequence
  Synthetic
gacauuuucuuccugugauaguggugg

SEQ ID NO: 591 - RNA, 27 nt, Artificial Sequence
  Synthetic
ugacauguucuuccugugauaguggug

SEQ ID NO: 592 - RNA, 27 nt, Artificial Sequence
  Synthetic
cugacuuguucuuccugugauaguggu

SEQ ID NO: 593 - RNA, 27 nt, Artificial Sequence
  Synthetic
acugauauguucuuccugugauagugg

SEQ ID NO: 594 - RNA, 27 nt, Artificial Sequence
  Synthetic
gacugucauguucuuccugugauagug

SEQ ID NO: 595 - RNA, 27 nt, Artificial Sequence
  Synthetic
agacuuacauguucuuccugugauagu

SEQ ID NO: 596 - RNA, 27 nt, Artificial Sequence
  Synthetic
aagacugacauguucuuccugugauag

SEQ ID NO: 597 - RNA, 27 nt, Artificial Sequence
  Synthetic
caagauugacauguucuuccugugaua

SEQ ID NO: 598 - RNA, 27 nt, Artificial Sequence
  Synthetic
ccaagucugacauguucuuccugugau

SEQ ID NO: 599 - RNA, 27 nt, Artificial Sequence
  Synthetic
accaauacugacauguucuuccuguga

SEQ ID NO: 600 - RNA, 27 nt, Artificial Sequence
  Synthetic
acgacuaagacugacauguucuuccug

SEQ ID NO: 601 - RNA, 27 nt, Artificial Sequence
  Synthetic
gacgaucaagacugacauguucuuccu

SEQ ID NO: 602 - RNA, 27 nt, Artificial Sequence
  Synthetic
agacguccaagacugacauguucuucc

SEQ ID NO: 603 - RNA, 27 nt, Artificial Sequence
  Synthetic
uagacuaccaagacugacauguucuuc

SEQ ID NO: 604 - RNA, 27 nt, Artificial Sequence
  Synthetic
uaguauaaugggauccuccgaugccaa

SEQ ID NO: 605 - RNA, 27 nt, Artificial Sequence
  Synthetic
cugguuaggccagcauuuggauaguau

SEQ ID NO: 606 - RNA, 27 nt, Artificial Sequence
  Synthetic
gcuuguucagaaggagccucugugcuu

SEQ ID NO: 607 - RNA, 27 nt, Artificial Sequence
  Synthetic
cucaguuggugcuuguucagaaggagc

SEQ ID NO: 608 - RNA, 27 nt, Artificial Sequence
  Synthetic
ucucauguggugcuuguucagaaggag

SEQ ID NO: 609 - RNA, 27 nt, Artificial Sequence
  Synthetic
uucucugguggugcuuguucagaagga

SEQ ID NO: 610 - RNA, 27 nt, Artificial Sequence
  Synthetic
uuucuuagguggugcuuguucagaagg

SEQ ID NO: 611 - RNA, 27 nt, Artificial Sequence
  Synthetic
uuuucucagguggugcuuguucagaag

SEQ ID NO: 612 - RNA, 27 nt, Artificial Sequence
  Synthetic
cuuuuuucagguggugcuuguucagaa

SEQ ID NO: 613 - RNA, 27 nt, Artificial Sequence
  Synthetic
gcuuuucucagguggugcuuguucaga

SEQ ID NO: 614 - RNA, 27 nt, Artificial Sequence
  Synthetic
ggcuuuucucagguggugcuuguucag

SEQ ID NO: 615 - RNA, 27 nt, Artificial Sequence
  Synthetic
gggcuuuucucagguggugcuuguuca

SEQ ID NO: 616 - RNA, 27 nt, Artificial Sequence
  Synthetic
caggguuuuucucagguggugcuuguu

SEQ ID NO: 617 - RNA, 27 nt, Artificial Sequence
  Synthetic
acaggucuuuucucagguggugcuugu

SEQ ID NO: 618 - RNA, 27 nt, Artificial Sequence
  Synthetic
cacagugcuuuucucagguggugcuug

SEQ ID NO: 619 - RNA, 27 nt, Artificial Sequence
  Synthetic
uccuguaccacagggcuuuucucaggu

SEQ ID NO: 620 - RNA, 27 nt, Artificial Sequence
  Synthetic
ugguaucaauccuggaccacagggcuu

SEQ ID NO: 621 - RNA, 27 nt, Artificial Sequence
  Synthetic
ccauguuagcaauccuggaccacaggg

SEQ ID NO: 622 - RNA, 27 nt, Artificial Sequence
  Synthetic
gguccuuccugugacagugguggagga

SEQ ID NO: 623 - RNA, 27 nt, Artificial Sequence
  Synthetic
aggucuuuccugugacagugguggagg

SEQ ID NO: 624 - RNA, 27 nt, Artificial Sequence
  Synthetic
aguguuguaucauagaugaccaagauu

SEQ ID NO: 625 - RNA, 27 nt, Artificial Sequence
  Synthetic
cagugugguaucauagaugaccaagau

SEQ ID NO: 626 - RNA, 27 nt, Artificial Sequence
  Synthetic
ccaguuugguaucauagaugaccaaga

SEQ ID NO: 627 - RNA, 27 nt, Artificial Sequence
  Synthetic
gccagugugguaucauagaugaccaag

SEQ ID NO: 628 - RNA, 27 nt, Artificial Sequence
  Synthetic
ugccauugugguaucauagaugaccaa

SEQ ID NO: 629 - RNA, 27 nt, Artificial Sequence
  Synthetic
augccugugugguaucauagaugacca

SEQ ID NO: 630 - RNA, 27 nt, Artificial Sequence
  Synthetic
gaugcuagugugguaucauagaugacc

SEQ ID NO: 631 - RNA, 27 nt, Artificial Sequence
  Synthetic
ugaugucagugugguaucauagaugac

SEQ ID NO: 632 - RNA, 27 nt, Artificial Sequence
  Synthetic
cugauuccagugugguaucauagauga

SEQ ID NO: 633 - RNA, 27 nt, Artificial Sequence
  Synthetic
ucugaugccagugugguaucauagaug

SEQ ID NO: 634 - RNA, 27 nt, Artificial Sequence
  Synthetic
cucuguugccagugugguaucauagau

SEQ ID NO: 635 - RNA, 27 nt, Artificial Sequence
  Synthetic
guccuuugaugccagugugguaucaua

SEQ ID NO: 636 - RNA, 27 nt, Artificial Sequence
  Synthetic
gggucuucugaugccagugugguauca

SEQ ID NO: 637 - RNA, 27 nt, Artificial Sequence
  Synthetic
uuugguuaguuuucugggguccucuga

SEQ ID NO: 638 - RNA, 27 nt, Artificial Sequence
  Synthetic
auuuguguaguuuucugggguccucug

SEQ ID NO: 639 - RNA, 27 nt, Artificial Sequence
  Synthetic
gaaucuggauuccugcaguaguucucg

SEQ ID NO: 640 - RNA, 27 nt, Artificial Sequence
  Synthetic
ccagauucuggauuccugcaguaguuc

SEQ ID NO: 641 - RNA, 27 nt, Artificial Sequence
  Synthetic
ucccauaaucuggauuccugcaguagu

SEQ ID NO: 642 - RNA, 27 nt, Artificial Sequence
  Synthetic
uucccugaaucuggauuccugcaguag

SEQ ID NO: 643 - RNA, 27 nt, Artificial Sequence
  Synthetic
uuuccuagaaucuggauuccugcagua

SEQ ID NO: 644 - RNA, 27 nt, Artificial Sequence
  Synthetic
guuucucagaaucuggauuccugcagu

SEQ ID NO: 645 - RNA, 27 nt, Artificial Sequence
  Synthetic
uguuuuccagaaucuggauuccugcag

SEQ ID NO: 646 - RNA, 27 nt, Artificial Sequence
  Synthetic
gguuguuucccagaaucuggauuccug

SEQ ID NO: 647 - RNA, 27 nt, Artificial Sequence
  Synthetic
uuguguaacaccaggguuguuucccag

SEQ ID NO: 648 - RNA, 27 nt, Artificial Sequence
  Synthetic
guuguuuaacaccaggguuguuuccca

SEQ ID NO: 649 - RNA, 27 nt, Artificial Sequence
  Synthetic
gauuguaguacucccaccucacacacg

SEQ ID NO: 650 - RNA, 27 nt, Artificial Sequence
  Synthetic
agauuucaguacucccaccucacacac

SEQ ID NO: 651 - RNA, 27 nt, Artificial Sequence
  Synthetic
ucagauugcaguacucccaccucacac

SEQ ID NO: 652 - RNA, 27 nt, Artificial Sequence
  Synthetic
gucaguuugcaguacucccaccucaca

SEQ ID NO: 653 - RNA, 27 nt, Artificial Sequence
  Synthetic
auucuuuuucugagcauugugucagau

SEQ ID NO: 654 - RNA, 27 nt, Artificial Sequence
  Synthetic
gauucuguuucugagcauugugucaga

SEQ ID NO: 655 - RNA, 27 nt, Artificial Sequence
  Synthetic
ugauuuuguuucugagcauugugucag

SEQ ID NO: 656 - RNA, 27 nt, Artificial Sequence
  Synthetic
cugauucuguuucugagcauuguguca

SEQ ID NO: 657 - RNA, 27 nt, Artificial Sequence
  Synthetic
ccugauucuguuucugagcauuguguc

SEQ ID NO: 658 - RNA, 27 nt, Artificial Sequence
  Synthetic
accuguuucuguuucugagcauugugu

SEQ ID NO: 659 - RNA, 27 nt, Artificial Sequence
  Synthetic
caccuuauucuguuucugagcauugug

SEQ ID NO: 660 - RNA, 27 nt, Artificial Sequence
  Synthetic
acaccugauucuguuucugagcauugu

SEQ ID NO: 661 - RNA, 27 nt, Artificial Sequence
  Synthetic
gacacuugauucuguuucugagcauug

SEQ ID NO: 662 - RNA, 27 nt, Artificial Sequence
  Synthetic
ggacaucugauucuguuucugagcauu

SEQ ID NO: 663 - RNA, 27 nt, Artificial Sequence
  Synthetic
uaggauaccugauucuguuucugagca

SEQ ID NO: 664 - RNA, 27 nt, Artificial Sequence
  Synthetic
ucuaguacaccugauucuguuucugag

SEQ ID NO: 665 - RNA, 27 nt, Artificial Sequence
  Synthetic
cucuaugacaccugauucuguuucuga

SEQ ID NO: 666 - RNA, 27 nt, Artificial Sequence
  Synthetic
ucucuuggacaccugauucuguuucug

SEQ ID NO: 667 - RNA, 27 nt, Artificial Sequence
  Synthetic
gucucuaggacaccugauucuguuucu

SEQ ID NO: 668 - RNA, 27 nt, Artificial Sequence
  Synthetic
agucuuuaggacaccugauucuguuuc

SEQ ID NO: 669 - RNA, 27 nt, Artificial Sequence
  Synthetic
ggaguuucuaggacaccugauucuguu

SEQ ID NO: 670 - RNA, 27 nt, Artificial Sequence
  Synthetic
ugggauucucuaggacaccugauucug

SEQ ID NO: 671 - RNA, 27 nt, Artificial Sequence
  Synthetic
agugguagucucuaggacaccugauuc

SEQ ID NO: 672 - RNA, 27 nt, Artificial Sequence
  Synthetic
caacauugggagucucuaggacaccug

SEQ ID NO: 673 - RNA, 27 nt, Artificial Sequence
  Synthetic
uggaauaacagugggagucucuaggac

SEQ ID NO: 674 - RNA, 27 nt, Artificial Sequence
  Synthetic
cuggaucaacagugggagucucuagga

SEQ ID NO: 675 - RNA, 27 nt, Artificial Sequence
  Synthetic
acugguacaacagugggagucucuagg

SEQ ID NO: 676 - RNA, 27 nt, Artificial Sequence
  Synthetic
aacuguaacaacagugggagucucuag

SEQ ID NO: 677 - RNA, 27 nt, Artificial Sequence
  Synthetic
gaacuugaacaacagugggagucucua

SEQ ID NO: 678 - RNA, 27 nt, Artificial Sequence
  Synthetic
ggaacuggaacaacagugggagucucu

SEQ ID NO: 679 - RNA, 27 nt, Artificial Sequence
  Synthetic
caguuugugcugcuucagaaugagccu

SEQ ID NO: 680 - RNA, 27 nt, Artificial Sequence
  Synthetic
ucaguuggugcugcuucagaaugagcc

SEQ ID NO: 681 - RNA, 27 nt, Artificial Sequence
  Synthetic
cucaguuggugcugcuucagaaugagc

SEQ ID NO: 682 - RNA, 27 nt, Artificial Sequence
  Synthetic
gcucauuuggugcugcuucagaaugag

SEQ ID NO: 683 - RNA, 27 nt, Artificial Sequence
  Synthetic
ugcucuguuggugcugcuucagaauga

SEQ ID NO: 684 - RNA, 27 nt, Artificial Sequence
  Synthetic
uugcuuaguuggugcugcuucagaaug

SEQ ID NO: 685 - RNA, 27 nt, Artificial Sequence
  Synthetic
uuugcucaguuggugcugcuucagaau

SEQ ID NO: 686 - RNA, 27 nt, Artificial Sequence
  Synthetic
guuuguucaguuggugcugcuucagaa

SEQ ID NO: 687 - RNA, 27 nt, Artificial Sequence
  Synthetic
gguuuucucaguuggugcugcuucaga

SEQ ID NO: 688 - RNA, 27 nt, Artificial Sequence
  Synthetic
ggguuugcucaguuggugcugcuucag

SEQ ID NO: 689 - RNA, 27 nt, Artificial Sequence
  Synthetic
gggguuugcucaguuggugcugcuuca

SEQ ID NO: 690 - RNA, 27 nt, Artificial Sequence
  Synthetic
ucuggucauuaccaugguagcacugcc

SEQ ID NO: 691 - RNA, 27 nt, Artificial Sequence
  Synthetic
cucuguccauuaccaugguagcacugc

SEQ ID NO: 692 - RNA, 27 nt, Artificial Sequence
  Synthetic
uuccuuugacagugguggagaaugugc

SEQ ID NO: 693 - RNA, 27 nt, Artificial Sequence
  Synthetic
uugacuuguccuuccugugacaguggu

SEQ ID NO: 694 - RNA, 27 nt, Artificial Sequence
  Synthetic
auugauauguccuuccugugacagugg

SEQ ID NO: 695 - RNA, 27 nt, Artificial Sequence
  Synthetic
gauugucauguccuuccugugacagug

SEQ ID NO: 696 - RNA, 27 nt, Artificial Sequence
  Synthetic
agauuuacauguccuuccugugacagu

SEQ ID NO: 697 - RNA, 27 nt, Artificial Sequence
  Synthetic
aagauugacauguccuuccugugacag

SEQ ID NO: 698 - RNA, 27 nt, Artificial Sequence
  Synthetic
caagauugacauguccuuccugugaca

SEQ ID NO: 699 - RNA, 27 nt, Artificial Sequence
  Synthetic
accaauauugacauguccuuccuguga

SEQ ID NO: 700 - RNA, 27 nt, Artificial Sequence
  Synthetic
gaccaugauugacauguccuuccugug

SEQ ID NO: 701 - RNA, 27 nt, Artificial Sequence
  Synthetic
ugaccuagauugacauguccuuccugu

SEQ ID NO: 702 - RNA, 27 nt, Artificial Sequence
  Synthetic
augacuaagauugacauguccuuccug

SEQ ID NO: 703 - RNA, 27 nt, Artificial Sequence
  Synthetic
gaugaucaagauugacauguccuuccu

SEQ ID NO: 704 - RNA, 27 nt, Artificial Sequence
  Synthetic
ggauguccaagauugacauguccuucc

SEQ ID NO: 705 - RNA, 27 nt, Artificial Sequence
  Synthetic
uggauuaccaagauugacauguccuuc

SEQ ID NO: 706 - RNA, 27 nt, Artificial Sequence
  Synthetic
auggaugaccaagauugacauguccuu

SEQ ID NO: 707 - RNA, 27 nt, Artificial Sequence
  Synthetic
caugguugaccaagauugacauguccu

SEQ ID NO: 708 - RNA, 27 nt, Artificial Sequence
  Synthetic
ucauguaugaccaagauugacaugucc

SEQ ID NO: 709 - RNA, 27 nt, Artificial Sequence
  Synthetic
gucauugaugaccaagauugacauguc

SEQ ID NO: 710 - RNA, 27 nt, Artificial Sequence
  Synthetic
ugucauggaugaccaagauugacaugu

SEQ ID NO: 711 - RNA, 27 nt, Artificial Sequence
  Synthetic
gugucuuggaugaccaagauugacaug

SEQ ID NO: 712 - RNA, 27 nt, Artificial Sequence
  Synthetic
gguguuauggaugaccaagauugacau

SEQ ID NO: 713 - RNA, 27 nt, Artificial Sequence
  Synthetic
uggugucauggaugaccaagauugaca

SEQ ID NO: 714 - RNA, 27 nt, Artificial Sequence
  Synthetic
gugguuucauggaugaccaagauugac

SEQ ID NO: 715 - RNA, 27 nt, Artificial Sequence
  Synthetic
uguggugucauggaugaccaagauuga

SEQ ID NO: 716 - RNA, 27 nt, Artificial Sequence
  Synthetic
guguguugucauggaugaccaagauug

SEQ ID NO: 717 - RNA, 27 nt, Artificial Sequence
  Synthetic
ggauuucugcaguaguucauugucagg

SEQ ID NO: 718 - RNA, 27 nt, Artificial Sequence
  Synthetic
uggauuccugcaguaguucauugucag

SEQ ID NO: 719 - RNA, 27 nt, Artificial Sequence
  Synthetic
cuggauuccugcaguaguucauuguca

SEQ ID NO: 720 - RNA, 27 nt, Artificial Sequence
  Synthetic
ucugguuuccugcaguaguucauuguc

SEQ ID NO: 721 - RNA, 27 nt, Artificial Sequence
  Synthetic
aucuguauuccugcaguaguucauugu

SEQ ID NO: 722 - RNA, 27 nt, Artificial Sequence
  Synthetic
caucuugauuccugcaguaguucauug

SEQ ID NO: 723 - RNA, 27 nt, Artificial Sequence
  Synthetic
gcaucuggauuccugcaguaguucauu

SEQ ID NO: 724 - RNA, 27 nt, Artificial Sequence
  Synthetic
aucgguaucuggauuccugcaguaguu

SEQ ID NO: 725 - RNA, 27 nt, Artificial Sequence
  Synthetic
uccauuguaaaacaccaagggccugua

SEQ ID NO: 726 - RNA, 27 nt, Artificial Sequence
  Synthetic
cuggguuccaugguaaaacaccaaggg

SEQ ID NO: 727 - RNA, 27 nt, Artificial Sequence
  Synthetic
gcugguguccaugguaaaacaccaagg

SEQ ID NO: 728 - RNA, 27 nt, Artificial Sequence
  Synthetic
ugcugugguccaugguaaaacaccaag

SEQ ID NO: 729 - RNA, 27 nt, Artificial Sequence
  Synthetic
augcuuggguccaugguaaaacaccaa

SEQ ID NO: 730 - RNA, 27 nt, Artificial Sequence
  Synthetic
gaugcugggguccaugguaaaacacca

SEQ ID NO: 731 - RNA, 27 nt, Artificial Sequence
  Synthetic
cugauucugggguccaugguaaaacac

SEQ ID NO: 732 - RNA, 27 nt, Artificial Sequence
  Synthetic
gcaucucgucagguugcaguacuccca

SEQ ID NO: 733 - RNA, 27 nt, Artificial Sequence
  Synthetic
agcauugcgucagguugcaguacuccc

SEQ ID NO: 734 - RNA, 27 nt, Artificial Sequence
  Synthetic
ugagcuucgcgucagguugcaguacuc

SEQ ID NO: 735 - RNA, 27 nt, Artificial Sequence
  Synthetic
cugaguaucgcgucagguugcaguacu

SEQ ID NO: 736 - RNA, 27 nt, Artificial Sequence
  Synthetic
ugucuuagcaucgcgucagguugcagu

SEQ ID NO: 737 - RNA, 27 nt, Artificial Sequence
  Synthetic
uucugugucugagcaucgcgucagguu

SEQ ID NO: 738 - RNA, 27 nt, Artificial Sequence
  Synthetic
cuucuuugucugagcaucgcgucaggu

SEQ ID NO: 739 - RNA, 27 nt, Artificial Sequence
  Synthetic
acaguuccuucugugucugagcaucgc

SEQ ID NO: 740 - RNA, 27 nt, Artificial Sequence
  Synthetic
cacagucccuucugugucugagcaucg

SEQ ID NO: 741 - RNA, 27 nt, Artificial Sequence
  Synthetic
agcgaucacagucccuucugugucuga

SEQ ID NO: 742 - RNA, 27 nt, Artificial Sequence
  Synthetic
ucccauaaucaaaugaagaggaugcac

SEQ ID NO: 743 - RNA, 27 nt, Artificial Sequence
  Synthetic
uucccucaaucaaaugaagaggaugca

SEQ ID NO: 744 - RNA, 27 nt, Artificial Sequence
  Synthetic
cuuccuacaaucaaaugaagaggaugc

SEQ ID NO: 745 - RNA, 27 nt, Artificial Sequence
  Synthetic
gcuucucacaaucaaaugaagaggaug

SEQ ID NO: 746 - RNA, 27 nt, Artificial Sequence
  Synthetic
ggcuuuccacaaucaaaugaagaggau

SEQ ID NO: 747 - RNA, 27 nt, Artificial Sequence
  Synthetic
ugagguuucccacaaucaaaugaagag

SEQ ID NO: 748 - RNA, 27 nt, Artificial Sequence
  Synthetic
uugagucuucccacaaucaaaugaaga

SEQ ID NO: 749 - RNA, 27 nt, Artificial Sequence
  Synthetic
cuugaugcuucccacaaucaaaugaag

SEQ ID NO: 750 - RNA, 27 nt, Artificial Sequence
  Synthetic
acuuguggcuucccacaaucaaaugaa

SEQ ID NO: 751 - RNA, 27 nt, Artificial Sequence
  Synthetic
cacuuuaggcuucccacaaucaaauga

SEQ ID NO: 752 - RNA, 27 nt, Artificial Sequence
  Synthetic
uucgguuccacuugaggcuucccacaa

SEQ ID NO: 753 - RNA, 27 nt, Artificial Sequence
  Synthetic
cuacauugcuuccaggacauuucuucg

SEQ ID NO: 754 - RNA, 27 nt, Artificial Sequence
  Synthetic
ccuacuaugcuuccaggacauuucuuc

SEQ ID NO: 755 - RNA, 27 nt, Artificial Sequence
  Synthetic
cccuauaaugcuuccaggacauuucuu

SEQ ID NO: 756 - RNA, 27 nt, Artificial Sequence
  Synthetic
cccccuacaaugcuuccaggacauuuc

SEQ ID NO: 757 - RNA, 27 nt, Artificial Sequence
  Synthetic
cccccuuacaaugcuuccaggacauuu

SEQ ID NO: 758 - RNA, 27 nt, Artificial Sequence
  Synthetic
agaagugcuuuccaaaccuuguucuga

SEQ ID NO: 759 - RNA, 27 nt, Artificial Sequence
  Synthetic
cagaauugcuuuccaaaccuuguucug

SEQ ID NO: 760 - RNA, 27 nt, Artificial Sequence
  Synthetic
acagaugugcuuuccaaaccuuguucu

SEQ ID NO: 761 - RNA, 27 nt, Artificial Sequence
  Synthetic
cacaguagugcuuuccaaaccuuguuc

SEQ ID NO: 762 - RNA, 27 nt, Artificial Sequence
  Synthetic
ccacauaagugcuuuccaaaccuuguu

SEQ ID NO: 763 - RNA, 27 nt, Artificial Sequence
  Synthetic
uccacugaagugcuuuccaaaccuugu

SEQ ID NO: 764 - RNA, 27 nt, Artificial Sequence
  Synthetic
cuccauagaagugcuuuccaaaccuug

SEQ ID NO: 765 - RNA, 27 nt, Artificial Sequence
  Synthetic
ggugcuuccacagaagugcuuuccaaa

SEQ ID NO: 766 - RNA, 27 nt, Artificial Sequence
  Synthetic
ucuggugauauuaaggugccuccacag

SEQ ID NO: 767 - RNA, 27 nt, Artificial Sequence
  Synthetic
cagcauccacucuggggauauuaaggu

SEQ ID NO: 768 - RNA, 27 nt, Artificial Sequence
  Synthetic
gucaguacccacucuggggauauuaag

SEQ ID NO: 769 - RNA, 27 nt, Artificial Sequence
  Synthetic
gugaguagcagucagcacccacucugg

SEQ ID NO: 770 - RNA, 27 nt, Artificial Sequence
  Synthetic
uugguuugcacccaggaugaccuugua

SEQ ID NO: 771 - RNA, 27 nt, Artificial Sequence
  Synthetic
guucauuucuuggugugcacccaggau

SEQ ID NO: 772 - RNA, 27 nt, Artificial Sequence
  Synthetic
ucgaguuucacuucuuggugugcaccc

SEQ ID NO: 773 - RNA, 27 nt, Artificial Sequence
  Synthetic
uagcuuuagcaaggcaauaucugcuug

SEQ ID NO: 774 - RNA, 27 nt, Artificial Sequence
  Synthetic
uuagcuuuagcaaggcaauaucugcuu

SEQ ID NO: 775 - RNA, 27 nt, Artificial Sequence
  Synthetic
cuuaguuuuagcaaggcaauaucugcu

SEQ ID NO: 776 - RNA, 27 nt, Artificial Sequence
  Synthetic
gcuuaucuuuagcaaggcaauaucugc

SEQ ID NO: 777 - RNA, 27 nt, Artificial Sequence
  Synthetic
ugcuuugcuuuagcaaggcaauaucug

SEQ ID NO: 778 - RNA, 27 nt, Artificial Sequence
  Synthetic
cugcuuagcuuuagcaaggcaauaucu

SEQ ID NO: 779 - RNA, 27 nt, Artificial Sequence
  Synthetic
ccugcuuagcuuuagcaaggcaauauc

SEQ ID NO: 780 - RNA, 27 nt, Artificial Sequence
  Synthetic
gcugguauuacuuugucagugaugacg

SEQ ID NO: 781 - RNA, 27 nt, Artificial Sequence
  Synthetic
cagccugugauguaacauucaguccug

SEQ ID NO: 782 - RNA, 27 nt, Artificial Sequence
  Synthetic
ccagcuagugauguaacauucaguccu

SEQ ID NO: 783 - RNA, 27 nt, Artificial Sequence
  Synthetic
cccagucagugauguaacauucagucc

SEQ ID NO: 784 - RNA, 27 nt, Artificial Sequence
  Synthetic
ccccauccagugauguaacauucaguc

SEQ ID NO: 785 - RNA, 27 nt, Artificial Sequence
  Synthetic
uccccugccagugauguaacauucagu

SEQ ID NO: 786 - RNA, 27 nt, Artificial Sequence
  Synthetic
ucuccucagccagugauguaacauuca

SEQ ID NO: 787 - RNA, 27 nt, Artificial Sequence
  Synthetic
cagucucaaagguaccuuggguuucuc

SEQ ID NO: 788 - RNA, 22 nt, Artificial Sequence
  Synthetic
uuuccugugacagugguggagg

SEQ ID NO: 789 - RNA, 22 nt, Artificial Sequence
  Synthetic
uauaacucuguccauuaccagg

SEQ ID NO: 790 - RNA, 22 nt, Artificial Sequence
  Synthetic
uaucgugucagguugcaguagg

SEQ ID NO: 791 - RNA, 22 nt, Artificial Sequence
  Synthetic
uaccaagcuuggcaaguucugg

SEQ ID NO: 792 - RNA, 22 nt, Artificial Sequence
  Synthetic
uugaccaagcuuggcaaguugg

SEQ ID NO: 793 - RNA, 22 nt, Artificial Sequence
  Synthetic
uagaugaccaagcuuggcaagg

SEQ ID NO: 794 - RNA, 22 nt, Artificial Sequence
  Synthetic
uccucgauaacucuguccaugg

SEQ ID NO: 795 - RNA, 22 nt, Artificial Sequence
  Synthetic
ugccucgauaacucuguccagg

SEQ ID NO: 796 - RNA, 22 nt, Artificial Sequence
  Synthetic
ugugguaucauagaugaccagg

SEQ ID NO: 797 - RNA, 22 nt, Artificial Sequence
  Synthetic
uaaucuggauuccugcaguagg

SEQ ID NO: 798 - RNA, 22 nt, Artificial Sequence
  Synthetic
uaugcuuccaggacauuucugg

SEQ ID NO: 799 - RNA, 22 nt, Artificial Sequence
  Synthetic
uuggauaauauucuguugucgg

SEQ ID NO: 800 - RNA, 22 nt, Artificial Sequence
  Synthetic
uccuugauaacucuguccaugg

SEQ ID NO: 801 - RNA, 22 nt, Artificial Sequence
  Synthetic
uuggauaauauucuguugucgg

SEQ ID NO: 802 - RNA, 22 nt, Artificial Sequence
  Synthetic
uccuugauaacucuguccaugg

SEQ ID NO: 803 - RNA, 22 nt, Artificial Sequence
  Synthetic
uuggauaauauucuguugucgg

SEQ ID NO: 804 - RNA, 19 nt, Artificial Sequence
  Synthetic
cugagcaaagccauguggu

SEQ ID NO: 805 - RNA, 19 nt, Artificial Sequence
  Synthetic
agcaaagccauguggucca

SEQ ID NO: 806 - RNA, 19 nt, Artificial Sequence
  Synthetic
aagccaugugguccaggau

SEQ ID NO: 807 - RNA, 19 nt, Artificial Sequence
  Synthetic
agccaugugguccaggauu

SEQ ID NO: 808 - RNA, 19 nt, Artificial Sequence
  Synthetic
gccaugugguccaggauug

SEQ ID NO: 809 - RNA, 19 nt, Artificial Sequence
  Synthetic
ccaugugguccaggauugc

SEQ ID NO: 810 - RNA, 19 nt, Artificial Sequence
  Synthetic
caugugguccaggauugcu

SEQ ID NO: 811 - RNA, 19 nt, Artificial Sequence
  Synthetic
augugguccaggauugcua

SEQ ID NO: 812 - RNA, 19 nt, Artificial Sequence
  Synthetic
ugugguccaggauugcuac

SEQ ID NO: 813 - RNA, 19 nt, Artificial Sequence
  Synthetic
ggugauggacagaguuauc

SEQ ID NO: 814 - RNA, 19 nt, Artificial Sequence
  Synthetic
uccaccacugucacaggaa

SEQ ID NO: 815 - RNA, 19 nt, Artificial Sequence
  Synthetic
ccaccacugucacaggaag

SEQ ID NO: 816 - RNA, 19 nt, Artificial Sequence
  Synthetic
cugucacaggaaggaccug

SEQ ID NO: 817 - RNA, 19 nt, Artificial Sequence
  Synthetic
ggaaggaccugccaagcuu

SEQ ID NO: 818 - RNA, 19 nt, Artificial Sequence
  Synthetic
gaaggaccugccaagcuug

SEQ ID NO: 819 - RNA, 19 nt, Artificial Sequence
  Synthetic
aggaccugccaagcuuggu

SEQ ID NO: 820 - RNA, 19 nt, Artificial Sequence
  Synthetic
ggaccugccaagcuugguc

SEQ ID NO: 821 - RNA, 19 nt, Artificial Sequence
  Synthetic
gaccugccaagcuugguca

SEQ ID NO: 822 - RNA, 19 nt, Artificial Sequence
  Synthetic
accugccaagcuuggucau

SEQ ID NO: 823 - RNA, 19 nt, Artificial Sequence
  Synthetic
ccugccaagcuuggucauc

SEQ ID NO: 824 - RNA, 19 nt, Artificial Sequence
  Synthetic
agcuuggucaucuaugaca

SEQ ID NO: 825 - RNA, 19 nt, Artificial Sequence
  Synthetic
gucaucuaugacaccacau

SEQ ID NO: 826 - RNA, 19 nt, Artificial Sequence
  Synthetic
cacagaaaacuacccaaau

SEQ ID NO: 827 - RNA, 19 nt, Artificial Sequence
  Synthetic
agaaaacuacccaaaugcu

SEQ ID NO: 828 - RNA, 19 nt, Artificial Sequence
  Synthetic
aaaacuacccaaaugcugg

SEQ ID NO: 829 - RNA, 19 nt, Artificial Sequence
  Synthetic
acccaaaugcuggcuugau

SEQ ID NO: 830 - RNA, 19 nt, Artificial Sequence
  Synthetic
cccaaaugcuggcuugauc

SEQ ID NO: 831 - RNA, 19 nt, Artificial Sequence
  Synthetic
gaacuacugcaggaaucca

SEQ ID NO: 832 - RNA, 19 nt, Artificial Sequence
  Synthetic
uacugcaggaauccagaug

SEQ ID NO: 833 - RNA, 19 nt, Artificial Sequence
  Synthetic
acugcaggaauccagaugc

SEQ ID NO: 834 - RNA, 19 nt, Artificial Sequence
  Synthetic
ugcaggaauccagaugcug

SEQ ID NO: 835 - RNA, 19 nt, Artificial Sequence
  Synthetic
aggugggaguacugcaacc

SEQ ID NO: 836 - RNA, 19 nt, Artificial Sequence
  Synthetic
aaugcucagacgcagaagg

SEQ ID NO: 837 - RNA, 19 nt, Artificial Sequence
  Synthetic
gacuguuaccccgguucca

SEQ ID NO: 838 - RNA, 19 nt, Artificial Sequence
  Synthetic
acuguuaccccgguuccaa

SEQ ID NO: 839 - RNA, 19 nt, Artificial Sequence
  Synthetic
cuguuaccccgguuccaag

SEQ ID NO: 840 - RNA, 19 nt, Artificial Sequence
  Synthetic
uguuaccccgguuccaagc

SEQ ID NO: 841 - RNA, 19 nt, Artificial Sequence
  Synthetic
guuaccccgguuccaagcc

SEQ ID NO: 842 - RNA, 19 nt, Artificial Sequence
  Synthetic
uuaccccgguuccaagccu

SEQ ID NO: 843 - RNA, 19 nt, Artificial Sequence
  Synthetic
uaccccgguuccaagccua

SEQ ID NO: 844 - RNA, 19 nt, Artificial Sequence
  Synthetic
gugcuaccaugguaaugga

SEQ ID NO: 845 - RNA, 19 nt, Artificial Sequence
  Synthetic
ugcuaccaugguaauggac

SEQ ID NO: 846 - RNA, 19 nt, Artificial Sequence
  Synthetic
gcuaccaugguaauggaca

SEQ ID NO: 847 - RNA, 19 nt, Artificial Sequence
  Synthetic
cuaccaugguaauggacag

SEQ ID NO: 848 - RNA, 19 nt, Artificial Sequence
  Synthetic
uaccaugguaauggacaga

SEQ ID NO: 849 - RNA, 19 nt, Artificial Sequence
  Synthetic
accaugguaauggacagag

SEQ ID NO: 850 - RNA, 19 nt, Artificial Sequence
  Synthetic
ccaugguaauggacagagu

SEQ ID NO: 851 - RNA, 19 nt, Artificial Sequence
  Synthetic
caugguaauggacagaguu

SEQ ID NO: 852 - RNA, 19 nt, Artificial Sequence
  Synthetic
augguaauggacagaguua

SEQ ID NO: 853 - RNA, 19 nt, Artificial Sequence
  Synthetic
ugguaauggacagaguuau

SEQ ID NO: 854 - RNA, 19 nt, Artificial Sequence
  Synthetic
gguaauggacagaguuauc

SEQ ID NO: 855 - RNA, 19 nt, Artificial Sequence
  Synthetic
guaauggacagaguuaucg

SEQ ID NO: 856 - RNA, 19 nt, Artificial Sequence
  Synthetic
ggcacauacuccaccacug

SEQ ID NO: 857 - RNA, 19 nt, Artificial Sequence
  Synthetic
cuuggucaucuaugacacc

SEQ ID NO: 858 - RNA, 19 nt, Artificial Sequence
  Synthetic
gucaucuaugacaccacac

SEQ ID NO: 859 - RNA, 19 nt, Artificial Sequence
  Synthetic
ucaucuaugacaccacacu

SEQ ID NO: 860 - RNA, 19 nt, Artificial Sequence
  Synthetic
caucuaugacaccacacuc

SEQ ID NO: 861 - RNA, 19 nt, Artificial Sequence
  Synthetic
gcacauacuccaccacugu

SEQ ID NO: 862 - RNA, 19 nt, Artificial Sequence
  Synthetic
agccccuuauuguuauacg

SEQ ID NO: 863 - RNA, 19 nt, Artificial Sequence
  Synthetic
gccccuuauuguuauacga

SEQ ID NO: 864 - RNA, 19 nt, Artificial Sequence
  Synthetic
ccaagccuagaggcuccuu

SEQ ID NO: 865 - RNA, 19 nt, Artificial Sequence
  Synthetic
aggcuccuucugaacaagc

SEQ ID NO: 866 - RNA, 19 nt, Artificial Sequence
  Synthetic
auggacagaguuaucaagg

SEQ ID NO: 867 - RNA, 19 nt, Artificial Sequence
  Synthetic
uggacagaguuaucaaggc

SEQ ID NO: 868 - RNA, 19 nt, Artificial Sequence
  Synthetic
ggacagaguuaucaaggca

SEQ ID NO: 869 - RNA, 19 nt, Artificial Sequence
  Synthetic
gacagaguuaucaaggcac

SEQ ID NO: 870 - RNA, 19 nt, Artificial Sequence
  Synthetic
acagaguuaucaaggcaca

SEQ ID NO: 871 - RNA, 19 nt, Artificial Sequence
  Synthetic
cagaguuaucaaggcacau

SEQ ID NO: 872 - RNA, 19 nt, Artificial Sequence
  Synthetic
uacccaaaugcuggcuuga

SEQ ID NO: 873 - RNA, 19 nt, Artificial Sequence
  Synthetic
ccaaaugcuggcuugauca

SEQ ID NO: 874 - RNA, 19 nt, Artificial Sequence
  Synthetic
ucaagaacuacugccgaaa

SEQ ID NO: 875 - RNA, 19 nt, Artificial Sequence
  Synthetic
caagaacuacugccgaaau

SEQ ID NO: 876 - RNA, 19 nt, Artificial Sequence
  Synthetic
aagaacuacugccgaaauc

SEQ ID NO: 877 - RNA, 19 nt, Artificial Sequence
  Synthetic
gaacuacugccgaaaucca

SEQ ID NO: 878 - RNA, 19 nt, Artificial Sequence
  Synthetic
cuacugccgaaauccagau

SEQ ID NO: 879 - RNA, 19 nt, Artificial Sequence
  Synthetic
uguggcagccccuuggugu

SEQ ID NO: 880 - RNA, 19 nt, Artificial Sequence
  Synthetic
guggcagccccuugguguu

SEQ ID NO: 881 - RNA, 19 nt, Artificial Sequence
  Synthetic
uggcagccccuugguguua

SEQ ID NO: 882 - RNA, 19 nt, Artificial Sequence
  Synthetic
ggcagccccuugguguuau

SEQ ID NO: 883 - RNA, 19 nt, Artificial Sequence
  Synthetic
gcagccccuugguguuaua

SEQ ID NO: 884 - RNA, 19 nt, Artificial Sequence
  Synthetic
cagccccuugguguuauac

SEQ ID NO: 885 - RNA, 19 nt, Artificial Sequence
  Synthetic
agccccuugguguuauaca

SEQ ID NO: 886 - RNA, 19 nt, Artificial Sequence
  Synthetic
gccccuugguguuauacaa

SEQ ID NO: 887 - RNA, 19 nt, Artificial Sequence
  Synthetic
ccccuugguguuauacaac

SEQ ID NO: 888 - RNA, 19 nt, Artificial Sequence
  Synthetic
ggugggaguacugcaaccu

SEQ ID NO: 889 - RNA, 19 nt, Artificial Sequence
  Synthetic
gugggaguacugcaaccug

SEQ ID NO: 890 - RNA, 19 nt, Artificial Sequence
  Synthetic
ggaguacugcaaccugaca

SEQ ID NO: 891 - RNA, 19 nt, Artificial Sequence
  Synthetic
gaguacugcaaccugacac

SEQ ID NO: 892 - RNA, 19 nt, Artificial Sequence
  Synthetic
guacugcaaccugacacga

SEQ ID NO: 893 - RNA, 19 nt, Artificial Sequence
  Synthetic
uacugcaaccugacacgau

SEQ ID NO: 894 - RNA, 19 nt, Artificial Sequence
  Synthetic
acugcaaccugacacgaug

SEQ ID NO: 895 - RNA, 19 nt, Artificial Sequence
  Synthetic
cugcaaccugacacgaugc

SEQ ID NO: 896 - RNA, 19 nt, Artificial Sequence
  Synthetic
ugcaaccugacacgaugcu

SEQ ID NO: 897 - RNA, 19 nt, Artificial Sequence
  Synthetic
caaccugacacgaugcuca

SEQ ID NO: 898 - RNA, 19 nt, Artificial Sequence
  Synthetic
accugacacgaugcucaga

SEQ ID NO: 899 - RNA, 19 nt, Artificial Sequence
  Synthetic
ccugacacgaugcucagau

SEQ ID NO: 900 - RNA, 19 nt, Artificial Sequence
  Synthetic
cugacacgaugcucagaug

SEQ ID NO: 901 - RNA, 19 nt, Artificial Sequence
  Synthetic
ugacacgaugcucagaugc

SEQ ID NO: 902 - RNA, 19 nt, Artificial Sequence
  Synthetic
gacacgaugcucagaugca

SEQ ID NO: 903 - RNA, 19 nt, Artificial Sequence
  Synthetic
acacgaugcucagaugcag

SEQ ID NO: 904 - RNA, 19 nt, Artificial Sequence
  Synthetic
cacgaugcucagaugcaga

SEQ ID NO: 905 - RNA, 19 nt, Artificial Sequence
  Synthetic
ugcuacuaccauuauggac

SEQ ID NO: 906 - RNA, 19 nt, Artificial Sequence
  Synthetic
gcuacuaccauuauggaca

SEQ ID NO: 907 - RNA, 19 nt, Artificial Sequence
  Synthetic
cuacuaccauuauggacag

SEQ ID NO: 908 - RNA, 19 nt, Artificial Sequence
  Synthetic
uacuaccauuauggacaga

SEQ ID NO: 909 - RNA, 19 nt, Artificial Sequence
  Synthetic
acuaccauuauggacagag

SEQ ID NO: 910 - RNA, 19 nt, Artificial Sequence
  Synthetic
cuaccauuauggacagagu

SEQ ID NO: 911 - RNA, 19 nt, Artificial Sequence
  Synthetic
uaccauuauggacagaguu

SEQ ID NO: 912 - RNA, 19 nt, Artificial Sequence
  Synthetic
accauuauggacagaguua

SEQ ID NO: 913 - RNA, 19 nt, Artificial Sequence
  Synthetic
gaggcacauacuccaccac

SEQ ID NO: 914 - RNA, 19 nt, Artificial Sequence
  Synthetic
cuccaccacugucacagga

SEQ ID NO: 915 - RNA, 19 nt, Artificial Sequence
  Synthetic
acaggaagaacuugccaag

SEQ ID NO: 916 - RNA, 19 nt, Artificial Sequence
  Synthetic
caggaagaacuugccaagc

SEQ ID NO: 917 - RNA, 19 nt, Artificial Sequence
  Synthetic
aggaagaacuugccaagcu

SEQ ID NO: 918 - RNA, 19 nt, Artificial Sequence
  Synthetic
ggaagaacuugccaagcuu

SEQ ID NO: 919 - RNA, 19 nt, Artificial Sequence
  Synthetic
gaagaacuugccaagcuug

SEQ ID NO: 920 - RNA, 19 nt, Artificial Sequence
  Synthetic
aagaacuugccaagcuugg

SEQ ID NO: 921 - RNA, 19 nt, Artificial Sequence
  Synthetic
agaacuugccaagcuuggu

SEQ ID NO: 922 - RNA, 19 nt, Artificial Sequence
  Synthetic
gaacuugccaagcuugguc

SEQ ID NO: 923 - RNA, 19 nt, Artificial Sequence
  Synthetic
aacuugccaagcuugguca

SEQ ID NO: 924 - RNA, 19 nt, Artificial Sequence
  Synthetic
acuugccaagcuuggucau

SEQ ID NO: 925 - RNA, 19 nt, Artificial Sequence
  Synthetic
cuugccaagcuuggucauc

SEQ ID NO: 926 - RNA, 19 nt, Artificial Sequence
  Synthetic
uugccaagcuuggucaucu

SEQ ID NO: 927 - RNA, 19 nt, Artificial Sequence
  Synthetic
ugccaagcuuggucaucua

SEQ ID NO: 928 - RNA, 19 nt, Artificial Sequence
  Synthetic
gcuuggucaucuaugacac

SEQ ID NO: 929 - RNA, 19 nt, Artificial Sequence
  Synthetic
uuggucaucuaugacacca

SEQ ID NO: 930 - RNA, 19 nt, Artificial Sequence
  Synthetic
uggucaucuaugacaccac

SEQ ID NO: 931 - RNA, 19 nt, Artificial Sequence
  Synthetic
gucaucuaugacaccacac

SEQ ID NO: 932 - RNA, 19 nt, Artificial Sequence
  Synthetic
caucuaugacaccacacca

SEQ ID NO: 933 - RNA, 19 nt, Artificial Sequence
  Synthetic
aucuaugacaccacaccag

SEQ ID NO: 934 - RNA, 19 nt, Artificial Sequence
  Synthetic
cuaugacaccacaccagca

SEQ ID NO: 935 - RNA, 19 nt, Artificial Sequence
  Synthetic
gucggaccccagaaaacua

SEQ ID NO: 936 - RNA, 19 nt, Artificial Sequence
  Synthetic
ucggaccccagaaaacuac

SEQ ID NO: 937 - RNA, 19 nt, Artificial Sequence
  Synthetic
gaaaacuacccaaaugcug

SEQ ID NO: 938 - RNA, 19 nt, Artificial Sequence
  Synthetic
gcugagauucgcccuuggu

SEQ ID NO: 939 - RNA, 19 nt, Artificial Sequence
  Synthetic
cugagauucgcccuuggug

SEQ ID NO: 940 - RNA, 19 nt, Artificial Sequence
  Synthetic
gagauucgcccuugguguu

SEQ ID NO: 941 - RNA, 19 nt, Artificial Sequence
  Synthetic
agauucgcccuugguguua

SEQ ID NO: 942 - RNA, 19 nt, Artificial Sequence
  Synthetic
uucgcccuugguguuacac

SEQ ID NO: 943 - RNA, 19 nt, Artificial Sequence
  Synthetic
cuugguguuacaccaugga

SEQ ID NO: 944 - RNA, 19 nt, Artificial Sequence
  Synthetic
uugguguuacaccauggau

SEQ ID NO: 945 - RNA, 19 nt, Artificial Sequence
  Synthetic
ugguguuacaccauggauc

SEQ ID NO: 946 - RNA, 19 nt, Artificial Sequence
  Synthetic
gguguuacaccauggaucc

SEQ ID NO: 947 - RNA, 19 nt, Artificial Sequence
  Synthetic
uguuacaccauggauccca

SEQ ID NO: 948 - RNA, 19 nt, Artificial Sequence
  Synthetic
gaaucaaguguccuugcaa

SEQ ID NO: 949 - RNA, 19 nt, Artificial Sequence
  Synthetic
aaucaaguguccuugcaac

SEQ ID NO: 950 - RNA, 19 nt, Artificial Sequence
  Synthetic
aucaaguguccuugcaacu

SEQ ID NO: 951 - RNA, 19 nt, Artificial Sequence
  Synthetic
auggacagaguuaucgagg

SEQ ID NO: 952 - RNA, 19 nt, Artificial Sequence
  Synthetic
uggacagaguuaucgaggc

SEQ ID NO: 953 - RNA, 19 nt, Artificial Sequence
  Synthetic
acaccacacuggcaucaga

SEQ ID NO: 954 - RNA, 19 nt, Artificial Sequence
  Synthetic
uugguguuauaccauggau

SEQ ID NO: 955 - RNA, 19 nt, Artificial Sequence
  Synthetic
ugguguuauaccauggauc

SEQ ID NO: 956 - RNA, 19 nt, Artificial Sequence
  Synthetic
gguguuauaccauggaucc

SEQ ID NO: 957 - RNA, 19 nt, Artificial Sequence
  Synthetic
guguuauaccauggauccc

SEQ ID NO: 958 - RNA, 19 nt, Artificial Sequence
  Synthetic
ucagaugggaguacugcaa

SEQ ID NO: 959 - RNA, 19 nt, Artificial Sequence
  Synthetic
gaugggaguacugcaaccu

SEQ ID NO: 960 - RNA, 19 nt, Artificial Sequence
  Synthetic
augggaguacugcaaccug

SEQ ID NO: 961 - RNA, 19 nt, Artificial Sequence
  Synthetic
ugggaguacugcaaccuga

SEQ ID NO: 962 - RNA, 19 nt, Artificial Sequence
  Synthetic
gggaguacugcaaccugac

SEQ ID NO: 963 - RNA, 19 nt, Artificial Sequence
  Synthetic
ggcuguuucugaacaagca

SEQ ID NO: 964 - RNA, 19 nt, Artificial Sequence
  Synthetic
guuucugaacaagcaccaa

SEQ ID NO: 965 - RNA, 19 nt, Artificial Sequence
  Synthetic
cuccaccacuguuacagga

SEQ ID NO: 966 - RNA, 19 nt, Artificial Sequence
  Synthetic
uccaccacuguuacaggaa

SEQ ID NO: 967 - RNA, 19 nt, Artificial Sequence
  Synthetic
ccaccacuguuacaggaag

SEQ ID NO: 968 - RNA, 19 nt, Artificial Sequence
  Synthetic
gacaccacacuggcaucag

SEQ ID NO: 969 - RNA, 19 nt, Artificial Sequence
  Synthetic
acaccacacuggcaucaga

SEQ ID NO: 970 - RNA, 19 nt, Artificial Sequence
  Synthetic
agaauacuacccaaauggu

SEQ ID NO: 971 - RNA, 19 nt, Artificial Sequence
  Synthetic
gaauacuacccaaauggug

SEQ ID NO: 972 - RNA, 19 nt, Artificial Sequence
  Synthetic
aauacuacccaaauggugg

SEQ ID NO: 973 - RNA, 19 nt, Artificial Sequence
  Synthetic
uccuucugaagaagcacca

SEQ ID NO: 974 - RNA, 19 nt, Artificial Sequence
  Synthetic
ccuucugaagaagcaccaa

SEQ ID NO: 975 - RNA, 19 nt, Artificial Sequence
  Synthetic
cuucugaagaagcaccaac

SEQ ID NO: 976 - RNA, 19 nt, Artificial Sequence
  Synthetic
uucugaagaagcaccaacu

SEQ ID NO: 977 - RNA, 19 nt, Artificial Sequence
  Synthetic
ucugaagaagcaccaacug

SEQ ID NO: 978 - RNA, 19 nt, Artificial Sequence
  Synthetic
cugaagaagcaccaacuga

SEQ ID NO: 979 - RNA, 19 nt, Artificial Sequence
  Synthetic
ugaagaagcaccaacugaa

SEQ ID NO: 980 - RNA, 19 nt, Artificial Sequence
  Synthetic
gaagaagcaccaacugaaa

SEQ ID NO: 981 - RNA, 19 nt, Artificial Sequence
  Synthetic
aagaagcaccaacugaaaa

SEQ ID NO: 982 - RNA, 19 nt, Artificial Sequence
  Synthetic
agaagcaccaacugaaaac

SEQ ID NO: 983 - RNA, 19 nt, Artificial Sequence
  Synthetic
gaagcaccaacugaaaaca

SEQ ID NO: 984 - RNA, 19 nt, Artificial Sequence
  Synthetic
aagcaccaacugaaaacag

SEQ ID NO: 985 - RNA, 19 nt, Artificial Sequence
  Synthetic
aggugauggacagaguuau

SEQ ID NO: 986 - RNA, 19 nt, Artificial Sequence
  Synthetic
cuccaccacuaucacagga

SEQ ID NO: 987 - RNA, 19 nt, Artificial Sequence
  Synthetic
uccaccacuaucacaggaa

SEQ ID NO: 988 - RNA, 19 nt, Artificial Sequence
  Synthetic
ccaccacuaucacaggaag

SEQ ID NO: 989 - RNA, 19 nt, Artificial Sequence
  Synthetic
caccacuaucacaggaaga

SEQ ID NO: 990 - RNA, 19 nt, Artificial Sequence
  Synthetic
accacuaucacaggaagaa

SEQ ID NO: 991 - RNA, 19 nt, Artificial Sequence
  Synthetic
ccacuaucacaggaagaac

SEQ ID NO: 992 - RNA, 19 nt, Artificial Sequence
  Synthetic
cacuaucacaggaagaaca

SEQ ID NO: 993 - RNA, 19 nt, Artificial Sequence
  Synthetic
acuaucacaggaagaacau

SEQ ID NO: 994 - RNA, 19 nt, Artificial Sequence
  Synthetic
cuaucacaggaagaacaug

SEQ ID NO: 995 - RNA, 19 nt, Artificial Sequence
  Synthetic
uaucacaggaagaacaugu

SEQ ID NO: 996 - RNA, 19 nt, Artificial Sequence
  Synthetic
aucacaggaagaacauguc

SEQ ID NO: 997 - RNA, 19 nt, Artificial Sequence
  Synthetic
ucacaggaagaacauguca

SEQ ID NO: 998 - RNA, 19 nt, Artificial Sequence
  Synthetic
cacaggaagaacaugucag

SEQ ID NO: 999 - RNA, 19 nt, Artificial Sequence
  Synthetic
acaggaagaacaugucagu

SEQ ID NO: 1000 - RNA, 19 nt, Artificial Sequence
  Synthetic
ggaagaacaugucagucuu

SEQ ID NO: 1001 - RNA, 19 nt, Artificial Sequence
  Synthetic
gaagaacaugucagucuug

SEQ ID NO: 1002 - RNA, 19 nt, Artificial Sequence
  Synthetic
aagaacaugucagucuugg

SEQ ID NO: 1003 - RNA, 19 nt, Artificial Sequence
  Synthetic
agaacaugucagucuuggu

SEQ ID NO: 1004 - RNA, 19 nt, Artificial Sequence
  Synthetic
ggcaucggaggaucccauu

SEQ ID NO: 1005 - RNA, 19 nt, Artificial Sequence
  Synthetic
acuauccaaaugcuggccu

SEQ ID NO: 1006 - RNA, 19 nt, Artificial Sequence
  Synthetic
gcacagaggcuccuucuga

SEQ ID NO: 1007 - RNA, 19 nt, Artificial Sequence
  Synthetic
uccuucugaacaagcacca

SEQ ID NO: 1008 - RNA, 19 nt, Artificial Sequence
  Synthetic
ccuucugaacaagcaccac

SEQ ID NO: 1009 - RNA, 19 nt, Artificial Sequence
  Synthetic
cuucugaacaagcaccacc

SEQ ID NO: 1010 - RNA, 19 nt, Artificial Sequence
  Synthetic
uucugaacaagcaccaccu

SEQ ID NO: 1011 - RNA, 19 nt, Artificial Sequence
  Synthetic
ucugaacaagcaccaccug

SEQ ID NO: 1012 - RNA, 19 nt, Artificial Sequence
  Synthetic
cugaacaagcaccaccuga

SEQ ID NO: 1013 - RNA, 19 nt, Artificial Sequence
  Synthetic
ugaacaagcaccaccugag

SEQ ID NO: 1014 - RNA, 19 nt, Artificial Sequence
  Synthetic
gaacaagcaccaccugaga

SEQ ID NO: 1015 - RNA, 19 nt, Artificial Sequence
  Synthetic
aacaagcaccaccugagaa

SEQ ID NO: 1016 - RNA, 19 nt, Artificial Sequence
  Synthetic
caagcaccaccugagaaaa

SEQ ID NO: 1017 - RNA, 19 nt, Artificial Sequence
  Synthetic
aagcaccaccugagaaaag

SEQ ID NO: 1018 - RNA, 19 nt, Artificial Sequence
  Synthetic
agcaccaccugagaaaagc

SEQ ID NO: 1019 - RNA, 19 nt, Artificial Sequence
  Synthetic
cugagaaaagcccuguggu

SEQ ID NO: 1020 - RNA, 19 nt, Artificial Sequence
  Synthetic
gcccugugguccaggauug

SEQ ID NO: 1021 - RNA, 19 nt, Artificial Sequence
  Synthetic
cugugguccaggauugcua

SEQ ID NO: 1022 - RNA, 19 nt, Artificial Sequence
  Synthetic
cuccaccacugucacagga

SEQ ID NO: 1023 - RNA, 19 nt, Artificial Sequence
  Synthetic
uccaccacugucacaggaa

SEQ ID NO: 1024 - RNA, 19 nt, Artificial Sequence
  Synthetic
ucuuggucaucuaugauac

SEQ ID NO: 1025 - RNA, 19 nt, Artificial Sequence
  Synthetic
cuuggucaucuaugauacc

SEQ ID NO: 1026 - RNA, 19 nt, Artificial Sequence
  Synthetic
uuggucaucuaugauacca

SEQ ID NO: 1027 - RNA, 19 nt, Artificial Sequence
  Synthetic
uggucaucuaugauaccac

SEQ ID NO: 1028 - RNA, 19 nt, Artificial Sequence
  Synthetic
ggucaucuaugauaccaca

SEQ ID NO: 1029 - RNA, 19 nt, Artificial Sequence
  Synthetic
gucaucuaugauaccacac

SEQ ID NO: 1030 - RNA, 19 nt, Artificial Sequence
  Synthetic
ucaucuaugauaccacacu

SEQ ID NO: 1031 - RNA, 19 nt, Artificial Sequence
  Synthetic
caucuaugauaccacacug

SEQ ID NO: 1032 - RNA, 19 nt, Artificial Sequence
  Synthetic
aucuaugauaccacacugg

SEQ ID NO: 1033 - RNA, 19 nt, Artificial Sequence
  Synthetic
ucuaugauaccacacuggc

SEQ ID NO: 1034 - RNA, 19 nt, Artificial Sequence
  Synthetic
cuaugauaccacacuggca

SEQ ID NO: 1035 - RNA, 19 nt, Artificial Sequence
  Synthetic
ugauaccacacuggcauca

SEQ ID NO: 1036 - RNA, 19 nt, Artificial Sequence
  Synthetic
auaccacacuggcaucaga

SEQ ID NO: 1037 - RNA, 19 nt, Artificial Sequence
  Synthetic
agaggaccccagaaaacua

SEQ ID NO: 1038 - RNA, 19 nt, Artificial Sequence
  Synthetic
gaggaccccagaaaacuac

SEQ ID NO: 1039 - RNA, 19 nt, Artificial Sequence
  Synthetic
agaacuacugcaggaaucc

SEQ ID NO: 1040 - RNA, 19 nt, Artificial Sequence
  Synthetic
acuacugcaggaauccaga

SEQ ID NO: 1041 - RNA, 19 nt, Artificial Sequence
  Synthetic
uacugcaggaauccagauu

SEQ ID NO: 1042 - RNA, 19 nt, Artificial Sequence
  Synthetic
acugcaggaauccagauuc

SEQ ID NO: 1043 - RNA, 19 nt, Artificial Sequence
  Synthetic
cugcaggaauccagauucu

SEQ ID NO: 1044 - RNA, 19 nt, Artificial Sequence
  Synthetic
ugcaggaauccagauucug

SEQ ID NO: 1045 - RNA, 19 nt, Artificial Sequence
  Synthetic
gcaggaauccagauucugg

SEQ ID NO: 1046 - RNA, 19 nt, Artificial Sequence
  Synthetic
ggaauccagauucugggaa

SEQ ID NO: 1047 - RNA, 19 nt, Artificial Sequence
  Synthetic
gggaaacaacccugguguu

SEQ ID NO: 1048 - RNA, 19 nt, Artificial Sequence
  Synthetic
ggaaacaacccugguguua

SEQ ID NO: 1049 - RNA, 19 nt, Artificial Sequence
  Synthetic
ugugugaggugggaguacu

SEQ ID NO: 1050 - RNA, 19 nt, Artificial Sequence
  Synthetic
gugugaggugggaguacug

SEQ ID NO: 1051 - RNA, 19 nt, Artificial Sequence
  Synthetic
gugaggugggaguacugca

SEQ ID NO: 1052 - RNA, 19 nt, Artificial Sequence
  Synthetic
ugaggugggaguacugcaa

SEQ ID NO: 1053 - RNA, 19 nt, Artificial Sequence
  Synthetic
cugacacaaugcucagaaa

SEQ ID NO: 1054 - RNA, 19 nt, Artificial Sequence
  Synthetic
ugacacaaugcucagaaac

SEQ ID NO: 1055 - RNA, 19 nt, Artificial Sequence
  Synthetic
gacacaaugcucagaaaca

SEQ ID NO: 1056 - RNA, 19 nt, Artificial Sequence
  Synthetic
acacaaugcucagaaacag

SEQ ID NO: 1057 - RNA, 19 nt, Artificial Sequence
  Synthetic
cacaaugcucagaaacaga

SEQ ID NO: 1058 - RNA, 19 nt, Artificial Sequence
  Synthetic
acaaugcucagaaacagaa

SEQ ID NO: 1059 - RNA, 19 nt, Artificial Sequence
  Synthetic
caaugcucagaaacagaau

SEQ ID NO: 1060 - RNA, 19 nt, Artificial Sequence
  Synthetic
aaugcucagaaacagaauc

SEQ ID NO: 1061 - RNA, 19 nt, Artificial Sequence
  Synthetic
augcucagaaacagaauca

SEQ ID NO: 1062 - RNA, 19 nt, Artificial Sequence
  Synthetic
ugcucagaaacagaaucag

SEQ ID NO: 1063 - RNA, 19 nt, Artificial Sequence
  Synthetic
cucagaaacagaaucaggu

SEQ ID NO: 1064 - RNA, 19 nt, Artificial Sequence
  Synthetic
cagaaacagaaucaggugu

SEQ ID NO: 1065 - RNA, 19 nt, Artificial Sequence
  Synthetic
agaaacagaaucagguguc

SEQ ID NO: 1066 - RNA, 19 nt, Artificial Sequence
  Synthetic
gaaacagaaucaggugucc

SEQ ID NO: 1067 - RNA, 19 nt, Artificial Sequence
  Synthetic
aaacagaaucagguguccu

SEQ ID NO: 1068 - RNA, 19 nt, Artificial Sequence
  Synthetic
aacagaaucagguguccua

SEQ ID NO: 1069 - RNA, 19 nt, Artificial Sequence
  Synthetic
cagaaucagguguccuaga

SEQ ID NO: 1070 - RNA, 19 nt, Artificial Sequence
  Synthetic
gaaucagguguccuagaga

SEQ ID NO: 1071 - RNA, 19 nt, Artificial Sequence
  Synthetic
aucagguguccuagagacu

SEQ ID NO: 1072 - RNA, 19 nt, Artificial Sequence
  Synthetic
gguguccuagagacuccca

SEQ ID NO: 1073 - RNA, 19 nt, Artificial Sequence
  Synthetic
ccuagagacucccacuguu

SEQ ID NO: 1074 - RNA, 19 nt, Artificial Sequence
  Synthetic
cuagagacucccacuguug

SEQ ID NO: 1075 - RNA, 19 nt, Artificial Sequence
  Synthetic
uagagacucccacuguugu

SEQ ID NO: 1076 - RNA, 19 nt, Artificial Sequence
  Synthetic
agagacucccacuguuguu

SEQ ID NO: 1077 - RNA, 19 nt, Artificial Sequence
  Synthetic
gagacucccacuguuguuc

SEQ ID NO: 1078 - RNA, 19 nt, Artificial Sequence
  Synthetic
agacucccacuguuguucc

SEQ ID NO: 1079 - RNA, 19 nt, Artificial Sequence
  Synthetic
gcucauucugaagcagcac

SEQ ID NO: 1080 - RNA, 19 nt, Artificial Sequence
  Synthetic
cucauucugaagcagcacc

SEQ ID NO: 1081 - RNA, 19 nt, Artificial Sequence
  Synthetic
ucauucugaagcagcacca

SEQ ID NO: 1082 - RNA, 19 nt, Artificial Sequence
  Synthetic
cauucugaagcagcaccaa

SEQ ID NO: 1083 - RNA, 19 nt, Artificial Sequence
  Synthetic
auucugaagcagcaccaac

SEQ ID NO: 1084 - RNA, 19 nt, Artificial Sequence
  Synthetic
uucugaagcagcaccaacu

SEQ ID NO: 1085 - RNA, 19 nt, Artificial Sequence
  Synthetic
ucugaagcagcaccaacug

SEQ ID NO: 1086 - RNA, 19 nt, Artificial Sequence
  Synthetic
cugaagcagcaccaacuga

SEQ ID NO: 1087 - RNA, 19 nt, Artificial Sequence
  Synthetic
ugaagcagcaccaacugag

SEQ ID NO: 1088 - RNA, 19 nt, Artificial Sequence
  Synthetic
gaagcagcaccaacugagc

SEQ ID NO: 1089 - RNA, 19 nt, Artificial Sequence
  Synthetic
aagcagcaccaacugagca

SEQ ID NO: 1090 - RNA, 19 nt, Artificial Sequence
  Synthetic
cagugcuaccaugguaaug

SEQ ID NO: 1091 - RNA, 19 nt, Artificial Sequence
  Synthetic
agugcuaccaugguaaugg

SEQ ID NO: 1092 - RNA, 19 nt, Artificial Sequence
  Synthetic
acauucuccaccacuguca

SEQ ID NO: 1093 - RNA, 19 nt, Artificial Sequence
  Synthetic
cacugucacaggaaggaca

SEQ ID NO: 1094 - RNA, 19 nt, Artificial Sequence
  Synthetic
acugucacaggaaggacau

SEQ ID NO: 1095 - RNA, 19 nt, Artificial Sequence
  Synthetic
cugucacaggaaggacaug

SEQ ID NO: 1096 - RNA, 19 nt, Artificial Sequence
  Synthetic
ugucacaggaaggacaugu

SEQ ID NO: 1097 - RNA, 19 nt, Artificial Sequence
  Synthetic
gucacaggaaggacauguc

SEQ ID NO: 1098 - RNA, 19 nt, Artificial Sequence
  Synthetic
ucacaggaaggacauguca

SEQ ID NO: 1099 - RNA, 19 nt, Artificial Sequence
  Synthetic
acaggaaggacaugucaau

SEQ ID NO: 1100 - RNA, 19 nt, Artificial Sequence
  Synthetic
caggaaggacaugucaauc

SEQ ID NO: 1101 - RNA, 19 nt, Artificial Sequence
  Synthetic
aggaaggacaugucaaucu

SEQ ID NO: 1102 - RNA, 19 nt, Artificial Sequence
  Synthetic
ggaaggacaugucaaucuu

SEQ ID NO: 1103 - RNA, 19 nt, Artificial Sequence
  Synthetic
gaaggacaugucaaucuug

SEQ ID NO: 1104 - RNA, 19 nt, Artificial Sequence
  Synthetic
aaggacaugucaaucuugg

SEQ ID NO: 1105 - RNA, 19 nt, Artificial Sequence
  Synthetic
aggacaugucaaucuuggu

SEQ ID NO: 1106 - RNA, 19 nt, Artificial Sequence
  Synthetic
ggacaugucaaucuugguc

SEQ ID NO: 1107 - RNA, 19 nt, Artificial Sequence
  Synthetic
gacaugucaaucuugguca

SEQ ID NO: 1108 - RNA, 19 nt, Artificial Sequence
  Synthetic
acaugucaaucuuggucau

SEQ ID NO: 1109 - RNA, 19 nt, Artificial Sequence
  Synthetic
caugucaaucuuggucauc

SEQ ID NO: 1110 - RNA, 19 nt, Artificial Sequence
  Synthetic
augucaaucuuggucaucc

SEQ ID NO: 1111 - RNA, 19 nt, Artificial Sequence
  Synthetic
ugucaaucuuggucaucca

SEQ ID NO: 1112 - RNA, 19 nt, Artificial Sequence
  Synthetic
gucaaucuuggucauccau

SEQ ID NO: 1113 - RNA, 19 nt, Artificial Sequence
  Synthetic
ucaaucuuggucauccaug

SEQ ID NO: 1114 - RNA, 19 nt, Artificial Sequence
  Synthetic
caaucuuggucauccauga

SEQ ID NO: 1115 - RNA, 19 nt, Artificial Sequence
  Synthetic
aaucuuggucauccaugac

SEQ ID NO: 1116 - RNA, 19 nt, Artificial Sequence
  Synthetic
aucuuggucauccaugaca

SEQ ID NO: 1117 - RNA, 19 nt, Artificial Sequence
  Synthetic
ugacaaugaacuacugcag

SEQ ID NO: 1118 - RNA, 19 nt, Artificial Sequence
  Synthetic
gacaaugaacuacugcagg

SEQ ID NO: 1119 - RNA, 19 nt, Artificial Sequence
  Synthetic
acaaugaacuacugcagga

SEQ ID NO: 1120 - RNA, 19 nt, Artificial Sequence
  Synthetic
caaugaacuacugcaggaa

SEQ ID NO: 1121 - RNA, 19 nt, Artificial Sequence
  Synthetic
aaugaacuacugcaggaau

SEQ ID NO: 1122 - RNA, 19 nt, Artificial Sequence
  Synthetic
augaacuacugcaggaauc

SEQ ID NO: 1123 - RNA, 19 nt, Artificial Sequence
  Synthetic
ugaacuacugcaggaaucc

SEQ ID NO: 1124 - RNA, 19 nt, Artificial Sequence
  Synthetic
cuacugcaggaauccagau

SEQ ID NO: 1125 - RNA, 19 nt, Artificial Sequence
  Synthetic
caggcccuugguguuuuac

SEQ ID NO: 1126 - RNA, 19 nt, Artificial Sequence
  Synthetic
cuugguguuuuaccaugga

SEQ ID NO: 1127 - RNA, 19 nt, Artificial Sequence
  Synthetic
uugguguuuuaccauggac

SEQ ID NO: 1128 - RNA, 19 nt, Artificial Sequence
  Synthetic
ugguguuuuaccauggacc

SEQ ID NO: 1129 - RNA, 19 nt, Artificial Sequence
  Synthetic
gguguuuuaccauggaccc

SEQ ID NO: 1130 - RNA, 19 nt, Artificial Sequence
  Synthetic
guguuuuaccauggacccc

SEQ ID NO: 1131 - RNA, 19 nt, Artificial Sequence
  Synthetic
guuuuaccauggaccccag

SEQ ID NO: 1132 - RNA, 19 nt, Artificial Sequence
  Synthetic
ggaguacugcaaccugacg

SEQ ID NO: 1133 - RNA, 19 nt, Artificial Sequence
  Synthetic
gaguacugcaaccugacgc

SEQ ID NO: 1134 - RNA, 19 nt, Artificial Sequence
  Synthetic
guacugcaaccugacgcga

SEQ ID NO: 1135 - RNA, 19 nt, Artificial Sequence
  Synthetic
uacugcaaccugacgcgau

SEQ ID NO: 1136 - RNA, 19 nt, Artificial Sequence
  Synthetic
ugcaaccugacgcgaugcu

SEQ ID NO: 1137 - RNA, 19 nt, Artificial Sequence
  Synthetic
ccugacgcgaugcucagac

SEQ ID NO: 1138 - RNA, 19 nt, Artificial Sequence
  Synthetic
cugacgcgaugcucagaca

SEQ ID NO: 1139 - RNA, 19 nt, Artificial Sequence
  Synthetic
gaugcucagacacagaagg

SEQ ID NO: 1140 - RNA, 19 nt, Artificial Sequence
  Synthetic
augcucagacacagaaggg

SEQ ID NO: 1141 - RNA, 19 nt, Artificial Sequence
  Synthetic
agacacagaagggacugug

SEQ ID NO: 1142 - RNA, 19 nt, Artificial Sequence
  Synthetic
gcauccucuucauuugauu

SEQ ID NO: 1143 - RNA, 19 nt, Artificial Sequence
  Synthetic
cauccucuucauuugauug

SEQ ID NO: 1144 - RNA, 19 nt, Artificial Sequence
  Synthetic
auccucuucauuugauugu

SEQ ID NO: 1145 - RNA, 19 nt, Artificial Sequence
  Synthetic
uccucuucauuugauugug

SEQ ID NO: 1146 - RNA, 19 nt, Artificial Sequence
  Synthetic
ccucuucauuugauugugg

SEQ ID NO: 1147 - RNA, 19 nt, Artificial Sequence
  Synthetic
cuucauuugauugugggaa

SEQ ID NO: 1148 - RNA, 19 nt, Artificial Sequence
  Synthetic
uucauuugauugugggaag

SEQ ID NO: 1149 - RNA, 19 nt, Artificial Sequence
  Synthetic
ucauuugauugugggaagc

SEQ ID NO: 1150 - RNA, 19 nt, Artificial Sequence
  Synthetic
cauuugauugugggaagcc

SEQ ID NO: 1151 - RNA, 19 nt, Artificial Sequence
  Synthetic
auuugauugugggaagccu

SEQ ID NO: 1152 - RNA, 19 nt, Artificial Sequence
  Synthetic
gugggaagccucaagugga

SEQ ID NO: 1153 - RNA, 19 nt, Artificial Sequence
  Synthetic
aagaaauguccuggaagca

SEQ ID NO: 1154 - RNA, 19 nt, Artificial Sequence
  Synthetic
agaaauguccuggaagcau

SEQ ID NO: 1155 - RNA, 19 nt, Artificial Sequence
  Synthetic
gaaauguccuggaagcauu

SEQ ID NO: 1156 - RNA, 19 nt, Artificial Sequence
  Synthetic
aauguccuggaagcauugu

SEQ ID NO: 1157 - RNA, 19 nt, Artificial Sequence
  Synthetic
auguccuggaagcauugua

SEQ ID NO: 1158 - RNA, 19 nt, Artificial Sequence
  Synthetic
agaacaagguuuggaaagc

SEQ ID NO: 1159 - RNA, 19 nt, Artificial Sequence
  Synthetic
gaacaagguuuggaaagca

SEQ ID NO: 1160 - RNA, 19 nt, Artificial Sequence
  Synthetic
aacaagguuuggaaagcac

SEQ ID NO: 1161 - RNA, 19 nt, Artificial Sequence
  Synthetic
acaagguuuggaaagcacu

SEQ ID NO: 1162 - RNA, 19 nt, Artificial Sequence
  Synthetic
caagguuuggaaagcacuu

SEQ ID NO: 1163 - RNA, 19 nt, Artificial Sequence
  Synthetic
aagguuuggaaagcacuuc

SEQ ID NO: 1164 - RNA, 19 nt, Artificial Sequence
  Synthetic
agguuuggaaagcacuucu

SEQ ID NO: 1165 - RNA, 19 nt, Artificial Sequence
  Synthetic
uggaaagcacuucugugga

SEQ ID NO: 1166 - RNA, 19 nt, Artificial Sequence
  Synthetic
guggaggcaccuuaauauc

SEQ ID NO: 1167 - RNA, 19 nt, Artificial Sequence
  Synthetic
cuuaauauccccagagugg

SEQ ID NO: 1168 - RNA, 19 nt, Artificial Sequence
  Synthetic
uaauauccccagagugggu

SEQ ID NO: 1169 - RNA, 19 nt, Artificial Sequence
  Synthetic
agagugggugcugacugcu

SEQ ID NO: 1170 - RNA, 19 nt, Artificial Sequence
  Synthetic
caaggucauccugggugca

SEQ ID NO: 1171 - RNA, 19 nt, Artificial Sequence
  Synthetic
ccugggugcacaccaagaa

SEQ ID NO: 1172 - RNA, 19 nt, Artificial Sequence
  Synthetic
gugcacaccaagaagugaa

SEQ ID NO: 1173 - RNA, 19 nt, Artificial Sequence
  Synthetic
agcagauauugccuugcua

SEQ ID NO: 1174 - RNA, 19 nt, Artificial Sequence
  Synthetic
gcagauauugccuugcuaa

SEQ ID NO: 1175 - RNA, 19 nt, Artificial Sequence
  Synthetic
cagauauugccuugcuaaa

SEQ ID NO: 1176 - RNA, 19 nt, Artificial Sequence
  Synthetic
agauauugccuugcuaaag

SEQ ID NO: 1177 - RNA, 19 nt, Artificial Sequence
  Synthetic
gauauugccuugcuaaagc

SEQ ID NO: 1178 - RNA, 19 nt, Artificial Sequence
  Synthetic
auauugccuugcuaaagcu

SEQ ID NO: 1179 - RNA, 19 nt, Artificial Sequence
  Synthetic
uauugccuugcuaaagcua

SEQ ID NO: 1180 - RNA, 19 nt, Artificial Sequence
  Synthetic
ucaucacugacaaaguaau

SEQ ID NO: 1181 - RNA, 19 nt, Artificial Sequence
  Synthetic
ggacugaauguuacaucac

SEQ ID NO: 1182 - RNA, 19 nt, Artificial Sequence
  Synthetic
gacugaauguuacaucacu

SEQ ID NO: 1183 - RNA, 19 nt, Artificial Sequence
  Synthetic
acugaauguuacaucacug

SEQ ID NO: 1184 - RNA, 19 nt, Artificial Sequence
  Synthetic
cugaauguuacaucacugg

SEQ ID NO: 1185 - RNA, 19 nt, Artificial Sequence
  Synthetic
ugaauguuacaucacuggc

SEQ ID NO: 1186 - RNA, 19 nt, Artificial Sequence
  Synthetic
aauguuacaucacuggcug

SEQ ID NO: 1187 - RNA, 19 nt, Artificial Sequence
  Synthetic
gaaacccaagguaccuuug

SEQ ID NO: 1188 - RNA, 36 nt, Artificial Sequence; identical to SEQ ID NO: 399
  Synthetic

SEQ ID NO: 1189 - RNA, 22 nt, Artificial Sequence
  Synthetic
uuggauaauauucuguugucgg

SEQ ID NO: 1190 - RNA, 19 nt, Artificial Sequence
  Synthetic
gacaacagaauauuaucca

SEQ ID NO: 1191 - RNA, 24 nt, Artificial Sequence
  Synthetic
cguuaaucgcguauaauacgcgua

SEQ ID NO: 1192 - RNA, 27 nt, Artificial Sequence
  Synthetic
auacgcguauuauacgcgauuaacgac

SEQ ID NO: 1193 - RNA, 25 nt, Artificial Sequence
  Synthetic
cauauugcgcguauagucgcguuag

SEQ ID NO: 1194 - RNA, 27 nt, Artificial Sequence
  Synthetic
cuaacgcgacuauacgcgcaauauggu

SEQ ID NO: 1195 - RNA, 24 nt, Artificial Sequence
  Synthetic
ggcgcguauagucgcgcguauagc

SEQ ID NO: 1196 - RNA, 27 nt, Artificial Sequence
  Synthetic
gacuauacgcgcgacuauacgcgccuc

SEQ ID NO: 1197 - RNA, 16 nt, Artificial Sequence
  Synthetic
gcagccgaaaggcugc